SEQUENCE LISTING (20 sequences)

SEQ ID NO: 1 - protein, 25 aa, Artificial Sequence
  N-terminally biotinylated NCoA3 coactivator peptide
ENQRGPLESKGHKKLLQLLTCSSDD

SEQ ID NO: 2 - protein, 306 aa, Artificial Sequence
  LXR -LBD protein sequence
EQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 3 - DNA, 882 bp, Artificial Sequence
  DNA-Sequence LXRalpha-LBD
cttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 4 - DNA, 1011 bp, Artificial Sequence
  DNA-sequence LXRbeta-LBD
gagcagtgcgtcctttctgaagaacagatccggaagaagaagattcggaaacaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctctgcttttatttaa

SEQ ID NO: 5 - DNA, 1011 bp, Artificial Sequence
  DNA-sequence SRC1-fragment
gttggcttctctgccagttctccagtcctcaggcagatgagctcacagaattcacctagcagattaaatatacaaccagcaaaagctgagtccaaagataacaaagagattgcctcaattttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagaacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgaccaaacccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctaaatagattacctgagctggaattggaagcaattgataaccaatttggacaaccaggaacaggcgattag

SEQ ID NO: 6 - DNA, 1225 bp, Artificial Sequence
  DNA-sequence NCoR-fragment
gataaagggcctcctccaaaatccagatatgaggaagagctaaggaccagagggaagactaccattactgcagctaacttcatagacgtgatcatcacccggcaaattgcctcggacaaggatgcgagggaacgtggctctcaaagttcagactcttctagtagcttatcttctcacaggtatgaaacacctagcgatgctattgaggtgataagtcctgccagctcacctgcgccaccccaggagaaactgcagacctatcagccagaggttgttaaggcaaatcaagcggaaaatgatcctaccagacaatatgaaggaccattacatcactatcgaccacagcaggaatcaccatctccccaacaacagctgcccccttcttcacaggcagagggaatggggcaagtgcccaggacccatcggctgatcacacttgctgatcacatctgtcaaattatcacacaagattttgctagaaatcaagtttcctcgcagactccccagcagcctcctacttctacattccagaactcaccttctgctttggtatctacacctgtgaggactaaaacatcaaaccgttacagcccagaatcccaggctcagtctgtccatcatcaaagaccaggttcaagggtctctacagaaaatcttgtggacaaatccaggggaagtaggcctggaaaatccccagagaggagtcacgtctcttcggagccctacgagcccatctccccaccccaggttccggttgtgcatgagaaacaggacagcttgctgctcttgtctcagaggggcgcagagcctgcagagcagaggaatgatgcccgctcaccagggagtataagctacttgccttcattcttcaccaagcttgaaaatacatcacccatggttaaatcaaagaagcaggagatttttcgtaagttgaactcctctggtggaggtgactctgatatggcagctgctcagccaggaactgagatctttaatctgccagcagttactacgtcaggctcagttagctctagaggccattcttttgctgatcctgccagtaatcttgggctggaagacattatcaggaaggctctcatgggaagctttgatgacaaagttgaggatcatggagttgtcatgtcccagcctatgggagtagtgcctggtactgccaacacctcagttgtgaccagtggtgagacacgaagagaggaaggggtga

SEQ ID NO: 7 - DNA, 1344 bp, Artificial Sequence
  LXRalpha - full length
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagtgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 8 - DNA, 1386 bp, Artificial Sequence
  LXRbeta - full length
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacaacagcagcaggagtcacagtcacagtcgcagtcacctgtggggccgcagggcagcagcagctcagcctctgggcctggggcttcccctggtggatctgaggcaggcagccagggctccggggaaggcgagggcgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 9 - DNA, 20 bp, Artificial Sequence
  Gene Fasn - forward primer
cccctctgttaattggctcc

SEQ ID NO: 10 - DNA, 20 bp, Artificial Sequence
  Gene Fasn - reverse primer
ttgtggaagtgcaggttagg

SEQ ID NO: 11 - DNA, 22 bp, Artificial Sequence
  Gene Fasn - sequence probe
caggctcagggtgtcccatgtt

SEQ ID NO: 12 - DNA, 20 bp, Artificial Sequence
  Gene Scd1 - forward primer
ctgacctgaaagccgagaag

SEQ ID NO: 13 - DNA, 20 bp, Artificial Sequence
  Gene Scd1 - reverse primer
agaaggtgctaacgaacagg

SEQ ID NO: 14 - DNA, 25 bp, Artificial Sequence
  Gene Scd1 - sequence probe
tgtttacaaaagtctcgccccagca

SEQ ID NO: 15 - DNA, 20 bp, Artificial Sequence
  Gene Srebp1c - forward primer
ccatcgactacatccgcttc

SEQ ID NO: 16 - DNA, 20 bp, Artificial Sequence
  Gene Srebp1c - reverse primer
gccctccatagacacatctg

SEQ ID NO: 17 - DNA, 24 bp, Artificial Sequence
  Gene Srebp1c - sequence probe
tctcctgcttgagcttctggttgc

SEQ ID NO: 18 - DNA, 21 bp, Artificial Sequence
  Gene Tpb - forward primer
caccaatgactcctatgaccc

SEQ ID NO: 19 - DNA, 23 bp, Artificial Sequence
  Gene Tpb - reverse primer
caagtttacagccaagattcacg

SEQ ID NO: 20 - DNA, 20 bp, Artificial Sequence
  Gene Tpb - sequence probe
actcctgccacaccagcctc

(19) United States
(12) Patent Application Publication
(10) Pub. No.: US 2020/0115357 A1
(43) Pub. Date: Apr. 16, 2020

(54) LIVER X RECEPTORS (LXR) MODULATORS

(71) Applicant: Phenex-FXR GmbH, Heidelberg (DE)

(72) Inventors: Christian Gege, Ehingen (DE); Manfred Birkel, Seeheim-Jugenheim (DE); Eva Hambruch, Mannheim (DE); Ulrich Deuschle, Speyer (DE); Claus Kremoser, Heidelberg (DE)

(73) Assignee: Phenex-FXR GmbH, Heidelberg (DE)

(21) Appl. No.: 16/603,870

(22) PCT Filed: Apr. 10, 2018
(86) PCT No.: PCT/EP2018/000188

§ 371 Date: Oct. 9, 2019

(30) Foreign Application Priority Data
Apr. 10, 2017 (EP) 17000610.0

(51) Int. Cl. C07D 307/52; A61P 1/16; C07C 311/19; C07D 405/12; C07D 407/12

(52) U.S. Cl. CPC C07D 307/52; A61P 1/16; C07D 407/12; C07D 405/12; C07C 311/19

(57) ABSTRACT

The present invention relates to sulfonamide-, sulfinamide- or sulfonimidamide containing compounds which bind to the liver X receptor (LXRa and/or LXRβ) and act preferably as inverse agonists of LXR.

The present invention relates to novel compounds which are Liver X Receptor modulators and pharmaceutical composition containing same. The present invention further relates to the use of said compounds in the prophylaxis and/or treatment of diseases which are associated with the modulation of the Liver X Receptor.

### BACKGROUND

The Liver X Receptors, LXRα (NR1H3) and LXRβ (NR1H2) are members of the nuclear receptor protein superfamily. Both receptors form heterodimeric complexes with Retinoid X Receptor (RXRα, β or γ) and bind to LXR response elements (e.g. DR4-type elements) located in the promoter regions of LXR responsive genes. Both receptors are transcription factors that are physiologically regulated by binding ligands such as oxysterols or intermediates of the cholesterol biosynthetic pathways, such as desmosterol. In the absence of a ligand, the LXR-RXR heterodimer is believed to remain bound to the DR4-type element in complex with co-repressors, such as NCOR1, resulting in repression of the corresponding target genes. Upon binding of an agonist ligand, either an endogenous one such as the oxysterols or steroid intermediates mentioned before or a synthetic, pharmacological ligand, the conformation of the heterodimeric complex is changed, leading to the release of corepressor proteins and to the recruitment of coactivator proteins such as NCOA1 (SRC1), resulting in transcriptional stimulation of the respective target genes. While LXRβ is expressed in most tissues, LXRα is expressed more selectively in cells of the liver, the intestine, adipose tissue and macrophages. The relative expression of LXRα and LXRβ at the mRNA or the protein level may vary between different tissues in the same species or between different species in a given tissue. The LXR's control reverse cholesterol transport, i.e. the mobilization of tissue-bound peripheral cholesterol into HDL and from there into bile and feces, through the transcriptional control of target genes such as ABCA1 and ABCG1 in macrophages and ABCG5 and ABCG8 in liver and intestine. This explains the anti-atherogenic activity of LXR agonists in dietary LDLR-KO mouse models. The LXRs, however, do also control the transcription of genes involved in lipogenesis (e.g. SREBF1, SCD, FASN, ACACA) which accounts for the liver steatosis observed following prolonged treatment with LXR agonists. The liver steatosis liability is considered a main barrier for the development of non-selective LXR agonists for atherosclerosis treatment.

Non-alcoholic fatty liver disease (NAFLD) is regarded as a manifestation of metabolic syndrome in the liver and NAFLD has reached epidemic prevalence worldwide (Marchesini et al., Curr. Opin. Lipidol. 2005; 16:421). The pathologies of NAFLD range from benign and reversible steatosis to steatohepatitis (nonalcoholic steatohepatitis, NASH) that can develop towards fibrosis, cirrhosis and potentially further towards hepatocellular carcinogenesis. Classically, a two-step model has been employed to describe the progression of NAFLD into NASH, with hepatic steatosis as an initiating first step sensitizing towards secondary signals (exogenous or endogenous) that lead to inflammation and hepatic damage (Day et al., Gastroenterology 1998; 114:842).

Notably, LXR expression was shown to correlate with the degree of fat deposition, as well as with hepatic inflammation and fibrosis in NAFLD patients (Ahn et al., Dig. Dis. Sci. 2014; 59:2975). Furthermore, serum and liver desmosterol levels are increased in patients with NASH but not in people with simple liver steatosis. Desmosterol has been characterized as a potent endogenous LXR agonist (Yang et al., J. Biol. Chem. 2006; 281:27816). NAFLD/NASH patients might therefore benefit from blocking the increased LXR activity observed in the livers of these patients through small molecule antagonists or inverse agonists that shut off LXRs' activity. While doing so it needs to be taken care that such LXR antagonists or inverse agonists do not interfere with LXRs in peripheral tissues or macrophages to avoid disruption of the anti-atherosclerotic reverse cholesterol transport governed by LXR in these tissues or cells.

Certain publications (e.g. Peet et al., Cell 1998; 93:693 and Schultz et al., Genes Dev. 2000; 14:2831) have highlighted the role of LXRα, in particular, for the stimulation of lipogenesis and hence establishment of NAFLD in the liver. They indicate that it is mainly LXRα being responsible for the hepatic steatosis, hence an LXRα-specific antagonist or inverse agonist might suffice or be desirable to treat just hepatic steatosis. These data, however, were generated only by comparing LXRα, LXRβ or double knockout with wild-type mice with regards to their susceptibility to develop steatosis on a high fat diet. They do not account for a major difference in the relative expression levels of LXRα and LXRβ in the human as opposed to the murine liver. Whereas LXRα is the predominant LXR subtype in the rodent liver, LXRβ is expressed to about the same if not higher levels in the human liver compared to LXRα. This was exemplified by testing an LXRβ selective agonist in human phase I clinical studies (Kirchgessner et al., Cell Metab. 2016; 24:223) which resulted in the induction of strong hepatic steatosis although it was shown to not activate human LXRα.

Hence it can be assumed that it should be desirable to have no strong preference of an LXR modulator designed to treat NAFLD or NASH for a particular LXR subtype. A certain degree of LXR subtype selectivity might be allowed if the pharmacokinetic profile of such a compound clearly ensures sufficient liver exposure and resident time to cover both LXRs in clinical use.

In summary, the treatment of diseases such as NAFLD or NASH would need LXR modulators that block LXRs in a hepato-selective fashion and this could be achieved through hepatotropic pharmacokinetic and tissue distribution properties that have to be built into such LXR modulators.

### PRIOR ART

WO2009/040289 describes novel biaryl sulfonamides of formula (A) as LXR agonists

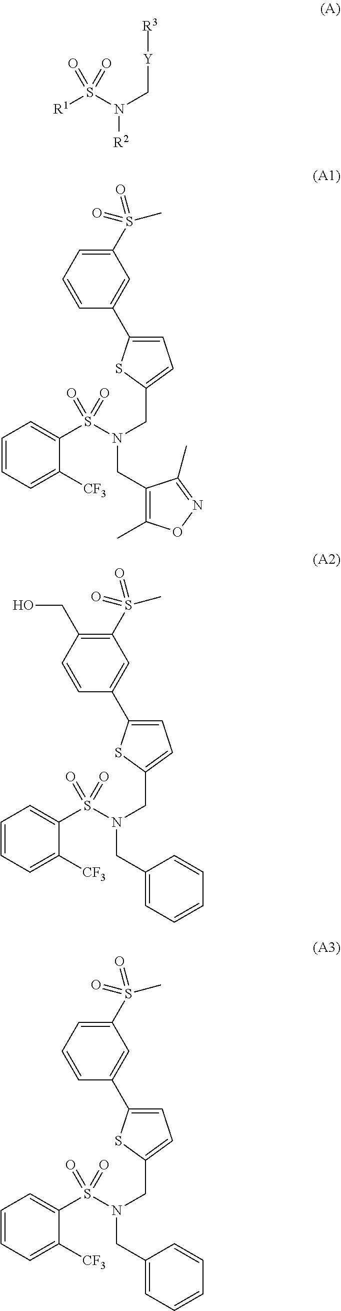

wherein,

Y is selected from (hetero)aryl; optionally substituted with 1 to 4 substituents selected from halogen, (fluoro)alkyl or O-(fluoro)alkyl;

R¹ is selected from (fluoro)alkyl, (hetero)aryl, (hetero)aryl-alkyl, cycloalkyl, cycloalkyl-alkyl; wherein (hetero)aryl and cycloalkyl is optionally substituted with 1 to 4 substituents selected from halogen, CN, (fluoro)alkyl, O-(fluoro)alkyl, alkyl-O—CO or phenyl;

R² is selected from alkyl, alkyl-O-alkyl, alkyl-O—CO-alkyl, NH₂CO-alkyl, cycloalkyl, (hetero)cycloalkyl-alkyl, (hetero)aryl-alkyl or (hetero)aryl-CO, wherein (hetero)aryl and (hetero)cycloalkyl is optionally substituted with 1 to 4 substituents selected from halogen, CN, (fluoro)alkyl, O-(fluoro)alkyl and alkyl-O—CO;

R³ is (hetero)aryl, which is substituted with alkyl-SO₂—, NR₂—SO₂—, alkyl-SO₂—NR— or NR₂—SO₂—NR— and wherein (hetero)aryl is optionally substituted with 1 to 3 substituents selected from halogen, CN, HO-alkyl-, (fluoro)alkyl, O-(fluoro)alkyl and alkyl-O—CO; and

R is selected from H and alkyl.

Remarkably, nearly all examples have a MeSO₂-group as required R³ substituent. Closest examples towards the claims from this application are (A1) to (A3).

Zuercher et al. describes with the tertiary sulfonamide GSK2033 the first potent, cell-active LXR antagonists (J. Med. Chem. 2010; 53:3412). Later, this compound was reported to display a significant degree of promiscuity, targeting a number of other nuclear receptors (Griffett and Burris, Biochem. Biophys. Res. Commun. 2016; 479:424). All potent examples have a MeSO₂-group and also the SO₂-group of the sulfonamide seems necessary for potency. It is stated, that GSK2033 showed rapid clearance (Clᵢₙₜ>1.0 mL/min/mg prot) in rat and human liver microsome assays and that this rapid hepatic metabolism of GSK2033 precludes its use in vivo. As such GSK2033 is an useful chemical probe for LXR in cellular studies only.

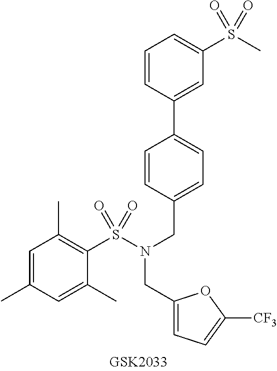

WO2014/085453 describes the preparation of small molecule LXR inverse agonists of structure (B) in addition to structure GSK2033 above,

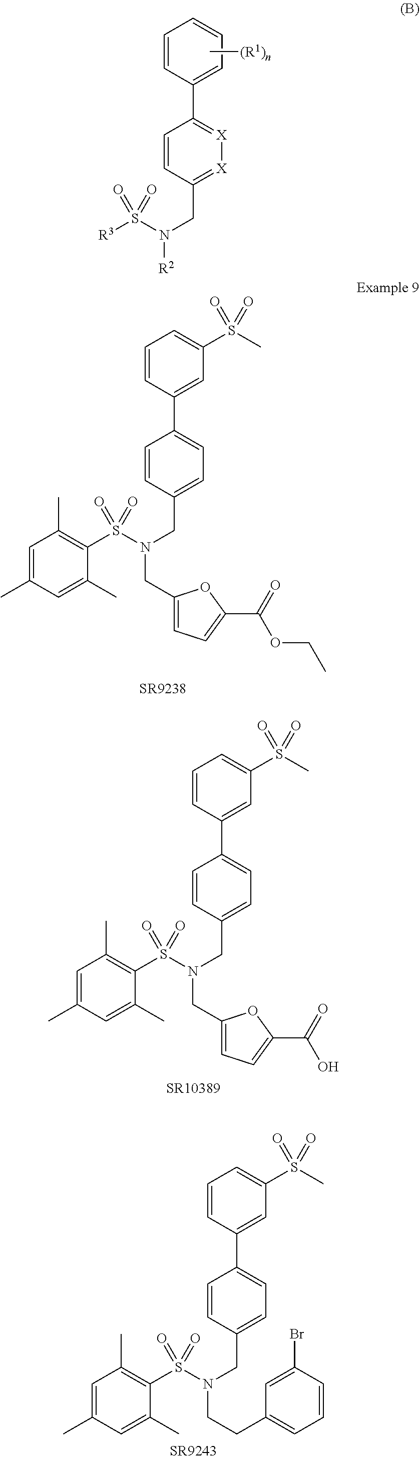

wherein

R¹ is selected from the group consisting of (halo)alkyl, cycloalkyl, (halo)alkoxy, halo, CN, NO₂, OR, SO_{q}R, CO₂R, CONR₂, OCONR₂, NRCONR₂, —SO₂alkyl, —SO₂NR-alkyl, —SO₂-aryl, —SO₂NR-aryl, heterocyclyl, heterocyclyl-alkyl or N- and C-bonded tetrazoyl;

R is selected from H, (halo)alkyl, cycloalkyl, cycloalkyl-alkyl, (hetero)aryl, (hetero)aryl-alkyl, heterocyclyl or heterocyclyl-alkyl;

n is selected from 1 to 3 and q is selected from 0 is 2;

X is selected from N or OH;

R² is selected from alkyl, alkenyl, alkynyl, cycloalkyl, alkyl-(═O)O-alkyl, aryl-alkyl-C(═O)O-alkyl, aryl-alkyl-O—C(═O)-alkyl, (hetero)aryl, (hetero)aryl-alkyl, heterocyclyl or heterocyclyl-alkyl, wherein all R² residues are substituted with 0 to 3 J-groups;

R³ is selected from alkyl, (hetero)aryl or (hetero)aryl-alkyl, wherein all R³ residues are substituted with 0 to 3 J-groups; and

J is selected from (halo)alkyl, cycloalkyl, heterocyclyl, (hetero)aryl, haloalkyoxy, halo, CN, NO₂, OR, SO_{q}R, CO₂R, CONR₂, O—CO₂R, OCONR₂, NRCONR₂ or NRCO₂R.

The following compounds from this application, in particular, are further described in some publications from the same group of inventors/authors: SR9238 is described as a liver-selective LXR inverse agonist that suppresses hepatic steatosis upon parenteral administration (Griffett et al., ACS Chem. Biol. 2013; 8:559). After ester saponification of SR9238 the LXR inactive acid derivative SR10389 is formed. This compound then has systemic exposure. In addition, it was described, that SR9238 suppresses fibrosis in a model of NASH again after parenteral administration (Griffett et al., Mol. Metab. 2015; 4:35). With a related SR9243 the effects on aerobic glycolysis (Warburg effect) and lipogenesis were described (Flaveny et al., Cancer Cell 2015; 28:42).

Remarkably, all these derivatives have a methyl sulfone group in the biphenyl portion and the SAR shown in WO2014/085453 suggests, that a replacement or orientation of the MeSO₂-group by other moieties (e.g. —CN, —CONH₂, N-linked tetrazoyl) is inferior for LXR potency. For all compounds shown, no oral bioavailability was reported.

As shown in the experimental section, we confirmed that neutral sulfonamide GSK2033 and SR9238 are not orally bioavailable and hepatoselective. In addition, when the ester in SR9238 is cleaved, the formed acid SR10389 is inactive on LXR.

WO2002/055484 describes the preparation of small molecules of structure (C), which can be used to increase the amount of low-density lipoprotein (LDL) receptor and are useful as blood lipid depressants for the treatment of hyperlipidemia, atherosclerosis or diabetes mellitus. In all examples, an acidic function can be found in the para-position of the diaryl moiety. Closest examples are (C1) and (C2).

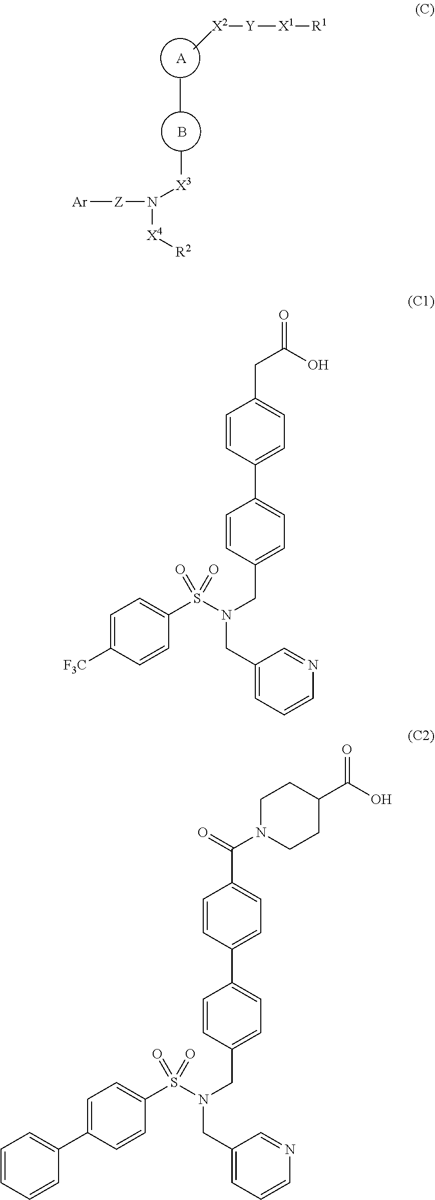

Claimed are structures of Formula (C), wherein

A and B represents independently an optionally substituted 5- or 6-membered aromatic ring;

R¹, R² and R³ is independently selected from H, an optionally substituted hydrocarbon group or an optionally substituted heterocycle;

X¹, X², X³ and X⁴ is independently selected from a bond or an optionally substituted divalent hydrocarbon group;

Y is selected from —NR³CO—, —CONR³—, —NR³—, —SO₂—, —SO₂R³— or —R³—CH₂—;

Z is selected from —CONH—, —CSNH—, —CO— or —SO₂—; and

Ar is selected from an optionally substituted cyclic hydrocarbon group or an optionally substituted heterocycle.

WO2006/009876 describes compounds of Formula (D) for modulating the activity of protein tyrosine phosphatases,

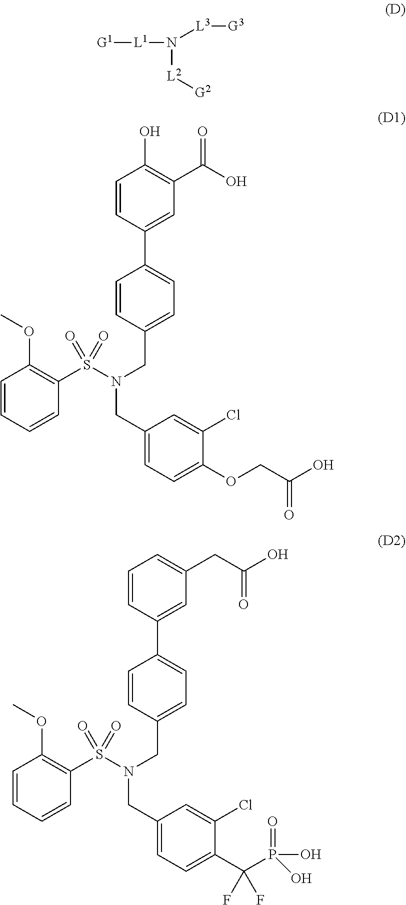

wherein

L¹, L², L³ is independently selected from a bond or an optionally substituted group selected from alkylene, alkenylene, alkynylene, cycloalkylene, oxocycloalkylene, amidocycloalkylene, heterocyclylene, heteroarylene, C═O, sulfonyl, alkylsulfonyl, alkenylsulfonyl, alkynylsulfonyl, amide, carboxamido, alkylamide, alkylcarboxamido and alkoxyoxo;

G¹, G², G³ is independently selected from alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl and sulfonyl; wherein each residue is optionally substituted with 1 to 3 substituents selected from H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, amido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl and phosphonatohaloalkyl.

From the huge range of possible substituents compound (D1) and (D2) are closest to the scope of the present invention. All shown examples have an acidic moiety in the non-biaryl part of the molecule.

Although numerous LXR modulators are disclosed to date, there is still a need to deliver improved LXR modulators, especially LXR inverse agonists with defined hepatoselectivity.

It is therefore the object of the present invention to provide improved LXR modulators with a defined hepatoselectivity.

### SUMMARY OF THE INVENTION

The present invention relates to compounds according to Formula (I)

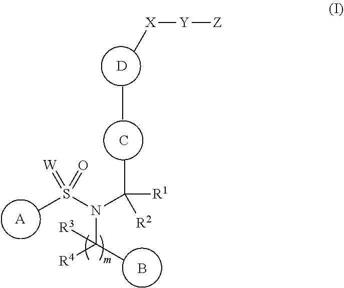

an enantiomer, diastereomer, tautomer, N-oxide, solvate, prodrug and pharmaceutically acceptable salt thereof,

wherein A, B, C, D, W, X, Y, Z, R¹ to R⁴ and m are defined as in claim 1.

We surprisingly found, that potent, orally bioavailable LXR modulators with hepatoselective properties can be obtained, when a carboxylic acid or a carboxylic acid isoster (see e.g. Ballatore et al., ChemMedChem 2013; 8:385, Lassalas et al., J. Med. Chem. 2016; 59:3183) is tethered covalently to the methylsulfon moiety of (GSK2033) or the methylsulfon moiety of (GSK2033) is replaced by another carboxylic acid- or carboxylic acid isoster-containing moiety. The compounds of the present invention have a similar or better LXR inverse agonistic, antagonistic or agonistic activity compared to the known LXR-modulators without an acidic moiety. Furthermore, the compounds of the present invention exhibit an advantageous liver/blood-ratio after oral administration so that disruption of the anti-atherosclerotic reverse cholesterol transport governed by LXR in peripheral macrophages can be avoided. The incorporation of an acidic moiety (or a bioisoster thereof) can improve additional parameters, e.g. microsomal stability, solubility and lipophilicity, in a beneficial way, in addition.

Thus, the present invention further relates to a pharmaceutical composition comprising a compound according to Formula (I) and at least one pharmaceutically acceptable carrier or excipient.

The present invention is further directed to compounds according to Formula (I) for use in the prophylaxis and/or treatment of diseases mediated by LXRs.

Accordingly, the present invention relates to the prophylaxis and/or treatment of non-alcoholic fatty liver disease, non-alcoholic steatohepatitis, obesity, insulin resistance, type II diabetes, metabolic syndrome, cancer, viral myocarditis and hepatitis C virus infection.

### DETAILED DESCRIPTION OF THE INVENTION

The desired properties of an LXR modulator in conjunction with hepatoselectivity, can be yielded with compounds that follow the structural pattern represented by Formula (I)

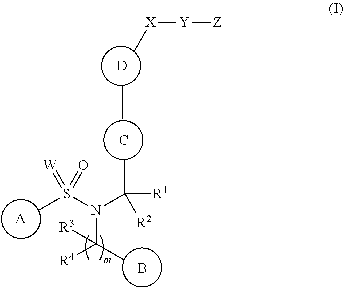

an enantiomer, diastereomer, tautomer, N-oxide, solvate, prodrug and pharmaceutically acceptable salt thereof,

wherein

R¹, R² are independently selected from H and C₁₋₄-alkyl,
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;

or R¹ and R² together are oxo, a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;

or R¹ and an adjacent residue from ring C form a saturated or partially saturated 5- to 8-membered cycloalkyl or a 5- to 8-membered heterocycloalkyl containing 1 to 4 heteroatoms independently selected from N, O and S,
wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;

R³, R⁴ are independently selected from H, C₁₋₄-alkyl and halo-C₁₋₄-alkyl;
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl, O-halo-C₁₋₄-alkyl;

or R³ and R⁴ together are oxo, a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;
or R³ and an adjacent residue from ring B form a partially saturated 5- to 8-membered cycloalkyl or a 5- to 8-membered heterocycloalkyl containing 1 to 4 heteroatoms independently selected from N, O and S,
wherein the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;

\textcircled{A} is selected from the group consisting of 3- to 10-membered cycloalkyl, 3- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, NO₂, OXO, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

\textcircled{B} is selected from the group consisting of 6- or 10-membered aryl and 5- to 10-membered heteroaryl containing 1 to 4 heteroatoms independently selected from N, O and S,
wherein aryl and heteroaryl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkylene-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

\textcircled{C} is selected from the group consisting of 3- to 10-membered cycloalkyl, 3- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, NO₂, OXO, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially saturated cycle optionally containing 1 to 3 heteroatoms independently selected from O, S or N, wherein this additional cycle is optionally substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

\textcircled{D} is selected from the group consisting of 3- to 10-membered cycloalkyl, 3- to 10-membered heterocycloalkyl containing 1 to 4 heteroatoms independently selected from N, O and S, 6- or 10-membered aryl and 5- to 10-membered heteroaryl containing 1 to 4 heteratoms independently selected from N, O and S,
wherein cycloalkyl, heterocycloalkyl, aryl and heteroaryl are unsubstituted or substituted with 1 to 4 substituents independently selected from the group consisting of halogen, CN, NO₂, oxo, CO₁₄-alkyl, C₀₋₆-alkylene-OR⁸¹, C₀₋₆-alkylene-(3- to 6-membered cycloalkyl), C₀₋₆-alkylene-(3- to 6-membered heterocycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹ and C₀₋₆-alkylene-NR⁸¹R⁸²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

W is selected from O, NR¹¹ or absent;

the residue X—Y—Z on ring D is linked in 1,3-orientation regarding the connection towards ring C;

X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—;

Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S,
wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;

Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═O)H(OH), —B(OH)₂;

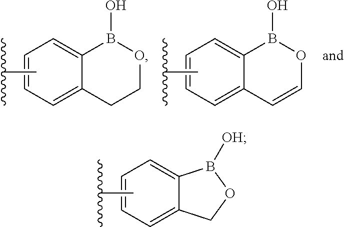

or X—Y—Z is selected from —SO₃H and —SO₂NHCOR⁹⁰;

or when X is not a bond then Z in addition can be selected from —CONR⁹¹R⁹², —S(═O)₂NR⁹¹R⁹²,

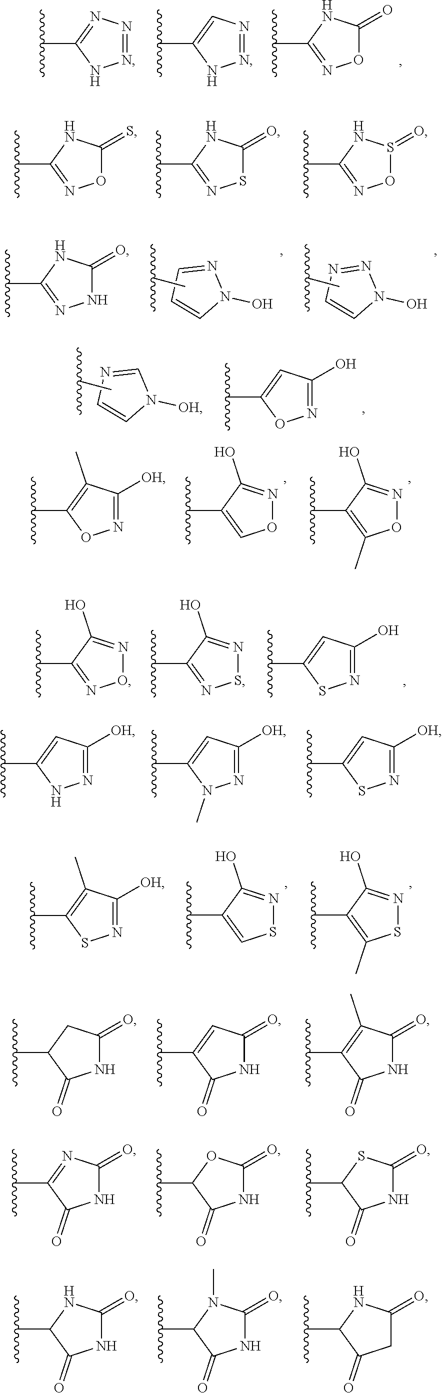

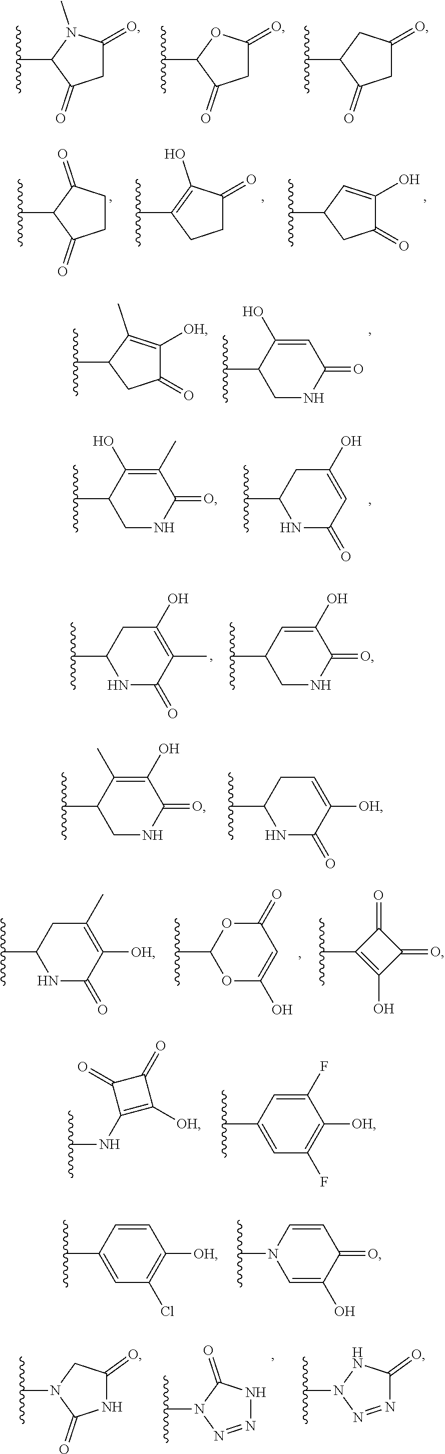

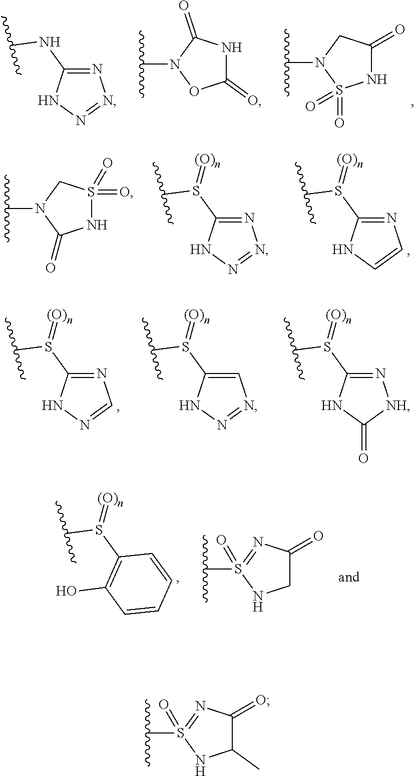

R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl and C(═O)—O-halo-C₁₋₄-alkyl;

R⁵¹, R⁵², R⁶¹, R⁶², R⁷¹, R⁷², R⁸¹, R⁸² are independently selected from H and C₁₋₄-alkyl,
wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
or R⁵¹ and R⁵², R⁶¹ and R⁶², R⁷¹ and R⁷², R⁸¹ and R⁸², respectively, when taken together with the nitrogen to which they are attached complete a 3- to 6-membered ring containing carbon atoms and optionally containing 1 or 2 heteroatoms independently selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;

R⁹⁰ is independently selected from C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;

R⁹¹, R⁹² are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;

or R⁹¹ and R⁹² when taken together with the nitrogen to which they are attached complete a 3-to 6-membered ring containing carbon atoms and optionally containing 1 or 2 heteroatoms selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;

n and m are independently selected from 0 to 2.

In a preferred embodiment in combination with any of the above or below embodiments R¹ and R² are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;

or R¹ and R² together are oxo, a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, and O-halo-C₁₋₄-alkyl;

or R¹ and an adjacent residue from ring C form a saturated or partially saturated 5- to 8-membered cycloalkyl or a 5- to 8-membered heterocycloalkyl containing 1 to 4 heteroatoms independently selected from N, O and S, the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl.

In a more preferred embodiment in combination with any of the above or below embodiments, R¹ and R² are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl.

In a most preferred embodiment in combination with any of the above and below embodiments, R¹ and R² are independently selected from H or Me.

In a preferred embodiment in combination with any of the above or below embodiments, R³ and R⁴ are independently selected from H and C₁₋₄-alkyl; wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl, O-halo-C₁₋₄-alkyl;

or R³ and R⁴ together are oxo, a 3- to 6-membered cycloalkyl or a 3- to 6-membered heterocycloalkyl, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, and O-halo-C₁₋₄-alkyl;

or R³ and an adjacent residue from ring B form a partially saturated 5- to 8-membered cycloalkyl or a 5- to 8-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl.

More preferably, in combination with any of the above and below embodiments, R³ and R⁴ are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl.

In a most preferred embodiment in combination with any of the above and below embodiments, R³ and R⁴ are independently selected from H or Me.

In a preferred embodiment in combination with any of the above or below embodiments W is selected from O, NR¹¹ or absent; more preferably W is O.

In a preferred embodiment in combination with any of the above or below embodiments m is selected from 0 to 2, more preferably m is 1 or 2. In a most preferred embodiment in combination with any of the above and below embodiments, m is 1.

In another preferred embodiment in combination with any of the above or below embodiments, R¹, R², R³ and R⁴ are independently selected from H or Me, and m is 1.

In another preferred embodiment in combination with any of the above or below embodiments, R¹, R², R³ and R⁴ are independently selected from H or Me, W is O and m is 1.

In a preferred embodiment in combination with any of the above or below embodiments R¹¹ is selected from H, CN, NO₂, Me, Et, C(═O)-Me, C(═O)-Et, C(═O)—O—CMe₃.

In a more preferred embodiment in combination with any of the above or below embodiments R¹¹ is H.

In a further preferred embodiment in combination with any of the above or below embodiments \textcircled{A} is selected from the group consisting of 3- to 10-membered cycloalkyl, 3- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S, wherein cycloalkyl, heterocycloalkyl, aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, CO₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹, C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially saturated cycle optionally containing 1 to 3 heteroatoms independently selected from O, S or N, wherein this additional cycle is optionally substituted with 1 to 4 substituents independently selected from halogen, CN, oxo, OH, C₁₋₄-alkyl, halo-C₁₋₄-alkyl, O—C₁₋₄-alkyl and O-halo-C₁₋₄₄-alkyl.

In a preferred embodiment in combination with any of the above and below embodiments, \textcircled{A} is selected from the group consisting of 6- or 10-membered aryl and 5- to 10-membered heteroaryl containing 1 to 4 heteroatoms independently selected from N, O and S, wherein aryl and heteroaryl are unsubstituted or substituted with 1 to 6 substituents independently selected from the group consisting of halogen, CN, NO₂, oxo, CO₁₋₄-alkyl, C₀₋₆-alkylene-OR⁵¹, C₀₋₆-alkylene-(3- to 6-membered cycloalkyl), C₀₋₆-alkylene-(3- to 6-membered heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, CO₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹, C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In a more preferred embodiment in combination with any of the above and below embodiments, \textcircled{A} is selected from the group consisting of 6- or 10-membered aryl and 5- to 10-membered heteroaryl containing 1 to 4 heteroatoms independently selected from N, O and S, wherein 6-membered aryl and 5- to 6-membered heteroaryl are substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; and wherein optionally two adjacent substituents in the aryl or heteroaryl moiety form a 5- to 6-membered partially saturated cycle optionally containing 1 to 3 heteroatoms independently selected from O, S or N, wherein this additional cycle is unsubstituted or substituted with 1 to 4 substituents independently selected from fluoro, CN, oxo, OH, Me, CF₃, CHF₂, OMe, OCF₃ and OCHF₂; or wherein

10-membered aryl and 8- to 10-membered heteroaryl are unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl.

In an even more preferred embodiment in combination with any of the above and below embodiments, \textcircled{A} is selected from the group consisting of phenyl, pyridyl, pyrimidinyl, naphthyl, benzo[b]thiophene, quinolinyl, isoquinolinyl, pyrazolo[1,5-a]pyrimidinyl and 1,5-naphthyridinyl wherein phenyl, pyridyl and pyrimidinyl are substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; and wherein optionally two adjacent substituents in the aryl or heteroaryl moiety form a 5- to 6-membered partially saturated cycle optionally containing 1 to 3 heteroatoms independently selected from O, S or N, wherein this additional cycle is unsubstituted or substituted with 1 to 4 substituents independently selected from fluoro, CN, oxo, OH, Me, CF₃, CHF₂, OMe, OCF₃ and OCHF₂; or wherein

naphthyl, benzo[b]thiophene, quinolinyl, isoquinolinyl, pyrazolo[1,5-a]pyrimidinyl and 1,5-naphthyridinyl are unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl.

In an even more preferred embodiment in combination with any of the above and below embodiments, is selected from the group consisting of phenyl, naphthyl and quinolinyl, wherein phenyl is substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; or wherein naphthyl or quinolinyl is unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl.

In an even more preferred embodiment in combination with any of the above and below embodiments, \textcircled{A} is selected from

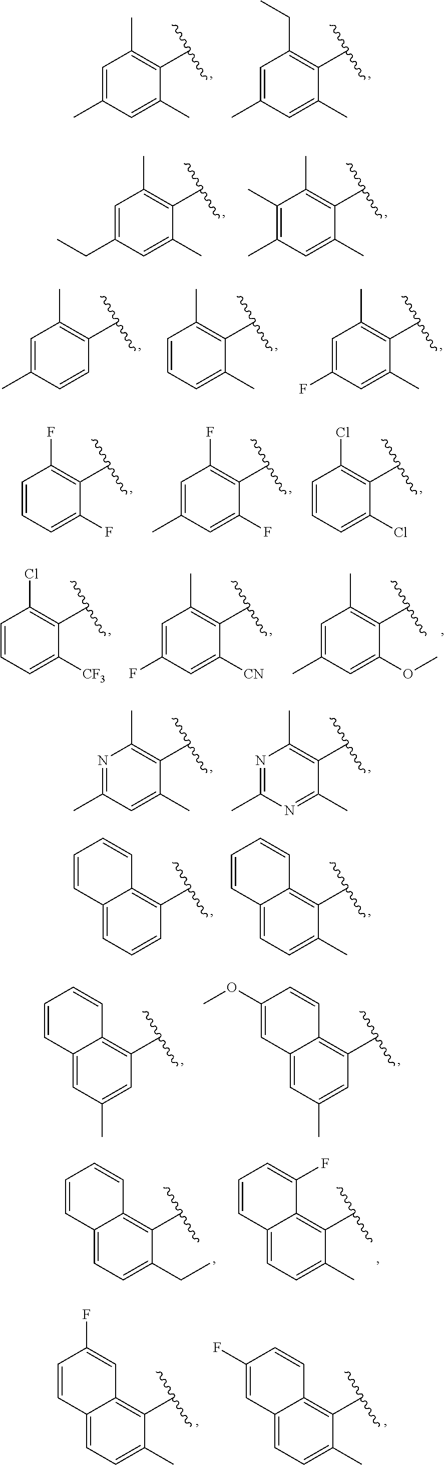

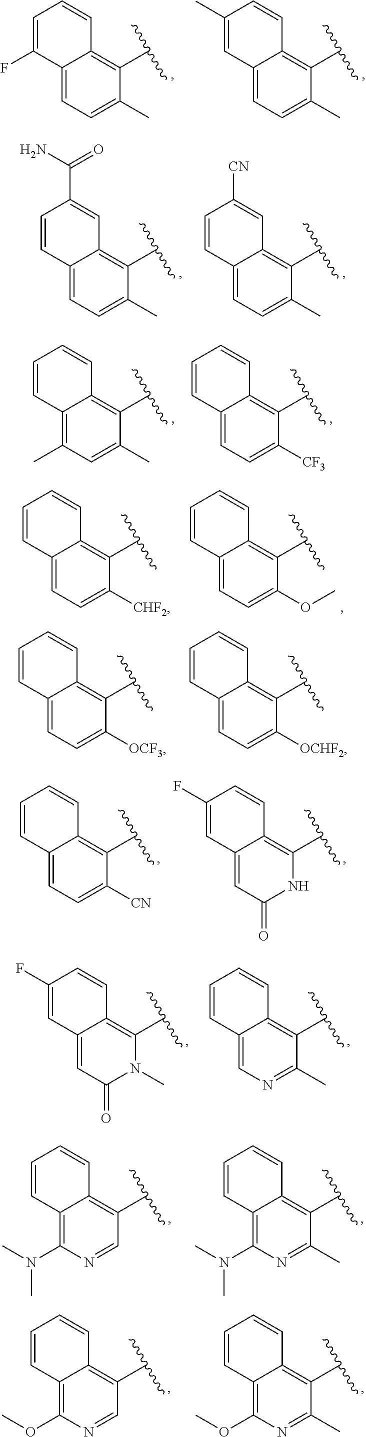

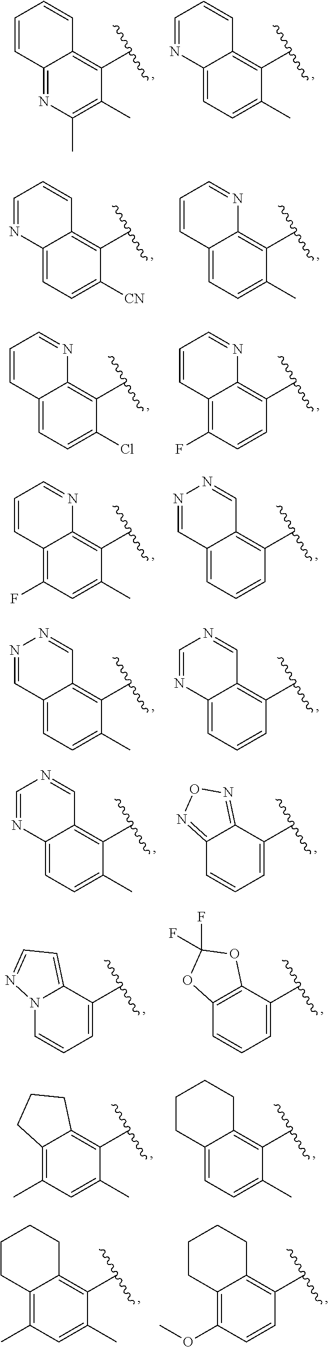

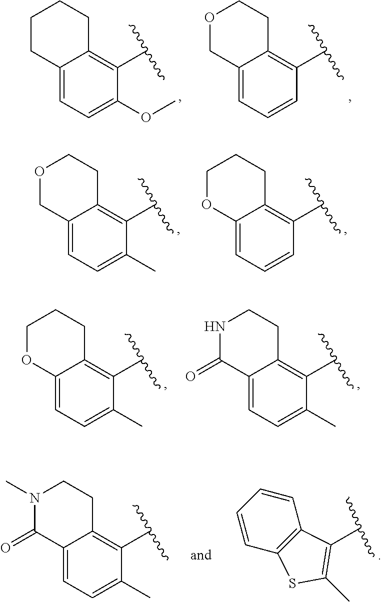

Even more preferred, \textcircled{A} is selected from

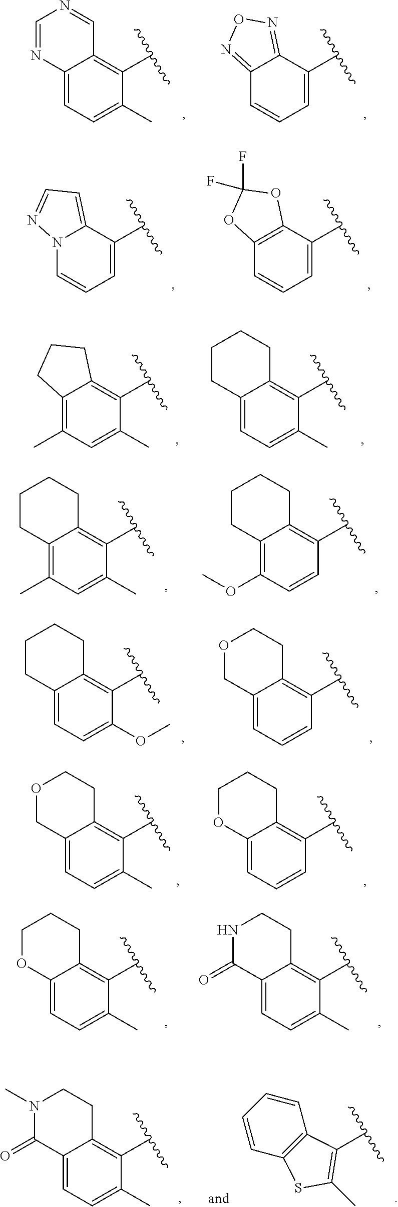

In a most preferred embodiment in combination with any of the above and below embodiments, \textcircled{A} is selected from

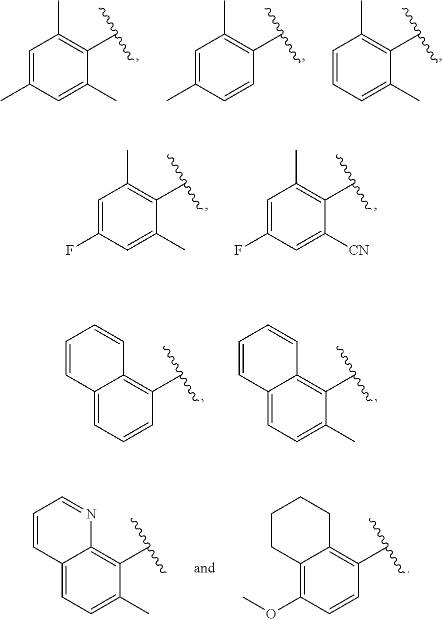

In a further preferred embodiment in combination with any of the above or below embodiments \textcircled{B} is selected from the group consisting of 6- or 10-membered aryl and 5- to 10-membered heteroaryl, wherein aryl and heteroaryl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkylene-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In a more preferred embodiment in combination with any of the above and below embodiments, \textcircled{B} is selected from the group consisting of phenyl, pyridinyl, pyrrolyl, thiazolyl, thiofuranyl or furanyl, wherein phenyl, pyridinyl, pyrrolyl, thiazolyl, thiofuranyl or furanyl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkylene-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹, C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the phenyl, pyridinyl, pyrrolyl, thiazolyl, thiofuranyl or furanyl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In an even more preferred embodiment in combination with any of the above and below embodiments, \textcircled{B} is selected from the group consisting of phenyl, pyridinyl, pyrrolyl, thiazolyl, thiofuranyl or furanyl, wherein phenyl, pyridinyl, pyrrolyl, thiazolyl, thiofuranyl or furanyl are substituted with 1 to 2 substituents independently selected from the group consisting of fluoro, chloro, bromo, CN, C₁₋₄-alkyl, —O—C₁₋₄-alkyl, fluoro-C₁₋₄-alkyl, —O-fluoro-C₁₋₄-alkyl, CONH₂, CONH(C₁₋₄-alkyl), CONH(fluoro-C₁₋₄-alkyl) and CON(C₁₋₄-alkyl)₂.

In an even more preferred embodiment in combination with any of the above and below embodiments, \textcircled{B} is selected from

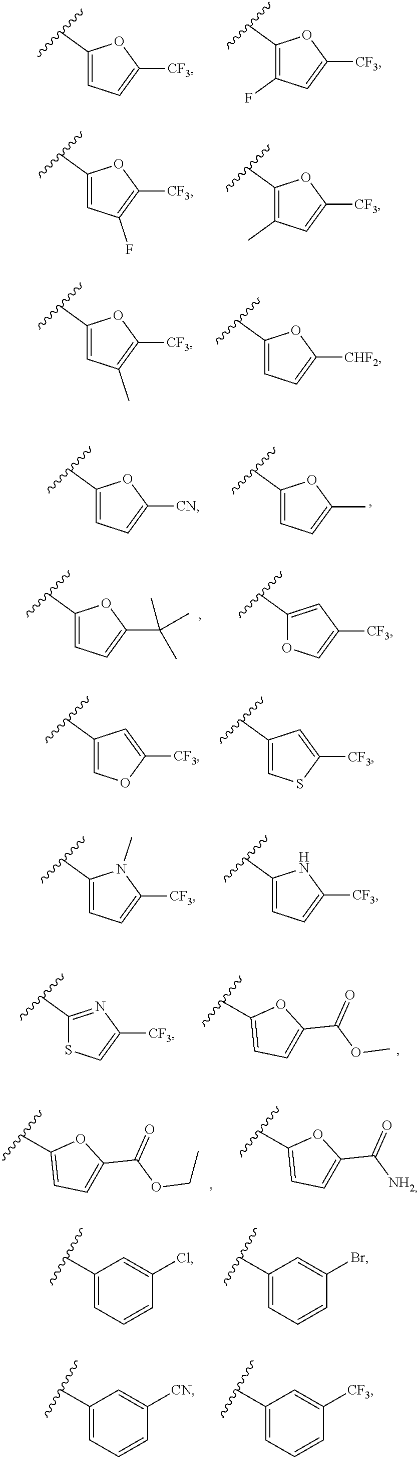

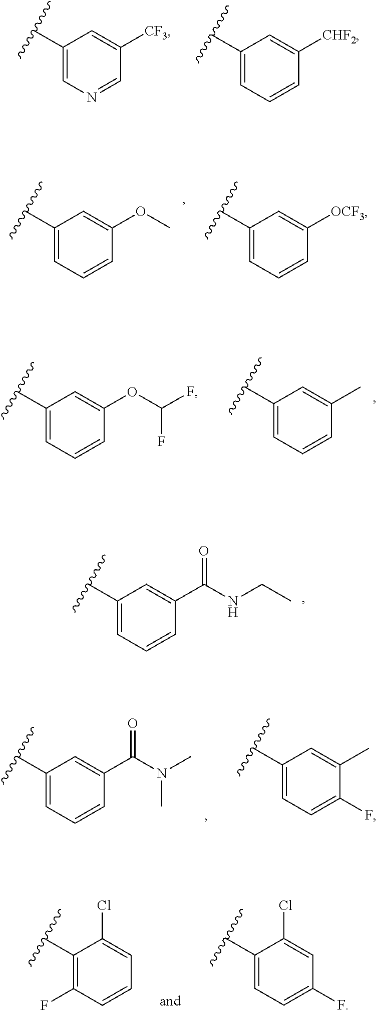

In an even more preferred embodiment in combination with any of the above and below embodiments, \textcircled{B} is selected from

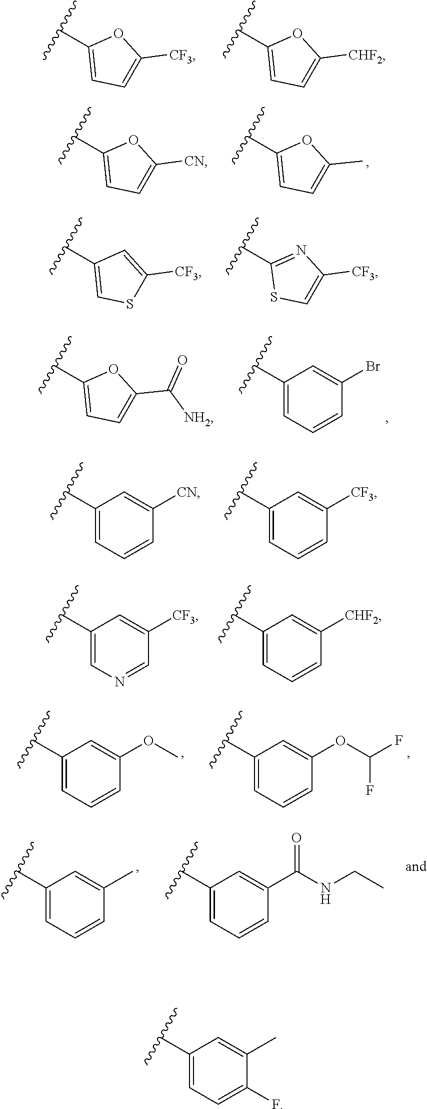

In a more preferred embodiment in combination with any of the above and below embodiments, \textcircled{B} is selected from

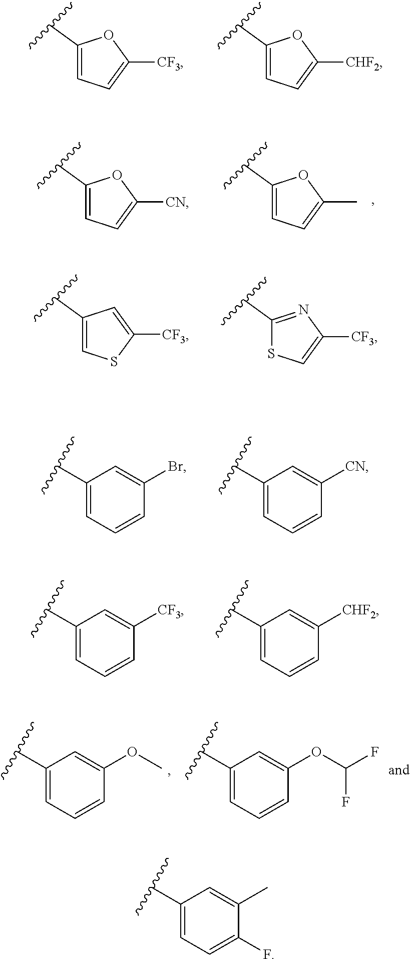

In most preferred embodiment in combination with any of the above and below embodiments, \textcircled{B} is

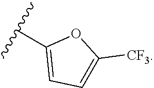 [SMILES: *c1ccc(C(F)(F)F)o1]

In a further preferred embodiment in combination with any of the above or below embodiments \textcircled{C} is selected from the group consisting of 3- to 6-membered cycloalkyl, 3- to 6-membered heterocycloalkyl, 6- or 10-membered aryl and 5- to 10-membered heteroaryl containing 1 to 4 heteroatoms independently selected from N, O and S, wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₁₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In preferred embodiment in combination with any of the above and below embodiments, \textcircled{C} is selected from the group consisting of phenyl, thiophenyl, thiazolyl and pyridinyl, wherein phenyl, thiophenyl, thiazolyl and pyridinyl are unsubstituted or substituted 1 to 4 substituents independently selected from the group consisting of halogen, CN, NO₂, oxo, C₁₋₄-alkyl, C₁₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl.

In a more preferred embodiment in combination with any of the above and below embodiments, \textcircled{C} is selected from the group consisting of phenyl, thiophenyl, thiazolyl and pyridinyl, wherein phenyl, thiophenyl, thiazolyl and pyridinyl are unsubstituted or substituted with 1 to 2 substituents independently selected from the group consisting of fluoro, chloro, CN, C₁₋₄-alkyl, —OC₁₋₄-alkyl, fluoro-C₁₋₄-alkyl and —O-fluoro-C₁₋₄-alkyl.

In an even more preferred embodiment in combination with any of the above and below embodiments,

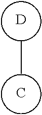

is selected from

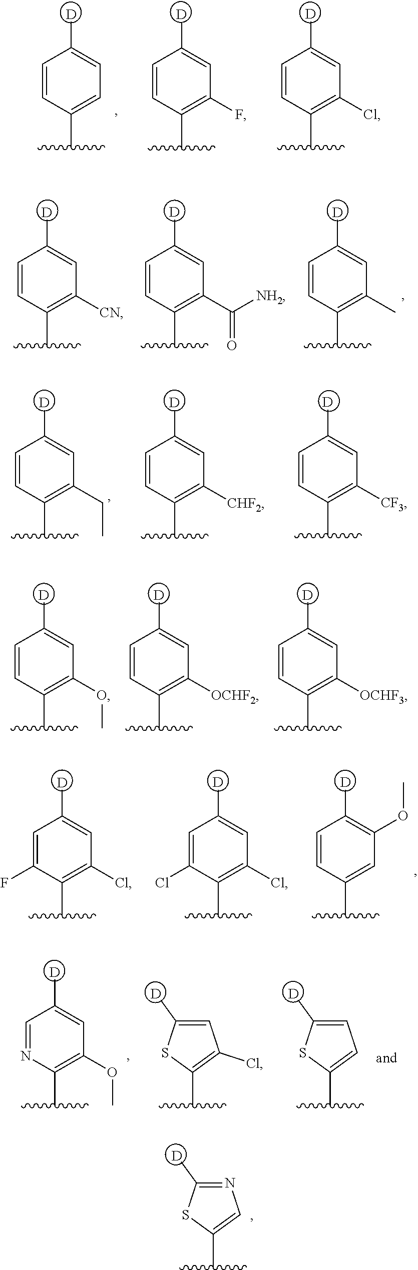

In an even more preferred embodiment in combination with any of the above and below embodiments,

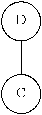

is selected from

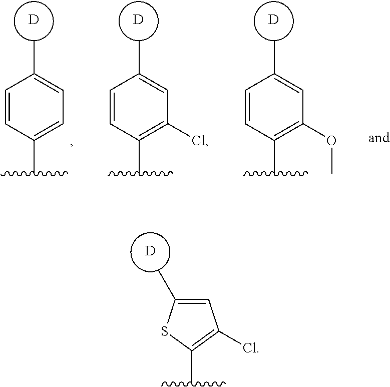

In a most preferred embodiment in combination with any of the above and below embodiments,

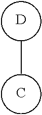

is selected from

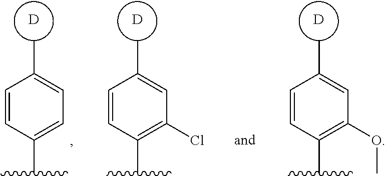

In a further preferred embodiment in combination with any of the above or below embodiments,

\textcircled{D} is selected from the group consisting of 3- to 6-membered cycloalkyl, 3- to 6-membered heterocycloalkyl, 6- or 10-membered aryl and 5- to 10-membered heteroaryl, wherein cycloalkyl, heterocycloalkyl, aryl and heteroaryl are unsubstituted or substituted with 1 to 4 substituents independently selected from the group consisting of halogen, CN, NO₂, C₁₋₄-alkyl, C₀₋₆-alkylene-OR⁸¹, C₀₋₆-alkylene-(3- to 6-membered cycloalkyl), C₀₋₆-alkylene-(3- to 6-membered heterocycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², oxo, C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹, C₀₋₆-alkylene-NR⁸¹R⁸², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In an even more preferred embodiment in combination with any of the above and below embodiments, \textcircled{D} is selected from the group consisting of phenyl, pyridinyl, thiophenyl or thiazolyl, wherein phenyl, pyridinyl, thiophenyl or thiazolyl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-(3- to 6-membered heterocycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², oxo, C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹, C₀₋₆-alkylene-NR⁸¹R⁸², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl.

In an even more preferred embodiment in combination with any of the above and below embodiments, \textcircled{D} is selected from the group consisting of phenyl, pyridinyl, thiophenyl or thiazolyl wherein phenyl, pyridinyl, thiophenyl or thiazolyl are unsubstituted or substituted with 1 to 2 substituents independently selected from the group consisting of fluoro, chloro, CN, OH, C₁₋₄-alkyl, —OC₁₋₄-alkyl, fluoro-C₁₋₄-alkyl, —O-fluoro-C₁₋₄-alkyl and C₁₋₃-alkylene-OH.

In an even more preferred embodiment in combination with any of the above and below embodiments, \textcircled{D} is selected from the group consisting of phenyl or pyridinyl, wherein phenyl or pyridinyl are unsubstituted or substituted with 1 to 2 substituents independently selected from the group consisting of fluoro, chloro, CN, OH, C₁₋₄-alkyl, —OC₁₋₄-alkyl, fluoro-C₁₋₄-alkyl, —O-fluoro-C₁₋₄-alkyl and C₁₋₃-alkylene-OH.

In an even more preferred embodiment in combination with any of the above and below embodiments,

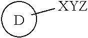

is selected from

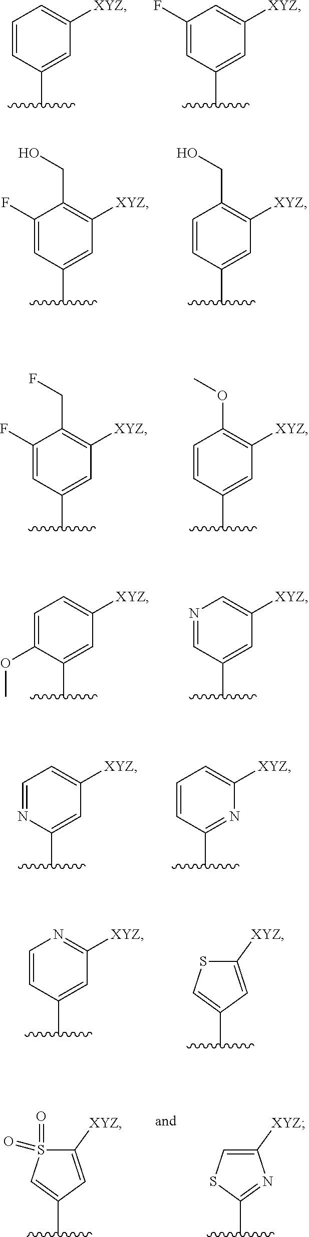

In an even more preferred embodiment in combination with any of the above and below embodiments,

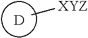

is selected from

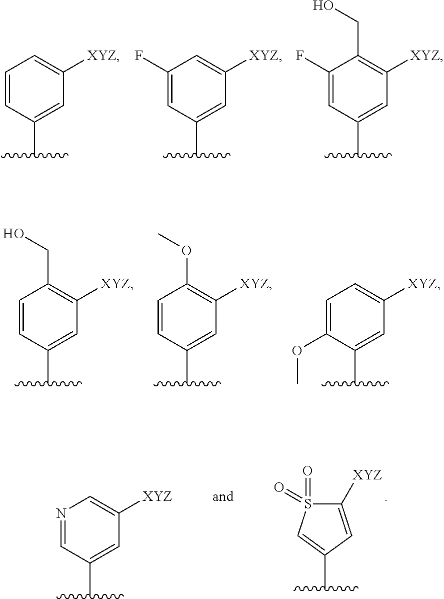

In a most preferred embodiment in combination with any of the above and below embodiments,

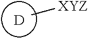

is selected from:

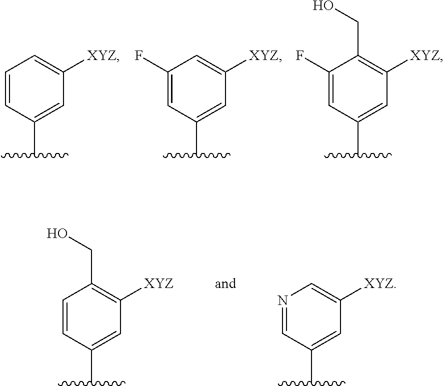

In a further preferred embodiment in combination with any of the above or below embodiments the residue X—Y—Z on ring D is linked in 1,3-orientation regarding the connection towards ring C;

X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹—, C₀₋₆-alkylene-S(═NR¹¹)—NR⁹¹—;
Y is selected from C₁₋₆-alkylene, C₂₋₆-alkenylene, C₂₋₆-alkinylene, 3- to 6-membered cycloalkylene, 3- to 6-membered heterocycloalkylene, wherein alkylene, alkenylene, alkinylene, cycloalkylene or heterocycloalkylene is unsubstituted or substituted with 1 to 6 substituent independently selected from halogen, CN, C₁₋₄-alkyl, halo-C₁₋₄-alkyl, C₃₋₆-cycloalkyl, halo-C₃₋₆-cycloalkyl, C₃₋₆-heterocycloalkyl, halo-C₃₋₆-heterocycloalkyl, OH, oxo, O—C₁₋₄-alkyl, O-halo-C₁₋₄-alkyl;

Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═O)H(OH), —B(OH)₂;

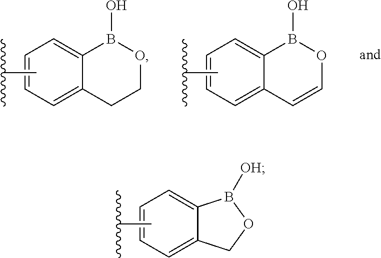

or X—Y—Z is selected from —SO₃H and —SO₂NHCOR⁹⁰;

or when X is not a bond then Z in addition can be selected from —CONR⁹¹R⁹², —S(═O)₂NR⁹¹R⁹²,

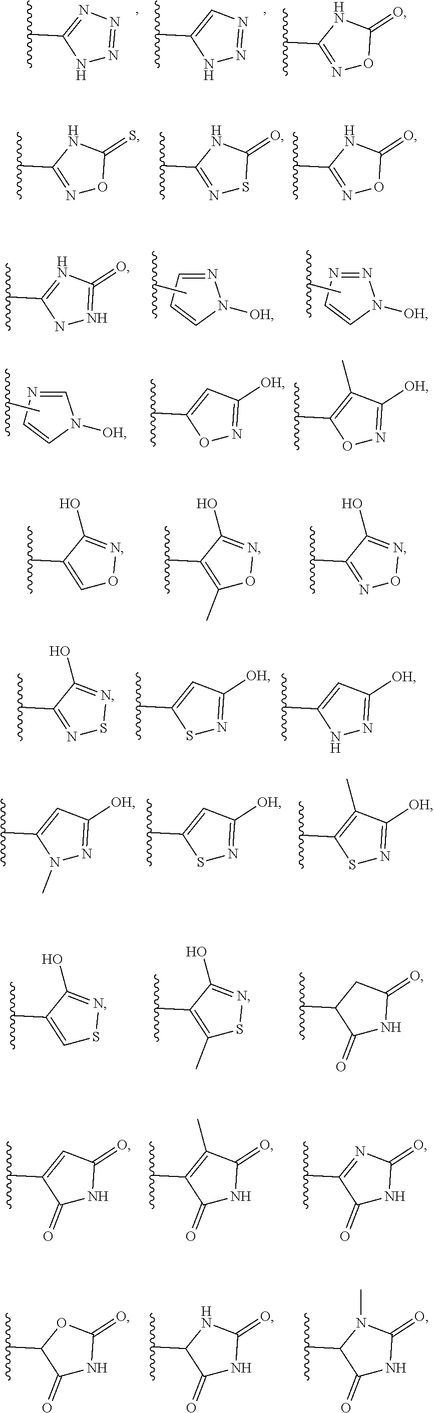

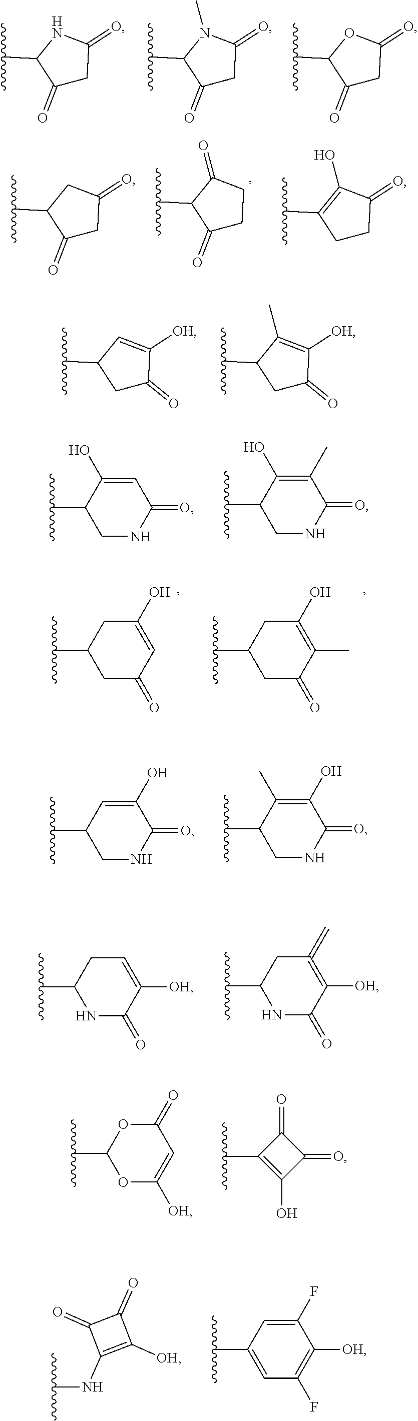

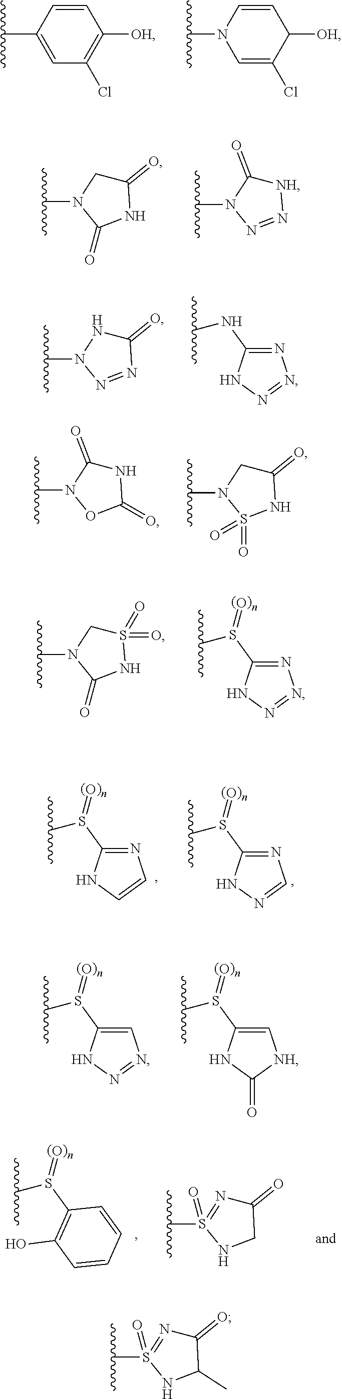

R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl or C(═O)—O-halo-C₁₋₄-alkyl;

R⁹⁰ is independently selected from C₁₋₄-alkyl and halo-C₁₋₄-alkyl, wherein alkyl is unsubstituted or substituted with 1 to 3 substituent independently selected from halogen, CN, C₁₋₄-alkyl, halo-C₁₋₄-alkyl, 3- to 6-membered-cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;

R⁹¹, R⁹² are independently selected from H and C₁₋₄-alkyl, wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;

R⁹¹ and R⁹² when taken together with the nitrogen to which they are attached complete a 3- to 6-membered ring containing carbon atoms and optionally containing 1 or 2 heteroatoms selected from O, S or N; and wherein the new formed cycle is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;

n is selected from 0 to 2.

In a more preferred embodiment in combination with any of the above and below embodiments, XYZ is selected from

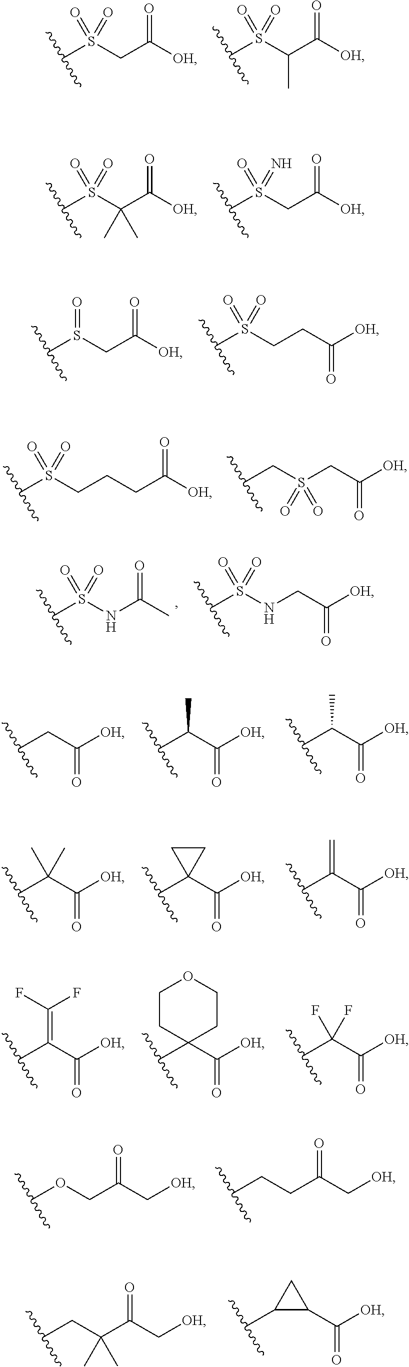

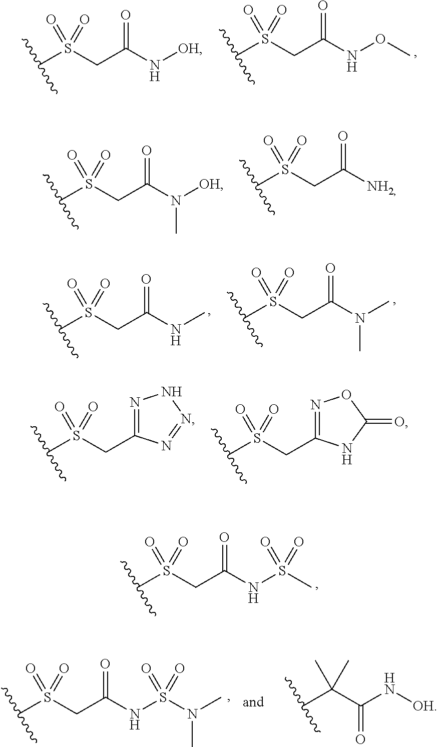

In a more preferred embodiment in combination with any of the above and below embodiments,

X is selected from a bond, O, S(═O) and S(═O)₂;

Y is selected from C₁₋₃-alkylene, 3- to 6-membered cycloalkylene and 3- to 6-membered heterocycloalkylene, wherein alkylene, cycloalkylene or heterocycloalkylene is unsubstituted or substituted with 1 to 2 substituent independently selected from fluoro, CN, C₁₋₄-alkyl, halo-C₁₋₄-alkyl, OH, oxo, O—C₁₋₄-alkyl and O-halo-C₁₋₄-alkyl;

Z is selected from —CO₂H and —CONHOH.

In another preferred embodiment in combination with any of the above and below embodiments

X is selected from a bond, S, S(═O) and S(═O)₂;

Y is selected from C₁₋₃-alkylene or C₃-cycloalkylene, wherein alkylene or cycloalkylene is unsubstituted or substituted with 1 to 2 substituent independently selected from halo or C₁₋₄-alkyl; and

Z is —CO₂H or an ester or pharmaceutically acceptable salt thereof.

In an even more preferred embodiment in combination with any of the above and below embodiments, XYZ is selected from

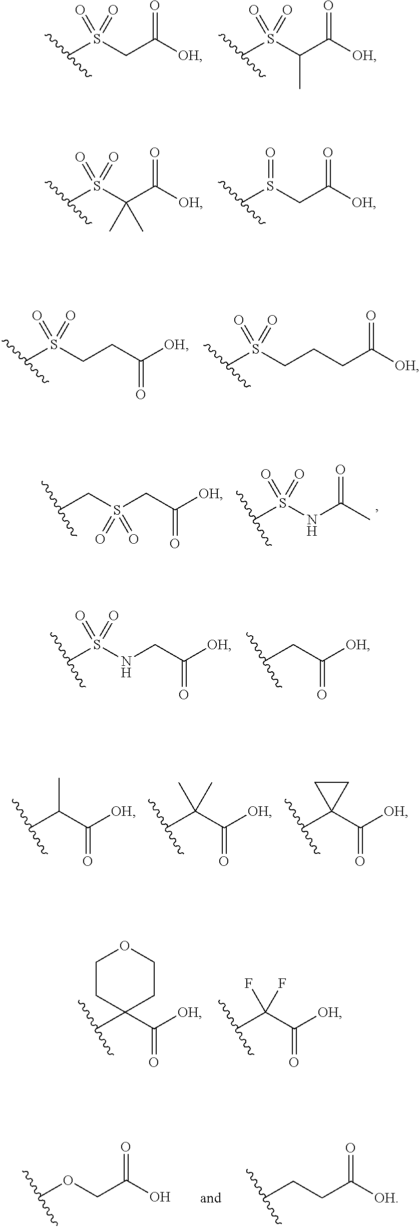

In a more preferred embodiment in combination with any of the above and below embodiments, XYZ is selected from

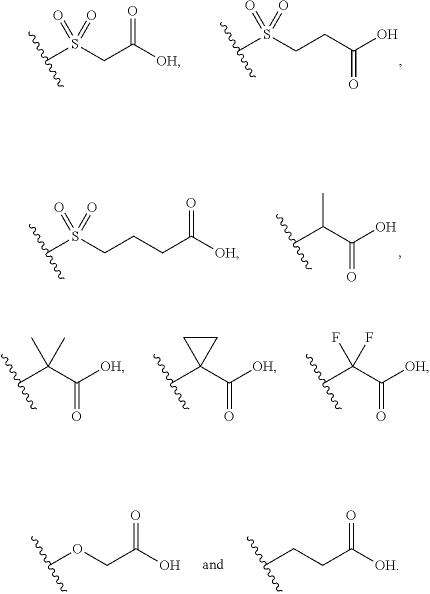

In an even more preferred embodiment in combination with any of the above and below embodiments, XYZ is

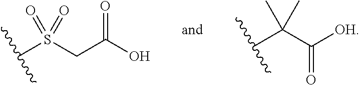

In a most preferred embodiment in combination with any of the above and below embodiments, XYZ is

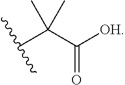 [SMILES: *C(C)(C)C(=O)O]

In a further preferred embodiment in combination with any of the above or below embodiments

X is selected from O, S(═O) and S(═O)₂;

Y is selected from C₁₋₃-alkylene, 3- to 6-membered cycloalkylene and 3- to 6-membered heterocycloalkylene, wherein alkylene, cycloalkylene or heterocycloalkylene is unsubstituted or substituted with 1 to 2 substituent independently selected from fluoro, CN, C₁₋₄-alkyl, halo-C₁₋₄-alkyl, OH, oxo, O—C₁₋₄-alkyl and O-halo-C₁₋₄-alkyl;

Z is selected from —CO₂H, —CONHOH, —CONR⁹¹R⁹², —S(═O)₂NR⁹¹R⁹²,

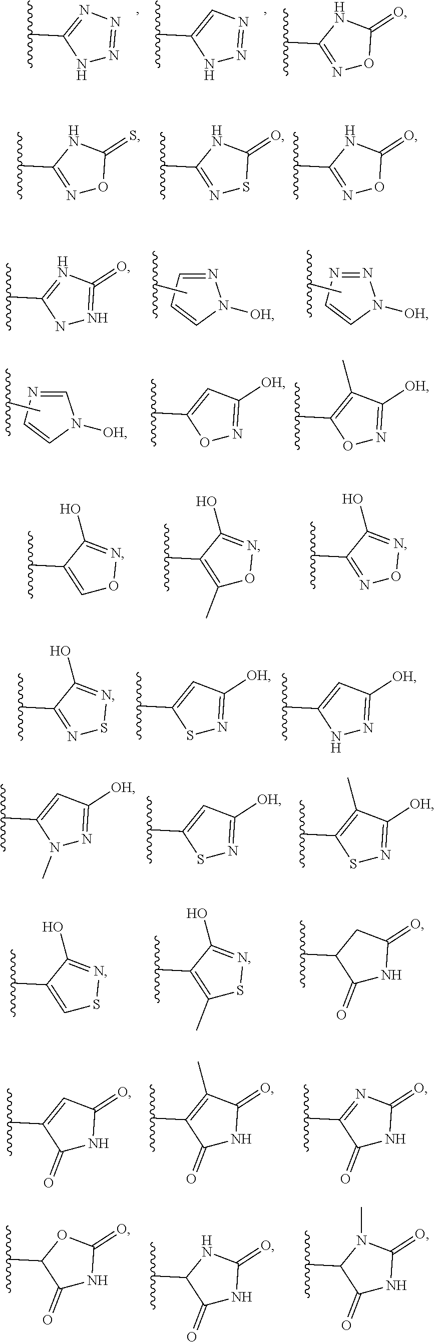

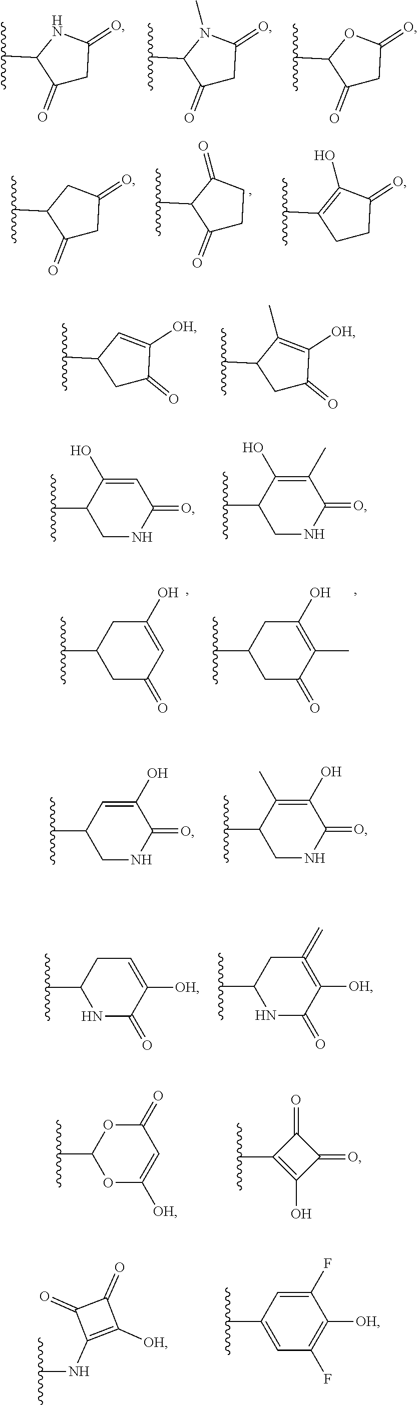

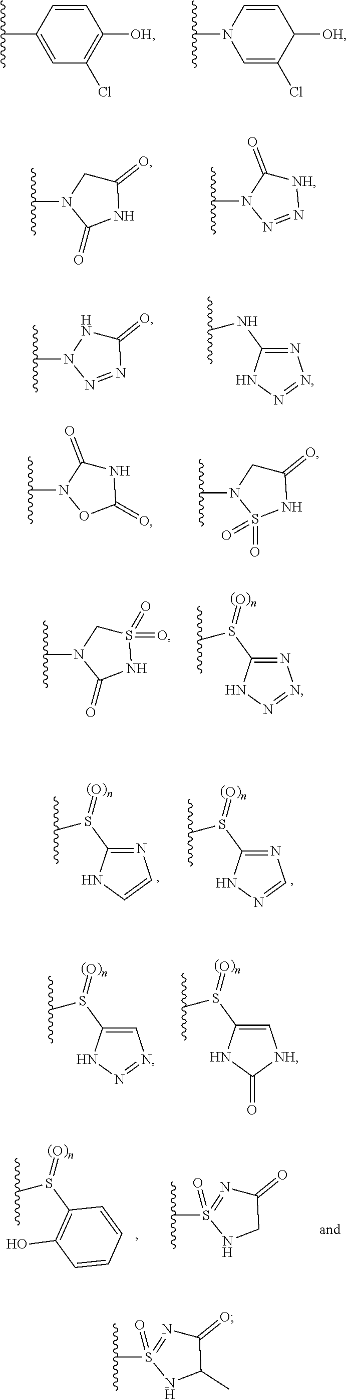

R⁹¹, R⁹² are independently selected from H, C₁₋₄-alkyl and halo-C₁₋₄-alkyl, wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3-to 6-membered heterocycloalkyl, halo-(3- to 6-membered heterocycloalkyl), OH, oxo, SO₃H, O—C₁₋₄-alkyl and O-halo-C₁₋₄-alkyl;

n is selected from 0 to 2.

In a further preferred embodiment in combination with any of the above or below embodiments \textcircled{A} is selected from

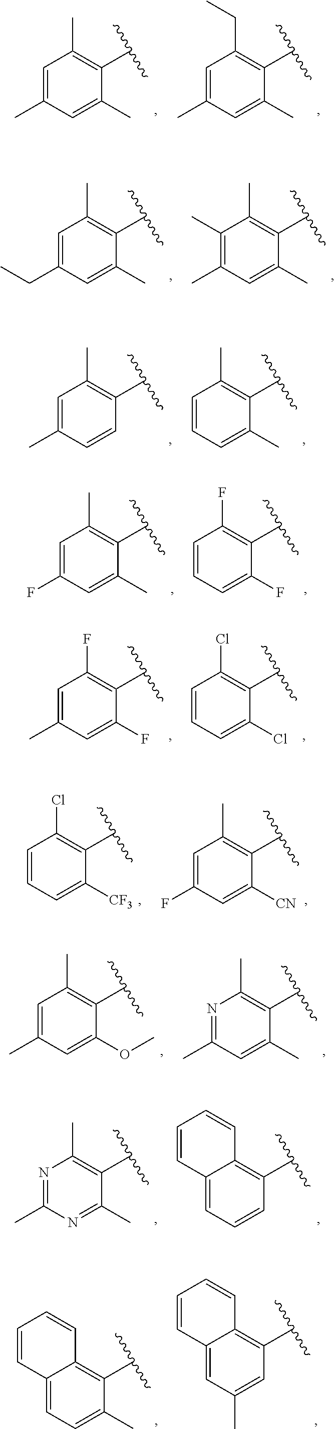

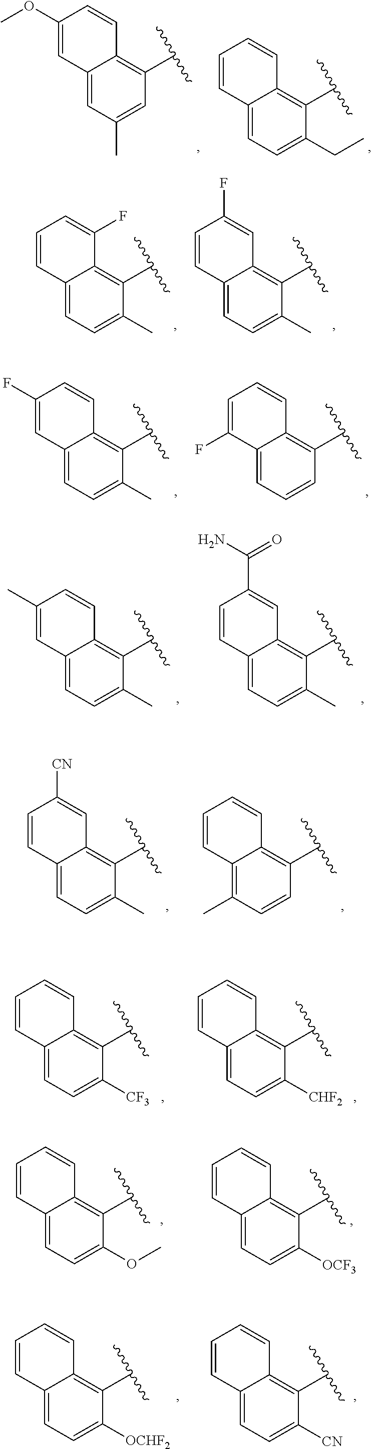

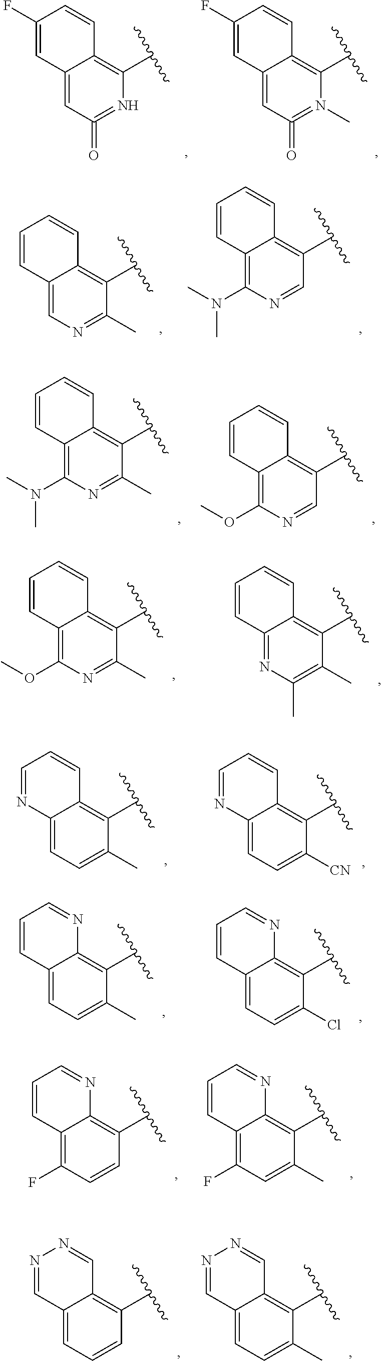

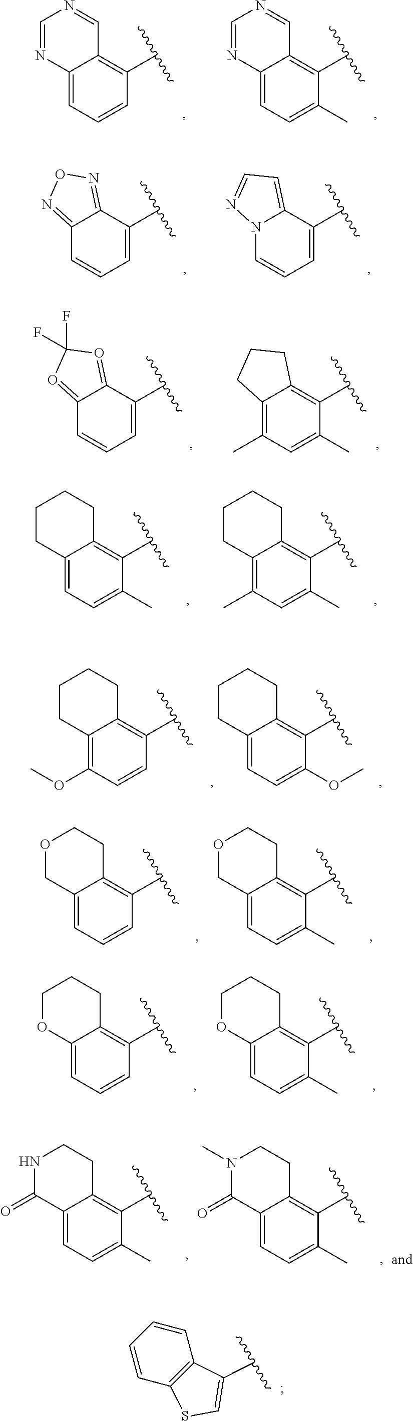

\textcircled{B} selected from

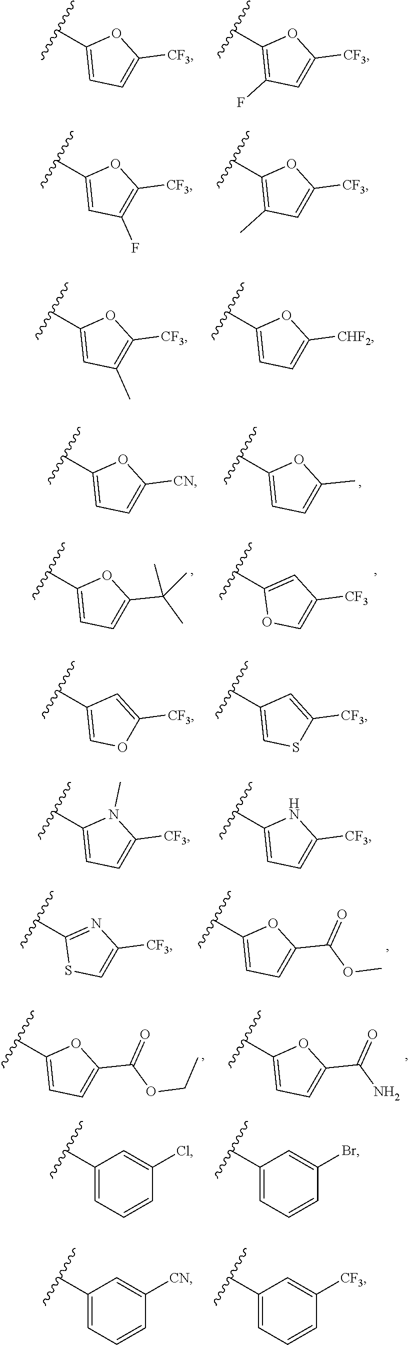

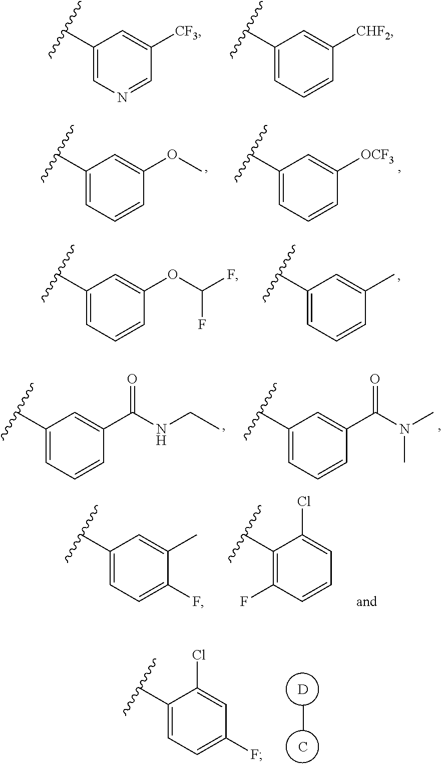

is selected from

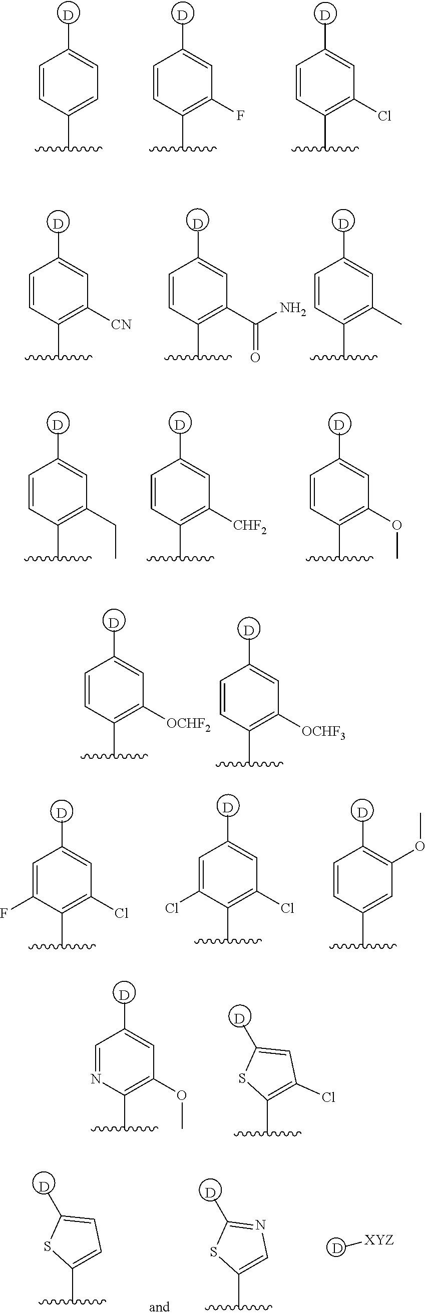

is selected from

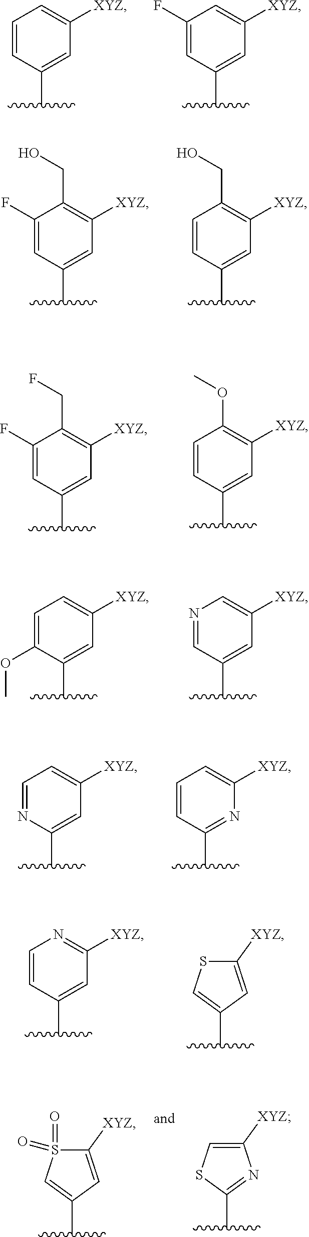

XYZ is selected from

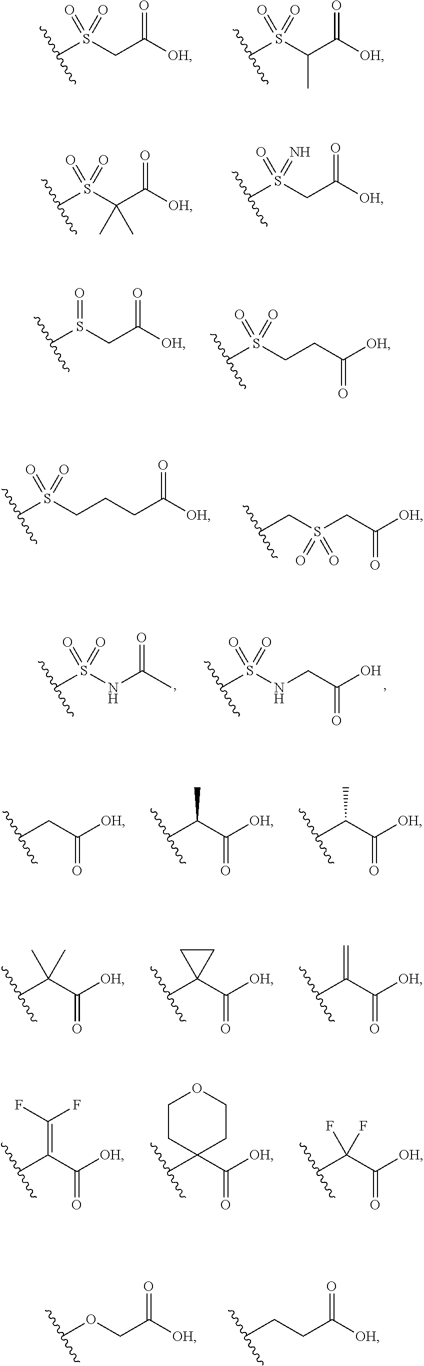

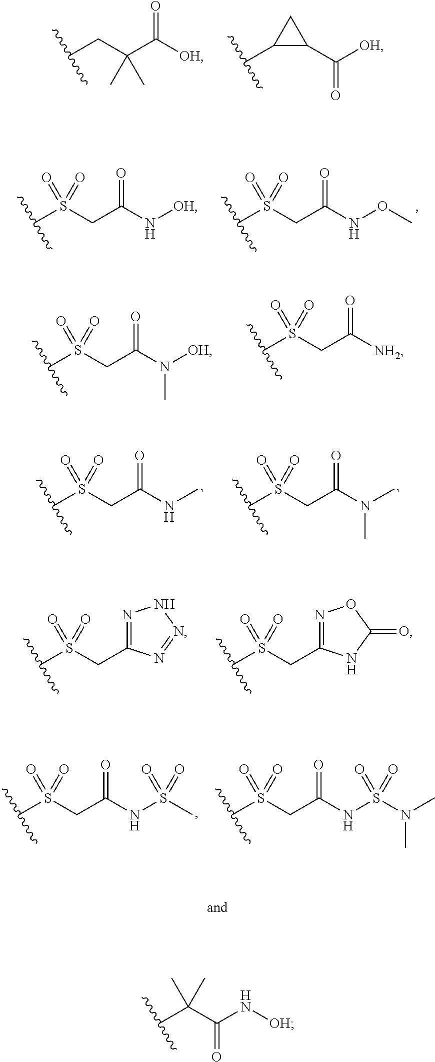

R¹, R², R³ and R⁴ are independently selected from H or Me;

W is O; and

m is selected from 1 or 2.

In an even more preferred embodiment in combination with any of the above and below embodiments, \textcircled{A} is selected from

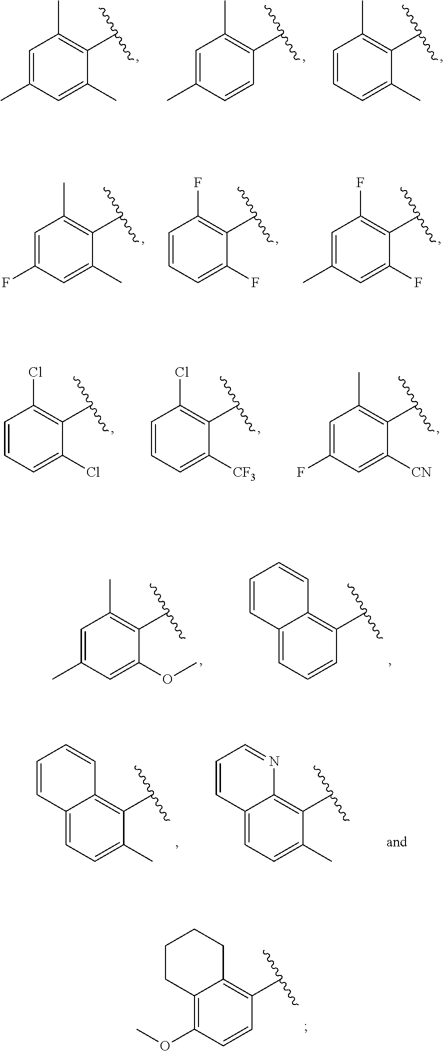

\textcircled{B} is selected from

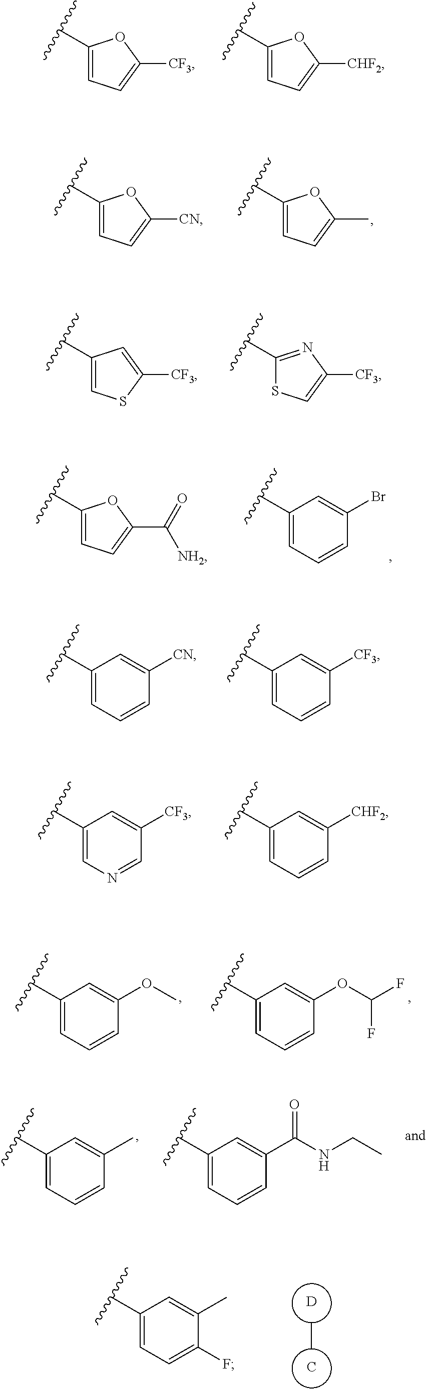

is selected from

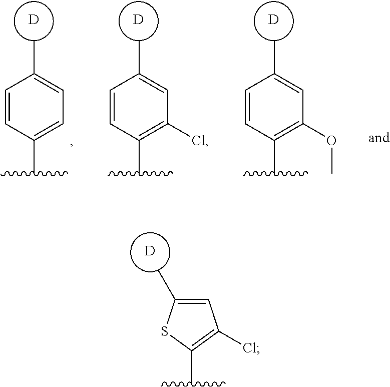

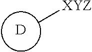

is selected from

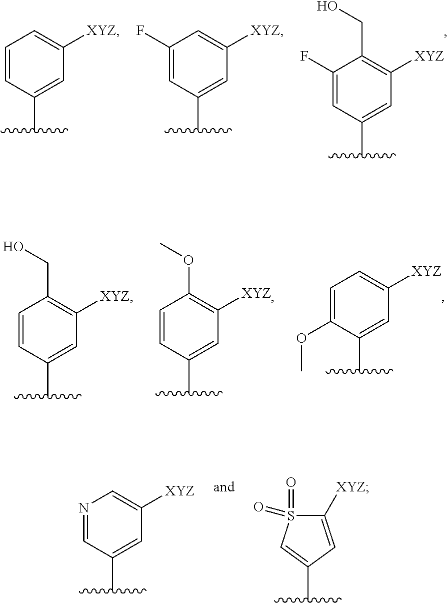

XYZ is selected from

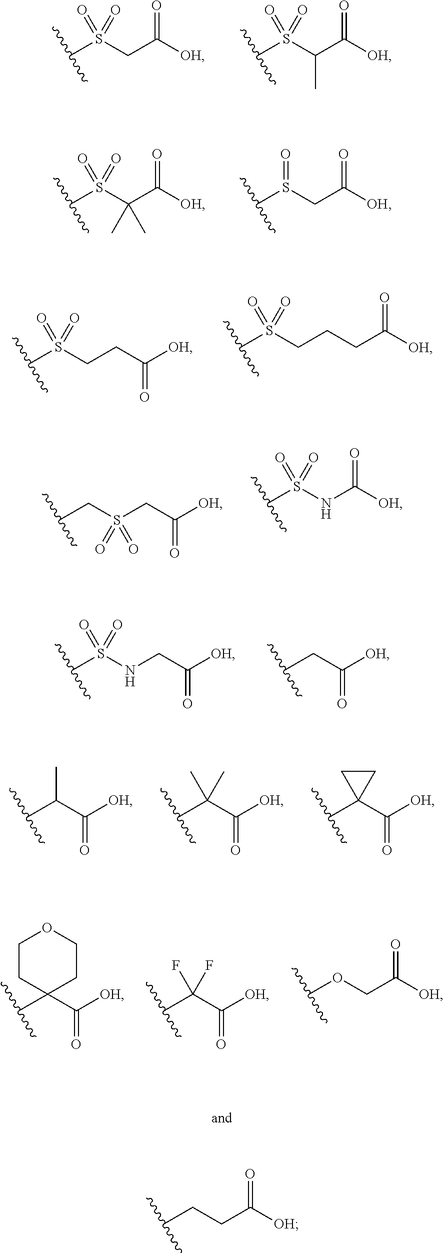

R¹, R², R³ and R⁴ are independently selected from H or Me;

W is O; and

m is selected from 1 or 2.

In an even more preferred embodiment in combination with any of the above and below embodiments, \textcircled{A} is selected from

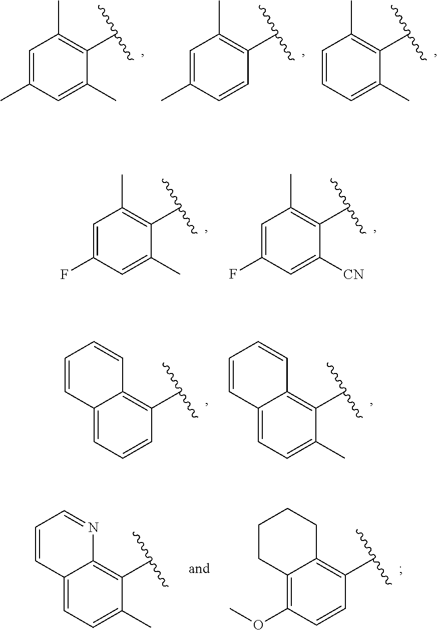

\textcircled{B} is selected from

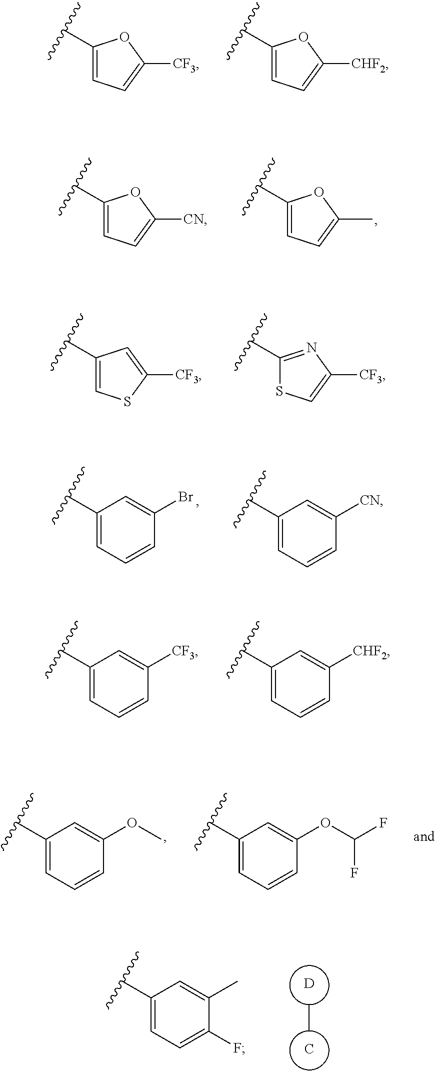

is selected from

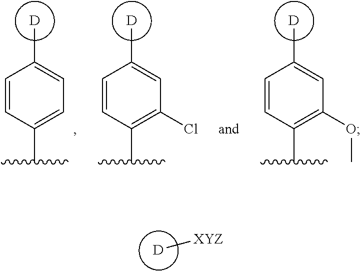

is selected from

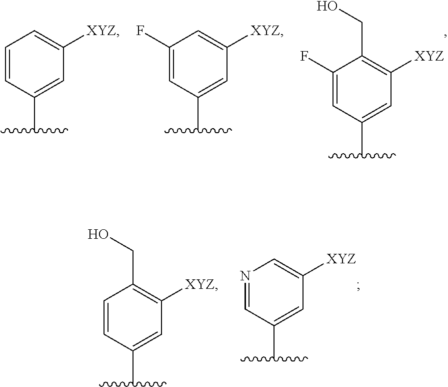

XYZ is selected from

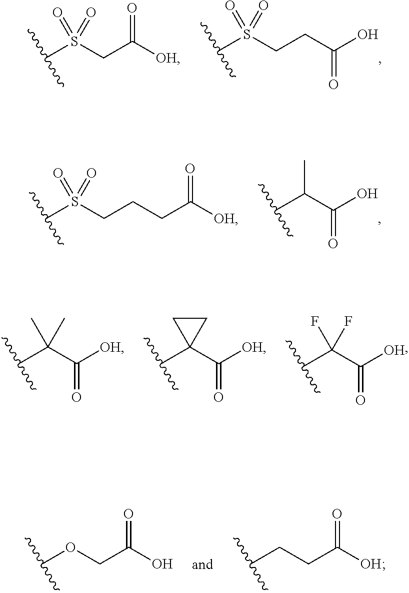

R¹, R², R³ and R⁴ are independently selected from H or Me;

W is O; and

m is 1.

In an even more preferred embodiment in combination with any of the above and below embodiments, is selected from the group consisting of phenyl, pyridyl, pyrimidinyl, naphthyl, benzo[b]thiophene, quinolinyl, isoquinolinyl, pyrazolo[1,5-a]pyrimidinyl and 1,5-naphthyridinyl wherein phenyl, pyridyl and pyrimidinyl are substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; and wherein optionally two adjacent substituents in the aryl or heteroaryl moiety form a 5- to 6-membered partially saturated cycle optionally containing 1 to 3 heteroatoms independently selected from O, S or N, wherein this additional cycle is unsubstituted or substituted with 1 to 4 substituents independently selected from fluoro, CN, oxo, OH, Me, CF₃, CHF₂, OMe, OCF₃ and OCHF₂; or wherein

naphthyl, benzo[b]thiophene, quinolinyl, isoquinolinyl, pyrazolo[1,5-a]pyrimidinyl and 1,5-naphthyridinyl are unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl.

In an even more preferred embodiment in combination with any of the above and below embodiments, \textcircled{A} is selected from the group consisting of phenyl, naphthyl and quinolinyl, wherein phenyl is substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; or wherein naphthyl or quinolinyl is unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl.

In another preferred embodiment in combination with any of the above or below embodiments,

R¹, R², R³ and R⁴ are independently selected from H or Me; and

m is 1;

W is selected from O, NR¹¹ or absent;

R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl and C(═O)—O-halo-C₁₋₄-alkyl;

\textcircled{A} is selected from the group consisting of phenyl, pyridyl, pyrimidinyl, naphthyl, benzo[b]thiophene, quinolinyl, isoquinolinyl, pyrazolo[1,5-a]pyrimidinyl and 1,5-naphthyridinyl wherein phenyl, pyridyl and pyrimidinyl are substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; and wherein optionally two adjacent substituents in the aryl or heteroaryl moiety form a 5- to 6-membered partially saturated cycle optionally containing 1 to 3 heteroatoms independently selected from O, S or N, wherein this additional cycle is unsubstituted or substituted with 1 to 4 substituents independently selected from fluoro, CN, oxo, OH, Me, CF₃, CHF₂, OMe, OCF₃ and OCHF₂; or wherein

naphthyl, benzo[b]thiophene, quinolinyl, isoquinolinyl, pyrazolo[1,5-a]pyrimidinyl and 1,5-naphthyridinyl are unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl;

\textcircled{B} is selected from the group consisting of phenyl, pyridinyl, pyrrolyl, thiazolyl, thiofuranyl or furanyl, wherein phenyl, pyridinyl, pyrrolyl, thiazolyl, thiofuranyl or furanyl are substituted with 1 to 2 substituents independently selected from the group consisting of fluoro, chloro, bromo, CN, C₁₋₄-alkyl, —O—C₁₋₄-alkyl, fluoro-C₁₋₄-alkyl, —O-fluoro-C₁₋₄-alkyl, CONH₂, CONH(C₁₋₄-alkyl), CONH(fluoro-C₁₋₄-alkyl) and CON(C₁₋₄-alkyl)₂;

\textcircled{C} is selected from the group consisting of phenyl, thiophenyl, thiazolyl and pyridinyl, wherein phenyl, thiophenyl, thiazolyl and pyridinyl are unsubstituted or substituted with 1 to 2 substituents independently selected from the group consisting of fluoro, chloro, CN, C₁₋₄-alkyl, —OC₁₋₄-alkyl, fluoro-C₁₋₄-alkyl and —O-fluoro-C₁₋₄-alkyl;

\textcircled{D} is selected from the group consisting of phenyl or pyridinyl, wherein phenyl or pyridinyl are unsubstituted or substituted with 1 to 2 substituents independently selected from the group consisting of fluoro, chloro, CN, OH, C₁₋₄-alkyl, —OC₁₋₄-alkyl, fluoro-C₁₋₄-alkyl, —O-fluoro-C₁₋₄-alkyl and C₁₋₃-alkylene-OH;

X is selected from a bond, S, S(═O) and S(═O)₂;

Y is selected from C₁₋₃-alkylene or C₃-cycloalkylene, wherein alkylene or cycloalkylene is optionally substituted with 1 to 2 substituent independently selected from halo or C₁₋₄-alkyl; and

Z is —CO₂H or an ester or pharmaceutically acceptable salt thereof.

In a more preferred embodiment in combination with any of the above or below embodiments,

R¹, R², R³ and R⁴ are independently selected from H or Me; and

m is 1;

W is selected from O, NR¹¹ or absent;

R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl and C(═O)—O-halo-C₁₋₄-alkyl;

\textcircled{A} is selected from the group consisting of phenyl, naphthyl and quinolinyl, wherein phenyl is substituted with 2 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl; or wherein naphthyl or quinolinyl is unsubstituted or substituted with 1 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl;

\textcircled{B} is selected from the group consisting of phenyl, pyridinyl, pyrrolyl, thiazolyl, thiofuranyl or furanyl, wherein phenyl, pyridinyl, pyrrolyl, thiazolyl, thiofuranyl or furanyl are substituted with 1 to 2 substituents independently selected from the group consisting of fluoro, chloro, bromo, CN, C₁₋₄-alkyl, —O—C₁₋₄-alkyl, fluoro-C₁₋₄-alkyl, —O-fluoro-C₁₋₄-alkyl, CONH₂, CONH(C₁₋₄-alkyl), CONH(fluoro-C₁₋₄-alkyl) and CON(C₁₋₄-alkyl)₂;

\textcircled{C} is selected from the group consisting of phenyl, thiophenyl, thiazolyl and pyridinyl, wherein phenyl, thiophenyl, thiazolyl and pyridinyl are unsubstituted or substituted with 1 to 2 substituents independently selected from the group consisting of fluoro, chloro, CN, C₁₋₄-alkyl, —OC₁₋₄-alkyl, fluoro-C₁₋₄-alkyl and —O-fluoro-C₁₋₄-alkyl;

\textcircled{D} is selected from the group consisting of phenyl or pyridinyl, wherein phenyl or pyridinyl are unsubstituted or substituted with 1 to 2 substituents independently selected from the group consisting of fluoro, chloro, CN, OH, C₁₋₄-alkyl, —OC₁₋₄-alkyl, fluoro-C₁₋₄-alkyl, —O-fluoro-C₁₋₄-alkyl and C₁₋₃-alkylene-OH;

X is selected from a bond, S, S(═O) and S(═O)₂;

Y is selected from C₁₋₃-alkylene or C₃-cycloalkylene, wherein alkylene or cycloalkylene is unsubstituted or substituted with 1 to 2 substituent independently selected from halo or C₁₋₄-alkyl; and

Z is —CO₂H or an ester or pharmaceutically acceptable salt thereof.

In a most preferred embodiment in combination with any of the above and below embodiments, the compound is selected from

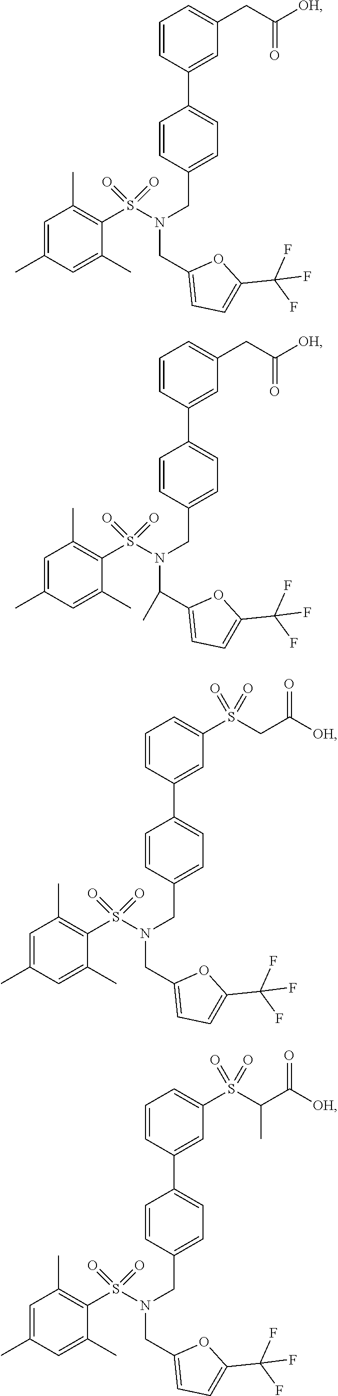

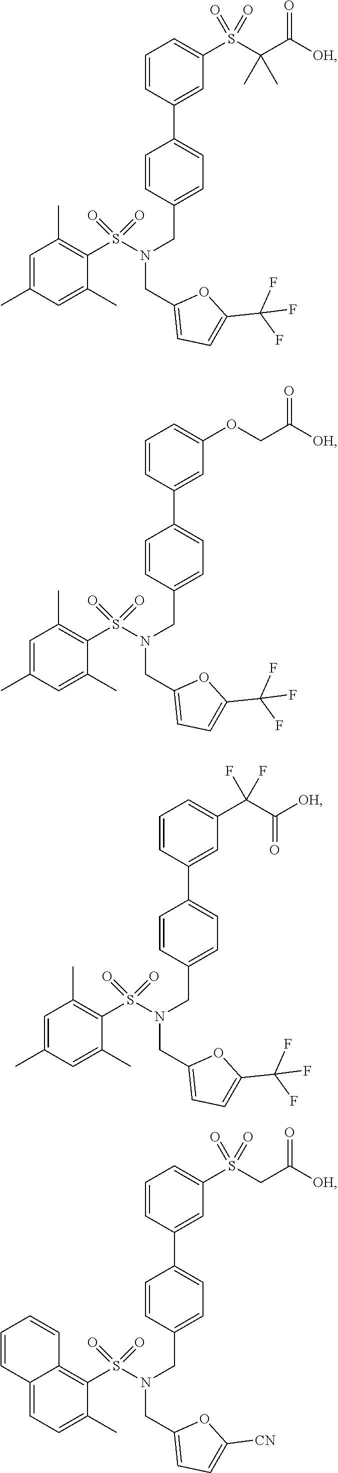

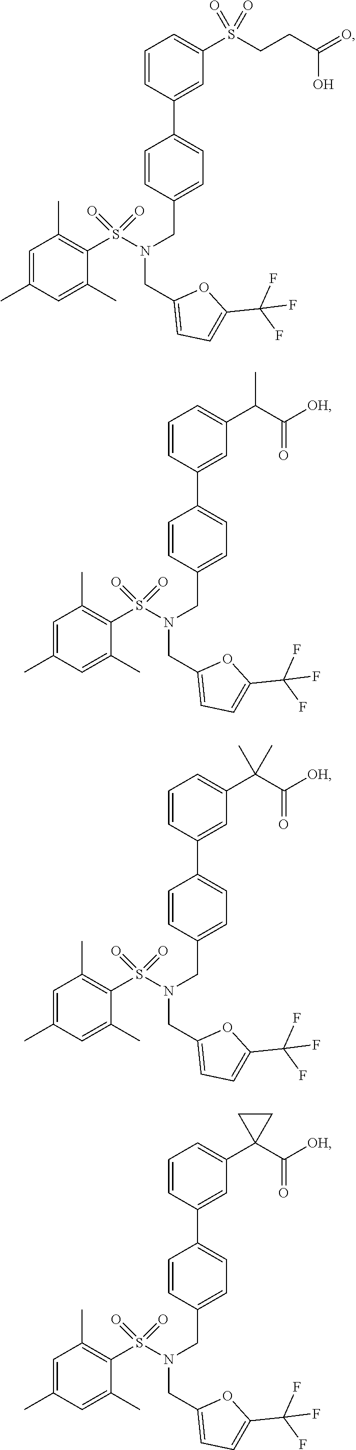

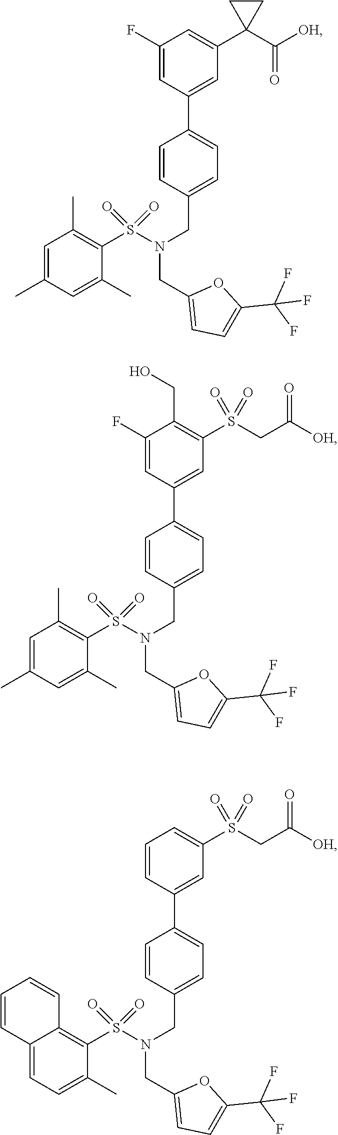 [SMILES: Cc1cc(C)c(S(=O)(=O)N(Cc2ccc(-c3cc(F)cc(C4(C(=O)O)CC4)c3)cc2)Cc2ccc(C(F)(F)F)o2)c(C)c1 ; Cc1cc(C)c(S(=O)(=O)N(Cc2ccc(-c3cc(F)c(CO)c(S(=O)(=O)CC(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1ccc(C(F)(F)F)o1]

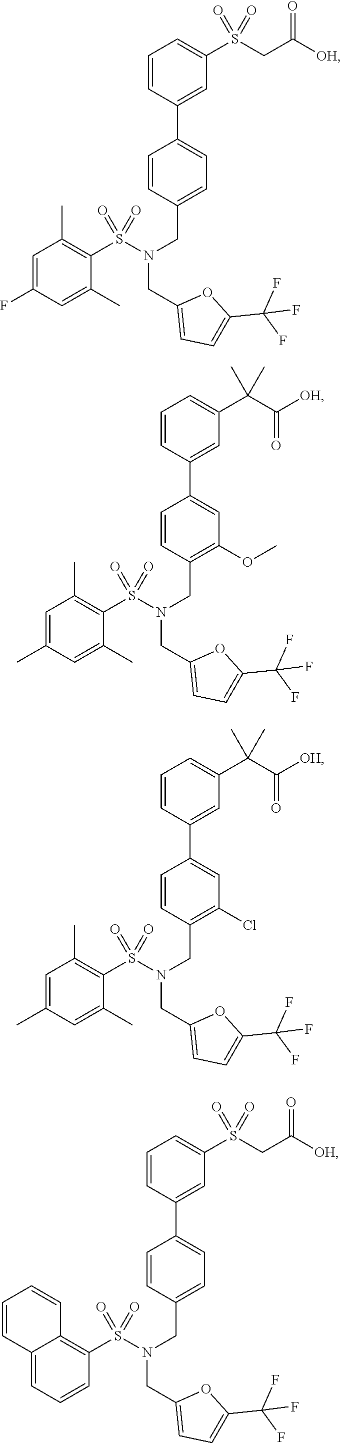

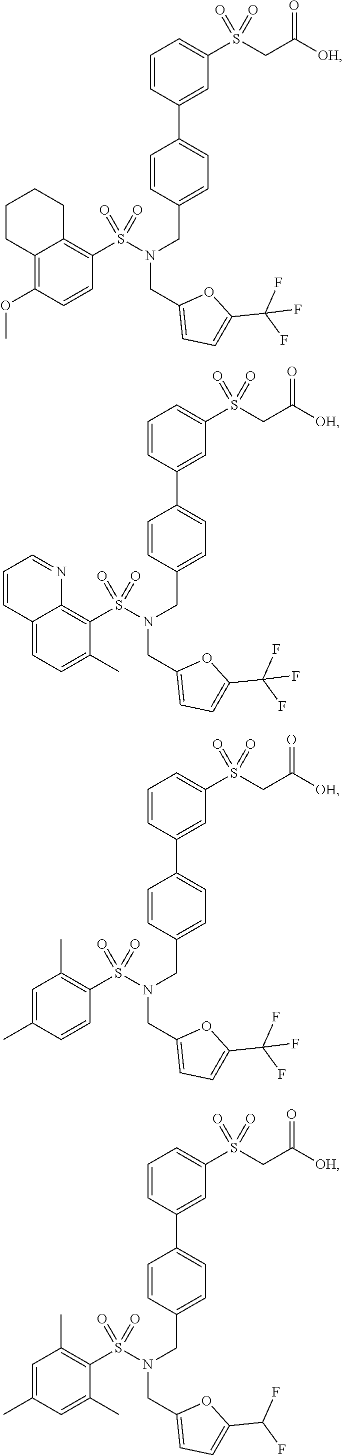

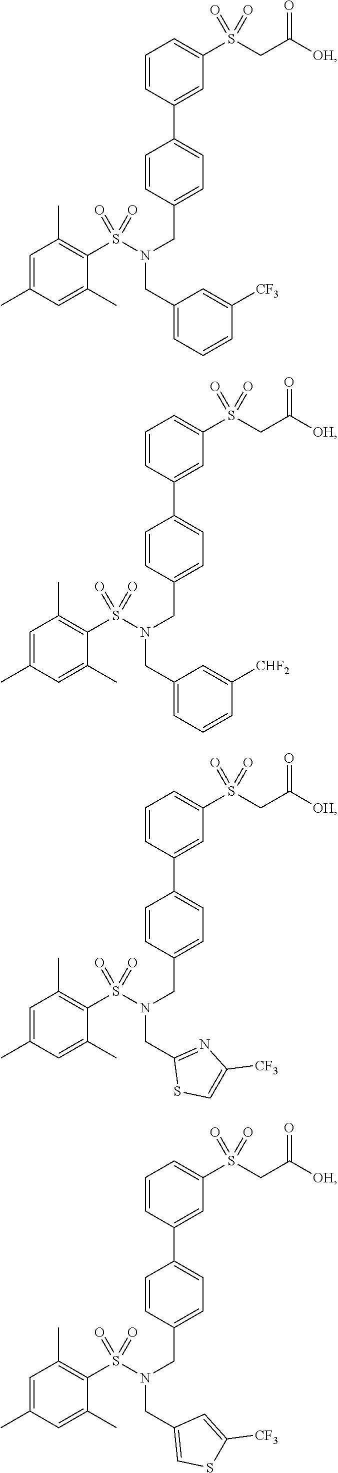

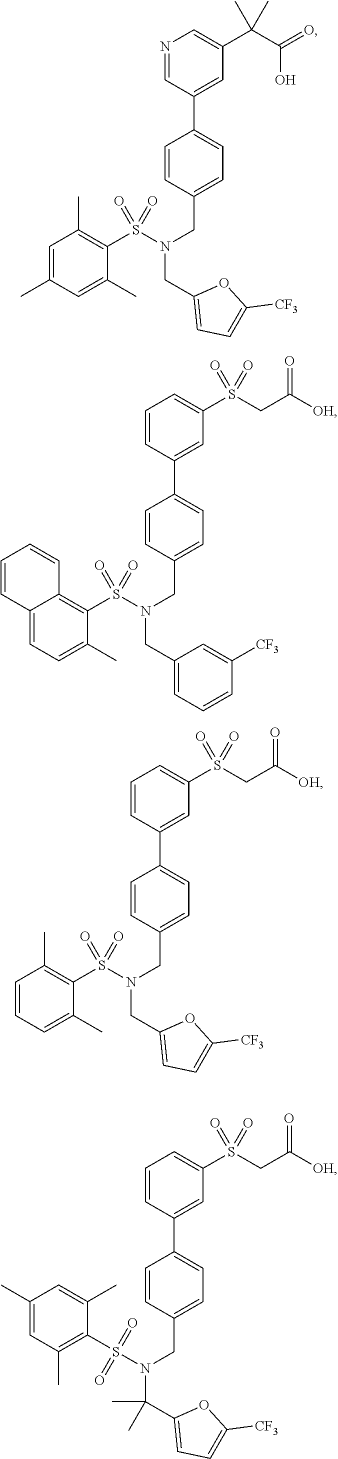

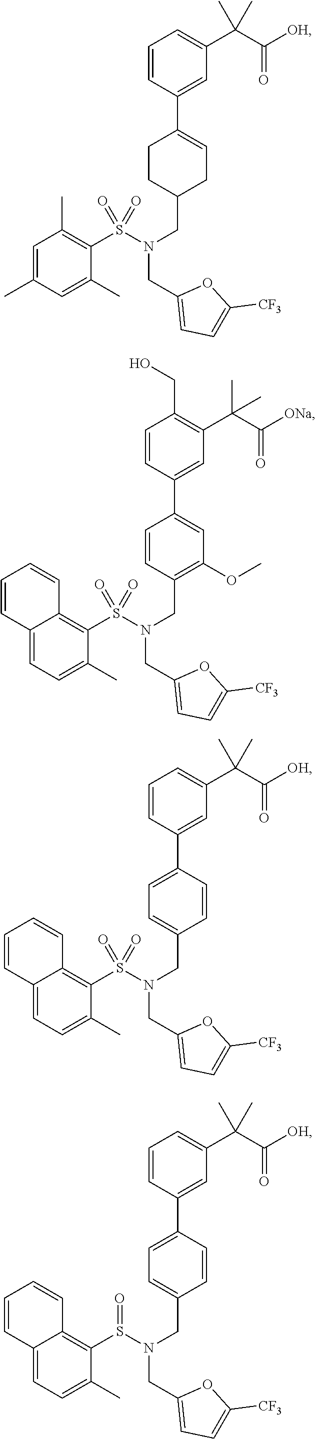

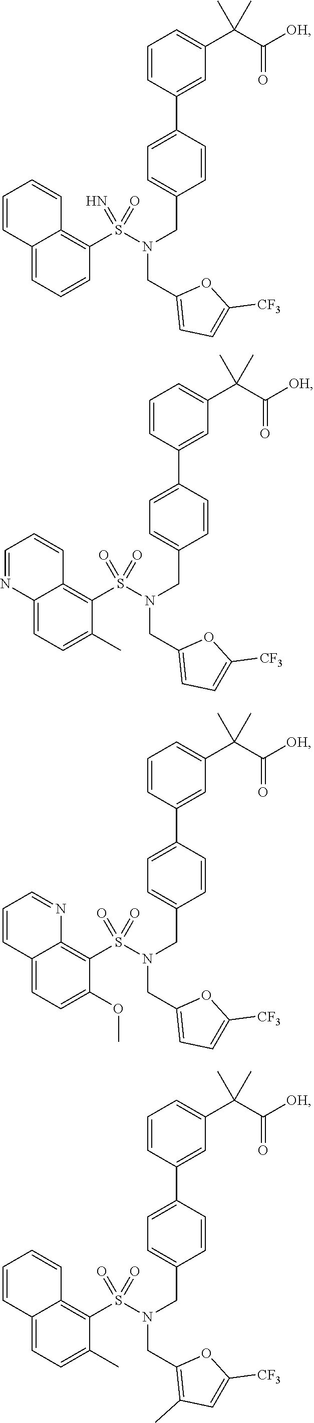

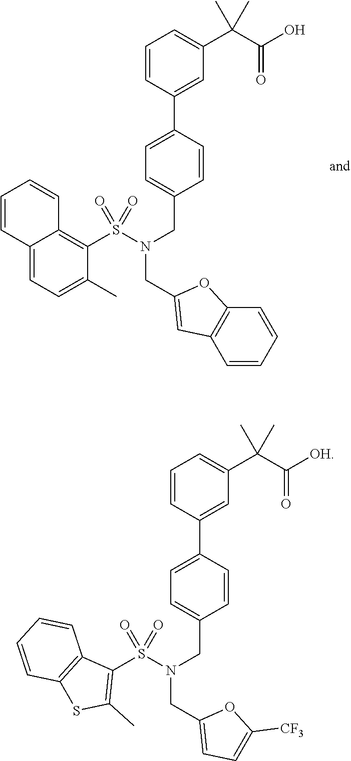 [SMILES: Cc1ccc2ccccc2c1S(=O)(=O)N(Cc1ccc(-c2cccc(C(C)(C)C(=O)O)c2)cc1)Cc1cc2ccccc2o1 ; "and" ; Cc1sc2ccccc2c1S(=O)(=O)N(Cc1ccc(-c2cccc(C(C)(C)C(=O)O)c2)cc1)Cc1ccc(C(F)(F)F)o1]

In an upmost preferred embodiment in combination with any of the above and below embodiments, the compound is selected from

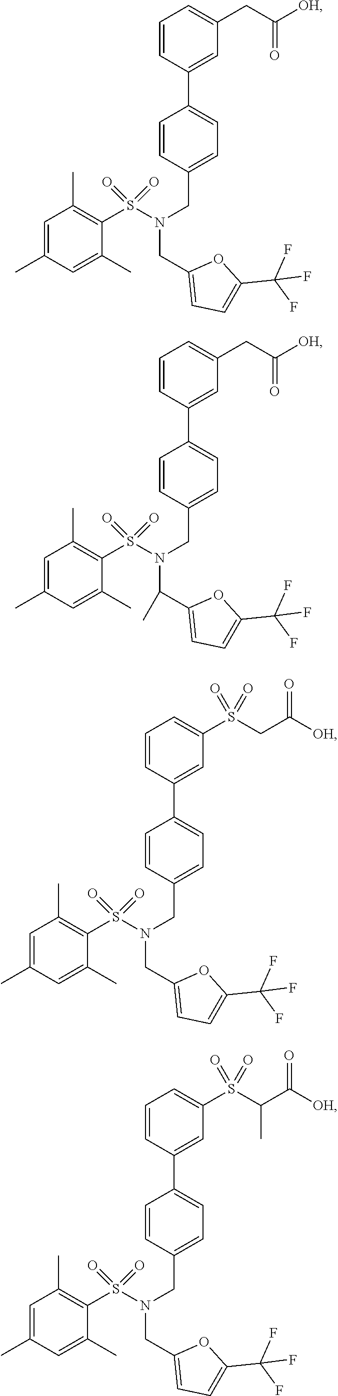

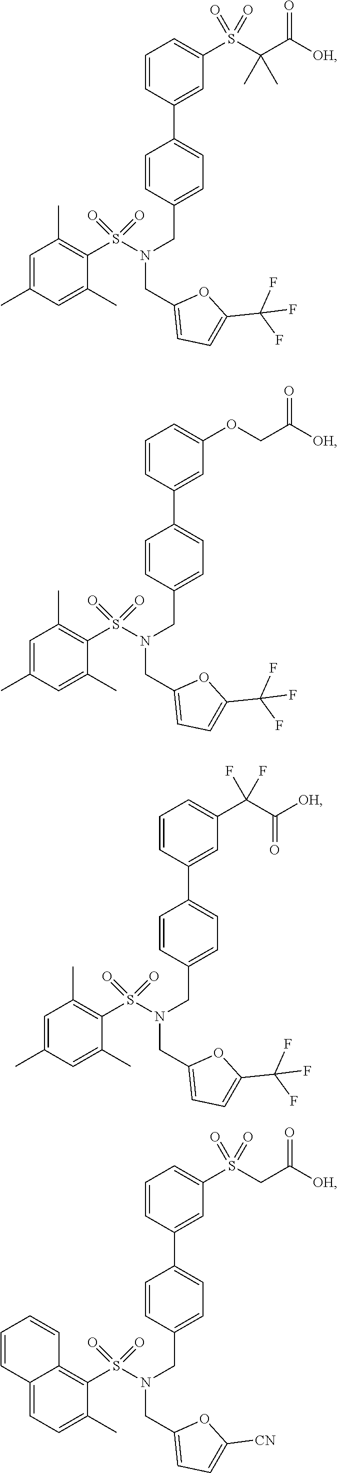

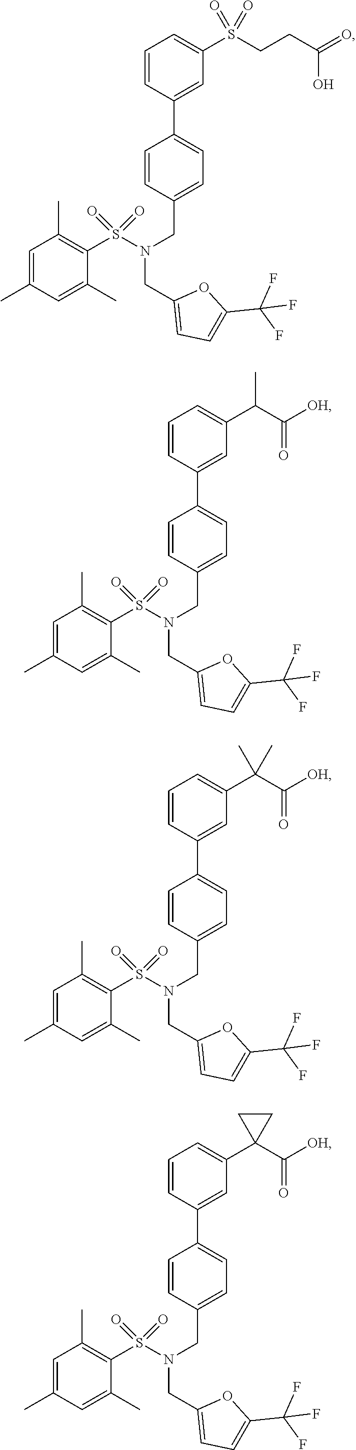

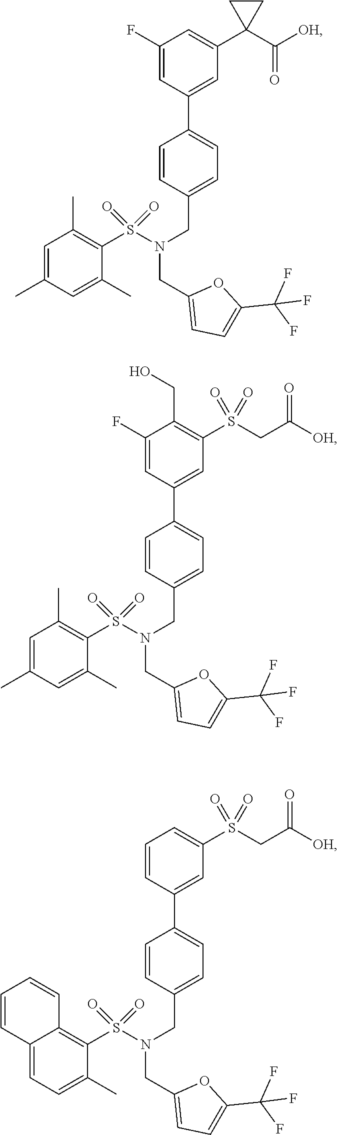 [SMILES: Cc1cc(C)c(S(=O)(=O)N(Cc2ccc(-c3cc(F)cc(C4(C(=O)O)CC4)c3)cc2)Cc2ccc(C(F)(F)F)o2)c(C)c1 ; Cc1cc(C)c(S(=O)(=O)N(Cc2ccc(-c3cc(F)c(CO)c(S(=O)(=O)CC(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1ccc(C(F)(F)F)o1]

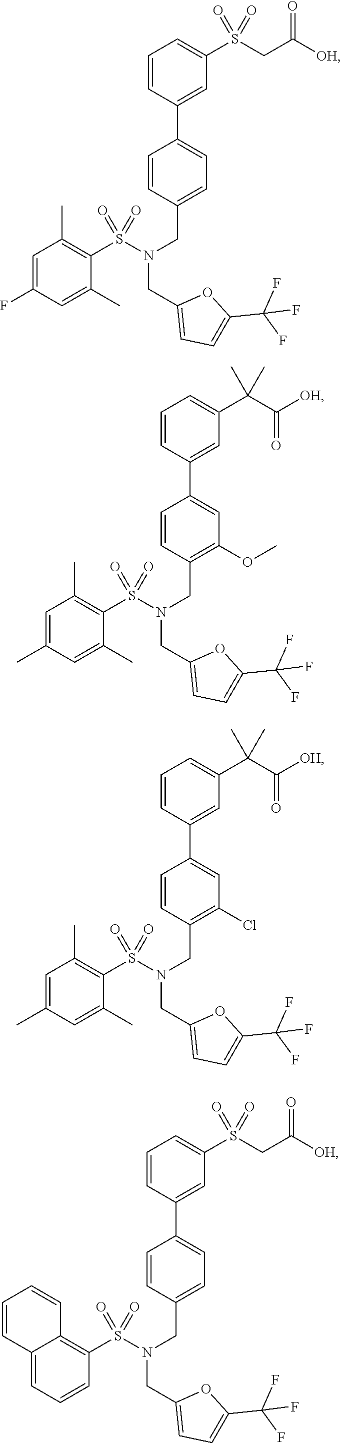

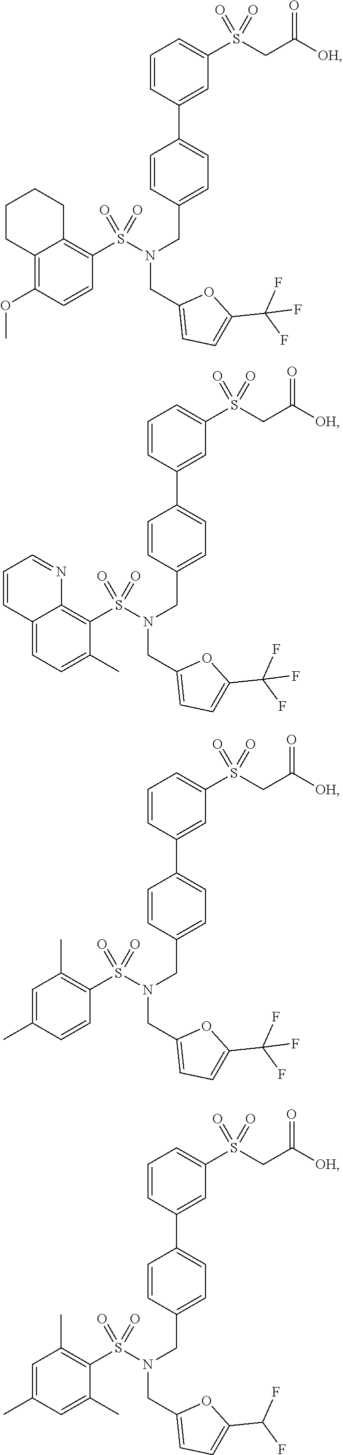

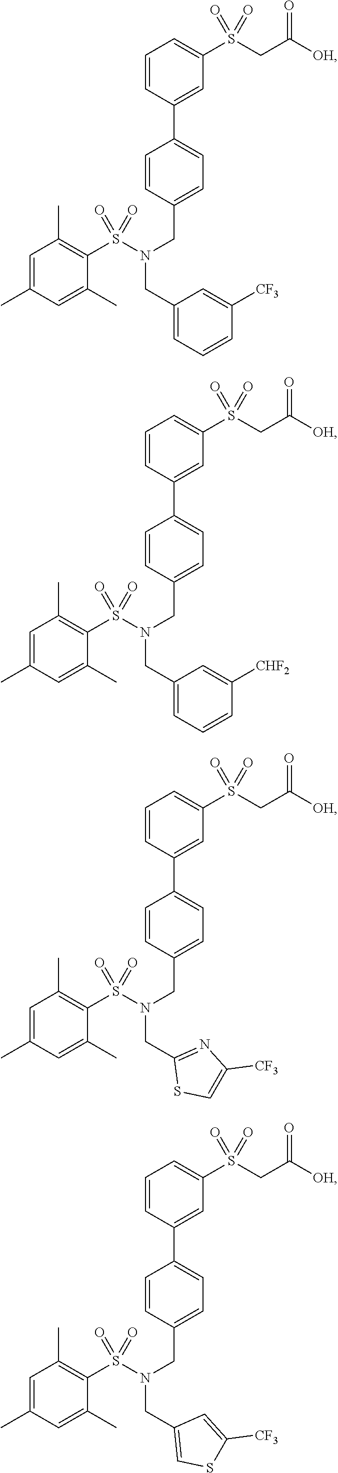

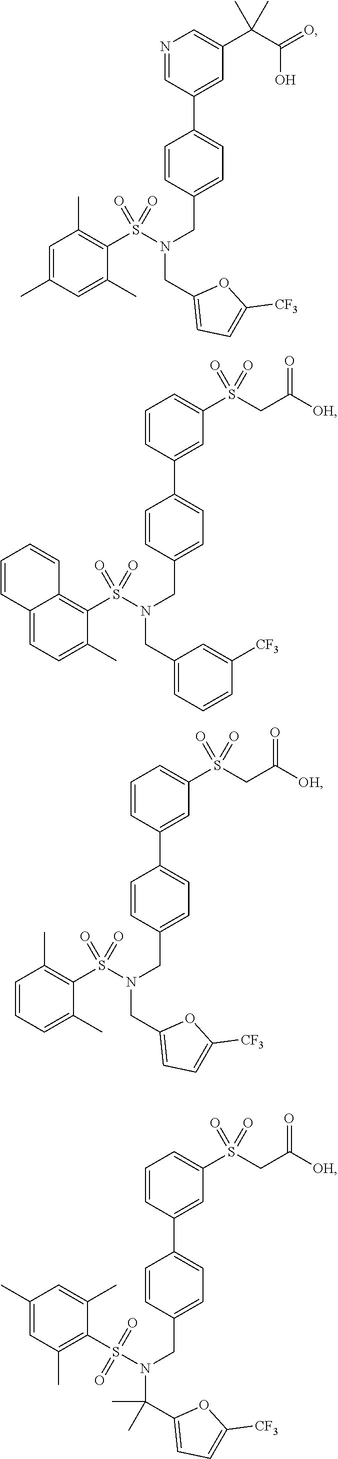

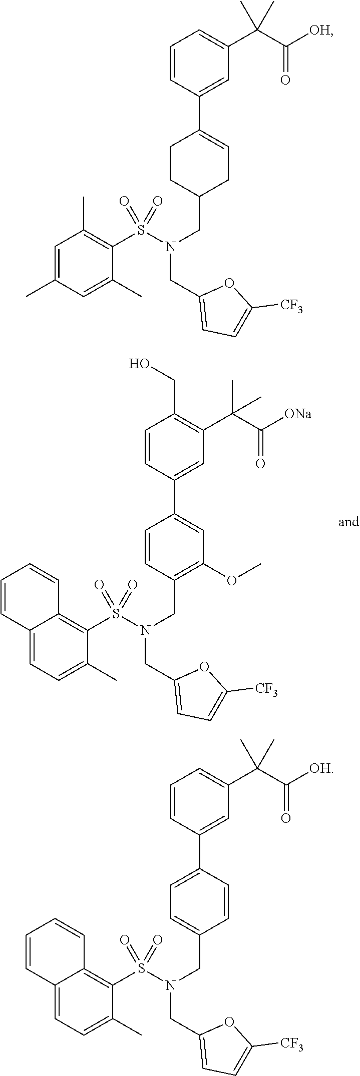

In an uppermost preferred embodiment in combination with any of the above and below embodiments, the compound is selected from

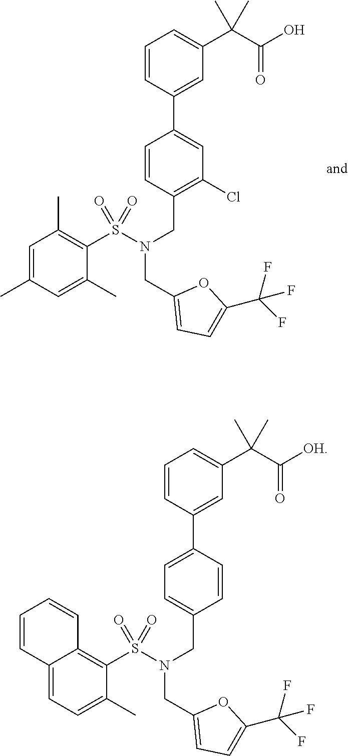 [SMILES: Cc1cc(C)c(S(=O)(=O)N(Cc2ccc(C(F)(F)F)o2)Cc2ccc(-c3cccc(C(C)(C)C(=O)O)c3)cc2Cl)c(C)c1 ; Cc1ccc2ccccc2c1S(=O)(=O)N(Cc1ccc(-c2cccc(C(C)(C)C(=O)O)c2)cc1)Cc1ccc(C(F)(F)F)o1]

The invention also provides the compound of the invention for use as a medicament.

Also provided is the compound of the present invention for use in the prophylaxis and/or treatment of diseases mediated by LXRs.

Also provided is the compound of the invention in treating a LXR mediated disease selected from non-alcoholic fatty liver disease, non-alcoholic steatohepatitis, liver inflammation, liver fibrosis, obesity, insulin resistance, type II diabetes, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

Also provided is a pharmaceutical composition comprising the compound of the invention and a pharmaceutically acceptable carrier or excipient.

In the context of the present invention “C₁₋₄-alkyl” means a saturated alkyl chain having 1 to 4 carbon atoms which may be straight chained or branched. Examples thereof include methyl, ethyl, propyl, isopropyl, n-butyl, isobutyl, and tert-butyl.

The term “halo-C₁₋₄-alkyl” means that one or more hydrogen atoms in the alkyl chain are replaced by a halogen. A preferred example thereof is CF₃.

A “C₀₋₆-alkylene” means that the respective group is divalent and connects the attached residue with the remaining part of the molecule. Moreover, in the context of the present invention, “C₀-alkylene” is meant to represent a bond, whereas C₁-alkylene means a methylene linker, C₂-alkylene means an ethylene linker or a methyl-substituted methylene linker and so on. In the context of the present invention, a C₀₋₆-alkylene preferably represents a bond, a methylene, an ethylene group or a propylene group.

Similarly, a “C₂₋₆-alkenylene” and a “C₂₋₆-alkinylene” means a divalent alkenyl or alkynyl group which connects two parts of the molecule.

A 3- to 10-membered cycloalkyl group means a saturated or partially unsaturated mono-, bi-, spiro- or multicyclic ring system comprising 3 to 10 carbon atoms. Examples include cyclopropyl, cyclobutyl, cyclopentyl, cyclohexyl, cyclohexenyl, bicyclo[2.2.2]octyl, bi-cyclo[3.2.1]octanyl, spiro[3.3]heptyl, bicyclo[2.2.1]heptyl, adamantyl and penta-cyclo[4.2.0.0^{2.5}0.0^{3.8}0.0^{4.7}]octyl. Consequently, a 3- to 6-membered cycloalkyl group means a saturated or partially unsaturated mono- bi-, or spirocyclic ring system comprising 3 to 6 carbon atoms whereas a 5- to 8-membered cycloalkyl group means a saturated or partially unsaturated mono-, bi-, or spirocyclic ring system comprising 5 to 8 carbon atoms.

A 3- to 10-membered heterocycloalkyl group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O, S, SO and SO₂. Examples thereof include epoxidyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom. An example for a S-linked heterocycloalkyl is the cyclic sulfonimidamide

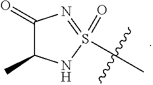 [SMILES: *S1(=O)=NC(=O)[C@H](C)N1]

A 5- to 10-membered mono- or bicyclic heteroaromatic ring system (within the application also referred to as heteroaryl) means an aromatic ring system containing up to 4 heteroatoms independently selected from N, O, S, SO and SO₂. Examples of monocyclic heteroaromatic rings include pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzofuranyl, benzoxazolyl, indolyl, indolizinyl and pyrazolo[1,5-a]pyrimidinyl. The nitrogen or sulphur atom of the heteroaryl system may also be optionally oxidized to the corresponding N-oxide, S-oxide or S,S-dioxide. If not stated otherwise, the heteroaryl system can be connected via a carbon or nitrogen atom. Examples for N-linked heterocycles are

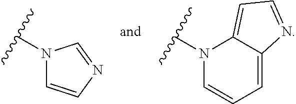 [SMILES: *n1ccnc1 ; "and" ; *n1cccc2nccc1-2]

A 6- to 10-membered mono- or bicyclic aromatic ring system (within the application also referred to as aryl) means an aromatic carbon cycle such as phenyl or naphthyl.

The term “N-oxide” denotes compounds, where the nitrogen in the heteroaromatic system (preferably pyridinyl) is oxidized. Such compounds can be obtained in a known manner by reacting a compound of the present invention (such as in a pyridinyl group) with H₂O₂ or a peracid in an inert solvent.

Halogen is selected from fluorine, chlorine, bromine and iodine, more preferably fluorine or chlorine and most preferably fluorine.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

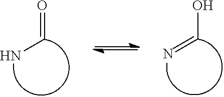

A cycloalkyl or heterocycloalkyl group can be connected straight or spirocyclic, e.g. when cyclohexane is substituted with the heterocycloalkyl group oxetane, the following structures are possible:

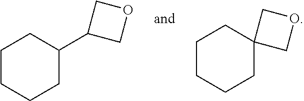 [SMILES: C1CCC(C2COC2)CC1 ; "and" ; C1CCC2(CC1)COC2]

The term “1,3-orientation” means that on a ring the substituents have at least one possibility, where 3 atoms are between the two substituents attached to the ring system, e.g.

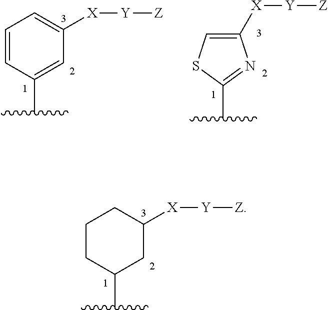

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

The compounds of the present invention can be in the form of a prodrug compound. “Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods. Other examples of the prodrug are compounds (referred to as “ester prodrug” in the application, wherein the carboxylate in a compound of the present invention is, for example, converted into an alkyl-, aryl-, arylalkylene-, amino-, choline-, acyloxyalkyl-, 1-((alkoxycarbonyl)oxy)-2-alkyl, or linolenoyl-ester. Exemplary structures for prodrugs of carboxylic acids are

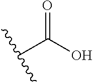 [SMILES: *C(=O)O]

prodrugs:

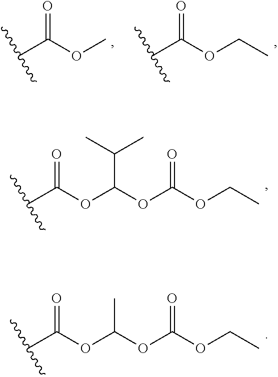

A ester prodrug can also be formed, when a carboxylic acid forms a lactone with a hydroxy group from the molecule. An exemplary example is

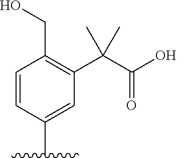 [SMILES: *c1ccc(CO)c(C(C)(C)C(=O)O)c1]

prodrug:

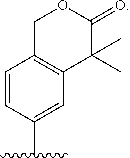 [SMILES: *c1ccc2c(c1)C(C)(C)C(=O)OC2]

The term “—CO₂H or an ester thereof” means that the carboxylic acid and the alkyl esters are intented, e.g.

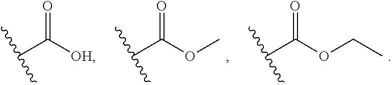

Metabolites of compounds of the present invention are also within the scope of the present invention.

Where tautomerism, like e.g. keto-enol tautomerism, of compounds of the present invention or their prodrugs may occur, the individual forms, like e.g. the keto and enol form, are each within the scope of the invention as well as their mixtures in any ratio. Same applies for stereoisomers, like e.g. enantiomers, cis/trans isomers, conformers and the like.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials. Another way to obtain pure enantiomers from racemic mixtures would use enantioselective crystallization with chiral counterions.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further the compounds of the present invention may be present in the form of solvates, such as those which include as solvate water, or pharmaceutically acceptable solvates, such as alcohols, in particular ethanol.

Furthermore, the present invention provides pharmaceutical compositions comprising at least one compound of the present invention, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier.

The pharmaceutical composition of the present invention may additionally comprise one or more other compounds as active ingredients like a prodrug compound or other nuclear receptor modulators.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

The compounds of the present invention act as LXR modulators.

Ligands to nuclear receptors including LXR ligands can either act as agonists, antagonists or inverse agonists. An agonist in this context means a small molecule ligand that binds to the receptor and stimulates its transcriptional activity as determined by e.g. an increase of mRNAs or proteins that are transcribed under control of an LXR response element. Transcriptional activity can also be determined in biochemical or cellular in vitro assays that employ just the ligand binding domain of LXRα or LXRβ but use the interaction with a cofactor (i.e. a corepressor or a coactivator), potentially in conjunction with a generic DNA-binding element such as the Gal4 domain, to monitor agonistic, antagonistic or inverse agonistic activity.

Whereas an agonist by this definition stimulates LXR- or LXR-Gal4-driven transcriptional activity, an antagonist is defined as a small molecule that binds to LXRs and thereby inhibits transcriptional activation that would otherwise occur through an endogenous LXR ligand.

An inverse agonist differs from an antagonist in that it not only binds to LXRs and inhibits transcriptional activity but in that it actively shuts down transcription directed by LXR, even in the absence of an endogenous agonist. Whereas it is difficult to differentiate between LXR antagonistic and inverse agonistic activity in vivo, given that there are always some levels of endogenous LXR agonist present, biochemical or cellular reporter assays can more clearly distinguish between the two activities. At a molecular level an inverse agonist does not allow for the recruitment of a coactivator protein or active parts thereof whereas it should lead to an active recruitment of corepressor proteins are active parts thereof. An LXR antagonist in this context would be defined as an LXR ligand that neither leads to coactivator nor to corepressor recruitment but acts just through displacing LXR agonists. Therefore, the use of assays such as the Gal4-mammalian-two-hybrid assay is mandatory in order to differentiate between coactivator or corepressor-recruiting LXR compounds (Kremoser et al., Drug Discov. Today 2007; 12:860; Gronemeyer et al., Nat. Rev. Drug Discov. 2004; 3:950).

Since the boundaries between LXR agonists, LXR antagonists and LXR inverse agonists are not sharp but fluent, the term “LXR modulator” was coined to encompass all compounds which are not clean LXR agonists but show a certain degree of corepressor recruitment in conjunction with a reduced LXR transcriptional activity. LXR modulators therefore encompass LXR antagonists and LXR inverse agonists and it should be noted that even a weak LXR agonist can act as an LXR antagonist if it prevents a full agonist from full transcriptional activation.

FIG. 1 shall illustrate the differences between LXR agonists, antagonists and inverse agonists here differentiated by their different capabilities to recruit coactivators or corepressors.

The compounds are useful for the prophylaxis and/or treatment of diseases which are mediated by LXRs. Preferred diseases are all disorders associated with steatosis, i.e. tissue fat accumulation. Such diseases encompass the full spectrum of non-alcoholic fatty liver disease including non-alcoholic steatohepatitis, liver inflammation and liver fibrosis, furthermore insulin resistance, metabolic syndrome and cardiac steatosis. An LXR modulator based medicine might also be useful for the treatment of hepatitis C virus infection or its complications and for the prevention of unwanted side-effects of long-term glucocorticoid treatment in diseases such as rheumatoid arthritis, inflammatory bowel disease and asthma.

A different set of applications for LXR modulators might be in the treatment of cancer. LXR antagonists or inverse agonists might useful to counteract the so-called Warburg effect which is associated with a transition from normal differentiated cells towards cancer cells (see Liberti et al., Trends Biochem. Sci. 2016; 41:211; Ward & Thompson, Cancer Cell 2012; 21:297-308). Furthermore, LXR is known to modulate various components of the innate and adaptive immune system. Oxysterols, which are known as endogenous LXR agonists were identified as mediators of an LXR-dependent immunosuppressive effect found in the tumor micro-environment (Traversari et al., Eur. J. Immunol. 2014; 44:1896). Therefore, it is reasonable to assume that LXR antagonists or inverse agonists might be capable of stimulating the immune system and antigen-presenting cells, in particular, to elicit an anti-tumor immune response. The latter effects of LXR antagonists or inverse agonists might be used for a treatment of late stage cancer, in general, and in particular for those types of cancerous solid tumors that show a poor immune response and highly elevated signs of Warburg metabolism.

In more detail, anti-cancer activity of the LXR inverse agonist SR9243 was shown to be mediated by interfering with the Warburg effect and lipogenesis in different tumor cells in vitro and SW620 colon tumor cells in athymic mice in vivo (see Flaveny et al. Cancer Cell. 2015; 28:42; Steffensen, Cancer Cell 2015; 28:3).

LXR modulators (preferably LXR inverse agonists) may counteract the diabetogenic effects of glucocorticoids without compromising the anti-inflammatory effects of glucocorticoids and could therefore be used to prevent unwanted side-effects of long-term glucocorticoid treatment in diseases such as rheumatoid arthritis, inflammatory bowel disease and asthma (Patel et al. Endocrinology 2017:in press; doi: 10.1210/en.2017-00094)

LXR modulators (preferably LXR inverse agonists) may be useful for the treatment of hepatitis C virus mediated liver steatosis (see Garcia-Mediavilla et al. Lab Invest. 2012; 92:1191).

LXR modulators (preferably LXR inverse agonists) may be useful for the treatment of viral myocarditis (see Papageorgiou et al. Cardiovasc Res. 2015; 107:78).

LXR modulators (preferably LXR inverse agonists) may be useful for the treatment of insulin resistance (see Zheng et al. PLoS One 2014; 9:e101269).

Experimental Section

The compounds of the present invention can be prepared by a combination of methods known in the art including the procedures described in Schemes I and II below.

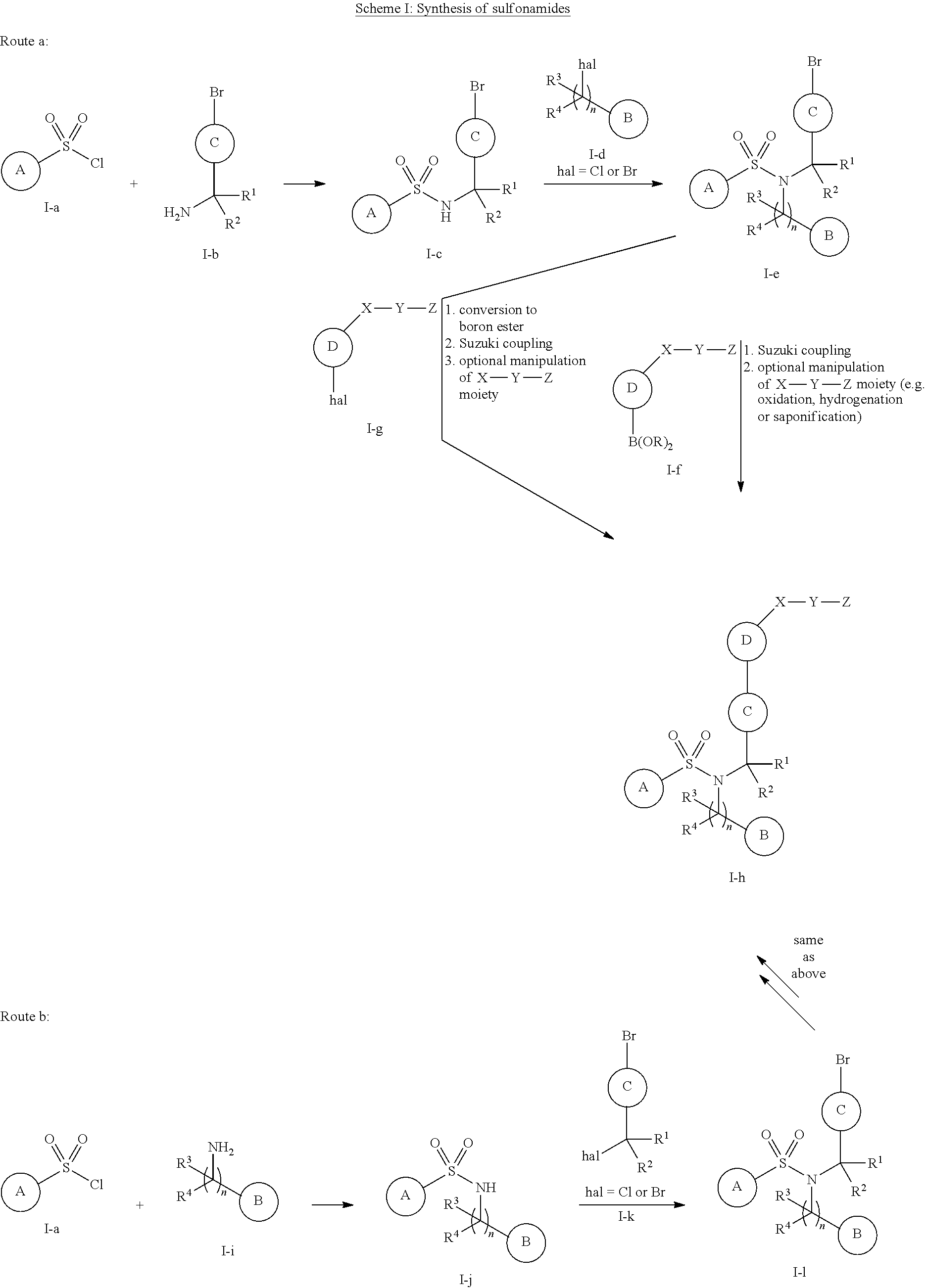

In case when W is not an oxygen atom, the compounds of the present invention can be prepared as outlined in Scheme II: Sulfonyl chloride II-a can get converted to sulfinic acid II-b. Activation with oxalyl chloride to the corresponding sulfinic acid chloride and then coupling to an amine (see Zhu et al. Tetrahedron:Asymmetry 2011; 22:387) affords an intermediate, which can be processed as outlined in Scheme I above to finally afford sulfinamide II-c.

Sulfinamide II-d can get protected with Boc₂O to tert-butyl carbamate II-e (see Maldonado et al. Tetrahedron 2012; 68:7456) and the activated with N-chlorosuccinimide and coupled to an amine (see Battula et al. Tetrahedron Lett. 2014; 55:517) to afford an intermediate, which can be processed as outlined in Scheme I above to finally afford sulfonimidamide II-f.

Sulfonyl chloride II-a can get converted to R¹¹-substituted sulfinamide II-g and then get activated with tert-butyl hypochlorite similar as outlined in US20160039846. Coupling to an amine affords an intermediate, which can be processed as outlined in Scheme I above to finally afford substituted sulfonimidamide II-h.

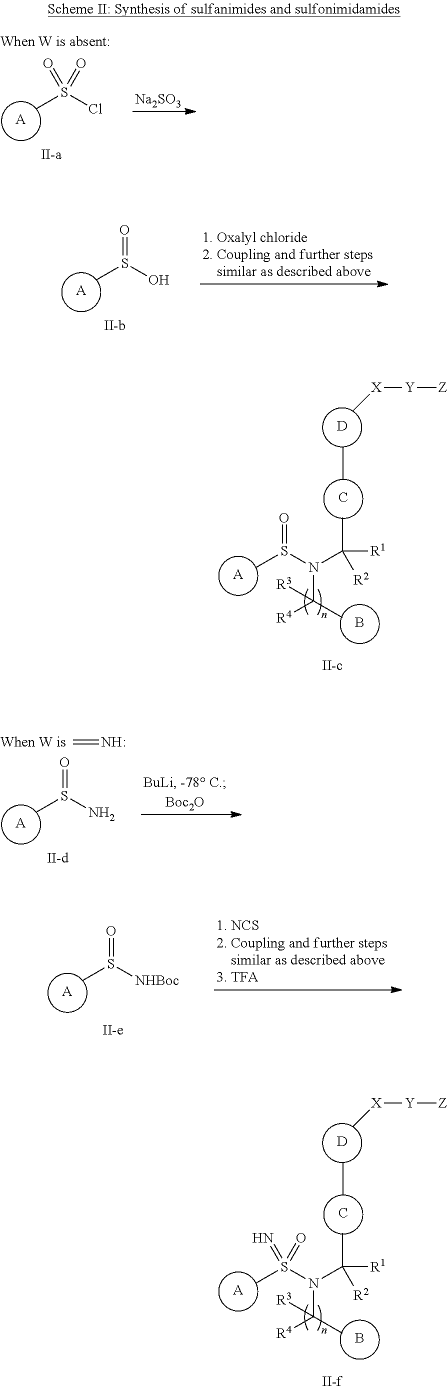

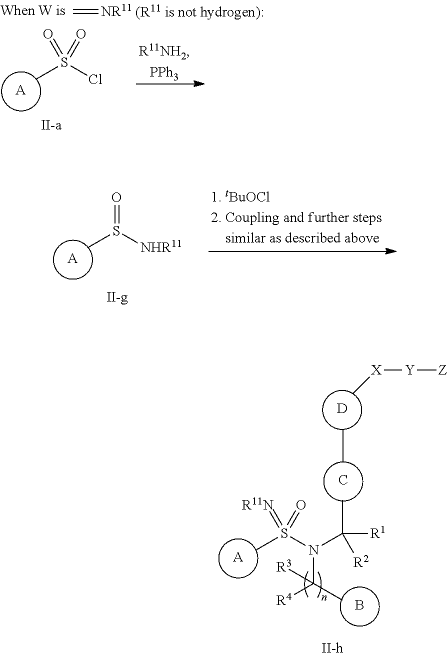

Abbreviations

Ac acetyl

ACN acetonitrile

BINAP 2,2′-bis(diphenylphosphino)-1,1′-binaphthyl

B₂Pin₂ 4,4,4′,4′,5,5,5′,5′-octamethyl-2,2′-bi-1,3,2-dioxaborolane

Boc N-tert-butoxycarbonyl

br broad (signal in NMR)

m-CPBA meta-chloroperbenzoic acid

dba dibenzylideneacetone

DCM dichloromethane

DMF N,N-dimethylformamide

dppf 1,1′-bis(diphenylphosphino)ferrocene

EA ethyl acetate

FCC flash column chromatography (on SiO₂)

NBS N-bromosuccinimide

NCS N-chlorosuccinimide

Pin pinacolato (OCMe₂CMe₂O)

PE petroleum ether

Pd/C Palladium on charcoal

rt room temperature

sat. saturated

s-phos 2-dicyclohexylphosphino-2′,6′-dimethoxybiphenyl

TBS tert-butyldimethylsilyl

TEA triethylamine

Tf trifluoromethanesulfonate (CF₃SO₃—)

TFA trifluoroacetic acid

THF tetrahydrofuran

TLC thin layer chromatography

TMS trimethylsilyl

X-phos 2-dicyclohexylphosphino-2′,4′,6′-triisopropylbiphenyl

Examples beginning with “C” (e.g. “C3/2”) are comparative examples.

### Preparative Example P1

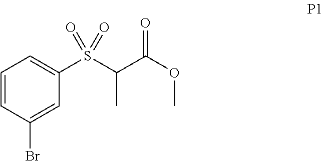 [SMILES: COC(=O)C(C)S(=O)(=O)c1cccc(Br)c1]

### Methyl 2-((3-bromophenyl)sulfonyl)propanoate (P1)

To a suspension of methyl 2-((3-bromophenyl)sulfonyl)acetate (500 mg, 1.71 mmol) and K₂CO₃ (354 mg, 2.57 mmol) in acetone (20 mL) was added MeI (0.11 mL, 1.71 mmol) at rt. The reaction mixture was stirred at 30° C. overnight and filtered. The filtrate was concentrated to give the crude compound P1 as a yellow oil. MS: 307 (M+1)⁺.

### Preparative Example P2

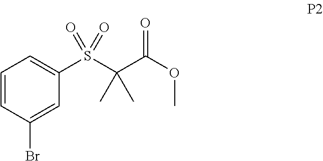 [SMILES: COC(=O)C(C)(C)S(=O)(=O)c1cccc(Br)c1]

### Methyl 2-((3-bromophenyl)sulfonyl)-2-methylpropanoate (P2)

A suspension of 2-((3-bromophenyl)sulfonyl)acetate (500 mg, 1.71 mmol) and NaH (152 mg, 60% on oil, 3.8 mmol) in dry DMF (10 mL) was stirred for 0.5 h at 0° C. and then MeI (0.7 mL, 3.77 mmol) was added to the solution at 0° C. The mixture was stirred at rt for 2 h, diluted with H₂O and extracted with EA (3×). The combined organic layer was washed with brine, dried over Na₂SO₄ and concentrated to give rude compound P2 as a yellow oil. MS: 321 (M+1)⁺.

### Preparative Example P3

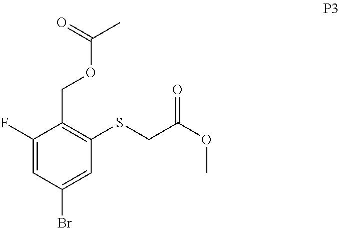 [SMILES: COC(=O)CSc1cc(Br)cc(F)c1COC(C)=O]

### Step 1: tert-Butyl 4-bromo-2,6-difluorobenzoate (P3a)

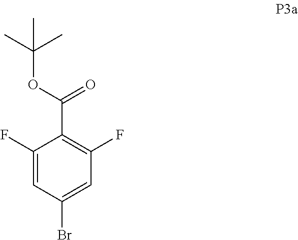 [SMILES: CC(C)(C)OC(=O)c1c(F)cc(Br)cc1F]

A mixture of 4-bromo-2,6-difluorobenzoic acid (25.0 g, 110 mmol), Boc₂O (50.0 g, 242 mmol) and 4-dimethylaminopyridine (1.3 g, 11 mmol) in tert-BuOH (200 mL) was stirred at 40° C. overnight, concentrated and purified by FCC (PE:EA=50:1) to give compound P3a as a yellow oil. MS: 292 (M+1)⁺.

### Step 2: tert-Butyl 4-bromo-2-fluoro-6-((2-methoxy-2-oxoethyl)thio)benzoate (P3b)

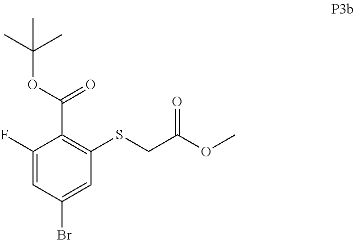 [SMILES: COC(=O)CSc1cc(Br)cc(F)c1C(=O)OC(C)(C)C]

To a solution of methyl 2-mercaptoacetate (11.2 g, 106 mmol) in dry DMF (50 mL) was added NaH (5.1 g, 60%, 127 mmol) at 0° C. The mixture was stirred 30 min. Then a solution of compound P3a (31 g, 106 mmol) in dry DMF (100 mL) was added to the mixture. The mixture was stirred at rt for 2 h, diluted with H₂O (1000 mL) and extracted with EA (3×). The combined organic layer was washed with H₂O and brine, concentrated and purified by FCC (PE:EA=10:1) to give compound P3b as a yellow oil. MS: 378 (M+1)⁺.

### Step 3: 4-Bromo-2-fluoro-6-((2-methoxy-2-oxoethyl)thio)benzoic acid (P3c)

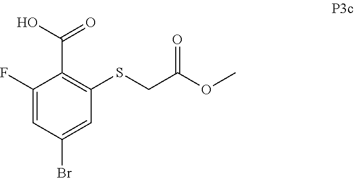 [SMILES: COC(=O)CSc1cc(Br)cc(F)c1C(=O)O]

A solution of compound P3b (18 g, 47.5 mmol) and TFA (30 mL) in DCM (60 mL) was stirred at rt overnight, concentrated in vacuo, diluted with Et₂O and stirred for 30 min. The mixture was filtered to give compound P3c as a white solid.

### Step 4: Methyl 2-((5-bromo-3-fluoro-2-(hydroxymethyl)phenyl)thio)acetate (P3d)

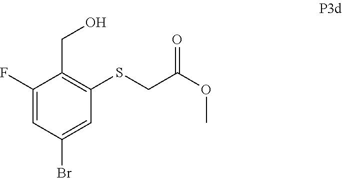 [SMILES: COC(=O)CSc1cc(Br)cc(F)c1CO]

To a solution of compound P3c (12 g, 37.3 mmol) in THF (100 mL) was added TEA (10 mL) at 0° C. Then isobutyl carbonochloridate (5.5 g, 41.0 mmol) was added slowly to the reaction mixture at 0° C. The mixture was stirred at 0° C. for 30 min, filtered and washed with THF (100 mL). The filtrate was cooled to 0° C. and NaBH₄ (2.8 g, 74.6 mmol) was added slowly. The mixture was allowed to warm to rt for 3 h. Sat. NH₄Cl (1000 mL) was added and the solution was extracted with EA (2×200 mL). The combined organic layer was successively washed with water (500 mL) and brine (200 mL), dried over Na₂SO₄, filtered, concentrated and purified by FCC (PE/EA=10:1) to give title compound P3d as a white solid. ¹H-NMR (CDCl₃, 300 MHz): δ 7.43 (t, J=1.6 Hz, 1H), 7.19 (dd, J=1.6, 8.4 Hz, 1H), 4.85 (d, J=2.0 Hz, 2H), 3.73 (s, 2H), 3.72 (s, 3H), 2.59 (br s, 1H). MS: 306.9/308.9 (M+1)⁺.

### Step 5: Methyl 2-((2-(acetoxymethyl)-5-bromo-3-fluorophenyl)thio)acetate (P3)

A solution of compound P3d (3.5 g, 11.4 mmol) in DCM (100 mL) was treated with catalytic amounts of 4-(dimethylamino)-pyridine (140 mg, 1.1 mmol) under N₂. To the mixture was added TEA (1.7 g, 17.1 mmol) and Ac₂O (1.4 g, 13.7 mmol) and the mixture was stirred at rt for 1 h, washed with 1N HCl (100 mL), water and brine, dried over Na₂SO₄, filtered and concentrated to give the crude compound P3 as a white solid which was used in the next step without further purification.

### Preparative Example P4

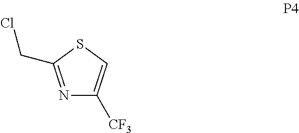 [SMILES: FC(F)(F)c1csc(CCl)n1]

### Step 1: Ethyl 4-(trifluoromethyl)thiazole-2-carboxylate (P4a)

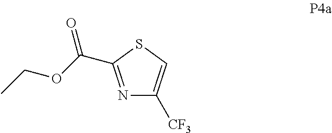

To a solution of 3-bromo-1,1,1-trifluoropropan-2-one (6.2 mL, 35 mmol) and ethyl 2-amino-2-thioxoacetate (8.0 g, 60 mmol) in EtOH (150 mL) was stirred at 85° C. overnight. The mixture was concentrated, diluted with water and extracted with EA. The organic layer was washed with brine, dried over Na₂SO₄, concentrated and purified by FCC (PE:EA=100:1 to 50:1) to give compound P4a as a yellow oil.

### Step 2: (4-(Trifluoromethyl)thiazol-2-yl)methanol (P4b)

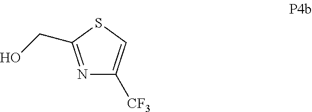 [SMILES: OCc1nc(C(F)(F)F)cs1]

To a solution of compound P4a (7.53 g, 33 mmol) in MeOH (30 mL) was added NaBH₄ (2.5 g, 66 mmol) at 0° C. The mixture was stirred for 2 h at 0° C., concentrated, diluted with water and extracted with EA. The organic layer was washed with brine, dried over Na₂SO₄, concentrated and purified by FCC (PE:EA=20:1 to 5:1) to give compound P4b as a yellow solid.

### Step 3: 2-(Chloromethyl)-4-(trifluoromethyl)thiazole (P4)

A solution of compound P4b (1.0 g, 5.5 mmol), PPh₃ (2.15 g, 8.2 mmol) and CCl₄ (10 mL) in toluene (30 mL) was stirred at 120° C. overnight, concentrated and purified by FCC (PE:EA=10:1) to give compound P4 as a yellow solid.

### Preparative Example P5

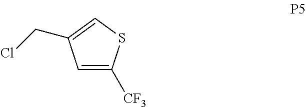 [SMILES: FC(F)(F)c1cc(CCl)cs1]

### 4-(Chloromethyl)-2-(trifluoromethyl)thiophene (P5)

To a solution of (5-(trifluoromethyl)thiophen-3-yl)methanol (500 mg, 2.74 mmol) in DCM (10 mL) was added SOCl₂ (0.60 mL, 8.22 mmol) at rt. The mixture was stirred for 8 h at rt and adjusted to pH˜8 with 1N Na₂CO₃. The organic layer was dried over Na₂SO₄, concentrated and purified by FCC (PE:EA=20:1) to give compound P5 as a yellow oil.

### Preparative Example P6

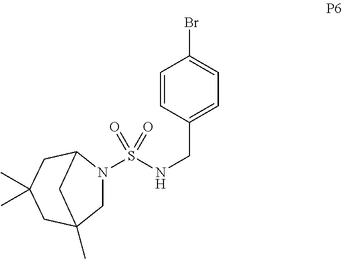 [SMILES: CC1(C)CC2CC(C)(CN2S(=O)(=O)NCc2ccc(Br)cc2)C1]

### Step 1: (4-Bromobenzyl)sulfamic acid (P6a)

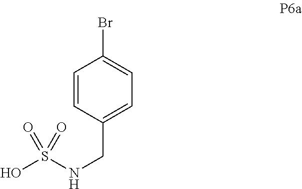 [SMILES: O=S(=O)(O)NCc1ccc(Br)cc1]

To a solution of (4-bromophenyl)methanamine (5.0 g, 26.9 mmol) in DCM (50 mL) was added HSO₃Cl (1.89 g, 16.2 mmol) at 0° C. and the mixture was stirred at rt for 0.5 h under N₂, filtered and the residue was washed with conc. HCl. The solid was dried to give the crude product P6a as a white solid.

### Step 2: (4-Bromobenzyl)sulfamoyl chloride (P6b)

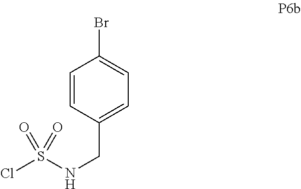 [SMILES: O=S(=O)(Cl)NCc1ccc(Br)cc1]

To a solution of crude compound P6a (5.0 g) in toluene (30 mL) was added PCl₅ (1.96 g, 9.43 mmol) and the mixture was stirred at 120° C. for 1.5 h, cooled and filtered. The filtrate was concentrated in vacuo and used for the next step directly.

### Step 3: N-(4-Bromobenzyl)-1,3,3-trimethyl-6-azabicyclo[3.2.1]octane-6-sulfonamide (P6)

To a solution of 1,3,3-trimethyl-6-azabicyclo[3.2.1]octane (600 mg, 3.92 mmol) in DCM (20 mL) was added TEA (400 mg, 3.92 mmol) and crude compound P6b. The mixture was stirred at rt overnight and filtered. The filtrate was concentrated and purified by FCC (PE:EA=5:1) to afford compound P6 as a white solid.

### Preparative Example P7 and P7-1

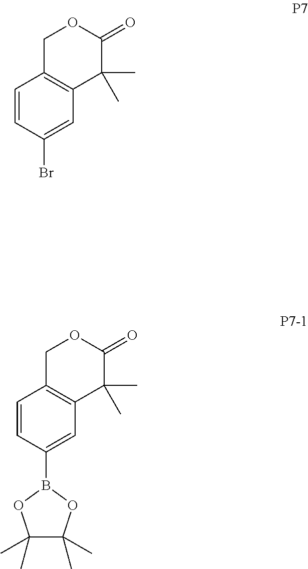

### Step 1: 4-Bromo-2-(bromomethyl)-1-methylbenzene (P7a)

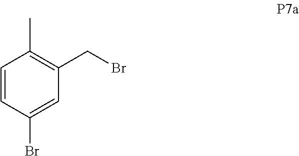

To a solution of (5-bromo-2-methylphenyl)methanol (2.7 g, 13.4 mmol) in THF (50 mL) was added PBr₃ (0.6 mL, 6.7 mmol) under ice-bath cooling. The mixture was stirred at 0° C. for 2 h, diluted with water (100 mL), basified to pH=7 with sat. NaHCO₃ and extracted with EA (3×50 mL). The combined organic layer was washed with brine (100 mL), dried over Na₂SO₄, filtered and concentrated to give compound P7a as a yellow oil.

### Step 2: 2-(5-Bromo-2-methylphenyl)acetonitrile (P7b)

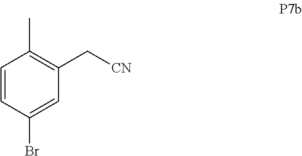

To a solution of compound P7a (3.5 g, 13.3 mmol) in DMF (50 mL) was added NaCN (715 mg, 14.6 mmol) at rt. The mixture was stirred at 60° C. for 5 h, diluted with water (100 mL) and extracted with EA (3×50 mL). The combined organic layer was washed with water (2×100 mL) and brine (100 mL), dried over Na₂SO₄, filtered and concentrated to give crude compound P7b as a white solid.

### Step 3: 2-(5-Bromo-2-methylphenyl)acetic acid (P7c)

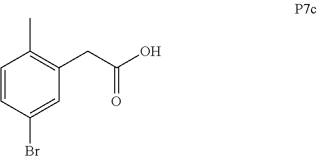

To a solution of compound P7b (1.6 g, 7.6 mmol) in water (50 mL) and EtOH (50 mL) was added KOH (4.3 g, 76 mmol) at rt. The mixture was stirred at reflux overnight, then the EtOH was evaporated and the solution was acidified to pH=3 with 1N HCl and extracted with EA (3×50 mL). The combined organic layer was washed with brine (100 mL), dried over Na₂SO₄, filtered and concentrated to give crude compound P7c as a white solid.

### Step 4: Methyl 2-(5-bromo-2-methylphenyl)acetate (P7d)

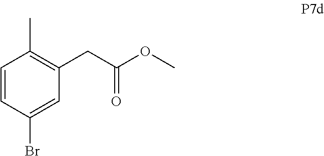

To a solution of compound P7c (1.5 g, 6.6 mmol) in MeOH (50 mL) was added conc. H₂SO₄ (0.3 mL) at rt. The mixture was stirred at reflux overnight, evaporated and dissolved in EA (50 mL) and water (20 mL). The mixture was basified to pH=7 with sat. NaHCO₃ and extracted with EA (2×50 mL). The combined organic layer was washed with brine (100 mL), dried over Na₂SO₄, filtered and concentrated to give crude compound P7d as a yellow oil.

### Step 5: Methyl 2-(5-bromo-2-methylphenyl)-2-methylpropanoate (P7e)

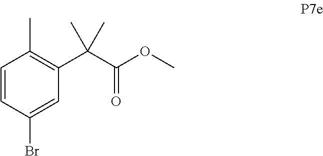

To a solution of compound P7d (9.5 g, 39.1 mmol) in dry DMF (100 mL) was added NaH (3.9 g, 60%, 98 mmol) under ice-bath cooling. The mixture was stirred for 10 min at 0° C., then 18-crown-6 (1.1 g, 7.8 mmol) and MeI (12.2 mL, 196 mmol) were added. The mixture was stirred at rt overnight, diluted with water (200 mL) and extracted with EA (3×100 mL). The combined organic layer was washed with water (2×200 mL) and brine (100 mL), dried over Na₂SO₄, filtered and evaporated. The procedure was repeated again and then the obtained residue was purified by FCC (PE:EA=20:1) to give crude compound P7e as a yellow oil.

### Step 6: Methyl 2-(5-bromo-2-(bromomethyl)phenyl)-2-methylpropanoate (P7f)

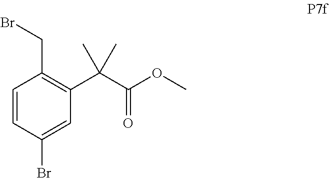 [SMILES: COC(=O)C(C)(C)c1cc(Br)ccc1CBr]

To a solution of compound P7e (9.0 g, 33.2 mmol) in CCl₄ (150 mL) was added NBS (6.5 g, 36.5 mmol) and benzoyl peroxide (799 mg, 3.3 mmol) at rt under N₂. The mixture was stirred at reflux overnight and concentrated. The residue was dissolved in EA (200 mL), washed with water (100 mL) and brine (100 mL), dried over Na₂SO₄, filtered and concentrated to give crude compound P7f as a yellow oil.

### Step 7: Methyl 2-(2-(acetoxymethyl)-5-bromophenyl)-2-methylpropanoate (P7q)

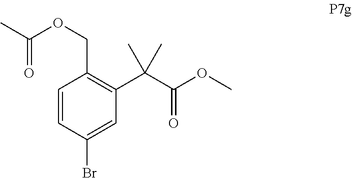 [SMILES: COC(=O)C(C)(C)c1cc(Br)ccc1COC(C)=O]

To a solution of compound P7f (11.0 g, 31.4 mmol) in DMF (100 mL) was added KOAc (6.2 g, 63 mmol) and KI (50 mg, 0.3 mmol) at rt. The mixture was stirred at rt for 2 h, diluted with water (200 mL) and extracted with EA (3×100 mL). The combined organic layer was washed with water (2×200 mL) and brine (100 mL), dried over Na₂SO₄, filtered, concentrated and purified by FCC (PE:EA=10:1) to give compound P7g as a yellow oil.

### Step 8: 6-Bromo-4,4-dimethylisochroman-3-one (P7)

To a solution of compound P7g (5.5 g, 16.7 mmol) in MeOH (50 mL) and water (50 mL) was added KOH (3.7 g, 63 mmol) at rt. The mixture was stirred at rt for 5 h and then concentrated. The residue was acidified to pH=5 with 1N HCl, stirred at rt for 1 h and then filtered. The filter cake was washed with PE/EA (20 mL, 10/1) to give compound P7 as a white solid. ¹H-NMR (CDCl₃, 400 MHz): δ 7.50 (d, J=2.0 Hz, 1H), 7.42 (dd, J=8.0, 1.6 Hz, 1H), 7.05 (d, J=8.0 Hz, 1H), 5.36 (s, 2H), 1.58 (s, 6H). MS: 255 (M+1)⁺.

### Step 9: 4,4-Dimethyl-6-(4,4,5,5-tetramethyl-1,3,2-dioxaborolan-2-yl)isochroman-3-one (P7-1)

To a solution of compound P7 (900 mg, 3.53 mmol), 4,4,4′,4′,5,5,5′,5′-octamethyl-2,2′-bi(1,3,2-dioxaborolane) (986 mg, 3.88 mmol) and KOAc (1.04 g, 10.6 mmol) in 1,4-dioxane (20 mL) was added Pd(dppf)Cl₂ (284 mg, 0.35 mmol) at rt under N₂. The mixture was stirred at 100° C. overnight, cooled, filtered, concentrated and purified by FCC (PE:EA=20:1) to give compound P7-1 as a white solid.

### Preparative Example P8

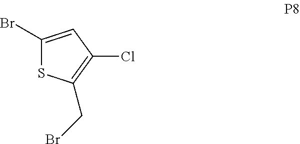

### 5-Bromo-2-(bromomethyl)-3-chlorothiophene (P8)

A mixture of (3-chlorothiophen-2-yl)methanol (500 mg, 3.36 mmol) in AcOH (30 mL) was stirred at 15° C. Then Br₂ (644 mg, 4.03 mmol) was added dropwise to the mixture. The mixture was diluted with water and extracted with EA (3×). The combined organic layer was washed with brine, dried over Na₂SO₄, filtered and concentrated to give compound P8 as a yellow oil.

### Preparative Example P9

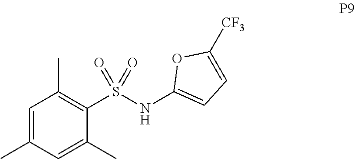 [SMILES: Cc1cc(C)c(S(=O)(=O)Nc2ccc(C(F)(F)F)o2)c(C)c1]

### Step 1: tert-Butyl (5-(trifluoromethyl)furan-2-yl)carbamate (P9a)

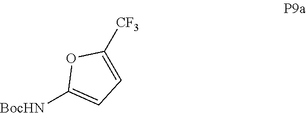 [SMILES: CC(C)(C)OC(=O)Nc1ccc(C(F)(F)F)o1]

A solution of 5-(trifluoromethyl)furan-2-carboxylic acid (1.0 g, 5.5 mmol), diphenylphosphoryl azide (2.4 mL, 11 mmol) and TEA (0.8 mL, 11 mmol) in tert-butanol (15 mL) was refluxed overnight, concentrated and purified by FCC (PE:EA=40:1) to give compound P9a as a yellow oil.

### Step 2: tert-Butyl (mesitylsulfonyl)(5-(trifluoromethyl)furan-2-yl)carbamate (P9b)

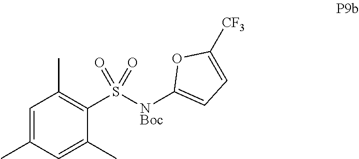 [SMILES: Cc1cc(C)c(S(=O)(=O)N(C(=O)OC(C)(C)C)c2ccc(C(F)(F)F)o2)c(C)c1]

To a suspension of NaH (180 mg, 60%, 4.4 mmol) in dry DMF (15 mL) was added compound P9a (550 mg, 2.2 mmol). After the mixture was stirred for 30 min, 2,4,6-trimethylbenzene-sulfonyl chloride (480 mg, 2.2 mmol) was added. The mixture was stirred at rt for 2 h, diluted with H₂O (100 mL) and extracted with EA (3×). The combined organic layer was washed with brine, dried over Na₂SO₄, filtered and purified by FCC (PE:EA=100:1) to give compound P9b as a yellow solid.

### Step 3: 2,4,6-Trimethyl-N-(5-(trifluoromethyl)furan-2-yl)benzenesulfonamide (P9)

To a mixture of compound P9b (138 mg, 0.32 mmol) in DCM (20 mL) was added TFA (1.5 mL). The mixture was stirred at rt for 2 h and concentrated to give compound P9 as a yellow oil which was used to the next step without further purification.

### Preparative Example P10

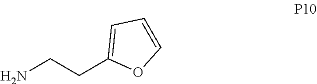

### Step 1: (E)-2-(2-Nitrovinyl)furan (P10a)

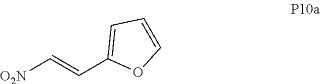

To a solution of furan-2-carbaldehyde (50 g, 0.52 mol) in MeOH (100 mL) was added nitro-methane (70 mL, 1.30 mol) and 1N NaOH (1.3 L) dropwise at 0° C. Then ice/water (250 mL) was added. The mixture was stirred at 0° C. for 30 min. The mixture was added slowly to 8.0M HCl (500 mL) at 0° C. until the reaction was completed. The mixture was filtered to afford compound P10a as a yellow solid.

### Step 2: 2-(Furan-2-yl)ethan-1-amine (P10)

To a solution of compound P10a (63.0 g, 0.45 mol) in dry THF (400 mL) was added LiAlH₄ (69 g, 1.81 mol) at 0° C. The mixture was stirred for 2 h at 0° C. To the mixture was added H₂O (69 mL), 10% NaOH (69 mL) and H₂O (207 mL) at 0° C. The mixture was filtered, concentrated and purified by FCC (PE:EA=5:1 to 1:1) to give compound P10 as yellow oil.

### Preparative Example P11

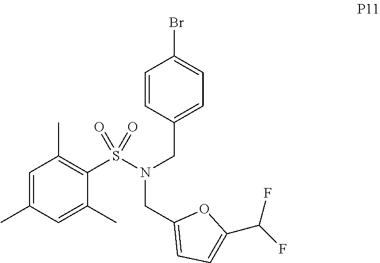 [SMILES: Cc1cc(C)c(S(=O)(=O)N(Cc2ccc(Br)cc2)Cc2ccc(C(F)F)o2)c(C)c1]

### Step 1: N-(4-Bromobenzyl)-N-((5-formylfuran-2-yl)methyl)-2,4,6-trimethylbenzenesulfonamide (P11a)

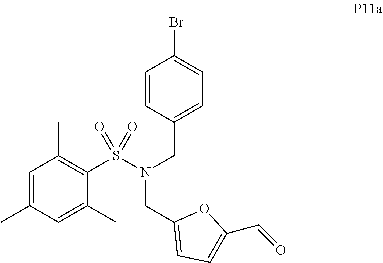 [SMILES: Cc1cc(C)c(S(=O)(=O)N(Cc2ccc(Br)cc2)Cc2ccc(C=O)o2)c(C)c1]

To a solution of 5-(chloromethyl)furan-2-carbaldehyde (310 mg, 2.14 mmol) and compound 1a (786 mg, 2.14 mmol) in ACN (20 mL) was added K₂CO₃ (591 mg, 4.28 mmol) and KI (355 mg, 2.14 mmol) at rt. The mixture was stirred at 80° C. overnight under N₂, cooled, filtered, concentrated and purified by FCC (PE:EA=20:1 to 10:1) to give compound P11a as a yellow solid.

### Step 2: N-(4-Bromobenzyl)-N-((5-(difluoromethyl)furan-2-yl)methyl)-2,4,6-trimethylbenzene-sulfonamide (P11)

To a solution of compound P11a (600 mg, 1.3 mmol) in DCM (20 mL) was added diethyl-aminosulfur trifluoride (1.6 mL, 12.6 mmol) at 0° C. The mixture was stirred at 0° C. for 0.5 h and then stirred at 30° C. overnight, quenched with NaHCO₃ and extracted with DCM. The organic layer was washed with brine, dried over Na₂SO₄, concentrated and purified by FCC (PE:EA=20:1) to give compound P11 as a yellow solid.

### Example 1

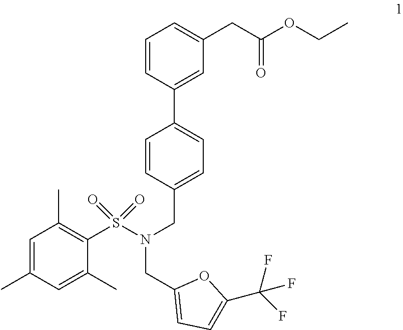 [SMILES: CCOC(=O)Cc1cccc(-c2ccc(CN(Cc3ccc(C(F)(F)F)o3)S(=O)(=O)c3c(C)cc(C)cc3C)cc2)c1]

### Step 1: N-(4-Bromobenzyl)-2,4,6-trimethylbenzenesulfonamide (1a)

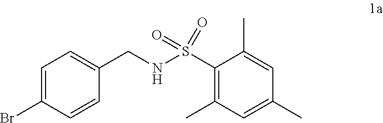 [SMILES: Cc1cc(C)c(S(=O)(=O)NCc2ccc(Br)cc2)c(C)c1]

To a solution of 2,4,6-trimethylbenzenesulfonyl chloride (5.86 g, 27 mmol) and TEA (4.1 g, 40 mmol) in DCM (100 mL) was added (4-bromophenyl)methanamine (5.0 g, 27 mmol) portionwise. The mixture was allowed to stir for 1 h at rt, washed with HCl (2N, 100 mL), water and brine. The organic layer was dried over Na₂SO₄ and concentrated to obtain compound 1a. ¹H-NMR (CDCl₃, 300 MHz): δ 7.38-7.35 (m, 2H), 7.05-7.02 (m, 2H), 6.94 (s, 2H), 4.76 (t, J=6.0 Hz, 1H), 4.04 (d, J=6.0 Hz, 2H), 2.62 (s, 6H), 2.31 (s, 3H).

### Step 2: Ethyl 2-(4′-(((2,4,6-trimethylphenyl)sulfonamido)methyl)-[1,1′-biphenyl]-3-yl)acetate (1b)

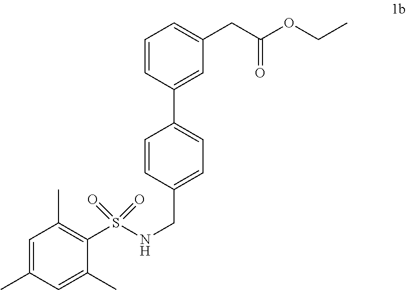 [SMILES: CCOC(=O)Cc1cccc(-c2ccc(CNS(=O)(=O)c3c(C)cc(C)cc3C)cc2)c1]

To a suspension of compound 1a (150 mg, 0.41 mmol), ethyl 2-(3-(4,4,5,5-tetramethyl-1,3,2-dioxaborolan-2-yl)phenyl)acetate (237 mg, 0.82 mmol), s-phos (33 mg, 80 μmol) and K₃PO₄ (354 mg, 1.63 mmol) in ethylene glycol dimethyl ether/H₂O (15 mL/0.5 mL) was added Pd₂dba₃ (9 mg, 10 μmol) under N₂. The mixture was stirred at 110° C. overnight, cooled, filtered, concentrated and purified by FCC (PE:EA=5:1) to afford compound 1b as a yellow oil. ¹H-NMR (CDCl₃, 300 MHz): δ 7.49-7.26 (m, 6H), 7.23 (d, J=8.4 Hz, 2H), 6.96 (s, 2H), 4.76 (t, J=6.0 Hz, 1H), 4.20-4.11 (m, 4H), 3.67 (s, 2H), 2.65 (s, 6H), 2.30 (s, 3H), 1.26 (t, J=7.2 Hz, 3H).

### Step 3: Ethyl 2-(4′-(((2,4,6-trimethyl-N-((5-(trifluoromethyl)furan-2-yl)methyl)phenyl)sulfon-amido)methyl)-[1,1′]-biphenyl]-3-yl)acetate (1)

A solution of compound 1b (113 mg, 0.25 mmol), 2-(bromomethyl)-5-(trifluoromethyl)furan (63 mg, 0.28 mmol) and Cs₂CO₃ (163 mg, 0.50 mmol) in DMF (50 mL) was stirred at rt overnight, diluted with water (50 mL) and extracted with EA (3×50 mL). The combined organic layer was washed with water (2×50 mL), dried over MgSO₄, concentrated and purified by FCC (PE:EA=10:1) to afford compound 1 as a yellow oil. ¹H-NMR (CDCl₃, 300 MHz): δ 7.53-7.34 (m, 6H), 7.19 (d, J=7.8 Hz, 2H), 6.99 (s, 2H), 6.65 (d, J=3.3 Hz, 1H), 6.22 (d, J=3.3 Hz, 1H), 4.36 (s, 2H), 4.27 (s, 2H), 4.17 (q, J=7.2 Hz, 2H), 3.67 (s, 2H), 2.64 (s, 6H), 2.32 (s, 3H), 1.27 (t, J=7.2 Hz, 3H). MS: 598.1 (M−1)⁻.

### Example 2

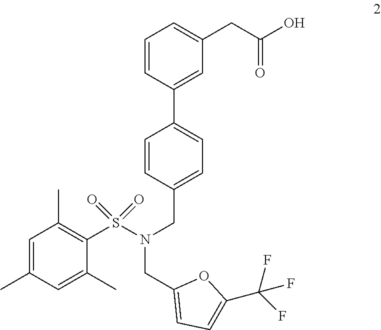 [SMILES: Cc1cc(C)c(S(=O)(=O)N(Cc2ccc(-c3cccc(CC(=O)O)c3)cc2)Cc2ccc(C(F)(F)F)o2)c(C)c1]

### 2-(4′-(((2,4,6-Trimethyl-N-((5-(trifluoromethyl)furan-2-yl)methyl)phenyl)sulfonamido)methyl)-[1,1′-biphenyl]-3-yl)acetic acid (2)

To a solution of compound 1 (116 mg, 0.19 mmol) in THF (10 mL) and water (4 mL) was added LiOH.H₂O (18 mg, 0.43 mmol) and the reaction was stirred at rt overnight, acidified with HCl (2N, 10 mL) and extracted with EA (3×10 mL). The combined organic layer was dried over Na₂SO₄ and concentrated to give compound 2 as a white solid. ¹H-NMR (DMSO-d₆, 300 MHz): δ 7.55 (d, J=6.3 Hz, 2H), 7.50 (s, 1H), 7.45 (d, J=5.7 Hz, 1H), 7.35 (t, J=5.7 Hz, 1H), 7.24 (s, 1H), 7.21 (d, J=6.3 Hz, 2H), 7.06 (s, 2H), 7.02 (d, J=2.2 Hz, 1H), 6.37 (d, J=2.2 Hz, 1H), 4.36 (s, 2H), 4.32 (s, 2H), 3.52 (s, 2H), 2.55 (s, 6H), 2.27 (s, 3H). MS: 570.1 (M−1)⁻.

### Example 2/1 to 2/4

The following Examples were prepared similar as described for Example 1 and 2 using the appropriate building blocks.

| # | building block | structure | analytical data |
|---|---|---|---|
| 2/1 | 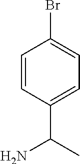 | 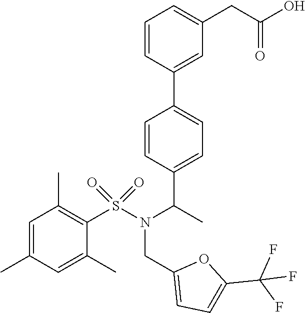 [SMILES: Cc1cc(C)c(S(=O)(=O)N(Cc2ccc(C(F)(F)F)o2)C(C)c2ccc(-c3cccc(CC(=O)O)c3)cc2)c(C)c1] | ¹H-NMR (DMSO-d₆, 300 MHz): δ 1.53 (d, J = 6.9 Hz, 3H), 2.26 (s, 3H), 2.55 (s, 6H), 3.64 (s, 2H), 4.33-4.46 (m, 2H), 5.08 (q, J = 6.9 Hz, 1H), 6.05 (d, J = 3.0 Hz, 1H), 6.81 (d, J = 1.8 Hz, 1H), 7.03 (s, 2H), 7.25 (d, J = 7.5 Hz, 1H), 7.32-7.43 (m, 3H), 7.48-7.55 (m, 4H), 12.28 (br s, 1H). MS: 584.1 (M − 1)⁻. |
| 2/2 | 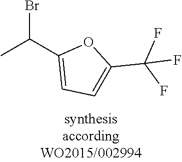 | 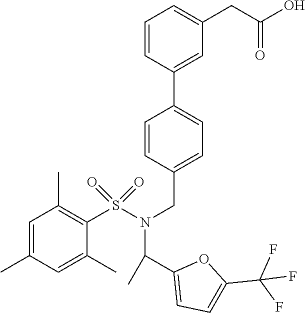 [SMILES: Cc1cc(C)c(S(=O)(=O)N(Cc2ccc(-c3cccc(CC(=O)O)c3)cc2)C(C)c2ccc(C(F)(F)F)o2)c(C)c1] | ¹H-NMR (CDCl₃, 400 MHz): δ 7.33-7.38 (m, 3H), 7.22-7.30 (m, 3H), 7.04 (d, J = 8.0 Hz, 2H), 6.89 (s, 2H), 6.55 (d, J = 1.6 Hz, 1H), 6.14 (d, J = 2.8 Hz, 1H), 5.19 (q, J = 7.2 Hz, 1H), 4.50 (d, J = 15.6 Hz, 1H), 4.17 (d, J = 15.6 Hz, 1H), 3.68 (s, 2H), 2.65 (s, 6H), 2.24 (s, 3H), 1.52 (d, J = 7.2 Hz, 3H). MS: 584.2 (M − H)⁻. |
| 2/3 | 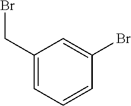 | 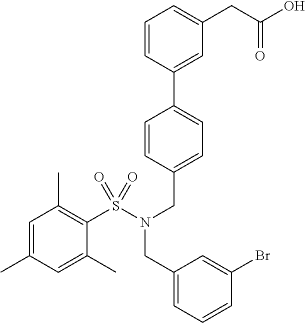 [SMILES: Cc1cc(C)c(S(=O)(=O)N(Cc2ccc(-c3cccc(CC(=O)O)c3)cc2)Cc2cccc(Br)c2)c(C)c1] | ¹H-NMR (DMSO-d₆, 300 MHz): δ 7.46-7.42 (m, 5H), 7.36 (t, J = 7.5 Hz, 1H), 7.26-7.21 (m, 2H), 7.14-7.04 (m, 6H), 4.31 (s, 2H), 4.26 (s, 2H), 3.55 (s, 2H), 2.55 (s, 6H), 2.30 (s, 3H). MS: 590.2/592.0 (M − 1)⁻. |
| 2/4 | 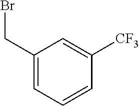 [SMILES: FC(F)(F)c1cccc(CBr)c1] | 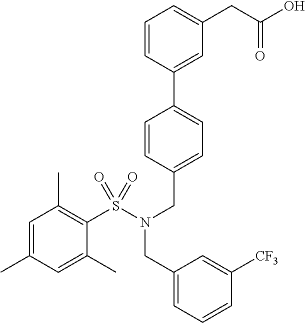 [SMILES: Cc1cc(C)c(S(=O)(=O)N(Cc2ccc(-c3cccc(CC(=O)O)c3)cc2)Cc2cccc(C(F)(F)F)c2)c(C)c1] | ¹H-NMR (CD₃OD, 300 MHz): δ 7.53-7.51 (m, 4H), 7.46-7.33 (m, 4H), 7.27 (d, J = 7.5 Hz, 1H), 7.20-7.13 (m, 3H), 7.08 (s, 2H), 4.37 (s, 2H), 4.32 (s, 2H), 3.67 (s, 2H), 2.63 (s, 6H), 2.33 (s, 3H). |

### Example 3

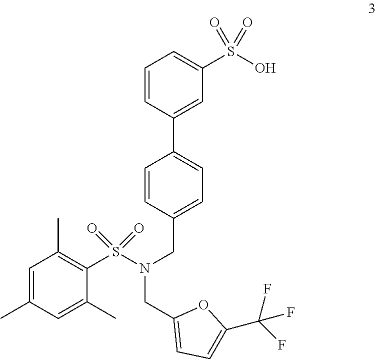 [SMILES: Cc1cc(C)c(S(=O)(=O)N(Cc2ccc(-c3cccc(S(=O)(=O)O)c3)cc2)Cc2ccc(C(F)(F)F)o2)c(C)c1]

### Step 1: N-(4-Bromobenzyl)-2,4,6-trimethyl-N-((5-(trifluoromethyl)furan-2-yl)methyl)benzene-sulfonamide (3a)

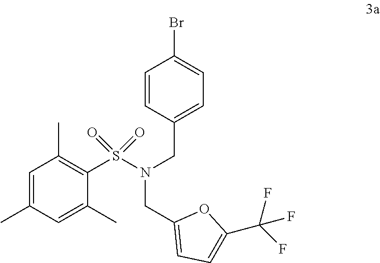 [SMILES: Cc1cc(C)c(S(=O)(=O)N(Cc2ccc(Br)cc2)Cc2ccc(C(F)(F)F)o2)c(C)c1]

A mixture of N-(4-bromobenzyl)-2,4,6-trimethylbenzenesulfonamid 1a (5.5 g, 14.9 mmol), 2-(bromomethyl)-5-(trifluoromethyl)furan (9.0 g, 43.3 mmol) and K₂CO₃ (4.0 g, 28.8 mmol) in acetone (100 mL) was heated to 65° C. overnight, cooled and filtered. The filtrate was concentrated and purified by FCC (PE:EA=20:1) to give compound 3a as a yellow solid.

### Step 2: 2,4,6-Trimethyl-N-(4-(4,4,5,5-tetramethyl-1,3,2-dioxaborolan-2-yl)benzyl)-N-((5-(tri-fluoromethyl)furan-2-yl)methyl)benzenesulfonamide (3b)

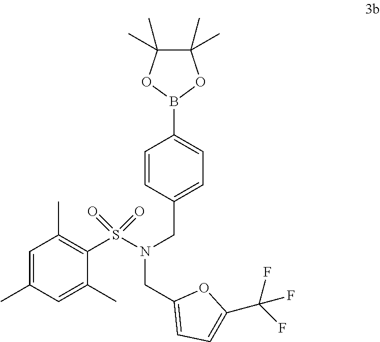 [SMILES: Cc1cc(C)c(S(=O)(=O)N(Cc2ccc(B3OC(C)(C)C(C)(C)O3)cc2)Cc2ccc(C(F)(F)F)o2)c(C)c1]

To a solution of compound 3a (500 mg, 0.97 mmol) in dioxane (10 mL) was added B₂Pin₂ (271 mg, 1.06 mmol), KOAc (285 mg, 2.90 mmol) and Pd(dppf)Cl₂ (71 mg, 0.10 mmol). The mixture was stirred at reflux under N₂ overnight, cooled to rt, concentrated and purified by FCC (PE:EA=20:1) to afford compound 3b as a white solid. ¹H-NMR (CDCl₃, 300 MHz): δ 7.73 (d, J=8.1 Hz, 2H), 7.09 (d, J=8.1 Hz, 2H), 6.96 (s, 2H), 6.64 (d, J=3.3 Hz, 1H), 6.22 (d, J=3.3 Hz, 1H), 4.31 (s, 2H), 4.22 (s, 2H), 2.61 (s, 6H), 2.31 (s, 3H), 1.33 (s, 12H).

### Step 3: 4′-(((2,4,6-Trimethyl-N-((5-(trifluoromethyl)furan-2-yl)methyl)phenyl)sulfon-amido)methyl)-[1,1′-biphenyl]-3-sulfonic acid (3)

To a solution of compound 3b (800 mg, 1.42 mmol), sodium 3-bromobenzenesulfonate (368 mg, 1.42 mmol) and Pd(PPh₃)₄ (160 mg 0.14 mmol) in dioxane (20 mL) and water (5 mL) was added Na₂CO₃ (451 mg, 4.25 mmol) under N₂. The mixture was refluxed overnight, cooled, adjusted pH to 4 with 1N HCl and extracted with EA (3×10 mL). The combined organic layer was washed with brine, dried over Na₂SO₄, concentrated and purified by prep-HPLC to afford compound 3 as a white solid. ¹H-NMR (DMSO-d₆, 300 MHz): δ 7.80 (s, 1H), 7.58-7.51 (m, 4H), 7.42-7.39 (m, 1H), 7.22-7.19 (m, 2H), 7.05-7.00 (m, 3H), 6.38 (d, J=3.9 Hz, 1H), 4.35 (s, 2H), 4.32 (s, 2H), 2.53 (s, 6H), 2.25 (s, 3H). MS: 594.1 (M+1)⁺.

### Example 3/1 and Comparative Example C3/2

The following Examples were prepared similar as described for Example 3 using the appropriate building blocks.

| # | building block | structure | analytical data |
|---|---|---|---|
| 3/1 | 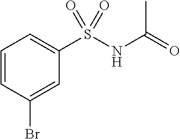 | 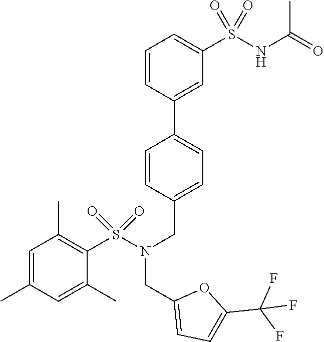 [SMILES: CC(=O)NS(=O)(=O)c1cccc(-c2ccc(CN(Cc3ccc(C(F)(F)F)o3)S(=O)(=O)c3c(C)cc(C)cc3C)cc2)c1] | ¹H-NMR (DMSO-d₆, 300 MHz): δ 12.11 (s, 1H), 8.07 (s, 1H), 7.97- 7.87 (m, 2H), 7.73-7.68 (m, 1H), 7.60-7.58 (m, 2H), 7.29-7.27 (m, 2H), 7.05-7.00 (m, 3H), 6.37 (d, J = 3.3 Hz, 1H), 4.39 (s, 2H), 4.32 (s, 2H), 2.54 (s, 6H), 2.25 (s, 3H), 1.92 (s, 3H). MS: 633.1 (M − 1)⁻. |
| C3/2 | 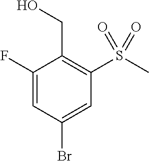 | 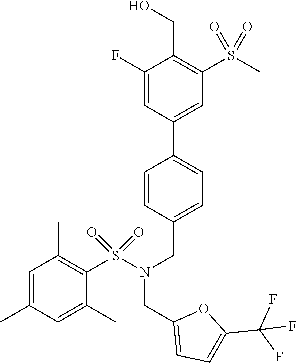 [SMILES: Cc1cc(C)c(S(=O)(=O)N(Cc2ccc(-c3cc(F)c(CO)c(S(C)(=O)=O)c3)cc2)Cc2ccc(C(F)(F)F)o2)c(C)c1] | ¹H-NMR (CD₃OD, 300 MHz): δ 8.11 (s, 1H), 7.78 (d, J = 10.2 Hz, 1H), 7.64-7.61 (m, 2H), 7.31 (d, J = 8.1 Hz, 2H), 7.05 (s, 2H), 6.79 (d, J = 1.8 Hz, 1H), 6.28 (d, J = 2.4 Hz, 1H), 5.10 (s, 2H), 4.45 (s, 2H), 4.33 (s, 2H), 3.36 (s, 3H), 2.62 (s, 6H), 2.31 (s, 3H). MS: 640.2 (M + 1)⁺. |

### Example 4

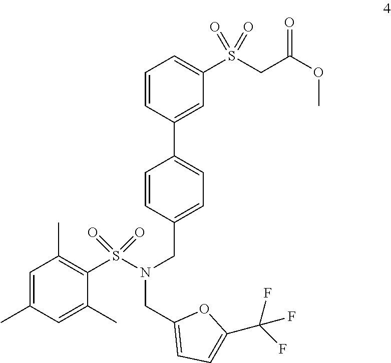

### Methyl 2-((4′-(((2,4,6-trimethyl-N-((5-(trifluoromethyl)furan-2-yl)methyl)phenyl)sulfon-amido)methyl)-[1,1′-biphenyl]-3-yl)sulfonyl)acetate (4)

A solution of compound 3b (732 mg, 1.30 mmol), methyl 2-((3-bromophenyl)sulfonyl)acetate (380 mg, 1.30 mmol), K₃PO₄ (839 mg, 3.90 mmol), PPh₃ (52 mg, 0.20 mmol) and Pd₂(dba)₃ (60 mg, 65 μmol) in dioxane (50 mL) under N₂ was refluxed at 120° C. overnight, cooled and filtered. The filtrate was concentrated and purified by FCC to obtain compound 4 as a yellow oil. ¹H-NMR (CDCl₃, 300 MHz): δ 8.13 (s, 1H), 7.87-7.94 (m, 2H), 7.67 (t, J=7.8 Hz, 1H), 7.56 (d, J=8.4 Hz, 2H), 7.26-7.28 (m, 2H), 7.00 (s, 2H), 6.66 (d, J=3.0 Hz, 1H), 6.22 (d, J=3.6 Hz, 1H), 4.40 (s, 2H), 4.27 (s, 2H), 4.17 (s, 2H), 3.73 (s, 3H), 2.65 (s, 6H), 2.33 (s, 3H). MS: 650.2 (M+1)⁺.

### Example 5

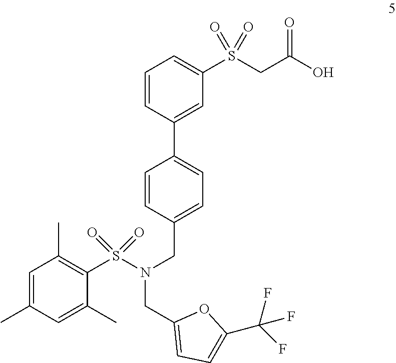 [SMILES: Cc1cc(C)c(S(=O)(=O)N(Cc2ccc(-c3cccc(S(=O)(=O)CC(=O)O)c3)cc2)Cc2ccc(C(F)(F)F)o2)c(C)c1]

### 2-((4′-(((2,4,6-Trimethyl-N-((5-(trifluoromethyl)furan-2-yl)methyl)phenyl)sulfonamido)methyl)-[1,1′-biphenyl]-3-yl)sulfonyl)acetic acid (5)

A solution of compound 4 (60 mg, 92 μmol) and LiOH.H₂O (7.7 mg, 184 μmol) in THF (10 mL) and water (10 mL) was stirred at rt overnight, concentrated, adjusted to pH 5-6 with 1N HCl and filtered to obtain compound 5 as a white solid. ¹H-NMR (DMSO-d₆, 300 MHz): δ 8.13 (s, 1H), 7.97-8.00 (m, 1H), 7.89 (d, J=7.5 Hz, 1H), 7.66-7.74 (m, 3H), 7.27-7.30 (m, 2H), 7.03-7.07 (m, 3H), 6.38-6.40 (m, 1H), 4.41 (s, 4H), 4.34 (s, 2H), 2.56 (s, 6H), 2.26 (s, 3H). MS: 590.1 (M−CO₂H)⁻.

### Example 5/1 to 5/5, Comparative Example C5/6 and Example 5/7

The following Examples were prepared similar as described for Example 4 using the appropriate building blocks and saponified as described in Example 5.

| # | building block(s) | structure | analytical data |
|---|---|---|---|
| 5/1 | 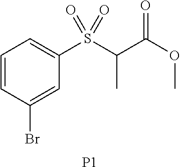 | 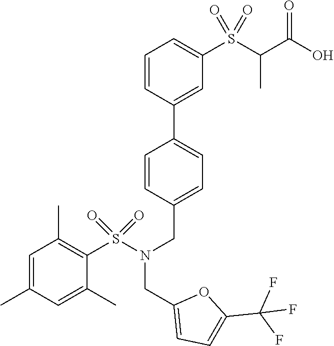 [SMILES: Cc1cc(C)c(S(=O)(=O)N(Cc2ccc(-c3cccc(S(=O)(=O)C(C)C(=O)O)c3)cc2)Cc2ccc(C(F)(F)F)o2)c(C)c1] | ¹H-NMR (CD₃OD, 400 MHz): δ 8.09 (t, J = 1.6 Hz, 1H), 7.94 (dd, J = 1.6, 7.6 Hz, 1H), 7.90-7.88 (m, 1H), 7.68 (t, J = 7.6 Hz, 1H), 7.58 (d, J = 8.8 Hz, 2H), 7.27 (d, J = 8.4 Hz, 2H), 7.04 (s, 2H), 6.79 (dd, J = 1.2, 3.2 Hz, 1H), 6.27 (d, J = 2.8 Hz, 1H), 4.42 (s, 2H), 4.32 (s, 2H), 4.19-4.16 (m, 1H), 2.61 (s, 6H), 2.30 (s, 3H), 1.51 (d, J = 7.2 Hz, 3H). MS: 650.1 (M + 1)⁺. |
| 5/2 | 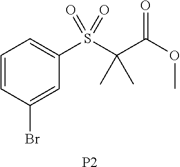 | 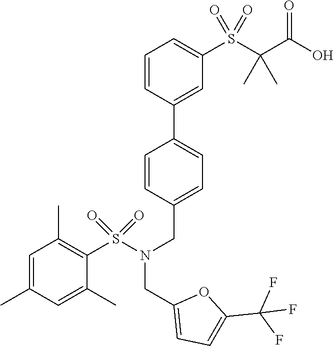 [SMILES: Cc1cc(C)c(S(=O)(=O)N(Cc2ccc(-c3cccc(S(=O)(=O)C(C)(C)C(=O)O)c3)cc2)Cc2ccc(C(F)(F)F)o2)c(C)c1] | ¹H-NMR (CD₃OD, 400 MHz): δ 8.04 (t, J = 1.6 Hz, 1H), 7.98-7.96 (m, 1H), 7.88-7.86 (m, 1H), 7.69 (d, J = 7.8 Hz, 2H), 7.59 (d, J = 8.0 Hz, 2H), 7.30 (d, J = 8.4 Hz, 2H), 7.05 (s, 2H), 6.80 (dd, J = 1.6, 3.2 Hz, 1H), 6.27 (d, J = 3.2 Hz, 1H), 4.44 (s, 2H), 4.34 (s, 2H), 2.62 (s, 6H), 2.31 (s, 3H), 1.59 (s, 6H). MS: 664.2 (M + 1)⁺. |
| 5/3 | 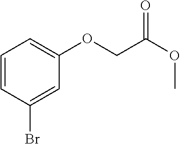 | 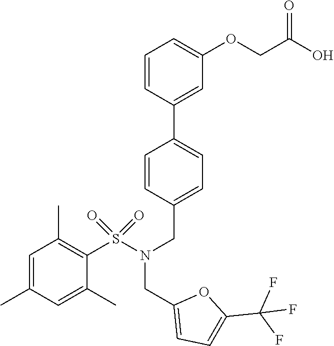 [SMILES: Cc1cc(C)c(S(=O)(=O)N(Cc2ccc(-c3cccc(OCC(=O)O)c3)cc2)Cc2ccc(C(F)(F)F)o2)c(C)c1] | ¹H-NMR (CD₃OD, 300 MHz): δ 7.54 (d, J = 8.1 Hz, 2H), 7.36 (t, J = 8.1 Hz, 1H), 7.22- 7.14 (m, 4H), 7.06 (s, 2H), 6.93 (dd, J = 1.5, 8.1 Hz, 1H), 6.80 (s, 1H), 6.28 (d, J = 2.7 Hz, 1H), 4.61 (s, 2H), 4.39 (s, 2H), 4.32 (s, 2H), 2.62 (s, 6H), 2.32 (s, 3H). MS: 586.1 (M − 1)⁻. |
| 5/4 | 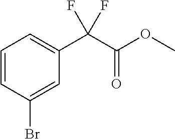 | 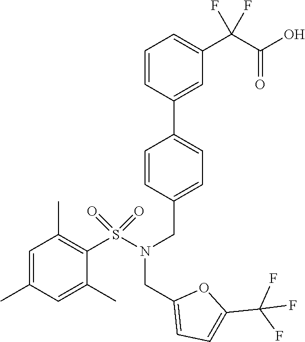 [SMILES: Cc1cc(C)c(S(=O)(=O)N(Cc2ccc(-c3cccc(C(F)(F)C(=O)O)c3)cc2)Cc2ccc(C(F)(F)F)o2)c(C)c1] | ¹H-NMR (CDCl₃, 400 MHz): δ 7.69 (s, 1H), 7.41 (br s, 2H), 7.35 (d, J = 8.0 Hz, 2H), 7.22-7.18 (m, 1H), 7.12 (d, J = 8.0 Hz, 2H), 6.90 (s, 2H), 6.53 (d, J = 2.4 Hz, 1H), 6.03 (d, J = 3.2 Hz, 1H), 4.29 (s, 2H), 4.06 (s, 2H), 2.53 (s, 6H), 2.25 (s, 3H). MS: 606.1 (M − 1)⁻. |
| 5/5 | 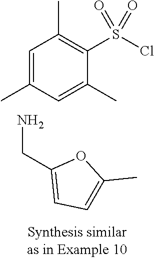 | 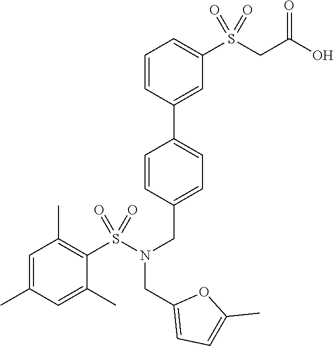 [SMILES: Cc1cc(C)c(S(=O)(=O)N(Cc2ccc(-c3cccc(S(=O)(=O)CC(=O)O)c3)cc2)Cc2ccc(C)o2)c(C)c1] | ¹H-NMR (CDCl₃, 400 MHz): δ 8.07 (s, 1H), 7.87-7.85 (m, 1H), 7.70 (d, J = 7.2 Hz, 1H), 7.48-7.43 (m, 3H), 7.20 (d, J = 8.0 Hz, 2H), 6.93 (s, 2H), 5.87 (d, J = 2.8 Hz, 1H), 5.77 (d, J = 2.4 Hz, 1H), 4.32 (s, 2H), 4.16 (br s, 2H), 4.07 (s, 2H), 2.58 (s, 6H), 2.28 (s, 3H), 2.13 (s, 3H). MS: 582.5 (M + 1)⁺. |
| C5/6 | 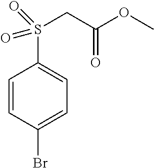 | 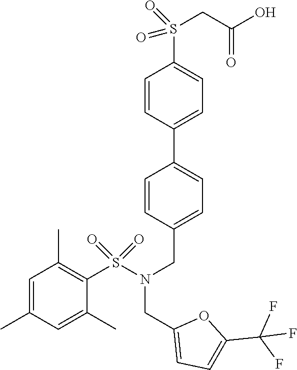 [SMILES: Cc1cc(C)c(S(=O)(=O)N(Cc2ccc(-c3ccc(S(=O)(=O)CC(=O)O)cc3)cc2)Cc2ccc(C(F)(F)F)o2)c(C)c1] | ¹H-NMR (CDCl₃, 400 MHz): δ 8.02 (d, J = 8.0 Hz, 2H), 7.74 (d, J = 8.0 Hz, 2H), 7.55 (d, J = 8.0 Hz, 2H), 7.29 (d, J = 8.0 Hz, 2H), 6.99 (s, 2H), 6.64 (s, 1H), 6.18 (s, 1H), 4.41 (s, 2H), 4.24 (s, 2H), 4.20 (s, 2H), 2.63 (s, 6H), 2.32 (s, 3H). MS: 636.2 (M + H)⁺. |
| 5/7 | 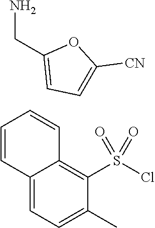 | 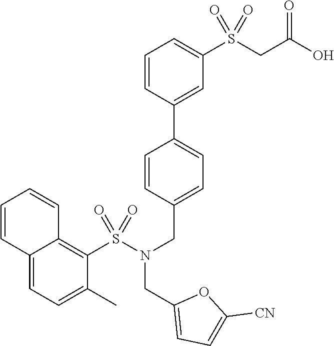 [SMILES: Cc1ccc2ccccc2c1S(=O)(=O)N(Cc1ccc(-c2cccc(S(=O)(=O)CC(=O)O)c2)cc1)Cc1ccc(C#N)o1] | ¹H-NMR (CDCl₃, 400 MHz): δ 8.69 (d, J = 8.8 Hz, 1H), 7.94 (s, 1H), 7.88 (d, J = 8.4 Hz, 1H), 7.81-7.78 (m, 2H), 7.56-7.47 (m, 3H), 7.34-7.26 (m, 4H), 6.99 (d, J = 8.0 Hz, 2H), 6.66 (d, J = 3.6 Hz, 1H), 5.91 (d, J = 3.6 Hz, 1H), 4.35 (s, 2H), 4.16 (s, 2H), 4.14 (s, 2H), 2.83 (s, 3H). MS: 615.0 (M + 1)⁺. |

### Comparative Example C6

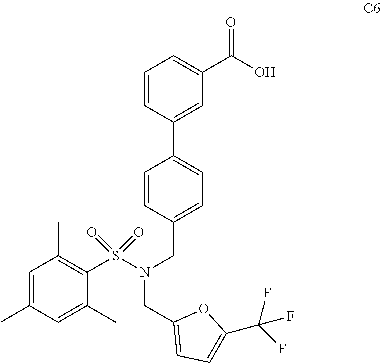 [SMILES: Cc1cc(C)c(S(=O)(=O)N(Cc2ccc(-c3cccc(C(=O)O)c3)cc2)Cc2ccc(C(F)(F)F)o2)c(C)c1]

### 4′-(((2,4,6-Trimethyl-N-((5-(trifluoromethyl)furan-2-yl)methyl)phenyl)sulfonamido)methyl)-[1,1′-biphenyl]-3-carboxylic acid (C6)

A solution of compound 3a (515 mg, 1.00 mmol), 3-(4,4,5,5-tetramethyl-1,3,2-dioxaborolan-2-yl)benzoic acid (298 mg, 1.20 mmol), K₃PO₄ (645 mg, 3.00 mmol), PPh₃ (39 mg, 0.15 mmol) and Pd₂(dba)₃ (46 mg, 50 μmol) in dioxane (50 mL) under N₂ was stirred at 120° C. overnight, cooled, adjusted to pH-4 with 1N HCl and filtered. The filtrate was concentrated and purified by prep-HPLC to obtain compound C6 as a white solid. ¹H-NMR (DMSO-d₆, 300 MHz): δ 8.15 (s, 1H), 7.87-7.95 (m, 2H), 7.57-7.63 (m, 3H), 7.27 (d, J=8.4 Hz, 2H), 7.01-7.06 (m, 3H), 6.38 (d, J=3.3 Hz, 1H), 4.40 (s, 2H), 4.33 (s, 2H), 2.55 (s, 6H), 2.27 (s, 3H). MS: 556.1 (M−1)⁻.

### Comparative Example C7

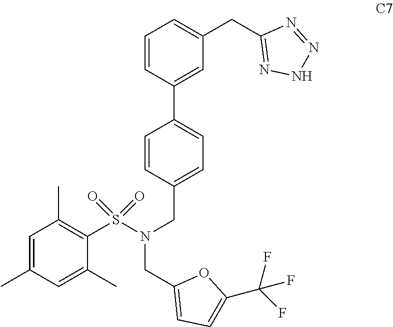 [SMILES: Cc1cc(C)c(S(=O)(=O)N(Cc2ccc(-c3cccc(Cc4nn[nH]n4)c3)cc2)Cc2ccc(C(F)(F)F)o2)c(C)c1]

### N-((3′-((2H-Tetrazol-5-yl)methyl)-[1,1′-biphenyl]-4-yl)methyl)-2,4,6-trimethyl-N-((5-(trifluoro-methyl)furan-2-yl)methyl)benzenesulfonamide (C7)

A solution of compound 3b (341 mg, 0.61 mmol), 5-(3-bromobenzyl)-2H-tetrazole (145 mg, 0.61 mmol), s-phos (25 mg, 60 μmol), Pd(OAc)₂ (7 mg, 30 μmol) and K₃PO₄ (324 mg, 1.52 mmol) in ACN/H₂O (9 mL/3 mL) under N₂ was heated to reflux overnight, cooled, filtered, concentrated and purified by prep-HPLC to give compound C7 as a yellow solid. ¹H-NMR (CD₃OD, 400 MHz): δ 7.53-7.51 (m, 4H), 7.41 (t, J=7.6 Hz, 1H), 7.25-7.21 (m, 3H), 7.04 (s, 2H), 6.79-6.78 (m, 1H), 6.26 (d, J=3.6 Hz, 1H), 4.40 (s, 2H), 4.38 (s, 2H), 4.32 (s, 2H), 2.61 (s, 6H), 2.30 (s, 3H). MS: 596.2 (M+1)⁺.

### Example 7/1 to 7/11

The following Examples were prepared similar as described for Example C7 using the appropriate building blocks and optionally saponified as described in Example 2.

| # | building block | structure | analytical data |
|---|---|---|---|
| 7/1 | 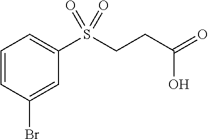 [SMILES: O=C(O)CCS(=O)(=O)c1cccc(Br)c1] | 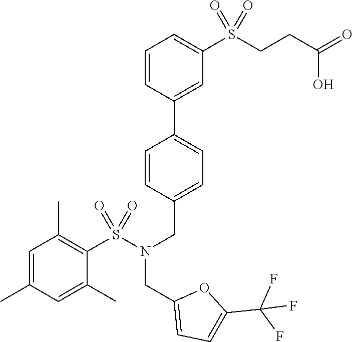 | ¹H-NMR (CD₃OD, 300 MHz): δ 8.12-8.11 (m, 1H), 7.99-7.91 (m, 2H), 7.73 (t, J = 7.5 Hz, 1H), 7.65-7.62 (m, 2H) 7.31-7.28 (m, 2H), 7.07 (s, 2H), 6.82 (dd, J = 0.8 Hz, 2.4 Hz, 1H), 6.31 (dd, J = 0.5 Hz, 3.0 Hz, 1H), 4.44 (d, J = 3.6 Hz, 2H), 4.36 (d, J = 3.6 Hz, 2H), 4.57-3.52 (m, 2H), 2.64-2.57 (m, 8H), 2.32 (d, J = 4.2 Hz, 3H). MS: 596.2 (M + 1)⁺. |
| 7/2 | 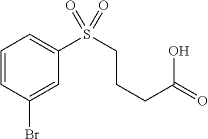 [SMILES: O=C(O)CCCS(=O)(=O)c1cccc(Br)c1] | 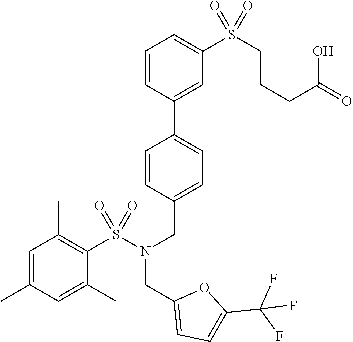 [SMILES: Cc1cc(C)c(S(=O)(=O)N(Cc2ccc(-c3cccc(S(=O)(=O)CCCC(=O)O)c3)cc2)Cc2ccc(C(F)(F)F)o2)c(C)c1] | ¹H-NMR (400 MHz, CDCl₃): δ 8.09 (s, 1H), 7.95 (d, J = 7.6 Hz, 1H), 7.90 (d, J = 7.6 Hz, 1H), 7.71 (t, J = 7.6 Hz, 1H), 7.60 (d, J = 7.6 Hz, 2H), 7.29 (d, J = 8.0 Hz, 2H), 7.04 (s, 2H), 6.79 (d, J = 2.4 Hz, 1H), 6.27 (d, J = 3.2 Hz, 1H), 4.43 (s, 2H), 4.33 (s, 2H), 3.36-3.32 (m, 2H), 2.61 (s, 6H), 2.42 (t, J = 6.8 Hz, 2H), 2.30 (s, 3H), 1.98-1.91 (m, 2H) MS: 664.2 (M + 1)⁺. |
| 7/3 | 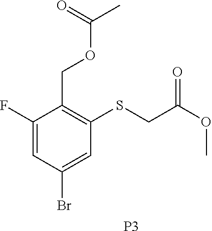 | 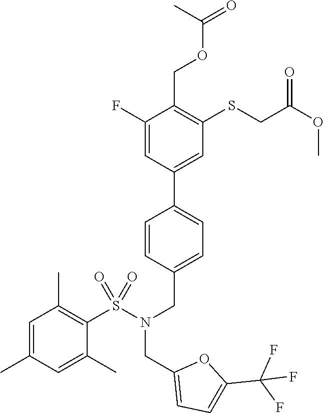 | MS: 708 (M + )⁺. |
| 7/4 | 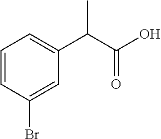 | 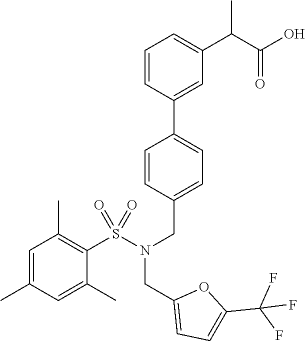 [SMILES: Cc1cc(C)c(S(=O)(=O)N(Cc2ccc(-c3cccc(C(C)C(=O)O)c3)cc2)Cc2ccc(C(F)(F)F)o2)c(C)c1] | ¹H-NMR (CD₃OD, 400 MHz): δ 7.55-7.52 (m, 3H), 7.46-7.44 (m, 1H), 7.38-7.30 (m, 2H), 7.21 (d, J = 8.4 Hz, 2H), 7.05 (s, 2H), 6.80 (dd, J = 3.4 Hz, 1.0 Hz, 1H), 6.28 (d, J = 2.8 Hz, 1H), 4.40 (s, 2H), 4.33 (s, 2H), 3.74 (q, J = 7.2 Hz, 1H), 2.62 (s, 6H), 2.31 (s, 3H), 1.48 (d, J = 7.2 Hz, 3H). MS: 584.1 (M − 1)⁻. |
| 7/5 | 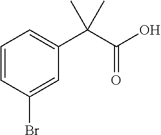 | 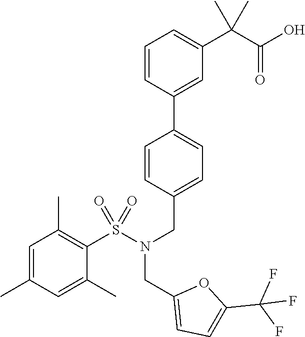 [SMILES: Cc1cc(C)c(S(=O)(=O)N(Cc2ccc(-c3cccc(C(C)(C)C(=O)O)c3)cc2)Cc2ccc(C(F)(F)F)o2)c(C)c1] | ¹H-NMR (DMSO-d₆, 400 MHz): δ 7.56-7.54 (m, 3H), 7.49-7.33 (m, 3H), 7.24 (d, J = 8.0 Hz, 2H), 7.08 (s, 2H), 7.03 (dd, J = 1.4 Hz, 3.4 Hz, 1H), 6.39 (d, J = 3.2 Hz, 1H), 4.38 (s, 2H), 4.32 (s, 2H), 2.56 (s, 6H), 2.27 (s, 3H), 1.52 (s, 6H). MS: 598.1 (M − 1)⁻. |
| 7/6 | 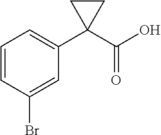 [SMILES: O=C(O)C1(c2cccc(Br)c2)CC1] | 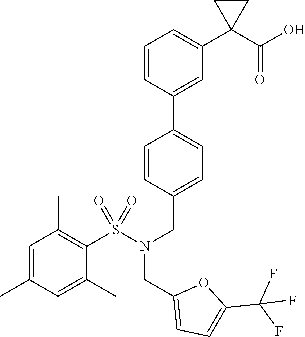 [SMILES: Cc1cc(C)c(S(=O)(=O)N(Cc2ccc(-c3cccc(C4(C(=O)O)CC4)c3)cc2)Cc2ccc(C(F)(F)F)o2)c(C)c1] | ¹H-NMR (CDCl₃, 300 MHz): δ 7.56-7.35 (m, 6H), 7.21 (d, J = 8.1 Hz, 2H), 7.00 (s, 2H), 6.67-6.66 (m, 1H), 6.23 (d, J = 3.0 Hz, 1H), 4.37 (s, 2H), 4.28 (s, 2H), 2.66 (s, 6H), 2.34 (s, 3H), 1.72-1.70 (m, 2H), 1.33-1.31 (m, 2H). MS: 596.1 (M − H)⁻. |
| 7/7 | 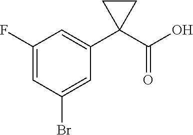 [SMILES: O=C(O)C1(c2cc(F)cc(Br)c2)CC1] | 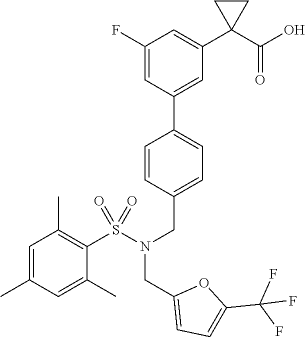 [SMILES: Cc1cc(C)c(S(=O)(=O)N(Cc2ccc(-c3cc(F)cc(C4(C(=O)O)CC4)c3)cc2)Cc2ccc(C(F)(F)F)o2)c(C)c1] | ¹H-NMR (CDCl₃, 400 MHz): δ 7.48 (d, J = 8.0 Hz, 2H), 7.33 (s, 1H), 7.20 (d, J = 8.0 Hz, 2H), 7.16 (d, J = 9.2 Hz, 1H), 7.06 (d, J = 9.6 Hz, 1H), 6.99 (s, 2H), 6.65 (d, J = 2.4 Hz, 1H), 6.21 (d, J = 2.8 Hz, 1H), 4.36 (s, 2H), 4.26 (s, 2H), 2.64 (s, 6H), 2.32 (s, 3H), 1.73-1.70 (m, 2H), 1.33-1.30 (m, 2H). MS: 614.1 (M − H)⁻. |
| 7/8 | 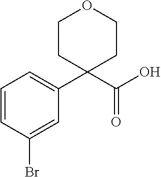 [SMILES: O=C(O)C1(c2cccc(Br)c2)CCOCC1] | 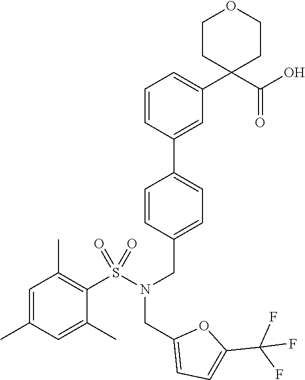 [SMILES: Cc1cc(C)c(S(=O)(=O)N(Cc2ccc(-c3cccc(C4(C(=O)O)CCOCC4)c3)cc2)Cc2ccc(C(F)(F)F)o2)c(C)c1] | ¹H-NMR (CDCl₃, 400 MHz): δ 7.59 (s, 1H), 7.51-7.42 (m, 5H), 7.22 (d, J = 8.0 Hz, 2H), 6.99 (s, 2H), 6.65 (d, J = 2.0 Hz, 1H), 6.21 (d, J = 3.2 Hz, 1H), 4.37 (s, 2H), 4.26 (s, 2H), 3.97-3-94 (m, 2H), 3.65 (t, J = 11.0 Hz, 2H), 2.64 (s, 6H), 2.58 (d, J = 14.0 Hz, 2H), 2.32 (s, 3H), 2.09-2.02 (m, 2H). MS: 664.2 (M + Na)⁺. |
| 7/9 | 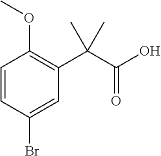 | 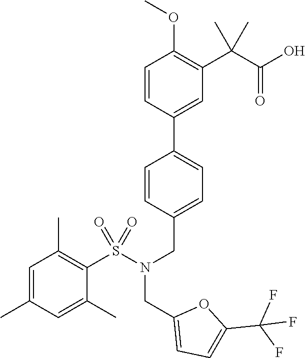 [SMILES: COc1ccc(-c2ccc(CN(Cc3ccc(C(F)(F)F)o3)S(=O)(=O)c3c(C)cc(C)cc3C)cc2)cc1C(C)(C)C(=O)O] | ¹H-NMR (CDCl₃, 400 MHz): δ 7.50-7.44 (m, 4H), 7.19 (d, J = 7.6 Hz, 2H), 6.99-6.94 (m, 3H), 6.65 (s, 1H), 6.21 (s, 1H), 4.36 (s, 2H), 4.27 (s, 2H), 3.85 (s, 3H), 2.64 (s, 6H), 2.32 (s, 3H), 1.61 (s, 6H). MS: 627.9 (M − H)⁻. |
| 7/10 | 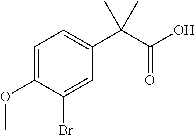 | 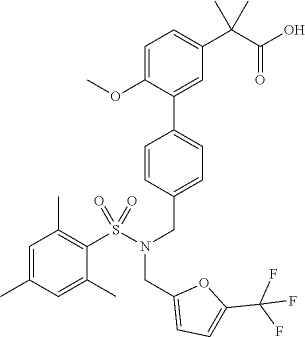 [SMILES: COc1ccc(C(C)(C)C(=O)O)cc1-c1ccc(CN(Cc2ccc(C(F)(F)F)o2)S(=O)(=O)c2c(C)cc(C)cc2C)cc1] | ¹H-NMR (CDCl₃, 400 MHz): δ 7.45 (d, J = 7.6 Hz, 2H), 7.35 (d, J = 8.4 Hz, 1H), 7.31 (s, 1H), 7.17 (d, J = 8.0 Hz, 2H), 6.98-6.93 (m, 3H), 6.65 (s, 1H), 6.23 (s, 1H), 4.36 (s, 2H), 4.30 (s, 2H), 3.79 (s, 3H), 2.64 (s, 6H), 2.31 (s, 3H), 1.62 (s, 6H). MS: 627.9 (M − H)⁻. |
| C7/11 | 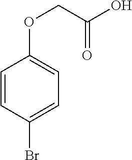 [SMILES: O=C(O)COc1ccc(Br)cc1] | 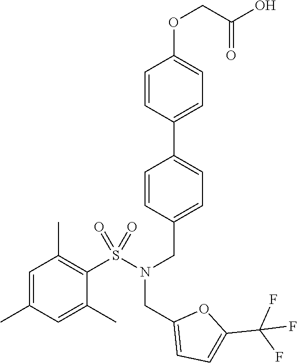 [SMILES: Cc1cc(C)c(S(=O)(=O)N(Cc2ccc(-c3ccc(OCC(=O)O)cc3)cc2)Cc2ccc(C(F)(F)F)o2)c(C)c1] | ¹H-NMR (CDCl₃, 400 MHz): δ 7.39-7.36 (m, 4H), 7.15 (d, J = 8.4 Hz, 2H), 6.94-6.88 (m, 4H), 6.58 (s, 1H), 6.12 (d, J = 2.8 Hz, 1H), 4.48 (s, 2H), 4.32 (s, 2H), 4.16 (s, 2H), 2.58 (s, 6H), 2.28 (s, 3H) MS: 586.1 (M − H)⁻. |

### Example 8

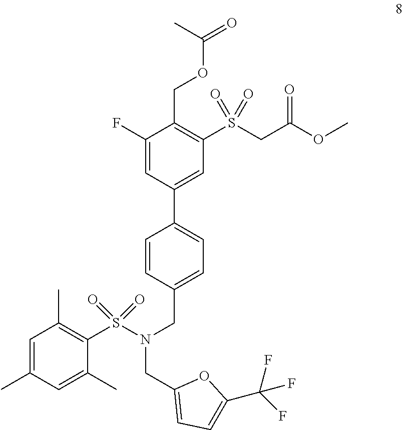

### Methyl 2-((4-(acetoxymethyl)-5-fluoro-4′-(((2,4,6-trimethyl-N-((5-(trifluoromethyl)furan-2-yl)methyl)phenyl)sulfonamido)methyl)-[1,1′-biphenyl]-3-yl)sulfonyl)acetate (8)

A mixture of compound 7/3 (350 mg, 0.49 mmol) and m-CPBA (269 mg, 1.3 mmol) in DCM (30 mL) was stirred at 35° C. overnight, cooled, washed with a NaHCO₃ solution and brine, dried over Na₂SO₄, filtered through silica gel and washed with PE/EA (20:1 to 10:1 to 3:1). The organic layer was concentrated to give compound 8 as a white solid. MS: 740 (M+1)⁺.

### Example 9

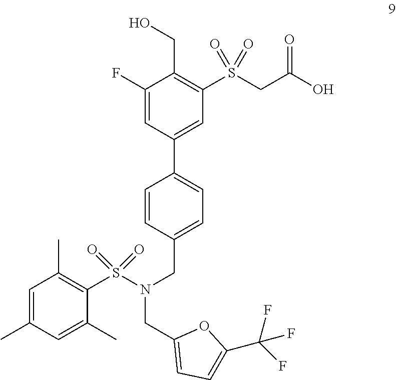 [SMILES: Cc1cc(C)c(S(=O)(=O)N(Cc2ccc(-c3cc(F)c(CO)c(S(=O)(=O)CC(=O)O)c3)cc2)Cc2ccc(C(F)(F)F)o2)c(C)c1]

### 2-((5-Fluoro-4-(hydroxymethyl)-4′-(((2,4,6-trimethyl-N-((5-(trifluoromethyl)furan-2-yl)methyl)phenyl)sulfonamido)methyl)-[1,1′-biphenyl]-3-yl)sulfonyl)acetic acid (9)

A solution of compound 8 (228 mg, 0.31 mmol) and LiOH.H₂O (24 mg, 0.57 mmol) in THF/H₂O (5 mL/3 mL) was stirred at rt overnight. The mixture was acidified with 1N HCl and extracted with EA (20 mL). The organic layer was concentrated to give compound 9 as a white solid. ¹H-NMR (CDCl₃, 400 MHz): δ 8.06 (s, 1H), 7.55-7.49 (m, 3H), 7.28-7.26 (m, 2H), 6.98 (s, 2H), 6.62 (s, 1H), 6.16 (d, J=2.8 Hz, 1H), 5.09 (s, 2H), 4.48 (s, 2H), 4.39 (s, 2H), 4.20 (s, 2H), 2.61 (s, 6H), 2.31 (s, 3H). MS: 684.1 (M+1)⁺.

### Example 10

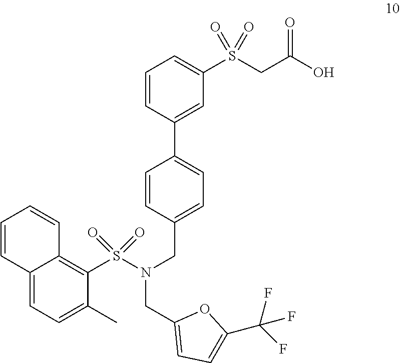 [SMILES: Cc1ccc2ccccc2c1S(=O)(=O)N(Cc1ccc(-c2cccc(S(=O)(=O)CC(=O)O)c2)cc1)Cc1ccc(C(F)(F)F)o1]

### Step 1: N-(4-Bromobenzyl)-2-methylnaphthalene-1-sulfonamide (10a)

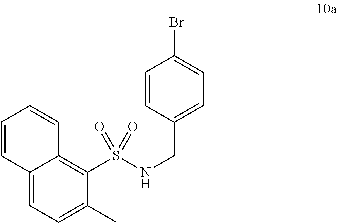 [SMILES: Cc1ccc2ccccc2c1S(=O)(=O)NCc1ccc(Br)cc1]

To a suspension of (4-bromophenyl)methanamine (500 mg, 2.70 mmol) and 2-methyl-naphthalene-1-sulfonyl chloride (716 mg, 2.97 mmol) in DCM (30 mL) was added TEA (546 mg, 5.40 mmol). The mixture was stirred at rt overnight and adjusted to pH=4 with 2N HCl. The organic layer was washed with brine, dried over Na₂SO₄, filtered, concentrated and triturated with PE to give crude compound 10a as a yellow solid.

### Step 2: N-(4-Bromobenzyl)-2-methyl-N-((5-(trifluoromethyl)furan-2-yl)methyl)naphthalene-1-sulfonamide (10b)

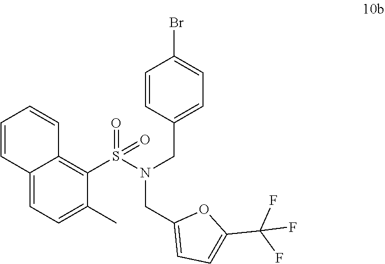 [SMILES: Cc1ccc2ccccc2c1S(=O)(=O)N(Cc1ccc(Br)cc1)Cc1ccc(C(F)(F)F)o1]

To a solution of compound 10a (389 mg, 1.00 mmol) and 2-(bromomethyl)-5-(trifluoro-methyl)furan (229 mg, 1.00 mmol) in ACN (30 mL) was added K₂CO₃ (276 mg, 2.00 mmol) and KI (166 mg, 1.00 mmol). The mixture was stirred at 70° C. overnight, cooled, filtered, concentrated and purified by FCC (PE:EA=50:1) to give compound 10b as a yellow solid.

### Step 3: Methyl 2-((4′-(((2-methyl-N-((5-(trifluoromethyl)furan-2-yl)methyl)naphthalene)-1-sulfonamido)methyl)-[1,1′-biphenyl]-3-yl)sulfonyl)acetate (10c)

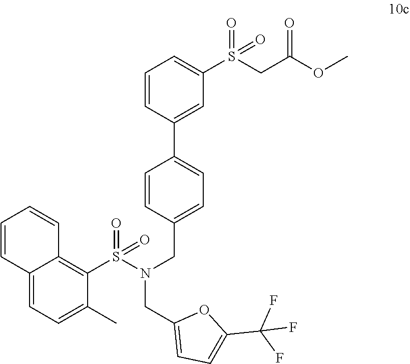 [SMILES: COC(=O)CS(=O)(=O)c1cccc(-c2ccc(CN(Cc3ccc(C(F)(F)F)o3)S(=O)(=O)c3c(C)ccc4ccccc34)cc2)c1]

To a solution of compound 10b (394 mg, 734 μmol), methyl 2-((3-(4,4,5,5-tetramethyl-1,3,2-dioxaborolan-2-yl)phenyl)sulfonyl)acetate (249 mg, 734 μmol), PPh₃ (58 mg, 220 μmol) and K₃PO₄ (473 mg, 2.20 mmol) in 1,4-dioxane (30 mL) was added Pd₂(dba)₃ (68 mg, 73 μmol). The mixture was stirred at 85° C. under N₂ for 10 h, cooled, filtered, concentrated and purified by FCC (PE:EA=10:1 to 2:1) to afford compound 10c as a colorless oil.

### Step 4: 2-((4′-(((2-Methyl-N-((5-(trifluoromethyl)furan-2-yl)methyl)naphthalene)-1-sulfonamido)methyl)[1,1′-biphenyl]-3-yl)sulfonyl)acetic acid (10)

To a solution of compound 10c (333 mg, 0.50 mmol) in THF (10 mL) and water (10 mL) was added LiOH.H₂O (42 mg, 1.00 mmol) at rt and the mixture was stirred at rt overnight, concentrated and adjusted to pH=6 with 2N HCl. The mixture was filtered and the residue was purified by prep-HPLC to give compound 10 as a white solid. ¹H-NMR (CDCl₃, 400 MHz): δ 8.77 (d, J=7.6 Hz, 1H), 7.98 (s, 1H), 7.85-7.76 (m, 3H), 7.55-7.50 (m, 2H), 7.44 (t, J=7.6 Hz, 1H), 7.34 (t, J=7.6 Hz, 1H), 7.27-7.25 (m, 3H), 6.97 (d, J=8.4 Hz, 2H), 6.42 (d, J=2.4 Hz, 1H), 5.89 (d, J=3.2 Hz, 1H), 4.33 (s, 2H), 4.21 (s, 2H), 4.16 (s, 2H), 2.83 (s, 3H). MS: 658.1 (M+1)⁺.

### Example 10/1 to 10/20

The following Examples were prepared similar as described for Example 10 using the appropriate building blocks.

| # | building block(s) | structure | analytical data |
|---|---|---|---|
| 10/1 | 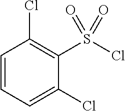 [SMILES: O=S(=O)(Cl)c1c(Cl)cccc1Cl] | 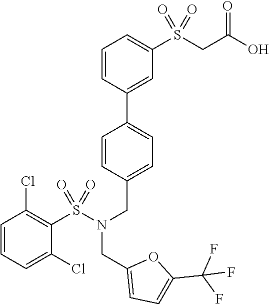 [SMILES: O=C(O)CS(=O)(=O)c1cccc(-c2ccc(CN(Cc3ccc(C(F)(F)F)o3)S(=O)(=O)c3c(Cl)cccc3Cl)cc2)c1] | ¹H-NMR (CDCl₃, 400 MHz): δ 8.03 (s, 1H), 7.83 (d, J = 7.6 Hz, 1H), 7.64 (d, J = 8.0 Hz, 1H), 7.43-7.39 (m, 5H), 7.32- 7.27 (m, 1H), 7.21 (d, J = 8.0 Hz, 2H), 6.52 (d, J = 2.0 Hz, 1H), 6.13 (d, J = 3.2 Hz, 1H), 4.51 (s, 2H), 4.28 (s, 2H), 4.18 (s, 2H). MS: 679.0 (M + 18)⁺. |
| 10/2 | 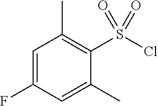 | 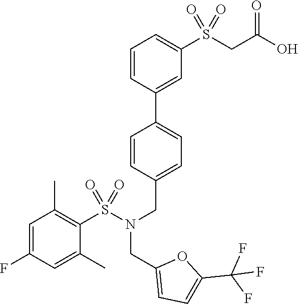 [SMILES: Cc1cc(F)cc(C)c1S(=O)(=O)N(Cc1ccc(-c2cccc(S(=O)(=O)CC(=O)O)c2)cc1)Cc1ccc(C(F)(F)F)o1] | ¹H-NMR (CDCl₃, 400 MHz): δ 10.13 (br s, 1H), 8.10 (s, 1H), 7.89 (d, J = 8.0 Hz, 1H), 7.74 (d, J = 8.0 Hz, 1H), 7.69 (br s, 1H), 7.55-7.50 (m, 3H), 7.24 (s, 1H), 6.85 (d, J = 8.8 Hz, 2H), 6.61 (d, J = 2.0 Hz, 1H), 6.15 (d, J = 3.6 Hz, 1H), 4.39 (s, 2H), 4.19 (s, 2H), 4.09 (s, 2H), 2.63 (s, 6H). MS: 640 (M + 1)⁺. |
| 10/3 | 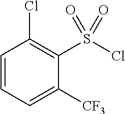 [SMILES: O=S(=O)(Cl)c1c(Cl)cccc1C(F)(F)F] | 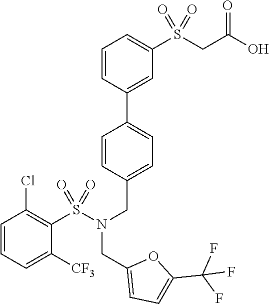 [SMILES: O=C(O)CS(=O)(=O)c1cccc(-c2ccc(CN(Cc3ccc(C(F)(F)F)o3)S(=O)(=O)c3c(Cl)cccc3C(F)(F)F)cc2)c1] | ¹H-NMR (CD₃OD, 400 MHz): δ 8.18 (s, 1H), 7.97 (t, J = 8.2 Hz, 3H), 7.84-7.82 (m, 1H), 7.72 (t, J = 8.0 Hz, 2H), 7.66 (d, J = 7.6 Hz, 2H), 7.44 (d, J = 8.0 Hz, 2H), 6.75 (d, J = 2.0 Hz, 1H), 6.27 (d, J = 2.8 Hz, 1H), 4.94 (s, 2H), 4.71 (s, 2H), 4.46 (s, 2H). MS: 713 (M + 18)⁺. |
| 10/4 | 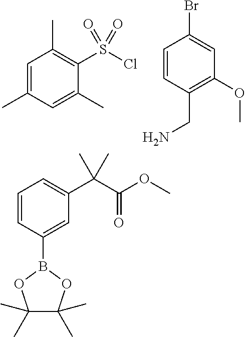 | 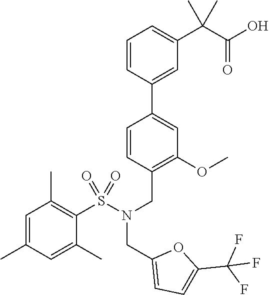 [SMILES: COc1cc(-c2cccc(C(C)(C)C(=O)O)c2)ccc1CN(Cc1ccc(C(F)(F)F)o1)S(=O)(=O)c1c(C)cc(C)cc1C] | ¹H-NMR (CDCl₃, 400 MHz): δ 7.53 (s, 1H), 7.39-7.34 (m, 3H), 7.18 (d, J = 8.0 Hz, 1H), 7.05 (d, J = 8.0 Hz, 1H), 6.93 (d, J = 2.8 Hz, 3H), 6.63 (d, J = 2.0 Hz, 1H), 6.23 (d, J = 3.2 Hz, 1H), 4.42 (s, 2H), 4.32 (s, 2H), 3.70 (s, 3H), 2.61 (s, 6H), 2.29 (s, 3H), 1.60 (s, 6H). MS: 628.1 (M − H)⁻. |
| 10/5 | 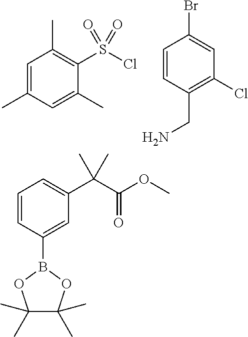 | 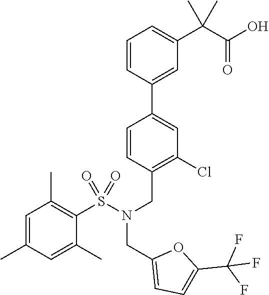 [SMILES: Cc1cc(C)c(S(=O)(=O)N(Cc2ccc(C(F)(F)F)o2)Cc2ccc(-c3cccc(C(C)(C)C(=O)O)c3)cc2Cl)c(C)c1] | ¹H-NMR (CDCl₃, 400 MHz): δ 7.52 (s, 1H), 7.49 (d, J = 1.6 Hz, 1H), 7.43-7.40 (m, 5H), 6.96 (s, 2H), 6.63 (d, J = 2.0 Hz, 1H), 6.23 (d, J = 3.2 Hz, 1H), 4.60 (s, 2H), 4.33 (s, 2H), 2.64 (s, 6H), 2.29 (s, 3H), 1.65 (s, 6H). MS: 634.1 (M + H)⁺. |
| 10/6 | 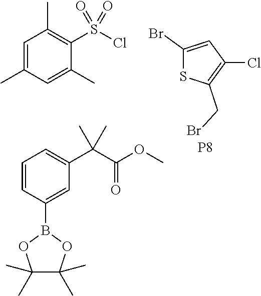 | 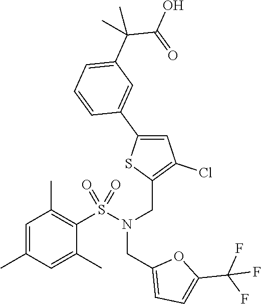 [SMILES: Cc1cc(C)c(S(=O)(=O)N(Cc2ccc(C(F)(F)F)o2)Cc2sc(-c3cccc(C(C)(C)C(=O)O)c3)cc2Cl)c(C)c1] | ¹H-NMR (CDCl₃, 400 MHz): δ 7.49 (s, 1H), 7.36-7.33 (m, 3H), 7.01 (s, 1H), 6.97 (s, 2H), 6.63 (d, J = 2.4 Hz, 1H), 6.30 (d, J = 3.6 Hz, 1H), 4.56 (s, 2H), 4.38 (s, 2H), 2.63 (s, 6H), 2.30 (s, 3H), 1.62 (s, 6H). MS. 657.0 (M + 18)⁺. |
| 10/7 | 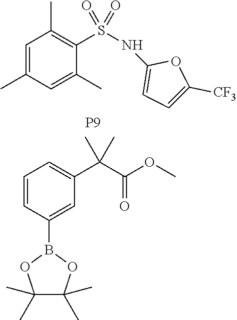 | 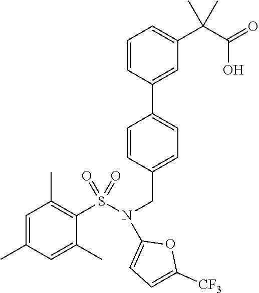 [SMILES: Cc1cc(C)c(S(=O)(=O)N(Cc2ccc(-c3cccc(C(C)(C)C(=O)O)c3)cc2)c2ccc(C(F)(F)F)o2)c(C)c1] | ¹H-NMR (CDCl₃, 400 MHz): δ 7.54 (s, 1H), 7.47 (d, J = 8.0 Hz, 2H), 7.42-7.37 (m, 3H), 7.28-7.26 (m, 2H), 6.96 (s, 2H), 6.56 (d, J = 2.0 Hz, 1H), 6.02 (d, J = 3.6 Hz, 1H), 4.81 (s, 2H), 2.56 (s, 6H), 2.31 (s, 3H), 1.63 (s, 6H). MS: 603.0 (M + 18)⁺. |
| 10/8 | 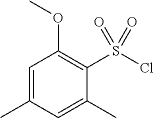 | 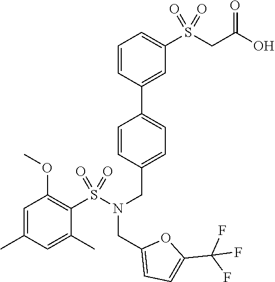 [SMILES: COc1cc(C)cc(C)c1S(=O)(=O)N(Cc1ccc(-c2cccc(S(=O)(=O)CC(=O)O)c2)cc1)Cc1ccc(C(F)(F)F)o1] | ¹H-NMR (CDCl₃, 400 MHz): δ 8.07 (s, 1H), 7.86 (d, J = 7.2 Hz, 1H), 7.71 (d, J = 8.0 Hz, 1H), 7.52-7.43 (m, 3H), 7.28- 7.26 (m, 2H), 6.64 (s, 1H), 6.59 (s, 1H), 6.50 (d, J = 2.0 Hz, 1H), 5.98 (d, J = 3.6 Hz, 1H), 4.50 (s, 2H), 4.27 (s, 2H), 4.17 (br s, 2H), 3.76 (s, 3H), 2.61 (s, 3H), 2.30 (s, 3H). MS: 651.9 (M + 1)⁺. |
| 10/9 | 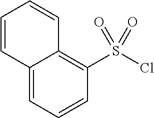 [SMILES: O=S(=O)(Cl)c1cccc2ccccc12] | 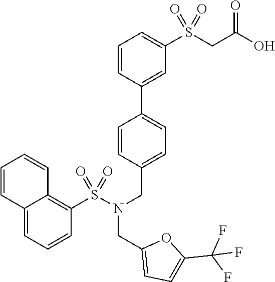 [SMILES: O=C(O)CS(=O)(=O)c1cccc(-c2ccc(CN(Cc3ccc(C(F)(F)F)o3)S(=O)(=O)c3cccc4ccccc34)cc2)c1] | ¹H-NMR (CDCl₃, 400 MHz): δ 8.65 (d, J = 8.4 Hz, 1H), 8.25 (dd, J = 1.0, J = 7.6 Hz, 1H), 8.11-8.08 (m, 2H), 7.97- 7.92 (m, 2H), 7.84 (d, J = 8.4 Hz, 1H), 7.68-7.62 (m, 3H), 7.52 (t, J = 7.8 Hz, 1H), 7.46 (d, J = 8.4 Hz, 2H), 7.22 (d, J = 8.0 Hz, 2H) 6.52 (dd, J = 0.8, J = 3.2 Hz, 1H), 6.03 (d, J = 3.2 Hz, 1H), 4.53 (s, 2H), 4.45 (s, 2H), 4.17 (s, 2H). MS: 643.9 (M + 1)⁺. |
| 10/10 | 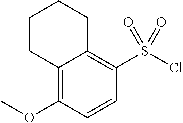 | 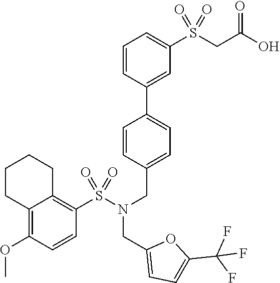 [SMILES: COc1ccc(S(=O)(=O)N(Cc2ccc(-c3cccc(S(=O)(=O)CC(=O)O)c3)cc2)Cc2ccc(C(F)(F)F)o2)c2c1CCCC2] | ¹H-NMR (CDCl₃, 400 MHz): δ 8.05 (s, 1H), 7.86-7.82 (m, 2H), 7.66 (d, J = 8.4 Hz, 1H), 7.45-7.40 (m, 3H), 7.19 (d, J = 7.2 Hz, 2H), 6.66 (d, J = 9.2 Hz, 1H), 6.57 (s, 1H), 6.06 (s, 1H), 4.33 (s, 2H), 4.20 (s, 2H), 4.17 (br s, 2H), 3.82 (s, 3H), 2.96 (s, 2H), 2.62 (s, 2H), 1.69 (s, 4H). MS: 677.9 (M + 1)⁺. |
| 10/11 | 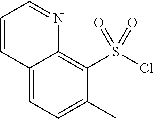 | 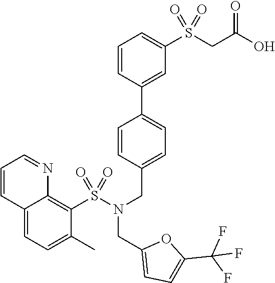 [SMILES: Cc1ccc2cccnc2c1S(=O)(=O)N(Cc1ccc(-c2cccc(S(=O)(=O)CC(=O)O)c2)cc1)Cc1ccc(C(F)(F)F)o1] | ¹H-NMR (CDCl₃, 400 MHz): δ 8.85 (dd, J = 1.8, J = 4.0 Hz, 1H), 8.06 (dd, J = 1.4, J = 8.2 Hz, 1H), 8.00 (s, 1H), 7.84- 7.82 (m, 1H), 7.79 (d, J = 8.4 Hz, 1H), 7.62 (d, J = 7.2 Hz, 1H), 7.44-7,40 (m, 1H), 7.37-7.32 (m, 2H), 7.29-7.27 (m, 2H), 7.16 (d, J = 8.4 Hz, 2H), 6.31 (d, J = 2.4 Hz, 1H), 5.89 (d, J = 3.2 Hz, 1H), 4.71 (s, 2H), 4.51 (s, 2H), 4.17 (br s, 2H), 2.91 (s, 3H). MS: 658.9 (M + 1)⁺. |
| 10/12 | 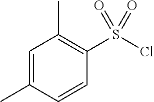 | 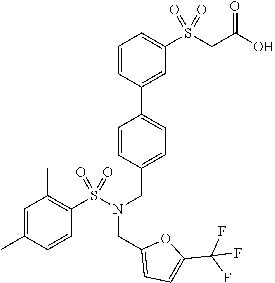 [SMILES: Cc1ccc(S(=O)(=O)N(Cc2ccc(-c3cccc(S(=O)(=O)CC(=O)O)c3)cc2)Cc2ccc(C(F)(F)F)o2)c(C)c1] | ¹H-NMR (CDCl₃, 400 MHz): δ 8.04 (s, 1H), 7.83 (d, J = 8.0 Hz, 1H), 7.78 (d, J = 8.0 Hz, 1H), 7.64 (d, J = 7.6 Hz, 1H), 7.42-7.39 (m, 3H), 7.20 (d, J = 8.0 Hz, 2H), 7.09 (s, 1H), 7.03 (d, J = 8.0 Hz, 1H), 6.53 (d, J = 2.4 Hz, 1H), 6.04 (d, J = 3.2 Hz, 1H), 4.35 (s, 2H), 4.17 (s, 4H), 2.49 (s, 3H), 2.33 (s, 3H). MS: 639.1 (M + 18)⁺. |
| 10/13 | 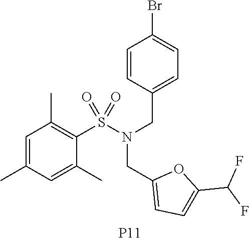 | 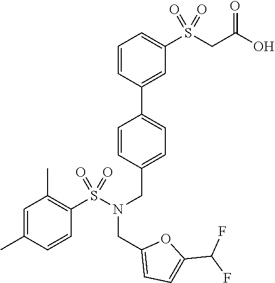 | ¹H-NMR (300 MHz, CDCl₃): δ 8.04 (s, 1H), 7.83 (d, J = 7.5 Hz, 1H), 7.62 (d, J = 7.5 Hz, 1H), 7.41-7.36 (m, 3H), 7.15 (d, J = 8.1 Hz, 2H), 6.93 (s, 2H), 6.59- 6.23 (m, 2H), 6.04 (d, J = 3.3 Hz, 1H), 4.29 (s, 2H), 4.17 (s, 2H), 4.10 (s, 2H), 2.56 (s, 6H), 2.26 (s, 3H). MS: 618.1 (M + 1)⁺. |
| 10/14 | 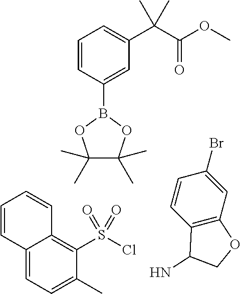 | 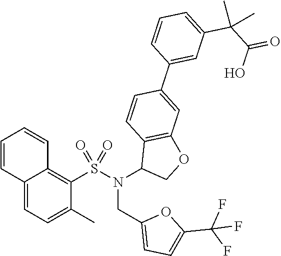 [SMILES: Cc1ccc2ccccc2c1S(=O)(=O)N(Cc1ccc(C(F)(F)F)o1)C1COc2cc(-c3cccc(C(C)(C)C(=O)O)c3)ccc21] | ¹H-NMR (DMSO-d₆, 400 MHz): δ 8.71 (d, J = 8.8 Hz, 1H), 8.15 (d, J = 8.8 Hz, 1H), 8.02 (d, J = 7.6 Hz, 1H), 7.69-7.62 (m, 2H), 7.51-7.48 (m, 2H), 7.41-7.34 (m, 3H), 7.02 (s, 1H), 6.96 (d, J = 7.6 Hz, 1H), 6.84 (d, J = 7.6 Hz, 1H), 6.72 (d, J = 2.4 Hz, 1H), 5.87 (d, J = 3.2 Hz, 1H), 5.81-5.79 (m, 1H), 4.69-4.65 (m, 1H), 4.42-4.38 (m, 1H), 4.33 (s, 2H), 2.88 (s, 3H), 1.48 (s, 6H), MS: 647.9 (M − H)⁻. |
| 10/15 | 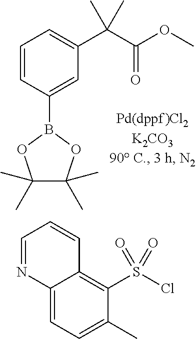 | 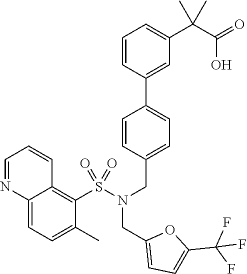 [SMILES: Cc1ccc2ncccc2c1S(=O)(=O)N(Cc1ccc(-c2cccc(C(C)(C)C(=O)O)c2)cc1)Cc1ccc(C(F)(F)F)o1] | ¹H-NMR (500 MHz, CD₃OD): δ 9.36 (d, J = 9.0 Hz, 1H), 8.90 (dd, J = 4.3, 1.3 Hz, 1H), 8.15 (d, J = 9.0 Hz, 1H), 7.72 (d, J = 9.0 Hz, 1H), 7.65 (dd, J = 9.3, 4.3 Hz, 1H), 7.53 (d, J = 0.5 Hz, 1H), 7.41-7.38 (m, 5H), 7.11 (d, J = 8.0 Hz, 2H), 6.73-6.72 (m, 1H), 6.22 (d, J = 3.5 Hz, 1H), 4.55 (s, 2H), 4.51 (s, 2H), 2.97 (s, 3H), 1.62 (s, 6H). MS: 623.2 (M + 1)⁺. |
| 10/16 | 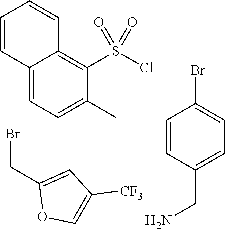 | 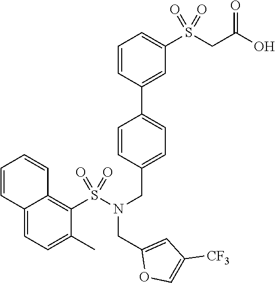 [SMILES: Cc1ccc2ccccc2c1S(=O)(=O)N(Cc1ccc(-c2cccc(S(=O)(=O)CC(=O)O)c2)cc1)Cc1cc(C(F)(F)F)co1] | ¹H-NMR (CDCl₃, 400 MHz): δ 8.73 (d, J = 8.8 Hz, 1H), 8.00 (s, 1H), 7.88-7.78 (m, 3H), 7.61-7.29 (m, 8H), 7.01 (d, J = 7.6 Hz, 2H), 5.87 (s, 1H), 4.38 (s, 2H), 4.20 (s, 2H), 4.14 (s, 2H), 2.85 (s, 3H). MS: 657.9 (M + 1)⁺. |
| 10/17 | 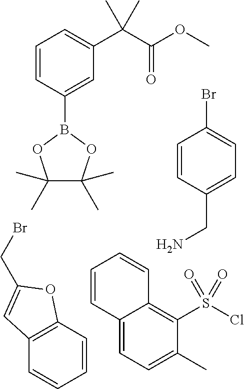 | 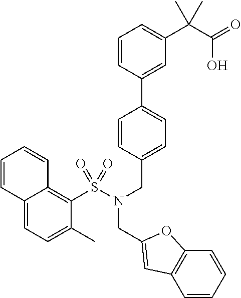 [SMILES: Cc1ccc2ccccc2c1S(=O)(=O)N(Cc1ccc(-c2cccc(C(C)(C)C(=O)O)c2)cc1)Cc1cc2ccccc2o1] | ¹H-NMR (CD₃OD, 400 MHz): δ 8.87 (d, J = 9.2 Hz, 1H), 7.97 (d, J = 8.4 Hz, 1H), 7.88 (d, J = 7.6 Hz, 1H), 7.62-7.58 (m, 1H), 7.54-7.49 (m, 2H), 7.41-7.31 (m, 7H), 7.20 (d, J = 4.0 Hz, 2H), 7.15- 7.11 (m, 1H), 7.04 (d, J = 8.0 Hz, 2H), 6.41 (s, 1H), 4.54 (s, 2H), 4.51 (s, 2H), 2.93 (s, 3H), 1.58 (s, 6H) MS: 602.2 (M − H)⁻. |
| 10/18 | 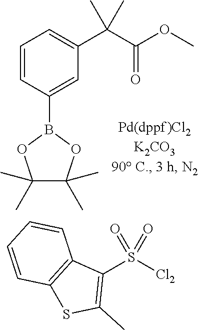 | 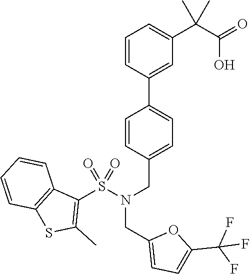 [SMILES: Cc1sc2ccccc2c1S(=O)(=O)N(Cc1ccc(-c2cccc(C(C)(C)C(=O)O)c2)cc1)Cc1ccc(C(F)(F)F)o1] | ¹H-NMR (400 MHz, CD₃OD): δ 8.22 (d, J = 8.0 Hz, 1H), 7.85 (d, J = 7.6 Hz, 1H), 7.54 (d, J = 0.8 Hz, 1H), 7.49-7.39 (m, 7H), 7.18 (d, J = 8.0 Hz, 2H), 6.72 (d, J = 2.0 Hz, 1H), 6.19 (d, J = 3.8 Hz, 1H), 4.54 (s, 2H), 4.53 (s, 2H), 2.88 (s, 3H), 1.62 (s, 6H). MS: 626.0 (M − H)⁻. |
| 10/19 | 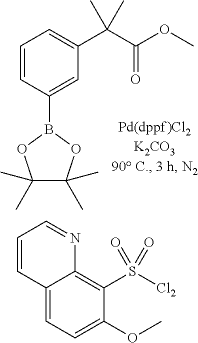 | 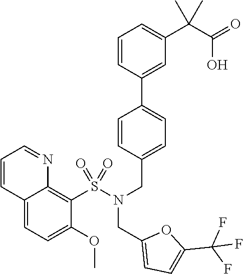 [SMILES: COc1ccc2cccnc2c1S(=O)(=O)N(Cc1ccc(-c2cccc(C(C)(C)C(=O)O)c2)cc1)Cc1ccc(C(F)(F)F)o1] | ¹H-NMR (400 MHz, CD₃OD): δ 8.97 (dd, J = 1.8, 8.2 Hz, 1H), 8.31 (dd, J = 1.6, 8.4 Hz, 1H), 8.17 (d, J = 9.6 Hz, 1H), 7.63-7.60 (m, 2H), 7.48-7.33 (m, 6H), 7.27 (d, J = 8.0 Hz, 2H), 6.68 (dd, J = 1.2, 3.2 Hz, 1H), 6.22 (d, J = 2.8 Hz, 1H), 4.73 (s, 2H), 4.70 (s, 2H), 4.13 (s, 3H), 1.57 (s, 6H). MS: 639.2 (M + 1)⁺. |
| 10/20 | 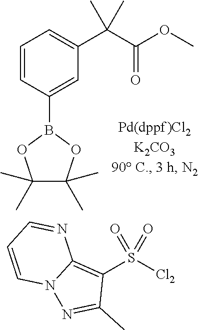 | 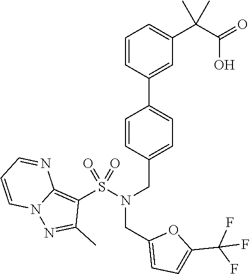 [SMILES: Cc1nn2cccnc2c1S(=O)(=O)N(Cc1ccc(-c2cccc(C(C)(C)C(=O)O)c2)cc1)Cc1ccc(C(F)(F)F)o1] | ¹H-NMR (400 MHz, CD₃OD): δ 8.94 (dd, J = 1.4, 7.0 Hz, 1H), 8.69 (dd, J = 1.6, 4.0 Hz, 1H), 7.61 (s, 1H), 7.49 (d, J = 0.8 Hz, 2H), 7.41-7.36 (m, 3H), 7.31 (d, J = 8.0 Hz, 2H), 7.20 (dd, J = 4.2, 7.0 Hz, 1H), 6.71 (d, J = 1.6 Hz, 1H), 6.27 (d, J = 3.2 Hz, 1H), 4.65 (s, 2H), 4.63 (s, 2H), 2.69 (s, 3H), 1.58 (s, 6H). MS: 613.3 (M + 1)⁺. |

### Example 11

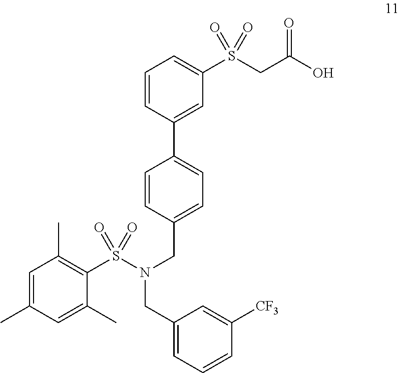 [SMILES: Cc1cc(C)c(S(=O)(=O)N(Cc2ccc(-c3cccc(S(=O)(=O)CC(=O)O)c3)cc2)Cc2cccc(C(F)(F)F)c2)c(C)c1]

### Step 1: 2,4,6-Trimethyl-N-(4-(4,4,5,5-tetramethyl-1,3,2-dioxaborolan-2-yl)benzyl)benzene-sulfonamide (11a)

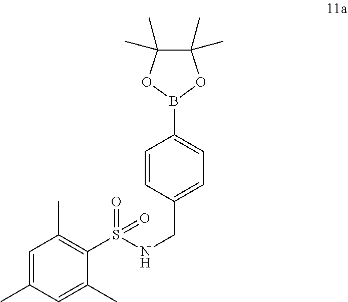

To a suspension of compound 1a (10.0 g, 27.0 mmol), B₂Pin₂ (10.4 g, 40.8 mmol) and K₃PO₄ (8.0 g, 81.6 mmol) in dioxane (300 mL) was added Pd(dppf)Cl₂ (2.2 g, 2.7 mmol) at rt under N₂. The mixture was stirred at 105° C. overnight, cooled, filtered, concentrated and purified by FCC (PE:EA=10:1) to give compound 11a as a white solid.

### Step 2: 2,4,6-Trimethyl-N-(4-(4,4,55-tetramethyl-1,3,2-dioxaborolan-2-yl)benzyl)-N-(3-(trifluoromethyl)benzyl)benzenesulfonamide (11b)

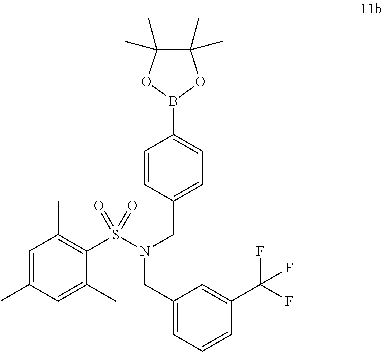 [SMILES: Cc1cc(C)c(S(=O)(=O)N(Cc2ccc(B3OC(C)(C)C(C)(C)O3)cc2)Cc2cccc(C(F)(F)F)c2)c(C)c1]

A suspension of compound 11a (500 mg, 1.20 mmol), 1-(bromomethyl)-3-(trifluoro-methyl)benzene (432 mg, 1.81 mmol) and K₂CO₃ (331 mg, 2.40 mmol) in ACN (200 mL) was stirred at 70° C. for 10 h, cooled, filtered, concentrated and purified by FCC (PE:EA=10:1) to give compound 11b as a white solid.

### Step 3: Methyl 2-((4′-(((2,4,6-trimethyl-N-(3-(trifluoromethyl)benzyl)phenyl)sulfon-amido)methyl)-[1,1′-biphenyl]-3-yl)sulfonyl)acetate (11c)

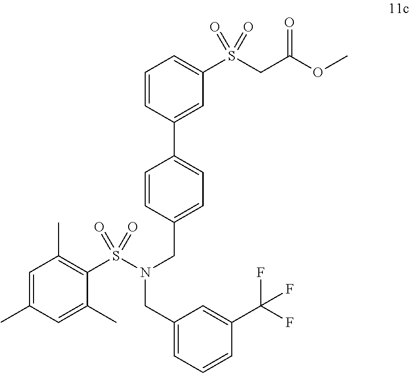

To a suspension of compound 11b (400 mg, 0.70 mmol), methyl 2-((3-bromo-phenyl)sulfonyl)acetate (225 mg, 0.77 mmol), PPh₃ (55 mg, 0.21 mmol) and K₃PO₄ (452 mg, 2.10 mmol) in dioxane (30 mL) was added Pd₂(dba)₃ (65 mg, 70 μmol) at rt under N₂. The mixture was stirred at 85° C. for 10 h, cooled, filtered, concentrated and purified by prep-HPLC to give compound 11c.

### Step 4: 2-((4′-(((2,4,6-Trimethyl-N-(3-(trifluoromethyl)benzyl)phenyl)sulfonamido)methyl)-[1,1′-biphenyl]-3-yl)sulfonyl)acetic acid (11)

Compound 11c was saponified as described for Example 9 to afford compound 11 as a white solid. ¹H-NMR (CDCl₃+few TFA, 400 MHz): δ 8.15 (s, 1H), 7.94 (t, J=8.4 Hz, 2H), 7.70 (t, J=7.8 Hz, 1H), 7.56-7.51 (m, 3H), 7.41 (t, J=7.8 Hz, 1H), 7.29-7.21 (m, 3H), 7.04-7.03 (m, 3H), 4.36 (s, 2H), 4.31 (s, 2H), 4.28 (s, 2H), 2.66 (s, 6H), 2.35 (s, 3H). MS: 646.2 (M+1)⁺.

### Example 11/1 to 11/19

The following Examples were prepared similar as described for Example 11 using the appropriate building blocks.

| # | building block | structure | analytical data |
|---|---|---|---|
| 11/1 | 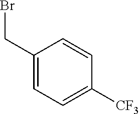 [SMILES: FC(F)(F)c1ccc(CBr)cc1] | 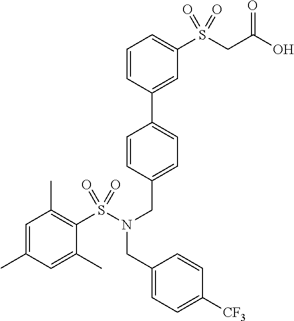 [SMILES: Cc1cc(C)c(S(=O)(=O)N(Cc2ccc(-c3cccc(S(=O)(=O)CC(=O)O)c3)cc2)Cc2ccc(C(F)(F)F)cc2)c(C)c1] | ¹H-NMR (CD₃OD, 400 MHz): δ 8.16 (s, 1H), 7.94 (dd, J = 1.2, 8.0 Hz, 2H), 7.69 (t, J = 7.8 Hz, 1H), 7.57 (t, J = 8.0 Hz, 4H), 7.29 (d, J = 8.0 Hz, 2H), 7.15 (d, J = 8.4 Hz, 2H), 7.08 (s, 2H), 4.42 (s, 2H), 4 34 (s, 2H), 4.32 (s, 2H), 2.63 (s, 6H), 2.33 (s, 3H). MS: 646.2 (M + 1)⁺. |
| 11/2 | 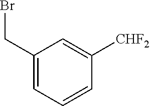 [SMILES: FC(F)c1cccc(CBr)c1] | 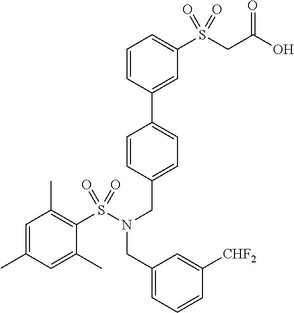 [SMILES: Cc1cc(C)c(S(=O)(=O)N(Cc2ccc(-c3cccc(S(=O)(=O)CC(=O)O)c3)cc2)Cc2cccc(C(F)F)c2)c(C)c1] | ¹H-NMR (CD₃OD + few TFA, 400 MHz): δ 8.16 (t, J = 1.8 Hz, 1H), 7.97-7.94 (m, 2H), 7.70 (t, J = 8.0 Hz, 1H), 7.59 (d, J = 8.4 Hz, 2H), 7.43-7.37 (m, 2H), 7.22-7.20 (m, 3H), 7.10-7.08 (m, 3H), 6.65 (t, J = 56.4 Hz, 1H), 4.36 (s, 2H), 4.35 (s, 2H), 4.34 (s, 2H), 2.63 (s, 6H), 2.33 (s, 3H). MS: 628.2 (M+1)⁺. |
| 11/3 | 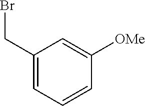 | 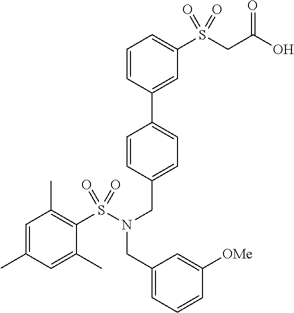 [SMILES: COc1cccc(CN(Cc2ccc(-c3cccc(S(=O)(=O)CC(=O)O)c3)cc2)S(=O)(=O)c2c(C)cc(C)cc2C)c1] | ¹H-NMR (CDCl₃ + few TFA, 400 MHz): δ 8.12 (s, 1H), 7.93 (t, J = 7.4 Hz, 2H), 7.69 (t, J = 8.2 Hz, 1H), 7.52 (d, J = 8.0 Hz, 2H), 7.22-7.19 (m, 3H), 7.04 (s, 2H), 6.84 (dd, J = 2.2, 8.2 Hz, 1H), 6.62 (d, J = 7.6 Hz, 1H), 6.50 (s, 1H), 4.36 (s, 2H), 4.30 (s, 2H), 4.20 (s, 2H), 3.74 (s, 3H), 2.66 (s, 6H), 2.35 (s, 3H). MS: 608.2 (M + 1)⁺. |
| 11/4 | 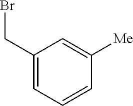 | 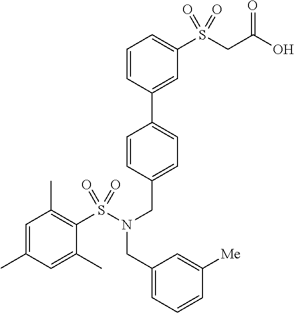 [SMILES: Cc1cccc(CN(Cc2ccc(-c3cccc(S(=O)(=O)CC(=O)O)c3)cc2)S(=O)(=O)c2c(C)cc(C)cc2C)c1] | ¹H-NMR (CDCl₃ + few TFA, 400 MHz): δ 8.16 (s, 1H), 7.95 (dd, J = 1.2, 7.6 Hz, 2H), 7.71 (t, J = 8.0 Hz, 1H), 7,55 (d, J = 8.4 Hz, 2H), 7.23-7.16 (m, 3H), 7.10-7.05 (m, 3H), 6.79 (d, J = 7.2 Hz, 1H), 6.71 (s, 1H), 4.37 (s, 2H), 4.34 (s, 2H), 4.20 (s, 2H), 2.65 (s, 6H), 2.36 (s, 3H), 2.27 (s, 3H). MS: 592.2 (M + 1)⁺. |
| 11/5 | 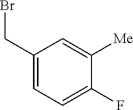 | 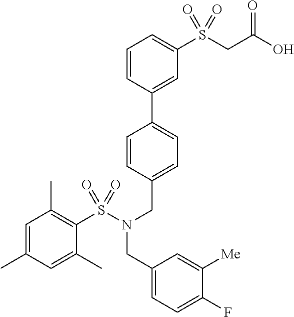 [SMILES: Cc1cc(C)c(S(=O)(=O)N(Cc2ccc(-c3cccc(S(=O)(=O)CC(=O)O)c3)cc2)Cc2ccc(F)c(C)c2)c(C)c1] | ¹H-NMR (CDCl₃ + few TFA, 400 MHz): δ 8.15 (s, 1H), 7.95 (dd, J = 2.0 Hz, 8.0 Hz, 2H), 7.72 (t, J = 7.8 Hz, 1H), 7.55 (d, J = 8.0 Hz, 2H), 7.21 (d, J = 8.0 Hz, 2H), 7.06 (s, 2H), 6.91 (t, J = 8.8 Hz, 1H), 6.79-6.77 (m, 1H), 6.71 (d, J = 7.2 Hz, 1H), 4.35 (s, 4H), 4.19 (s, 2H), 2.64 (s, 6H), 2.37 (s, 3H), 2.18 (d, J = 0.8 Hz, 3H). MS: 610.2 (M + 1)⁺. |
| 11/6 | 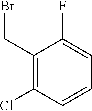 | 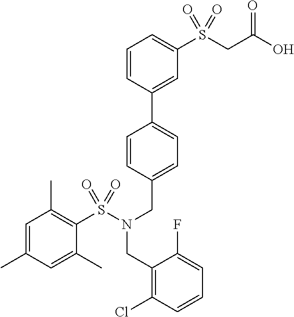 [SMILES: Cc1cc(C)c(S(=O)(=O)N(Cc2ccc(-c3cccc(S(=O)(=O)CC(=O)O)c3)cc2)Cc2c(F)cccc2Cl)c(C)c1] | ¹H-NMR (CDCl₃ + few TFA, 400 MHz): δ 8.09 (s, 1H), 7.92 (d, J = 8.0 Hz, 1H), 7.87 (d, J = 8.0 Hz, 1H), 7.66 (t, J = 8.2 Hz, 1H), 7.42 (d, J = 8.4 Hz, 2H), 7.26-7.23 (m, 2H). 7.11-7.05 (m, 1H), 6.99 (d, J = 8.0 Hz, 1H), 6.93 (s, 2H), 6.76 (t, J = 8.6 Hz, 1H), 4.52 (s, 2H), 4.51 (s, 2H), 4.28 (s, 2H), 2.65 (s, 6H), 2.29 (s, 3H). MS: 630.1 (M + 1)⁺. |
| 11/7 | 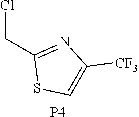 | 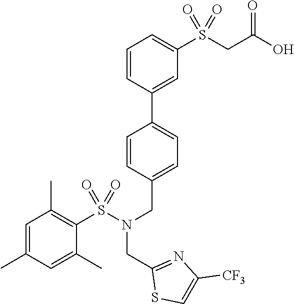 [SMILES: Cc1cc(C)c(S(=O)(=O)N(Cc2ccc(-c3cccc(S(=O)(=O)CC(=O)O)c3)cc2)Cc2nc(C(F)(F)F)cs2)c(C)c1] | ¹H-NMR (CDCl₃ + few TFA, 400 MHz): δ 8.09 (s, 1H), 7.91-7.89 (m, 1H), 7.84-7.83 (m, 1H), 7.70 (s, 1H), 7.62-7.60 (m, 1H), 7.49-7.47 (m, 2H), 7.20 (d, J = 7.6 Hz, 2H), 6.99 (s, 2H), 4.59 (s, 2H), 4.42 (s, 2H), 4.21 (s, 2H), 2.64 (s, 6H), 2.32 (s, 3H). MS: 653.1 (M + 1)⁺. |
| 11/8 | 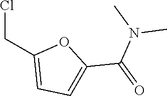 | 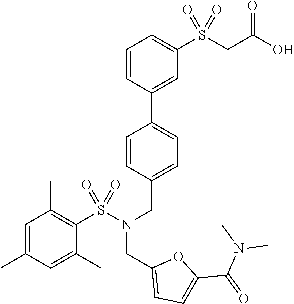 [SMILES: Cc1cc(C)c(S(=O)(=O)N(Cc2ccc(-c3cccc(S(=O)(=O)CC(=O)O)c3)cc2)Cc2ccc(C(=O)N(C)C)o2)c(C)c1] | ¹H-NMR (CDCl₃, 400 MHz): δ 8.03 (s, 1H), 7.86 (d, J = 7.2 Hz, 1H), 7.68 (d, J = 8.4 Hz, 1H), 7.49-7.46 (m, 1H), 7.39 (d, J = 7.6 Hz, 2H), 7.09 (d, J = 8.0 Hz, 2H), 6.96 (s, 2H), 6.83 (d, J = 3.2 Hz, 1H), 6.05 (d, J = 3.6 Hz, 1H), 4.26 (s, 2H), 4.25 (s, 2H), 4.12 (s, 2H), 3.18 (br s, 3H), 3.01 (br s, 3H), 2.61 (s, 6H), 2.29 (s, 3H). MS: 639.1 (M + 1)⁺. |
| 11/9 | 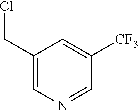 [SMILES: FC(F)(F)c1cncc(CCl)c1] | 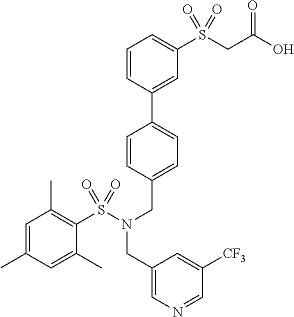 [SMILES: Cc1cc(C)c(S(=O)(=O)N(Cc2ccc(-c3cccc(S(=O)(=O)CC(=O)O)c3)cc2)Cc2cncc(C(F)(F)F)c2)c(C)c1] | ¹H-NMR (CDCl₃ + few TFA, 400 MHz): δ 8.97 (s, 1H), 8.79 (s, 1H), 8.56 (s, 1H), 8.02 (s, 1H), 7.96 (d, J = 7.6 Hz, 1H), 7.86 (d, J = 8.0 Hz, 1H), 7.71 (t, J = 7.8 Hz, 1H), 7.45 (d, J = 8.0 Hz, 2H), 7.20 (d, J = 8 0 Hz, 2H), 7.08 (s, 2H), 4.72 (s, 2H), 4.37 (s, 2H), 4.32 (s, 2H), 2.67 (s, 6H), 2.36 (s, 3H). MS: 647.1 (M + 1)⁺_{.} |
| 11/10 | 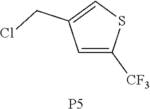 | 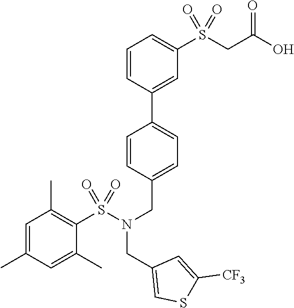 [SMILES: Cc1cc(C)c(S(=O)(=O)N(Cc2ccc(-c3cccc(S(=O)(=O)CC(=O)O)c3)cc2)Cc2csc(C(F)(F)F)c2)c(C)c1] | ¹H-NMR (CDCl₃ + few TFA, 400 MHz): δ 8.13 (s, 1H), 7.95-7.93 (m, 2H), 7.72 (t, J = 7.4 Hz, 1H), 7.55 (d, J = 7.6 Hz, 2H), 7.23- 7.17 (m, 3H), 7.06 (s, 2H), 6.83 (s, 1H), 4.38 (s, 2H), 4.34 (s, 2H), 4.25 (s, 2H), 2.64 (s, 6H), 2.36 (s, 3H). MS: 652.1 (M + 1)⁺. |
| 11/11 | 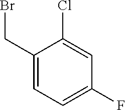 | 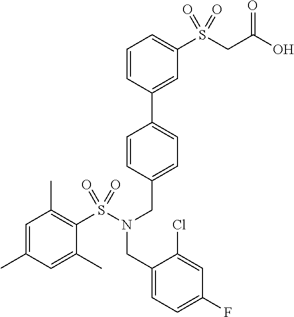 [SMILES: Cc1cc(C)c(S(=O)(=O)N(Cc2ccc(-c3cccc(S(=O)(=O)CC(=O)O)c3)cc2)Cc2ccc(F)cc2Cl)c(C)c1] | ¹H-NMR (CDCl₃ + few TFA, 400 MHz): δ 8.13 (t, J = 1.6 Hz, 1H), 7.95 (td, J = 1.5, 8.0 Hz, 2H), 7.71 (t, J = 7.8 Hz, 1H), 7.48 (d, J = 8.8 Hz, 2H), 7.29-7.25 (m, 1H), 7.11 (d, J = 8.0 Hz, 2H), 7.05-7.02 (m, 3H), 6.95-6.91 (m, 1H), 4.48 (s, 2H), 4.40 (s, 2H), 4.35 (s, 2H), 2.66 (s, 6H), 2.34 (s, 3H). MS: 630.1 (M + 1)⁺. |
| 11/12 | 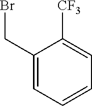 [SMILES: FC(F)(F)c1ccccc1CBr] | 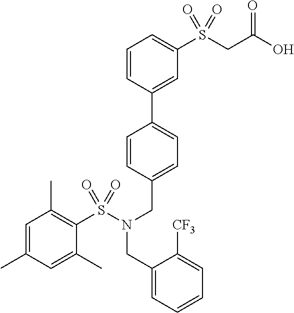 [SMILES: Cc1cc(C)c(S(=O)(=O)N(Cc2ccc(-c3cccc(S(=O)(=O)CC(=O)O)c3)cc2)Cc2ccccc2C(F)(F)F)c(C)c1] | ¹H-NMR (CDCl₃ + few TFA, 400 MHz): δ 8.09 (s, 1H), 7.93 (d, J = 7.6 Hz, 1H), 7.88 (d, J = 8.0 Hz, 1H), 7.69-7.62 (m, 3H), 7.56 (t, J = 7.4 Hz, 1H), 7.41-7.36 (m, 3H), 7.04 (s, 2H), 6.90 (d, J = 8.0 Hz, 2H), 4.68 (s, 2H), 4.36 (s, 2H), 4.30 (s, 2H), 2.67 (s, 6H), 2.35 (s, 3H). MS: 646.2 (M + 1)⁺. |
| 11/13 | 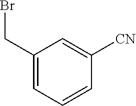 | 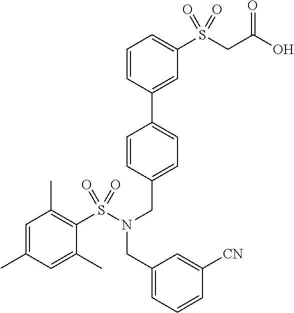 | ¹H-NMR (CDCl₃ + few TFA, 400 MHz): δ 8.16 (s, 1H), 7.95 (t, J = 9.4 Hz, 2H), 7.71 (t, J = 7.8 Hz, 1H), 7.59-7.53 (m, 3H), 7.48- 7.44 (m, 2H), 7.13 (d, J = 8.0 Hz, 2H), 7.09- 7.07 (m, 3H), 4.35 (s, 2H), 4.34 (s, 2H), 4.31 (s, 2H), 2.65 (s, 6H), 2.37 (s, 3H). MS: 644.2 (M + 1)⁺. |
| 11/14 | 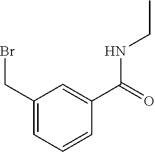 | 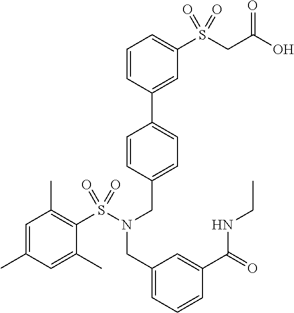 [SMILES: CCNC(=O)c1cccc(CN(Cc2ccc(-c3cccc(S(=O)(=O)CC(=O)O)c3)cc2)S(=O)(=O)c2c(C)cc(C)cc2C)c1] | ¹H-NMR (CDCl₃ + few TFA, 400 MHz): δ 8.09 (s, 1H), 7.93 (t, J = 9.4 Hz, 2H), 7.72- 7.66 (m, 2H), 7.62 (br s, 1H), 7.47 (d, J = 8.0 Hz, 2H), 7.42 (t, J = 7.6 Hz, 1H), 7.30- 7.27 (m, 1H), 7.08 (s, 2H), 7.01 (d, J = 7.6 Hz, 2H), 4.44 (s, 2H), 4.34 (s, 2H). 4.23 (s, 2H), 3.52 (q, J = 7.2 Hz, 2H), 2.66 (s, 6H), 2.37 (s, 3H), 1.28 (t, J = 7.2 Hz, 3H). MS: 649.2 (M + 1)⁺. |
| 11/15 | 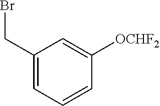 [SMILES: FC(F)Oc1cccc(CBr)c1] | 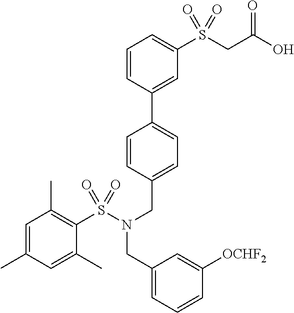 [SMILES: Cc1cc(C)c(S(=O)(=O)N(Cc2ccc(-c3cccc(S(=O)(=O)CC(=O)O)c3)cc2)Cc2cccc(OC(F)F)c2)c(C)c1] | ¹H-NMR (CDCl₃ + few TFA, 400 MHz): δ 8.16 (s, 1H), 7.97-7.94 (m, 2H), 7.72 (t, J = 7.8 Hz, 1H), 7.55 (d, J = 8.0 Hz, 2H), 7.30 (t, J = 8.0 Hz, 1H), 7.20 (d, J = 8.0 Hz, 2H), 7.06-7.03 (m, 3H), 6.92 (d, J = 7.6 Hz, 1H), 6.67 (s, 1H), 6.43 (t, J = 73.6 Hz, 1H), 4.36 (s, 4H), 4.25 (s, 2H), 2.66 (s, 6H), 2.36 (s, 3H). MS: 644.2 (M + 1)⁺. |
| 11/16 | 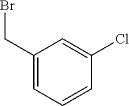 | 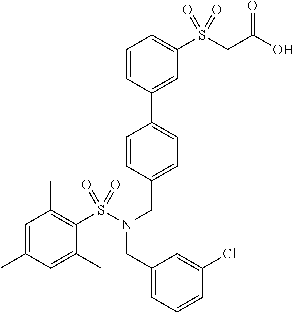 [SMILES: Cc1cc(C)c(S(=O)(=O)N(Cc2ccc(-c3cccc(S(=O)(=O)CC(=O)O)c3)cc2)Cc2cccc(Cl)c2)c(C)c1] | ¹H-NMR (CDCl₃ + few TFA, 400 MHz): δ 8.16 (d, J = 2.0 Hz, 1H), 7.95 (d, J = 7.2 Hz, 2H), 7.72 (t, J = 7.8 Hz, 1H), 7.55 (d, J = 8.0 Hz, 2H), 7.25-7.20 (m, 4H), 7.06 (s, 2H), 6.95 (d, J = 7.6 Hz, 1H), 6.81 (s, 1H), 4.37 (s, 2H), 4.34 (s, 2H), 4.22 (s, 2H), 2.65 (s, 6H), 2.37 (s, 3H) MS: 612.1 (M + 1)⁺. |
| 11/17 | 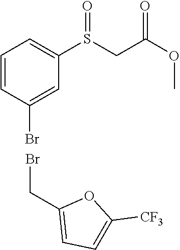 | 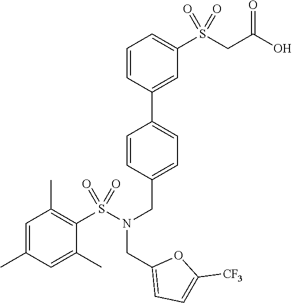 [SMILES: Cc1cc(C)c(S(=O)(=O)N(Cc2ccc(-c3cccc(S(=O)(=O)CC(=O)O)c3)cc2)Cc2ccc(C(F)(F)F)o2)c(C)c1] | ¹H-NMR (CD₃OD, 400 MHz): δ 7.97 (t, J = 1.4 Hz, 1H), 7.83-7.81 (m, 1H), 7.73-7.71 (m, 1H), 7. 67 (d, J = 8.0 Hz, 1H), 7.61 (d, J = 8.0 Hz, 2H), 7.28 (d, J = 8.0 Hz, 2H), 7.05 (s, 2H), 6.80-6.79 (m, 1H), 6.27 (d, J = 3.2 Hz, 1H), 4.43 (s, 2H), 4.33 (s, 2H), 3.95- 3.89 (m, 2H), 2.62 (s, 6H), 2.31 (s, 3H). MS 637.2 (M + 18)⁺. |
| 11/18 | 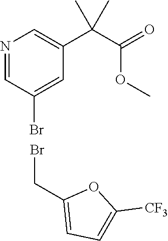 | 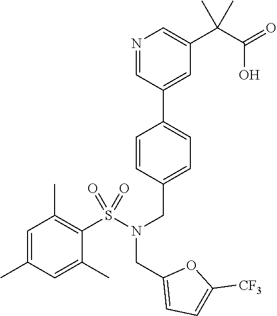 [SMILES: Cc1cc(C)c(S(=O)(=O)N(Cc2ccc(-c3cncc(C(C)(C)C(=O)O)c3)cc2)Cc2ccc(C(F)(F)F)o2)c(C)c1] | ¹H-NMR (CDCl₃, 400 MHz): δ 8.65 (s, 2H), 7.89 (s, 1H), 7.51-7.47 (m, 2H), 7.26-7.24 (m, 2H), 6.98 (s, 2H), 6.63 (s, 1H), 6.18 (s, 1H), 4.38 (s, 2H), 4.23 (s, 2H), 2.62 (s, 6H), 2.31 (s, 3H), 1.64 (s, 6H). MS: 601.0 (M + 1)⁺. |
| 11/19 | 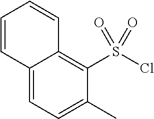 | 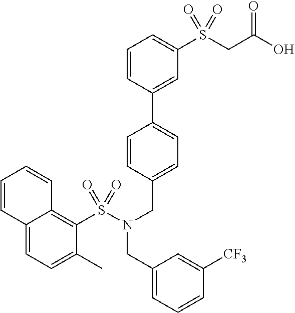 [SMILES: Cc1ccc2ccccc2c1S(=O)(=O)N(Cc1ccc(-c2cccc(S(=O)(=O)CC(=O)O)c2)cc1)Cc1cccc(C(F)(F)F)c1] | ¹H-NMR (CDCl₃, 400 MHz): δ 7.79 (d, J = 9.2 Hz, 1H), 8.01 (s, 1H), 7.87-7.79 (m, 3H), 7.59-7.47 (m, 3H), 7.38 (t, J = 8.4 Hz, 1H), 7.30-7.25 (m, 4H), 7.18-7.14 (m, 1H), 7.02-6.92 (m, 3H), 6.81 (s, 1H), 4.30 (s, 2H), 4.22 (s, 2H), 4.17 (s, 2H), 2.84 (s, 3H). MS: 667.9 (M + 1)⁺. |

### Example 12

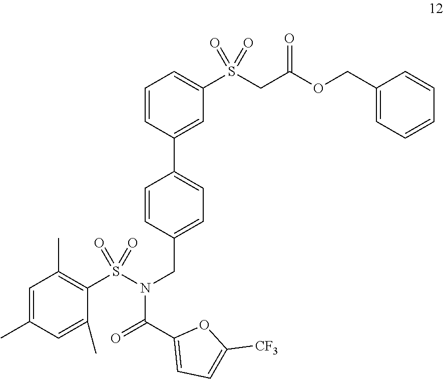 [SMILES: Cc1cc(C)c(S(=O)(=O)N(Cc2ccc(-c3cccc(S(=O)(=O)CC(=O)OCc4ccccc4)c3)cc2)C(=O)c2ccc(C(F)(F)F)o2)c(C)c1]

### Step 1: Benzyl 2-((3-bromophenyl)thio)acetate (12a)

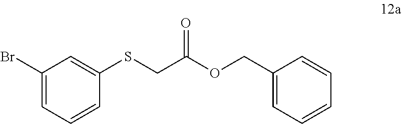 [SMILES: O=C(CSc1cccc(Br)c1)OCc1ccccc1]

To a solution of benzyl 2-bromoacetate (13.3 g, 58.2 mmol) and K₂CO₃ (14.6 g, 106 mmol) in ACN (120 mL) was added 3-bromobenzenethiol (10.0 g, 52.9 mmol). The mixture was stirred at 80° C. overnight under N₂, cooled, filtered and concentrated to afford compound 12a as a yellow oil. MS: 337.

### Step 2: Benzyl 2-((3-bromophenyl)sulfonyl)acetate (12b)

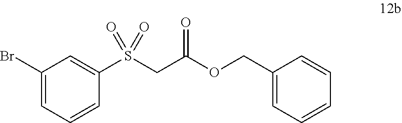 [SMILES: O=C(CS(=O)(=O)c1cccc(Br)c1)OCc1ccccc1]

To a solution of compound 12a (2.0 g, 5.97 mmol) in DCM (40 mL) was added m-CPBA (1.13 g, 5.97 mmol) at 0° C. The mixture was stirred at rt for 0.5 h. Then another m-CPBA (1.13 g, 5.97 mmol) was added and the mixture was stirred at 30° C. overnight, diluted with a Na₂CO₃ solution and extracted with CH₂Cl₂. The organic layer was washed with brine, dried over Na₂SO₄, concentrated and purified by FCC (PE:EA=5:1) to afford compound 12b as a yellow oil. ¹H-NMR (CDCl₃, 400 MHz): δ 8.03 (t, 1H), 7.74-7.78 (m, 2H), 7.37-7.37 (m, 4H), 7.26-7.29 (m, 2H), 5.13 (s, 2H), 4.17 (s, 2H).

### Step 3: Benzyl 2-((3-(4,4,5,5-tetramethyl-1,3,2-dioxaborolan-2-yl)phenyl)sulfonyl)acetate (12c)

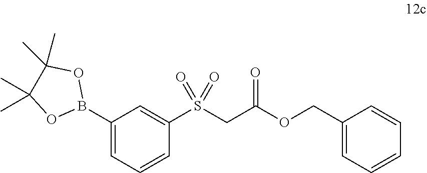 [SMILES: CC1(C)OB(c2cccc(S(=O)(=O)CC(=O)OCc3ccccc3)c2)OC1(C)C]

A solution of compound 12b (1.8 g, 4.91 mmol), B₂Pin₂ (1.62 g, 6.38 mmol), Pd₂(dba)₃ (135 mg, 0.15 mmol), X-phos (211 mg, 0.44 mmol) and KOAc (1.44 g, 14.7 mmol) in dioxane (100 mL) was stirred at 90° C. for 2 h under N₂, cooled and filtered. The filtrate was diluted with water and extracted with EA. The organic layer was washed with brine, dried over Na₂SO₄, concentrated and purified by FCC (PE:EA=5:1) to afford compound 12c as a yellow oil.

### Step 4: 5-(Trifluoromethyl)furan-2-carbonyl chloride (12d)

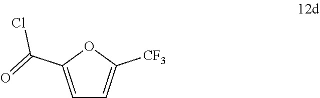 [SMILES: O=C(Cl)c1ccc(C(F)(F)F)o1]

To a mixture of 5-(trifluoromethyl)furan-2-carboxylic acid (500 mg, 2.78 mmol) in DCM (15 mL) was added (COCl)₂ (3.53 g, 27.8 mmol) and the mixture was stirred at 40° C. for 5 h and concentrated to afford compound 12d which was used in the next step directly.

### Step 5: N-(4-Bromobenzyl)-N-(mesitylsulfonyl)-5-(trifluoromethyl)furan-2-carboxamide (12e)

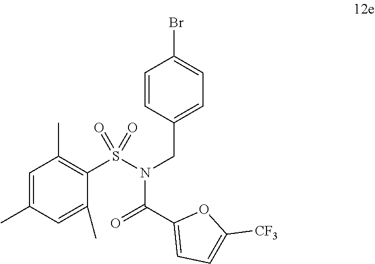 [SMILES: Cc1cc(C)c(S(=O)(=O)N(Cc2ccc(Br)cc2)C(=O)c2ccc(C(F)(F)F)o2)c(C)c1]

To a solution of compound 12d (1.1 g, 3.06 mmol) in dry THF (20 mL) was added NaH (80 mg, 95%, 3.34 mmol) at 0° C. After stirring for 0.5 h, a solution of compound 1a in dry DMF was added and the mixture was heated to 40° C. for 6 h, poured into ice water (150 mL) and extracted with EA. The organic layer was washed with brine, dried over Na₂SO₄, concentrated and purified by FCC (PE:EA=10:1) to afford compound 12e as a white solid. ¹H-NMR (CDCl₃, 400 MHz): δ 7.41 (d, J=8.8 Hz, 2H), 7.24 (d, J=8.8 Hz, 2H), 7.00-6.98 (m, 3H), 6.75 (d, J=2.8 Hz, 1H), 5.32 (s, 2H), 2.69 (s, 6H), 2.30 (s, 3H). MS: 530.

### Step 6: Benzyl 2-((4′-((N-(mesitylsulfonyl)-5-(trifluoromethyl)furan-2-carboxamido)methyl)-[1,1′-biphenyl]-3-yl)sulfonyl)acetate (12)

A mixture of compound 12e (250 mg, 0.47 mmol) and compound 12c (255 mg, 0.61 mmol), Pd₂(dba)₃ (43 mg, 50 μmol), PPh₃ (37 mg, 140 μmol) and K₃PO₄ (304 mg, 1.42 mmol) in dioxane (30 mL) was stirred at 85° C. for 6 h under N₂, cooled, filtered, concentrated and purified by FCC (PE:EA=5:1) to afford compound 12 as a yellow oil. ¹H-NMR (CDCl₃, 300 MHz): δ 8.04 (s, 1H), 7.80-7.81 (m, 2H), 7.51-7.57 (m, 2H), 7.47 (s, 4H), 7.29-7.33 (m, 4H), 6.99-7.00 (m, 3H), 6.76-6.74 (m, 1H), 5.44 (s, 2H), 5.11 (s, 2H), 4.19 (s, 2H), 2.72 (s, 6H), 2.31 (s, 3H).

### Example 13

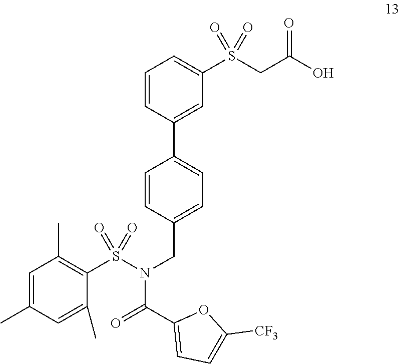 [SMILES: Cc1cc(C)c(S(=O)(=O)N(Cc2ccc(-c3cccc(S(=O)(=O)CC(=O)O)c3)cc2)C(=O)c2ccc(C(F)(F)F)o2)c(C)c1]

### 2-((4′-((N-(Mesitylsulfonyl)-5-(trifluoromethyl)furan-2-carboxamido)methyl)-[1,1′-biphenyl]-3-yl)sulfonyl)acetic acid (13)

To a solution of compound 12 (50 mg, 68 μmol) and 4-methylmorpholine (7 mg, 68 μmol) in EtOH/EA (8 mL/2 mL) was added 10% Pd/C (25 mg). The mixture was stirred at rt for 10 min under H₂, filtered, concentrated and purified by prep-HPLC to afford compound 13 as a white solid. ¹H-NMR (DMSO-d₆, 300 MHz): δ 8.13 (d, J=1.2 Hz, 1H), 7.96 (d, J=7.8 Hz, 1H), 7.86 (d, J=8.1 Hz, 1H), 7.76 (d, J=8.1 Hz, 2H), 7.68 (t, J=7.5 Hz, 1H), 7.47 (d, J=8.4 Hz, 2H), 7.37-7.32 (m, 2H), 7.20-7.10 (m, 3H), 5.45 (br s, 2H), 4.24 (br s, 2H), 2.62 (s, 6H), 2.28 (s, 3H). MS: 650.1 (M+1)⁺.

### Example 14

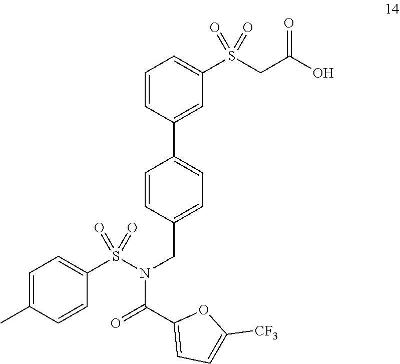 [SMILES: Cc1ccc(S(=O)(=O)N(Cc2ccc(-c3cccc(S(=O)(=O)CC(=O)O)c3)cc2)C(=O)c2ccc(C(F)(F)F)o2)cc1]

### 2-((4′-(((4-Methyl-N-((5-(trifluoromethyl)furan-2-yl)methyl)phenyl)sulfonamido)methyl)-[1,1′-biphenyl]-3-yl)sulfonyl)acetic acid (14)

Similar as described for Example 11, however in a different order, (4-(4,4,5,5-tetramethyl-1,3,2-dioxaborolan-2-yl)phenyl)methanamine was reacted with 2-(bromomethyl)-5-(trifluoro-methyl)furan and then the product was reacted in the next step with 4-methylbenzenesulfonyl chloride. This intermediate was coupled and saponified as described in Example 11, Step 3 and 4, to give compound 14 as a white solid. ¹H-NMR (CDCl₃, 400 MHz): δ 8.04 (s, 1H), 7.83 (d, J=7.6 Hz, 1H), 7.64 (d, J=8.0 Hz, 3H), 7.42-7.40 (m, 3H), 7.23 (d, J=8.4 Hz, 4H), 6.49 (d, J=2.0 Hz, 1H), 6.04 (d, J=3.2 Hz, 1H), 4.25 (s, 2H), 4.25 (s, 2H), 4.16 (s, 2H), 2.38 (s, 3H). MS: 608.0 (M+1)⁺, 625.1 (M+18)⁺.

### Example 14/1 to 14/3

The following Examples were prepared similar as described for Example 14 using the appropriate building blocks.

| # | building block | structure | analytical data |
|---|---|---|---|
| 14/1 | 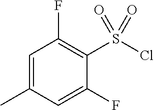 | 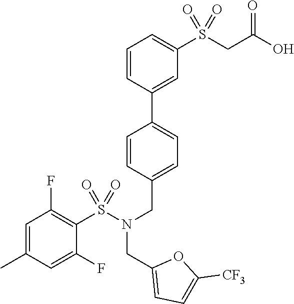 [SMILES: Cc1cc(F)c(S(=O)(=O)N(Cc2ccc(-c3cccc(S(=O)(=O)CC(=O)O)c3)cc2)Cc2ccc(C(F)(F)F)o2)c(F)c1] | ¹H-NMR (CDCl₃, 400 MHz): δ 8.01 (s, 1H), 7.79 (d, J = 7.6 Hz, 1H), 7.59 (d, J = 7.6 Hz, 1H), 7.39-7.33 (m, 3H), 7.20 (d, J = 8.4 Hz, 2H), 6.75 (d, J = 10.0 Hz, 2H), 6.49 (d, J = 2.4 Hz, 1H), 6.11 (d, J = 3.6 Hz, 1H), 4.39 (s, 2H), 4.29 (s, 2H), 4.17 (s, 2H), 2.34 (s, 3H). MS: 661.0 (M + 18)⁺. |
| 14/2 | 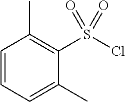 | 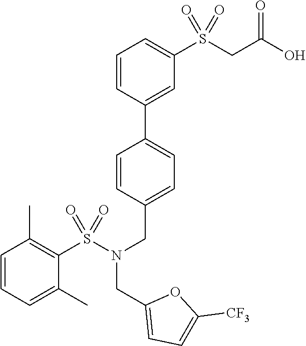 [SMILES: Cc1cccc(C)c1S(=O)(=O)N(Cc1ccc(-c2cccc(S(=O)(=O)CC(=O)O)c2)cc1)Cc1ccc(C(F)(F)F)o1] | ¹H-NMR (CDCl₃, 300 MHz): δ 8.04 (s, 1H), 7.84 (d, J = 8.1 Hz, 1H), 7.64 (d, J = 7.8 Hz, 1H), 7.42 (d, J = 7.8 Hz, 2H), 7.28 (s, 1H), 7.16-7.11 (m, 4H), 6.56 (br s, 1H), 6.08 (d, J = 3.0 Hz, 1H), 4.32 (s, 2H), 4.16 (s, 2H), 4.13 (s, 2H), 2.60 (s, 6H). MS: 622.1 (M + 1)⁺, 639.1 (M + 18)⁺. |
| 14/3 | 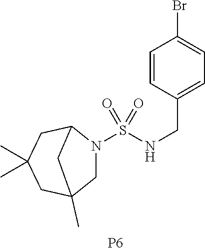 | 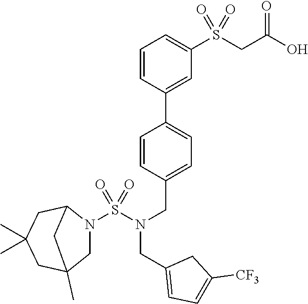 [SMILES: CC1(C)CC2CC(C)(CN2S(=O)(=O)N(CC2=CC=C(C(F)(F)F)C2)Cc2ccc(-c3cccc(S(=O)(=O)CC(=O)O)c3)cc2)C1] | ¹H-NMR (CDCl₃, 300 MHz): δ 8.07 (s, 1H), 7.85 (d, J = 7.8 Hz, 1H), 7.71 (d, J = 7.6 Hz, 1H), 7.49-7.45 (m, 3H), 7.34 (d, J = 8.4 Hz, 2H), 6.68-6.66 (m, 1H), 6.26 (d, J = 3.3 Hz, 1H), 4.32-3.28 (m, 2H), 4.23-4.09 (m, 5H), 3.20 (dd, J = 9.0 Hz, 0.6 Hz, 1H), 3.00 (dd, J = 9.3 Hz, 0.9 Hz, 1H), 1.84-1.23 (m, 6H), 1.17 (s, 3H), 1.04 (s, 3H), 0.91 (s, 3H). MS: 669.1 (M + 1)⁺. |

### Example 15

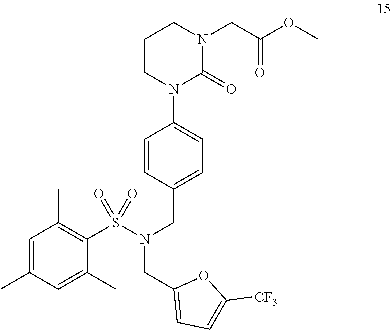

### Methyl 2-(2-oxo-3-(4-(((2,4,6-trimethyl-N-((5-(trifluoromethyl)furan-2-yl)methyl)phenyl)sulfon-amido)methyl)phenyl)tetrahydropyrimidin-1(2H)-yl)acetate (15)

To a solution of compound 3a (200 mg, 0.58 mmol), methyl 2-(2-oxotetrahydropyrimidin-1(2H)-yl)acetate (120 mg, 0.69 mmol), Cs₂CO₃ (378 mg, 1.1 mmol) and BINAP (33 mg, 50 μmol) in dioxane (20 mL) was added Pd₂(dba)₃ (26 mg, 30 μmol). The mixture was stirred at 100° C. under N₂ overnight, cooled, filtered, concentrated and purified by FCC (PE:EA=10:1 to 1:1) to give compound 15 as a colorless oil. MS: 608.

### Example 15/1 to 15/2

The following Examples were prepared similar as described for Example 15 using the appropriate building blocks.

| # | building block | structure | analytical data |
|---|---|---|---|
| 15/1 | 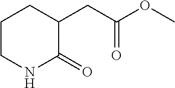 | 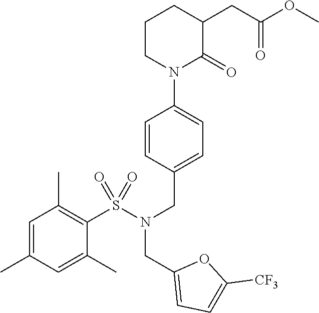 | MS: 607 (M + 1)⁺. |
| 15/2 | 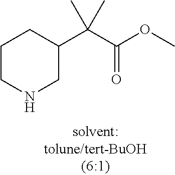 | 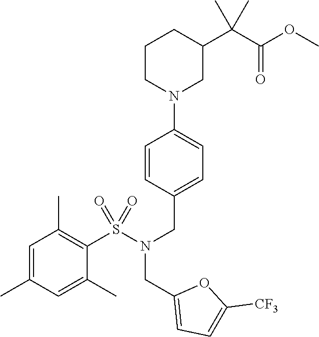 | MS: 621 (M + 1)⁺. |

### Example 16

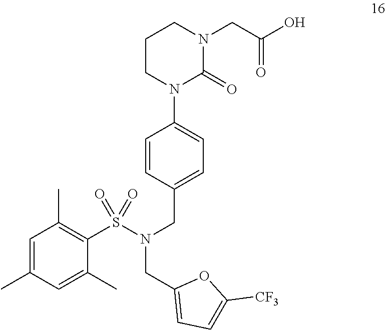 [SMILES: Cc1cc(C)c(S(=O)(=O)N(Cc2ccc(N3CCCN(CC(=O)O)C3=O)cc2)Cc2ccc(C(F)(F)F)o2)c(C)c1]

### 2-(2-Oxo-3-(4-(((2,4,6-trimethyl-N-((5-(trifluoromethyl)furan-2-yl)methyl)phenyl)sulfon-amido)methyl)phenyl)tetrahydropyrimidin-1(2H)-yl)acetic acid (16)

Compound 15 (200 mg, 0.30 mmol) was saponified as described for Example 10, Step 4 to obtain compound 16 as a white solid. ¹H-NMR (CDCl₃, 400 MHz): δ 7.18 (d, J=8.0 Hz, 2H), 8.11 (d, J=8.0 Hz, 2H), 6.95 (s, 2H), 6.61 (s, 1H), 6.16 (s, 1H), 4.29 (s, 2H), 4.17 (s, 2H), 3.91 (s, 2H), 3.66 (t, J=5.0 Hz, 2H), 3.44 (t, J=5.2 Hz, 2H), 2.58 (s, 6H), 2.30 (s, 3H), 2.12-2.08 (m, 2H). MS: 594.0 (M+H)⁺.

### Example 16/1 to 16/2

The following Examples were prepared similar as described for Example 16.

| # | educt | structure | analytical data |
|---|---|---|---|
| 16/1 | 15/1 | 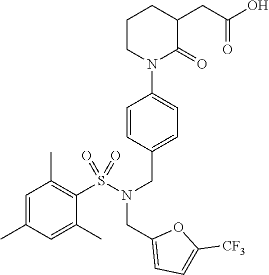 [SMILES: Cc1cc(C)c(S(=O)(=O)N(Cc2ccc(N3CCCC(CC(=O)O)C3=O)cc2)Cc2ccc(C(F)(F)F)o2)c(C)c1] | ¹H-NMR (CDCl₃, 400 MHz): δ 7.23-7.17 (m, 4H), 6.97 (s, 2H), 6.64 (d, J = 1.4 Hz, J = 3.4 Hz, 1H), 6.19 (d, J = 3.6 Hz, 1H), 4.34 (s, 2H), 4.25 (s, 2H), 3.73-3.69 (m, 1H), 3.64-3.60 (m, 1H), 2.93-2.85 (m, 2H), 2.62-2.56 (m, 7H), 2.31 (s, 3H), 2.17-2.14 (m, 1H), 2.06-2.01 (m, 2H), 1.82-1.72 (m, 1H). MS: 593.0 (M + 1)⁺. |
| 16/2 | 15/2 | 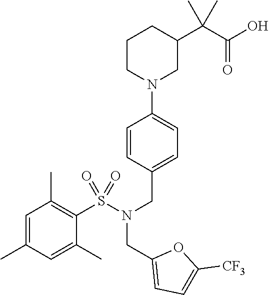 [SMILES: Cc1cc(C)c(S(=O)(=O)N(Cc2ccc(N3CCCC(C(C)(C)C(=O)O)C3)cc2)Cc2ccc(C(F)(F)F)o2)c(C)c1] | ¹H-NMR (CDCl₃, 400 MHz): δ 6.99-6.97 (m, 4H), 6.83 (d, J = 8.0 Hz, 2H), 6.65 (d, J = 2.4 Hz, 1H), 6.22 (d, J = 3.2 Hz, 1H), 4.21 (s, 2H), 4.21 (s, 2H), 3.67-3 64 (m, 2H), 2.66-2.58 (m, 8H), 2.32 (s, 3H), 2.00-1.96 (m, 1H), 1.84-1.78 (m, 2H), 1.68-1.63 (m, 1H), 1.31-1.25 (m, 7H). MS: 607.0 (M + 1)⁺. |

### Example 17

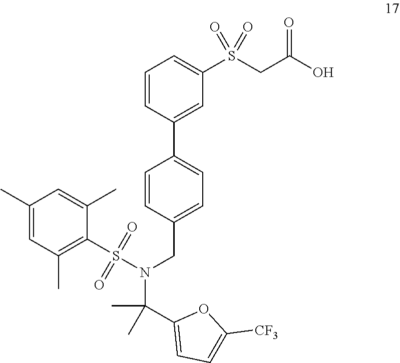 [SMILES: Cc1cc(C)c(S(=O)(=O)N(Cc2ccc(-c3cccc(S(=O)(=O)CC(=O)O)c3)cc2)C(C)(C)c2ccc(C(F)(F)F)o2)c(C)c1]

### Step 1: N-(2-(Furan-2-yl)propan-2-yl)-2,4,6-trimethylbenzenesulfonamide (17a)

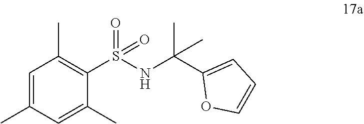 [SMILES: Cc1cc(C)c(S(=O)(=O)NC(C)(C)c2ccco2)c(C)c1]

To a solution of 2-(furan-2-yl)propan-2-amine hydrogen chloride (550 mg, 3.41 mmol) and 2,4,6-trimethylbenzenesulfonyl chloride (1.49 g, 6.81 mmol) in DCM (50 mL) was added TEA (3.0 mL) under ice cooling and under N₂. The mixture was stirred at rt overnight, diluted with water (50 mL) and extracted with EA (3×50 mL). The combined organic layer was washed with water (2×100 mL) and brine (100 mL), dried over Na₂SO₄, filtered, concentrated and purified by FCC (PE:EA=8:1) to give compound 17a as a white solid.

### Step 2: 2,4,6-Trimethyl-N-(2-(5-(trifluoromethyl)furan-2-yl)propan-2-yl)benzenesulfonamide (17b)

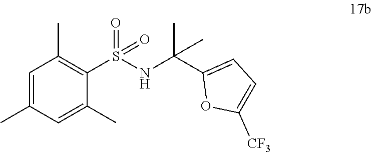 [SMILES: Cc1cc(C)c(S(=O)(=O)NC(C)(C)c2ccc(C(F)(F)F)o2)c(C)c1]

To a solution of compound 17a (250 mg, 0.81 mmol), PhI(OAc)₂ (786 mg, 2.44 mmol) and AgF (52 mg, 0.41 mmol) in DMSO (13 mL) was added TMSCF₃ (347 mg, 2.44 mmol) at rt under N₂. The mixture was stirred at rt overnight, diluted with water (50 mL) and extracted with EA (3×50 mL). The combined organic layer was washed with water (2×100 mL), sat. Na₂S₂O₃ (50 mL) and brine (100 mL), dried over Na₂SO₄, filtered, concentrated and purified by FCC (PE:EA=10:1) to give compound 17b as a white solid.

### Step 3: N-(4-Bromobenzyl)-2,4,6-trimethyl-N-(2-(5-(trifluoromethyl)furan-2-yl)propan-2-yl)benzenesulfonamide (17c)

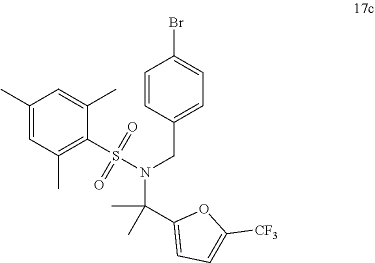 [SMILES: Cc1cc(C)c(S(=O)(=O)N(Cc2ccc(Br)cc2)C(C)(C)c2ccc(C(F)(F)F)o2)c(C)c1]

To a solution of compound 17b (200 mg, 0.53 mmol) in dry DMF (15 mL) was added NaH (32 mg, 60%, 0.80 mmol) under ice cooling and under N₂. The mixture was stirred at 0° C. for 10 min, then 1-bromo-4-(bromomethyl)benzene (160 mg, 0.64 mmol) was added and the mixture was stirred at rt overnight, diluted with water (50 mL) and extracted with EA (3×50 mL). The combined organic layer was washed with water (2×100 mL) and brine (100 mL), dried over Na₂SO₄, filtered, concentrated and purified by FCC (PE:EA=20:1) to give compound 17c as a white solid.

### Step 4: Methyl 2-((4′-(((2,4,6-trimethyl-N-(2-(5-(trifluoromethyl)furan-2-yl)propan-2-yl)phenyl)sulfonamido)methyl)-[1,1′-biphenyl]-3-yl)sulfonyl)acetate (17d)

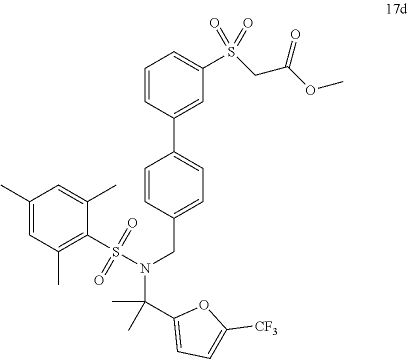

To a suspension of compound 17c (200 mg, 0.37 mmol), methyl 2-((3-(4,4,5,5-tetramethyl-1,3,2-dioxaborolan-2-yl)phenyl)sulfonyl)acetate (137 mg, 0.40 mmol), PPh₃ (29 mg, 110 μmol) and K₃PO₄ (239 mg, 1.11 mmol) in dioxane (20 mL) was added Pd₂dba₃ (34 mg, 40 μmol) at rt under N₂. The mixture was stirred at 85° C. for 10 h, filtered, concentrated and purified by FCC (PE:EA=4:1) to give compound 17d as a yellow oil.

### Step 5: 2-((4′-(((2,4,6-Trimethyl-N-(2-(5-(trifluoromethyl)furan-2-yl)propan-2-yl)phenyl)sulfon-amido)methyl)-[1,1′-biphenyl]-3-yl)sulfonyl)acetic acid (17)

Compound 17d (170 mg, 0.25 mmol) was saponified as described in Example 9 and purified by prep-HPLC to give compound 17 as a white solid. ¹H-NMR (CDCl₃, 400 MHz): δ 8.10 (s, 1H), 7.88 (d, J=7.2 Hz, 1H), 7.76 (d, J=8.0 Hz, 1H), 7.52 (t, J=7.6 Hz, 1H), 7.45 (d, J=8.0 Hz, 2H), 7.37 (d, J=8.0 Hz, 2H), 6.90 (s, 2H), 6.52 (d, J=2.8 Hz, 1H), 6.16 (d, J=2.8 Hz, 1H), 4.50 (s, 2H), 4.18 (s, 2H), 2.59 (s, 6H), 2.26 (s, 3H), 1.52 (s, 6H). MS: 581.2 (M+18)⁺.

### Example 17/1 to 17/3

The following Examples were prepared similar as described for Example 17.

| # | educt | structure | analytical data |
|---|---|---|---|
| 17/1 | 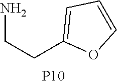 | 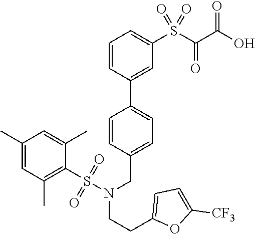 | ¹H-NMR (CDCl₃, 400 MHz): δ 8.01 (s, 1H), 7.81 (d, J = 7.6 Hz. 1H), 7.60 (d, J = 7.6 Hz, 1H), 7.40-7.37 (m, 3H), 7.16 (d, J = 8.0 Hz, 2H), 6.90 (s, 2H), 6.52 (d, J = 2.4 Hz, 1H), 5.89 (d, J = 2.8 Hz, 1H), 4.30 (s, 2H), 4.15 (br s, 2H), 3.30 (t, J = 7.2 Hz, 2H), 2.68 (t, J = 7.2 Hz, 2H), 2.55 (s, 6H), 2.25 (s, 3H). MS: 649.8 (M + H)⁺. |
| 17/2 | 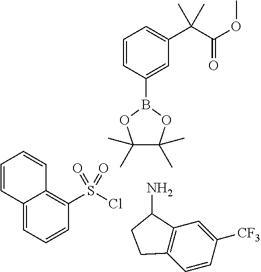 | 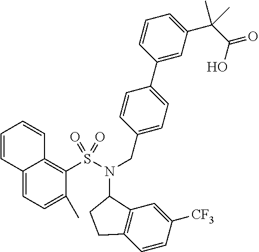 [SMILES: Cc1ccc2ccccc2c1S(=O)(=O)N(Cc1ccc(-c2cccc(C(C)(C)C(=O)O)c2)cc1)C1CCc2ccc(C(F)(F)F)cc21] | ¹H-NMR (DMSO-d₆, 400 MHz): δ 8.81 (d, J = 8.8 Hz, 1H), 8.06 (d, J = 8.4 Hz, 1H), 8.03 (d, J = 7.6 Hz, 1H), 7.75-7.71 (m, 1H), 7.66-7.62 (m, 1H), 7.47-7.23 (m, 7H), 7.09 (d, J = 8.0 Hz, 2H), 7.01 (s, 1H), 6.85 (d, J = 8.0 Hz, 2H), 5.69 (t, J = 7.6 Hz, 1H), 4.39 (d, J = 16.4 Hz, 1H), 4.28 (d, J = 16.4 Hz, 1H), 2.90-2.78 (m, 5H), 2.34-2.29 (m, 1H), 2.03-1.98 (m, 1H), 1.45 (s, 6H). MS: 656.0 (M − H)⁻. |
| 17/3 | 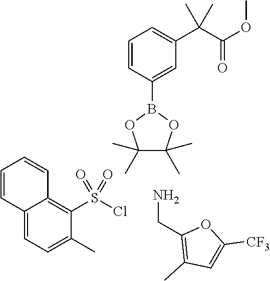 | 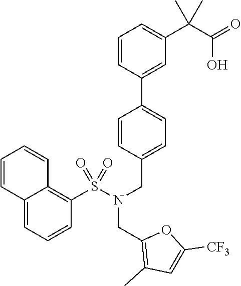 | ¹H-NMR (CD₃OD, 400 MHz): δ 8.88 (d, J = 8.8 Hz, 1H), 8.01 (d, J = 8.0 Hz, 1H), 7.91 (d, J = 7.6 Hz, 1H), 7.63-7.55 (m, 2H), 7.49 (s, 1H), 7.42 (d, J = 8.4 Hz, 1H), 7.36-7.31 (m, 5H), 7.04 (d, J = 8.0 Hz, 2H), 6.53 (s, 1H), 4.45 (s, 2H), 4.44 (s, 2H), 2.92 (s, 3H), 1.69 (s, 3H), 1.58 (s, 6H). MS: 633.9 (M − H)⁻. |

### Example 18

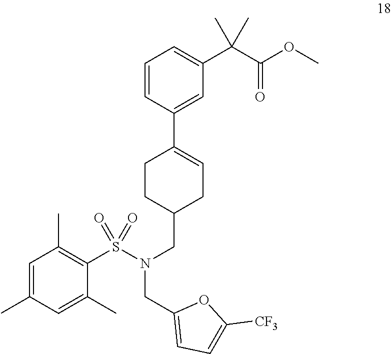

### Step 1: 2,4,6-Trimethyl-N-((4-oxocyclohexyl)methyl)-N-((5-(trifluoromethyl)furan-2-yl)methyl)benzenesulfonamide (18a)

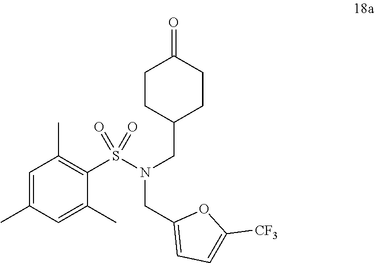 [SMILES: Cc1cc(C)c(S(=O)(=O)N(Cc2ccc(C(F)(F)F)o2)CC2CCC(=O)CC2)c(C)c1]

Compound 18a was prepared similar as described in Example 10 using 2,4,6-trimethyl-benzenesulfonyl chloride, 4-(aminomethyl)cyclohexan-1-one and 2-(bromomethyl)-5-(trifluoro-methyl)furan as building blocks.

### Step 2: 4-(((2,4,6-Trimethyl-N-((5-(trifluoromethyl)furan-2-yl)methyl)phenyl)sulfon-amido)methyl)cyclohex-1-en-1-yl trifluoromethanesulfonate (18b)

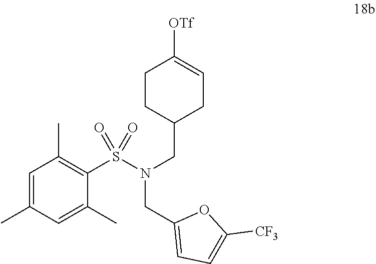 [SMILES: Cc1cc(C)c(S(=O)(=O)N(Cc2ccc(C(F)(F)F)o2)CC2CC=C(OS(=O)(=O)C(F)(F)F)CC2)c(C)c1]

To a solution of compound 18a (580 mg, 1.3 mmol) in DCM (50 mL) was added diisopropyl-ethylamine (1.0 g, 7.8 mmol) and (Tf)₂O (0.43 mL, 2.6 mmol) at 0° C. The mixture was allowed to warm to rt overnight, diluted with water and extracted with DCM (3×). The combined organic layer was washed with water and concentrated to give the crude compound 18b, which was used in the next step without further purification.

### Step 3: Methyl 2-methyl-2-(4′-(((2,4,6-trimethyl-N-((5-(trifluoromethyl)furan-2-yl)methyl)phenyl)sulfonamido)methyl)-2′,3′,4′,5′-tetrahydro-[1,1′-biphenyl]-3-yl)propanoate (18)

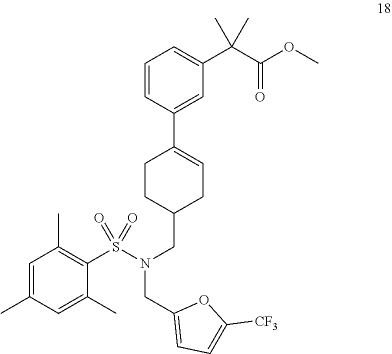

A mixture of compound 18b (crude, 1.3 mmol), methyl 2-methyl-2-(3-(4,4,5,5-tetramethyl-1,3,2-dioxaborolan-2-yl)phenyl)propanoate (395 mg, 1.3 mmol), Pd(PPh₃)₄ (137 mg, 100 μmol) and K₂CO₃ (540 mg, 3.9 mmol) in 1,4-dioxane/H₂O (30 mL/1 mL) was heated to 80° C. under N₂ overnight. The mixture was cooled, filtered, concentrated and purified by TLC (PE:EA=5:1) to give compound 18 as a yellow oil. MS: 618 (M+H)⁺.

### Example 19

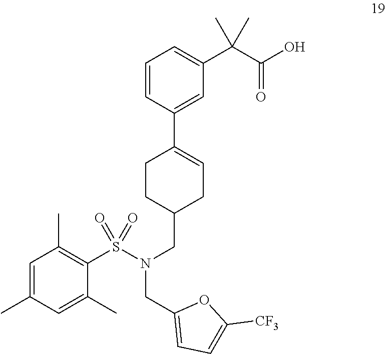 [SMILES: Cc1cc(C)c(S(=O)(=O)N(Cc2ccc(C(F)(F)F)o2)CC2CC=C(c3cccc(C(C)(C)C(=O)O)c3)CC2)c(C)c1]

### 2-Methyl-2-(4′-(((2,4,6-trimethyl-N-((5-(trifluoromethyl)furan-2-yl)methyl)phenyl)sulfon-amido)methyl)-2′,3′,4′,5′-tetrahydro-[1,1′-biphenyl]-3-yl)propanoic acid (19)

A solution of compound 18 (40 mg, 70 μmol) and NaOH (16 mg, 0.35 mmol) in MeOH/H₂O (10 and 3 mL) was stirred at reflux overnight. The MeOH was evaporated and the resulting solution was acidified with 1N HCl to pH ˜2 and extracted with EA (3×). The combined organic layer was washed with brine, dried over Na₂SO₄, filtered, concentrated and purified by prep-HPLC to afford compound 19 as a white solid. ¹H-NMR (CDCl₃, 400 MHz): δ 7.32 (s, 1H), 7.23 (d, J=4.8 Hz, 2H), 7.15-7.13 (m, 1H), 6.90 (s, 2H), 6.67 (d, J=2.0 Hz, 1H), 6.29 (d, J=3.2 Hz, 1H), 5.88 (s, 1H), 4.49-4.37 (m, 2H), 3.11 (d, J=7.2 Hz, 2H), 2.58 (s, 6H), 2.32-2.19 (m, 6H), 1.99-1.96 (m, 1H), 1.83-1.77 (m, 1H), 1.59-1.57 (m, 1H), 1.56 (s, 6H), 1.27-1.24 (m, 1H). MS: 604.0 (M+H)⁺.

### Example 19/1 to 19/2

The following Examples were prepared similar as described for Example 19.

| # | educt | structure | analytical data |
|---|---|---|---|
| 19/1 | 20 | 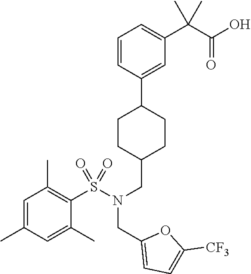 [SMILES: Cc1cc(C)c(S(=O)(=O)N(Cc2ccc(C(F)(F)F)o2)CC2CCC(c3cccc(C(C)(C)C(=O)O)c3)CC2)c(C)c1] | ¹H-NMR (CDCl₃, 400 MHz): δ 7.26-7.19 (m, 2H), 7.09 (s, 1H), 6.93 (s, 2H), 6.85 (d, J = 7.2 Hz, 1H), 6.71 (d, J = 2.0 Hz, 1H), 6.39 (d, J = 3.6 Hz, 1H), 4.48 (s, 2H), 3.15 (d, J = 8 0 Hz, 2H), 2.62 (s, 6H), 2.44-2.38 (m, 1H), 2.19 (s, 3H), 2.10-2.08 (m, 1H), 1.59 (s, 6H), 1.56-1.43 (m, 6H), 1.10-1.02 (m, 2H). MS: 604.0 (M − H)⁻. |
| 19/2 | 21 | 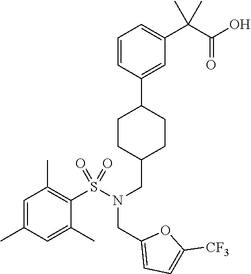 [SMILES: Cc1cc(C)c(S(=O)(=O)N(Cc2ccc(C(F)(F)F)o2)CC2CCC(c3cccc(C(C)(C)C(=O)O)c3)CC2)c(C)c1] | ¹H-NMR (CDCl₃, 400 MHz): δ 7.20 (t, J = 8.0 Hz, 1H), 6.94 (s, 3H), 6.88 (d, J = 8.0 Hz, 1H), 6.78 (d, J = 8.4 Hz, 1H), 6.68 (d, J = 2.4 Hz, 1H), 6.29 (d, J = 3.2 Hz, 1H), 4.41 (s, 2H), 3.54 (d, J = 12.0 Hz, 2H), 3.07 (d, J = 7.2 Hz, 2H), 2.63-2.59 (m, 8H), 2.30 (s, 3H), 1.69 (d, J = 9.2 Hz, 3H), 1.57 (s, 6H), 1.17-1.11 (m, 2H). MS: 607.2 (M + H)⁺. |

### Example 20

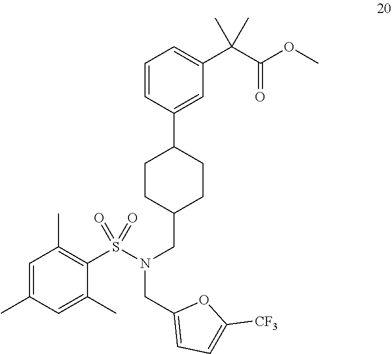

### Methyl 2-methyl-2-(3-(4-(((2,4,6-trimethyl-N-((5-(trifluoromethyl)furan-2-yl)methyl)phenyl)sulfonamido)methyl)cyclohexyl)phenyl)propanoate (20)

To a solution of compound 18 (50 mg, 80 μmol) in MeOH/THF (5 mL/5 mL) was added Pd/C (10 mg) at rt. The mixture was stirred at rt for 8 h under H₂ (1 atm), filtered, concentrated and purified by FCC (PE:EA=20:1) to give compound 20 as a yellow oil. MS: 620 (M+H)⁺.

### Example 21

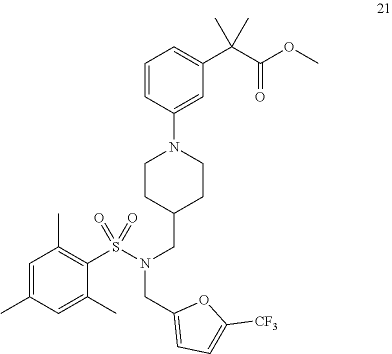

### Step 1: tert-Butyl 4-(((2,4,6-trimethyl-N-((5-(trifluoromethyl)furan-2-yl)methyl)phenyl)sulfon-amido)methyl)piperidine-1-carboxylate (21a)

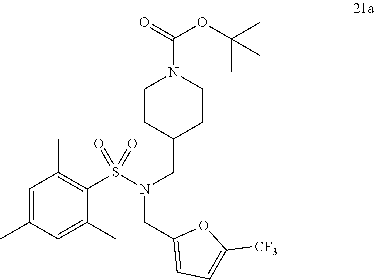 [SMILES: Cc1cc(C)c(S(=O)(=O)N(Cc2ccc(C(F)(F)F)o2)CC2CCN(C(=O)OC(C)(C)C)CC2)c(C)c1]

Compound 21a was prepared similar as described in Example 10 using 2,4,6-trimethyl-benzenesulfonyl chloride, tert-butyl 4-(aminomethyl)piperidine-1-carboxylate and 2-(bromo-methyl)-5-(trifluoromethyl)furan as building blocks.

### Step 2: 2,4,6-Trimethyl-N-(piperidin-4-ylmethyl)-N-((5-(trifluoromethyl)furan-2-yl)methyl)benzenesulfonamide (21b)

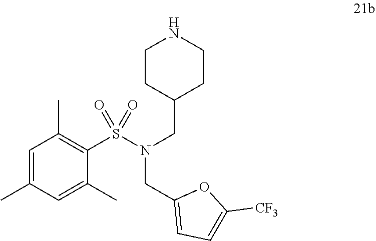 [SMILES: Cc1cc(C)c(S(=O)(=O)N(Cc2ccc(C(F)(F)F)o2)CC2CCNCC2)c(C)c1]

To a solution of compound 21a (500 mg, 0.9 mmol) in DCM (20 mL) was added TFA (10 mL) at rt. Th mixture was stirred at rt for 2 h, concentrated, diluted with sat. Na₂CO₃ to adjust the pH to ˜10 and extracted with EA (3×). The combined organic layer was washed with brine, dried over Na₂SO₄, filtered and concentrated to give compound 21b as a yellow oil.

### Step 3: Methyl 2-methyl-2-(3-(4-(((2,4,6-trimethyl-N-((5-(trifluoromethyl)furan-2-yl)methyl)phenyl)sulfonamido)methyl)piperidin-1-yl)phenyl)propanoate (21)

A mixture of compound 21b (319 mg, 0.7 mmol), methyl 2-(3-bromophenyl)-2-methyl-propanoate (203 mg, 0.8 mmol), Pd₂(dba)₃ (34 mg, 0.1 mmol), X-phos (86 mg, 0.2 mmol) and Cs₂CO₃ (585 mg, 1.8 mmol) in toluene/tert-BuOH (30 mL/5 mL) was heated to 110° C. overnight under N₂. The mixture was cooled, filtered, concentrated and purified by FCC (PE:EA=10:1) to give compound 21 as a yellow oil.

### Example 22

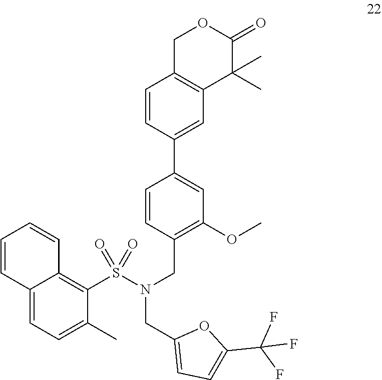 [SMILES: COc1cc(-c2ccc3c(c2)C(C)(C)C(=O)OC3)ccc1CN(Cc1ccc(C(F)(F)F)o1)S(=O)(=O)c1c(C)ccc2ccccc12]

### N-(4-(4,4-Dimethyl-3-oxoisochroman-6-yl)-2-methoxybenzyl)-2-methyl-N-((5-(trifluoro-methyl)furan-2-yl)methyl)naphthalene-1-sulfonamide (22)

Using 2-methylnaphthalene-1-sulfonyl chloride, (4-bromo-2-methoxyphenyl)methanamine, 2-(bromomethyl)-5-(trifluoromethyl)furan and compound P7-1 similar as described for Example 10, Step 1 to 3, compound 22 was prepared as a white solid.

### Example 23

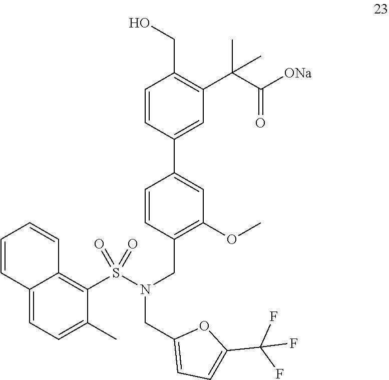

### Sodium 2-(4-(hydroxymethyl)-3′-methoxy-4′-(((2-methyl-N-((5-(trifluoromethyl)furan-2-yl)methyl)naphthalene)-1-sulfonamido)methyl)-[1,1′-biphenyl]-3-yl)-2-methylpropanoate (23)

To a solution of compound 22 (170 mg, 0.26 mmol) in MeOH (20 mL) and water (20 mL) was added NaOH (21 mg, 0.52 mmol) at rt. The mixture was stirred at rt overnight and then the MeOH was evaporated. The residue was washed with H₂O and then lyophilized to get compound 23 as a white solid. ¹H-NMR (CD₃OD, 400 MHz): δ 8.80 (d, J=8.8 Hz, 1H), 7.95 (d, J=8.4 Hz, 1H), 7.85 (d, J=8.0 Hz, 1H), 7.61-7.57 (m, 1H), 7.53-7.50 (m, 2H), 7.47-7.44 (m, 1H), 7.39-7.36 (m, 1H), 7.33-7.30 (m, 1H), 6.95-6.81 (m, 3H), 6.76-6.74 (m, 1H), 6.24 (d, J=3.2 Hz, 1H), 5.51 (s, 1H), 4.68 (s, 1H), 4.58 (d, J=9.2 Hz, 2H), 4.46 (d, J=9.2 Hz, 2H), 3.52 (d, J=15.6 Hz, 3H), 2.90 (s, 3H), 1.62 (s, 3H), 1.56 (s, 3H). MS: 704.0 (M+H)⁺. The spectra indicates, that some compound 23 has cyclised back to compound 22.

### Example 24

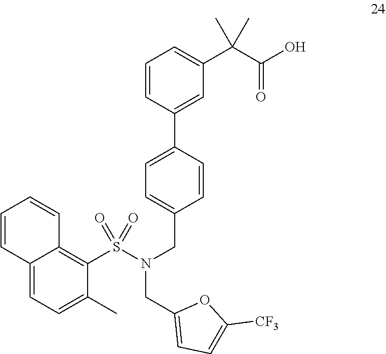 [SMILES: Cc1ccc2ccccc2c1S(=O)(=O)N(Cc1ccc(-c2cccc(C(C)(C)C(=O)O)c2)cc1)Cc1ccc(C(F)(F)F)o1]

### Step 1: Methyl 2-(4′-(((tert-butoxycarbonyl)amino)methyl)-[1,1′-biphenyl]-3-yl)-2-methyl-propanoate (24a)

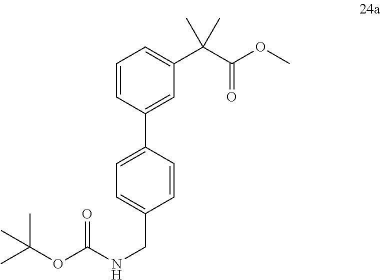 [SMILES: COC(=O)C(C)(C)c1cccc(-c2ccc(CNC(=O)OC(C)(C)C)cc2)c1]

To a solution of tert-butyl (4-(4,4,5,5-tetramethyl-1,3,2-dioxaborolan-2-yl)benzyl)carbamate (1.46 g, 4.40 mmol) in 1,2-dioxane (20 mL) and water (2 mL) was added methyl 2-(3-bromo-phenyl)-2-methylpropanoate (1.13 g, 4.40 mmol), Na₂CO₃ (1.20 g, 8.80 mmol) and Pd(dppf)Cl₂ (150 mg) and the mixture was stirred at 90° C. for 3 h under N₂, cooled, diluted with water (40 mL) and extracted with EA (3×20 mL). The combined organic layer was washed with brine (30 mL), dried over Na₂SO₄, filtered, concentrated and purified by FCC (PE:EA=10:1) to give compound 24a as a white solid.

### Step 2: Methyl 2-(4′-(aminomethyl)-[1,1′-biphenyl]-3-yl)-2-methylpropanoate (24b)

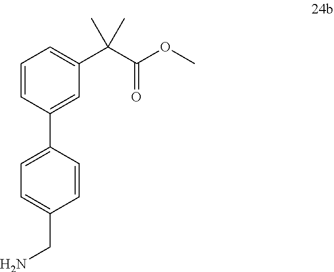 [SMILES: COC(=O)C(C)(C)c1cccc(-c2ccc(CN)cc2)c1]

To a solution of the compound 24a (220 mg, 0.57 mmol) in 1,4-dioxane (10 mL) was added HCl (5 mL, 6M in 1,4-dioxane) and the mixture was stirred at rt for 2 h, diluted with water (50 mL), adjusted to pH ˜8 with NaHCO₃ and extracted with EA (3×30 mL). The combined organic layer was washed with brine (40 mL), dried over Na₂SO₄, filtered and concentrated to give compound 24b as a yellow oil.

### Step 3: Methyl 2-methyl-2-(4′-(((2-methylnaphthalene)-1-sulfonamido)methyl)-[1,1′-biphenyl]-3-yl)propanoate (24c)

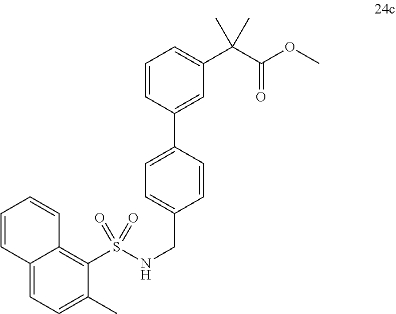 [SMILES: COC(=O)C(C)(C)c1cccc(-c2ccc(CNS(=O)(=O)c3c(C)ccc4ccccc34)cc2)c1]

To a solution of the compound 24b (160 mg, 0.56 mmol) in CH₂Cl₂ (5 mL) was added 2-methylnaphthalene-1-sulfonyl chloride (160 mg, 0.67 mmol) and Et₃N (113 mg, 1.1 mmol) and the mixture was stirred at rt for 12 h, diluted with water (50 mL) and extracted with EA (3×30 mL). The combined organic layer was washed with brine (30 mL), dried over Na₂SO₄, filtered, concentrated and purified by FCC (PE:EA=3:1) to give compound 24c as a colorless oil.

### Step 4: Methyl 2-methyl-2-(4′-(((2-methyl-N-((5-(trifluoromethyl)furan-2-yl)methyl)naphthalene)-1-sulfonamido)methyl)-[1,1′-biphenyl]-3-yl)propanoate (24d)

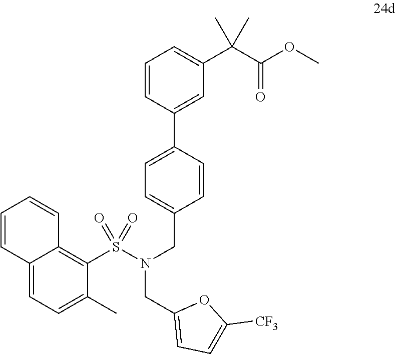 [SMILES: COC(=O)C(C)(C)c1cccc(-c2ccc(CN(Cc3ccc(C(F)(F)F)o3)S(=O)(=O)c3c(C)ccc4ccccc34)cc2)c1]

To a solution of the compound 24c (220 mg, 0.45 mmol) in DMF (5 mL) was added 2-(bromo-methyl)-5-(trifluoromethyl)furan (90 mg, 0.45 mmol) and Cs₂CO₃ (293 mg, 0.90 mmol) and the mixture was stirred at rt for 12 h, diluted with water (50 mL) and extracted with EA (3×20 mL). The combined organic layer was washed with brine (30 mL), dried over Na₂SO₄, filtered, concentrated and purified by FCC (PE:EA=10:1) to give compound 24d as a colorless oil.

### Step 5: 2-Methyl-2-(4′-(((2-methyl-N-((5-(trifluoromethyl)furan-2-yl)methyl)naphthalene)-1-sulfonamido)methyl)-[1,1′-biphenyl]-3-yl)propanoic acid (24)

To a mixture of compound 24d (150 mg, 0.24 mmol) in MeOH (2 mL) and THF (1 mL) was added LiOH (2M, 0.3 mL) and the mixture was stirred at rt overnight, neutralized with 1M HCl and extracted with EA (3×). The combined organic layer was washed with brine (30 mL), dried over Na₂SO₄, filtered, concentrated and purified by prep-HPLC to give compound 24 as a white solid. ¹H-NMR (500 MHz, CD₃OD): δ: 8.87 (d, J=9.0 Hz, 1H), 8.03 (d, J=8.5 Hz, 1H), 7.93 (d, J=7.5 Hz, 1H), 7.67-7.64 (m, 1H), 7.59-7.56 (m, 1H), 7.51 (d, J=1.0 Hz, 1H), 7.45-7.38 (m, 4H), 7.34 (d, J=8.0 Hz, 2H), 7.03 (d, J=8.0 Hz, 2H), 6.72 (dd, J=3.5 Hz, J=1.0 Hz, 1H), 6.16 (d, J=3.5 Hz, 1H), 4.50 (s, 2H), 4.48 (s, 2H), 2.94 (s, 3H), 1.61 (s, 6H). MS: 619.7 (M−H)⁻.

### Example 25

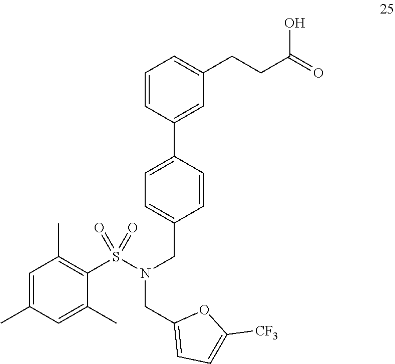 [SMILES: Cc1cc(C)c(S(=O)(=O)N(Cc2ccc(-c3cccc(CCC(=O)O)c3)cc2)Cc2ccc(C(F)(F)F)o2)c(C)c1]

### 3-(4′-(((2,4,6-Trimethyl-N-((5-(trifluoromethyl)furan-2-yl)methyl)phenyl)sulfonamido)methyl)-[1,1′-biphenyl]-3-yl)propanoic acid (25)

A solution of 2,4,6-trimethyl-N-(4-(4,4,5,5-tetramethyl-1,3,2-dioxaborolan-2-yl)benzyl)-N-((5-(trifluoromethyl)furan-2-yl)methyl)benzenesulfonamide (prepared as described in Example 11, 300 mg, 0.53 mmol), 3-(3-bromophenyl)propanoic acid (123 mg, 0.53 mmol), s-phos (22 mg, 50 μmol), Pd(OAc)₂ (6 mg, 30 μmol) and K₃PO₄ (283 mg, 1.34 mmol) in ACN/H₂O (15 mL/5 mL) under N₂ was heated to reflux overnight, cooled, filtered, concentrated and purified by prep-HPLC to give compound 25 as a white solid. ¹H-NMR (CD₃OD, 400 MHz): δ 7.53 (d, J=8.0 Hz, 2H), 7.46 (s, 1H), 7.41-7.39 (m, 1H), 7.34 (t, J=7.6 Hz, 1H), 7.23-7.20 (m, 3H), 7.05 (s, 2H), 6.80 (dd, J=3.2 Hz, J=1.2 Hz, 1H), 6.27 (d, J=2.8 Hz, 1H), 4.40 (s, 2H), 4.33 (s, 2H), 2.97 (t, J=7.6 Hz, 2H), 2.62-7.59 (m, 8H), 2.32 (s, 3H). MS: 584.1 (M−H)⁻.

### Example 25/1 to 25/3

The following Examples were prepared similar as described for Example 25.

| # | educt | structure | analytical data |
|---|---|---|---|
| 25/1 | 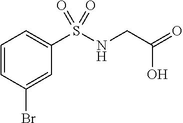 [SMILES: O=C(O)CNS(=O)(=O)c1cccc(Br)c1] | 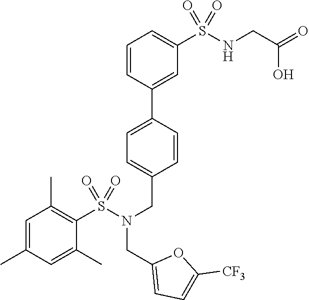 [SMILES: Cc1cc(C)c(S(=O)(=O)N(Cc2ccc(-c3cccc(S(=O)(=O)NCC(=O)O)c3)cc2)Cc2ccc(C(F)(F)F)o2)c(C)c1] | ¹H-NMR (CD₃OD, 400 MHz): δ 8.07 (t, J = 1.6 Hz, 1H), 7.85-7.82 (m, 2H), 7.64-7.59 (m, 3H), 7.26 (d, J = 8.4 Hz, 2H), 7.05 (s, 2H), 6.81- 6.80 (m, 1H), 6.29 (d, J = 2.8 Hz, 1H), 4.42 (s, 2H), 4.35 (s, 2H), 3.48 (s, 2H), 2.62 (s, 6H), 2.31 (s, 3H) MS: 649.1 (M − H)⁻. |
| 25/2 | 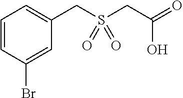 [SMILES: O=C(O)CS(=O)(=O)Cc1cccc(Br)c1] | 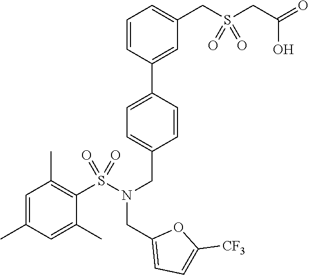 [SMILES: Cc1cc(C)c(S(=O)(=O)N(Cc2ccc(-c3cccc(CS(=O)(=O)CC(=O)O)c3)cc2)Cc2ccc(C(F)(F)F)o2)c(C)c1] | ¹H-NMR (CDCl₃ + few TFA, 300 MHz): δ 7.66- 7.47 (m, 6H), 7.25-7.22 (m, 2H), 7.00 (s, 2H), 6.65 (d, J = 2.1 Hz, 1H), 6.21 (d, J = 3.3 Hz, 1H), 4.62 (s, 2H), 4.38 (s, 2H), 4.26 (s, 2H), 3.94 (s, 2H), 2.63 (s, 6H), 2.33 (s, 3H). MS: 667.2 (M + 18)⁺. |
| 25/3 | 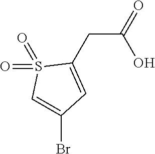 [SMILES: O=C(O)CC1=CC(Br)=CS1(=O)=O] | 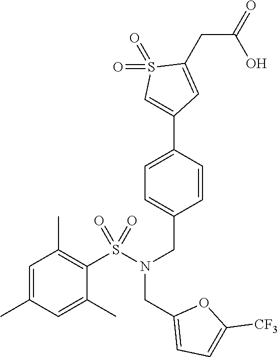 [SMILES: Cc1cc(C)c(S(=O)(=O)N(Cc2ccc(C3=CS(=O)(=O)C(CC(=O)O)=C3)cc2)Cc2ccc(C(F)(F)F)o2)c(C)c1] | ¹H-NMR (CDCl₃, 400 MHz): δ 8.02 (d, J = 1.2 Hz, 1H), 7.53 (d, J = 8.4 Hz, 2H), 7.31 (d, J = 8.0 Hz, 2H), 6.99 (s, 2H), 6.65-6.64 (m, 1H), 6.30 (s, 1H), 6.17 (d, J = 3.2 Hz, 1H), 4.14 (s, 2H), 4.26 (s, 2H), 4.22 (s, 2H), 2.62 (s, 6H), 2.33 (s, 3H). MS: 608.1 (M − H)⁻. |

### Example 26

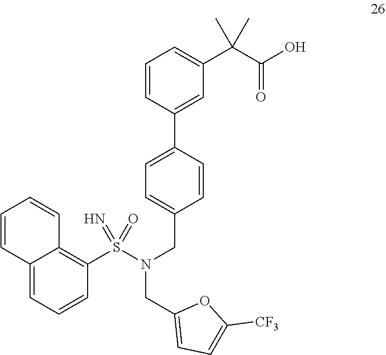 [SMILES: CC(C)(C(=O)O)c1cccc(-c2ccc(CN(Cc3ccc(C(F)(F)F)o3)S(=N)(=O)c3cccc4ccccc34)cc2)c1]

### Step 1: Methyl 2-(4′-(((tert-butoxycarbonyl)amino)methyl)-[1,1′-biphenyl]-3-yl)-2-methylpropanoate (26a)

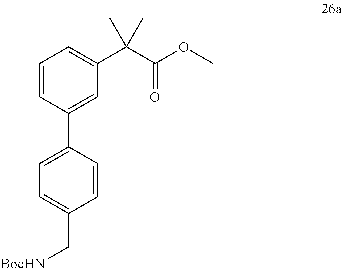 [SMILES: COC(=O)C(C)(C)c1cccc(-c2ccc(CNC(=O)OC(C)(C)C)cc2)c1]

To a solution of tert-butyl (4-(4,4,5,5-tetramethyl-1,3,2-dioxaborolan-2-yl)benzyl)carbamate (1.46 g, 4.40 mmol) in 1,4-dioxane (20 mL) and water (2 mL) was added methyl 2-(3-bromo-phenyl)-2-methylpropanoate (1.13 mg, 4.40 mmol), Na₂CO₃ (1.2 g, 8.8 mmol) and Pd(dppf)Cl₂ (150 mg) and the mixture was stirred at 90° C. for 3 h under N₂, diluted with water (40 mL) and extracted with EA (3×20 mL). The combined organic layer was washed with brine (30 mL), dried over Na₂SO₄, filtered, concentrated and purified by FCC (PE:EA=10:1) to afford compound 26a as a white solid.

### Step 2: Methyl 2-(4′-(((tert-butoxycarbonyl)((5-(trifluoromethyl)furan-2-yl)methyl)amino)methyl)-[1,1′-biphenyl]-3-yl)-2-methylpropanoate (26b)

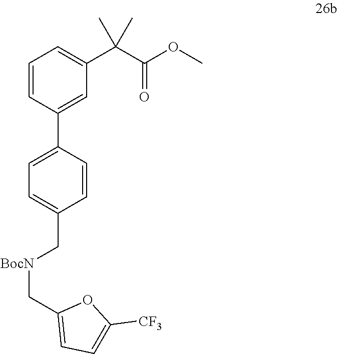 [SMILES: COC(=O)C(C)(C)c1cccc(-c2ccc(CN(Cc3ccc(C(F)(F)F)o3)C(=O)OC(C)(C)C)cc2)c1]

To a DMF solution (20 mL) of compound 26a (957 mg, 2.50 mmol) was added NaH (200 mg, 5.0 mmol, 60% in oil) and 2-(bromomethyl)-5-(trifluoromethyl)furan (570 mg, 2.50 mmol) at 0° C. and the mixture was stirred at rt overnight, diluted with water (200 mL) and extracted with EA (3×30 mL). The combined organic layer was washed with brine (30 mL), dried over Na₂SO₄, filtered, concentrated and purified by FCC (PE:EA=50:1) to afford compound 26b as a colorless oil.

### Step 3: Methyl 2-methyl-2-(4′-((((5-(trifluoromethyl)furan-2-yl)methyl)amino)methyl)-[1,1′-biphenyl]-3-yl)propanoate (26c)

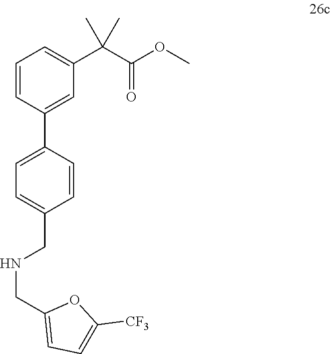 [SMILES: COC(=O)C(C)(C)c1cccc(-c2ccc(CNCc3ccc(C(F)(F)F)o3)cc2)c1]

To a solution of the compound 26b (1.2 g, 2.3 mmol) in 1,4-dioxane (10 mL) was added HCl (5 mL, 6M in 1,4-dioxane) and the mixture was stirred at rt for 2 h, diluted with water (50 mL), adjusted to pH=8 with NaHCO₃ and extracted with EA (3×30 mL). The combined organic layer was washed with brine (30 mL), dried over Na₂SO₄, filtered and concentrated to give compound 26c as a yellow oil.

### Step 4: Methyl 2-(4′-((N′-(tert-butyldimethylsilyl)-N-((5-(trifluoromethyl)furan-2-yl)methyl)naphthalene-1-sulfonoamidimidamido)methyl)-[1,1′-biphenyl]-3-yl)-2-methyl-propanoate (26d)

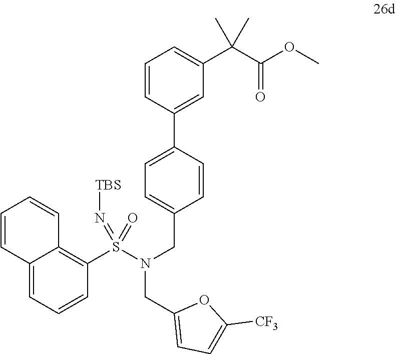

To a stirred suspension of PPh₃Cl₂ (667 mg, 2.0 mmol) in dry CHCl₃ (3 mL) under a N₂ atmosphere was added NEt₃ (0.70 mL, 5.0 mmol). The mixture was stirred for 10 min at rt, cooled to 0° C. and a solution of (tert-butyldimethylsilyl)(naphthalen-1-ylsulfonyl)-λ²-azane (641 mg, 2.00 mmol) in dry CHCl₃ (2.0 mL) was added. The mixture was stirred for 20 min at 0° C., after 5 min a clear solution formed. No attempt was made to isolate the sulfonimidoyl chloride intermediate. To the mixture was added a solution of compound 26c (200 mg, 0.46 mmol) in dry CHCl₃ (4 mL) in one portion. The mixture was stirred at 0° C. for 30 min, then warmed to rt overnight, concentrated and purified by prep-TLC (EA:PE=1:1) to afford compound 26d as a light yellow oil.

### Step 5: 2-Methyl-2-(4′-((N-((5-(trifluoromethyl)furan-2-yl)methyl)naphthalene-1-sulfonoamid-imidamido)methyl)-[1,1′-biphenyl]-3-yl)propanoic acid (26)

To the mixture of compound 26d (130 mg, 0.18 mmol) in MeOH (20 mL) and THF (10 mL) was added LiOH.H₂O (40 mg, 0.9 mmol) and the mixture was stirred at rt fo 4 h, neutralized with 1N HCl and stirred at rt for 20 min and extracted with EA (3×). The combined organic layer was washed with brine (30 mL), dried over Na₂SO₄, filtered, concentrated and purified by prep-HPLC to afford compound 26 as a white solid. ¹H-NMR (500 MHz, CD₃OD) δ: 8.90 (d, J=9.0 Hz, 1H), 8.22-8.20 (m, 2H), 8.05 (d, J=8.0 Hz, 1H), 7.74-7.40 (m, 9H), 7.25 (d, J=8.5 Hz, 2H), 6.70 (d, J=3.0 Hz, 1H), 6.20 (d, J=3.0 Hz, 1H), 4.75-4.58 (m, 4H), 1.63 (s, 6H). MS: 607.0 (M+1)⁺.

### Example 27

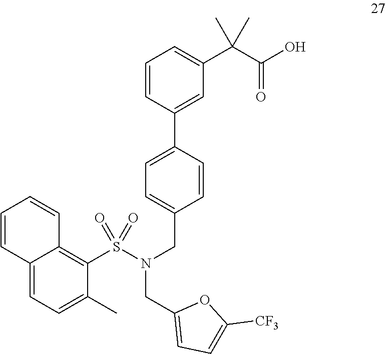 [SMILES: Cc1ccc2ccccc2c1S(=O)(=O)N(Cc1ccc(-c2cccc(C(C)(C)C(=O)O)c2)cc1)Cc1ccc(C(F)(F)F)o1]

### Step 1: N-(4-Bromobenzyl)-2-methylnaphthalene-1-sulfinamide (27a)

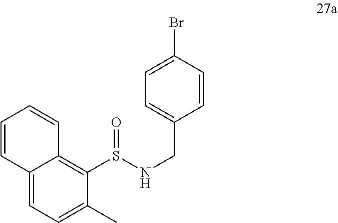 [SMILES: Cc1ccc2ccccc2c1S(=O)NCc1ccc(Br)cc1]

To a solution of (4-bromophenyl)methanamine (555 mg, 3.00 mmol) in DCM (20 mL) was added PPh₃ (786 mg, 3.00 mmol), TEA (606 mg, 6.00 mmol) and the mixture was stirred at 0° C. Then 2-methylnaphthalene-1-sulfonyl chloride (720 mg, 3.00 mmol) was added. The mixture was stirred at rt overnight, diluted with water (200 mL) and extracted with EA (3×50 mL). The combined organic layer was washed with brine (80 mL), dried over Na₂SO₄, filtered, concentrated and purified by FCC (PE:EA=5:1) to give compound 27a as a white solid.

### Step 2: N-(4-Bromobenzyl)-2-methyl-N-((5-(trifluoromethyl)furan-2-yl)methyl)naphthalene-1-sulfinamide (27b)

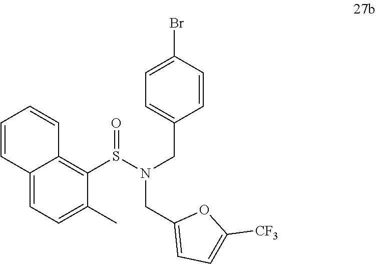 [SMILES: Cc1ccc2ccccc2c1S(=O)N(Cc1ccc(Br)cc1)Cc1ccc(C(F)(F)F)o1]

To a DMF solution (10 mL) of compound 27a (373 mg, 1.00 mmol) was added NaH (160 mg, 4.00 mmol, 60% in oil) at 0° C. and the mixture was stirred for 30 min, then 2-(bromomethyl)-5-(trifluoromethyl)furan (274 mg, 1.20 mmol) was added and the mixture was stirred for 1 h, diluted with water (100 mL) and extracted with EA (3×30 mL). The combined organic layer was washed with brine (80 mL), dried over Na₂SO₄, filtered, concentrated and purified by FCC (PE:EA=5:1) to give compound 27b as a colorless oil.

### Step 3: 2-Methyl-2-(4′-((((2-methylnaphthalen-1-yl)sulfinyl)((5-(trifluoromethyl)furan-2-yl)methyl)amino)methyl)-[1,1′-biphenyl]-3-yl)propanoic acid (27)

Compound 27b and methyl 2-methyl-2-(3-(4,4,5,5-tetramethyl-1,3,2-dioxaborolan-2-yl)phenyl)propanoate was treated as described in Example 24, Step 1 and then the obtained intermediate was dissolved in MeOH (2 mL) and THF (1 mL), followed by addition of NaOH (2N, 0.3 mL). The mixture was stirred at rt overnight, neutralized with 1N HCl and extracted with EA (3×). The combined organic layer was washed with brine, dried over Na₂SO₄, filtered, concentrated and purified by prep-HPLC to give compound 27 as a white solid. ¹H-NMR (500 MHz, CD₃OD) δ: 9.14 (d, J=6.5 Hz, 1H), 7.95 (d, J=8.0 Hz, 1H), 7.91 (d, J=7.5 Hz, 1H), 7.61-7.52 (m, 3H), 7.44-7.32 (m, 6H), 7.07 (d, J=8.5 Hz, 2H), 6.76 (dd, J=0.8, 3.3 Hz, 1H), 6.17 (d, J=3.0 Hz, 1H), 4.61 (d, J=15.0 Hz, 1H), 4.52 (d, J=16.0 Hz, 1H), 4.42-4.38 (m, 2H), 2.78 (s, 3H), 1.55 (s, 6H). MS: 603.8 (M−1)⁻.

### Example 28

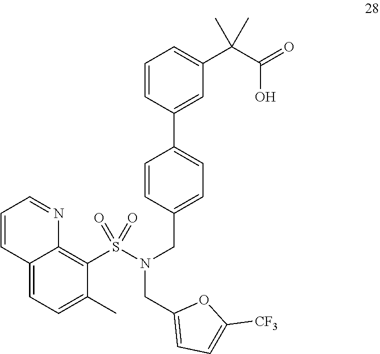 [SMILES: Cc1ccc2cccnc2c1S(=O)(=O)N(Cc1ccc(-c2cccc(C(C)(C)C(=O)O)c2)cc1)Cc1ccc(C(F)(F)F)o1]

### Step 1: N-(4-Bromobenzyl)-7-methyl-N-((5-(trifluoromethyl)furan-2-yl)methyl)quinoline-8-sulfonamide (28a)

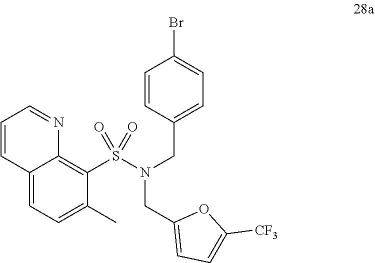 [SMILES: Cc1ccc2cccnc2c1S(=O)(=O)N(Cc1ccc(Br)cc1)Cc1ccc(C(F)(F)F)o1]

To a solution of N-(4-bromobenzyl)-1-(5-(trifluoromethyl)furan-2-yl)methanamine (333 mg, 1.00 mmol) in DCM (10 mL) was added TEA (0.30 g, 3.0 mmol) and 7-methylquinoline-8-sulfonyl chloride (241 mg, 1.00 mmol) and the mixture was stirred at rt for 4 h, concentrated and purified by FCC (PE:EA=2:1) to give compound 28a as a white solid.

### Step 2: Methyl 2-methyl-2-(4′-(((7-methyl-N-((5-(trifluoromethyl)furan-2-yl)methyl)quinoline)-8-sulfonamido)methyl)-[1,1′-biphenyl]-3-yl)propanoate (28b)

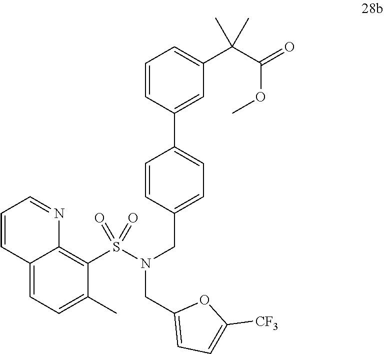 [SMILES: COC(=O)C(C)(C)c1cccc(-c2ccc(CN(Cc3ccc(C(F)(F)F)o3)S(=O)(=O)c3c(C)ccc4cccnc34)cc2)c1]

To a solution of compound 28a (320 mg, 0.59 mmol) in dioxane (10 mL) and water (1 mL) was added methyl 2-methyl-2-(3-(4,4,5,5-tetramethyl-1,3,2-dioxaborolan-2-yl)phenyl)propanoate (215 mg, 0.71 mmol), K₂CO₃ (163 mg, 1.18 mmol) and Pd(dppf)Cl₂ (40 mg) and the mixture was stirred at 90° C. for 3 h under N₂, cooled, diluted with water (100 mL) and extracted with EA (3×50 mL). The combined organic layer was washed with brine (100 mL), dried over Na₂SO₄, filtered, concentrated and purified by FCC (PE:EA=2:1) to give compound 28b as a white solid.

### Step 3: 2-Methyl-2-(4′-(((7-methyl-N-((5-(trifluoromethyl)furan-2-yl)methyl)quinoline)-8-sulfon-amido)methyl)-[1,1′-biphenyl]-3-yl)propanoic acid (28)

To a mixture of compound 28b (259 mg, 0.41 mmol) in MeOH (5 mL) and THF (2 mL) was added LiOH (2N, 3 mL) and the mixture was at rt overnight, neutralized with 1N HCl and extracted with EA (3×). The combined organic layer was washed with brine, dried over Na₂SO₄, filtered and concentrated to afford compound 28 as a white solid.

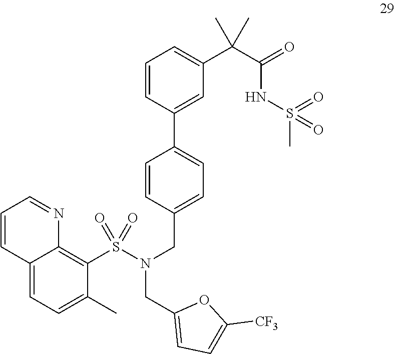 [SMILES: Cc1ccc2cccnc2c1S(=O)(=O)N(Cc1ccc(-c2cccc(C(C)(C)C(=O)NS(C)(=O)=O)c2)cc1)Cc1ccc(C(F)(F)F)o1]

### Example 29

### 2-Methyl-2-(4′-(((7-methyl-N-((5-(trifluoromethyl)furan-2-yl)methyl)quinoline)-8-sulfon-amido)methyl)-[1,1′-biphenyl]-3-yl)-N-(methylsulfonyl)propanamide (29)

To a mixture of compound 28 (100 mg, 0.16 mmol) in DCM (5 mL) was added methanesulfon-amide (23 mg, 0.24 mmol), EDCl.HCl (46 mg, 0.24 mmol) and DMAP (20 mg, 0.16 mmol). The mixture was stirred at rt overnight, poured into water and extracted with DCM (3×). The combined organic layer was washed with brine, dried over Na₂SO₄, filtered, concentrated and purified by prep-HPLC to afford compound 29 as a white solid. ¹H-NMR (400 MHz, CD₃OD) δ: 9.06 (dd, J=4.6, 1.8 Hz, 1H), 8.51 (d, J=8.0 Hz, 1H), 8.13 (d, J=8.4 Hz, 1H), 7.70-7.65 (m, 2H), 7.49-7.31 (m, 6H), 7.22 (d, J=8.0 Hz, 2H), 6.70 (d, J=2.0 Hz, 1H), 6.26 (d, J=2.4 Hz, 1H), 4.78 (s, 2H), 4.73 (s, 2H), 3.30 (s, 3H), 3.00 (s, 3H), 1.63 (s, 6H). MS: 700.0 (M+1)⁺.

### Example 30

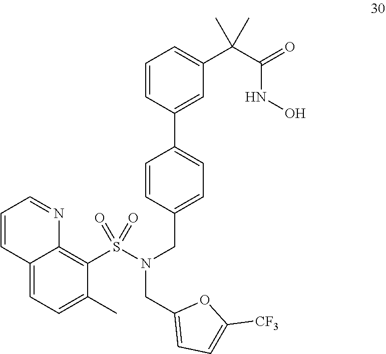 [SMILES: Cc1ccc2cccnc2c1S(=O)(=O)N(Cc1ccc(-c2cccc(C(C)(C)C(=O)NO)c2)cc1)Cc1ccc(C(F)(F)F)o1]

### N-Hydroxy-2-methyl-2-(4′-(((7-methyl-N-((5-(trifluoromethyl)furan-2-yl)methyl)quinoline)-8-sulfonamido)methyl)-[1,1′-biphenyl]-3-yl)propanamide (30)

To the mixture of compound 28 (100 mg, 0.16 mmol) in DMF (5 mL) was added hydroxyl-amine hydrochloride (17 mg, 0.24 mmol), HATU (91 mg, 0.24 mmol) and DIPEA (41 mg, 0.32 mmol). The mixture was stirred at rt for 2 h, poured into water and extracted with EA (3×). The combined organic layer was washed with brine, dried over Na₂SO₄, filtered, concentrated and purified by prep-HPLC to afford compound 30 as a white solid. ¹H-NMR (400 MHz, CD₃OD) δ: 9.05 (dd, J=4.4, 1.6 Hz, 1H), 8.51 (d, J=7.2 Hz, 1H), 8.15-8.13 (m, 1H), 7.68-7.20 (m, 10H), 6.69 (d, J=2.4 Hz, 1H), 6.25 (d, J=2.8 Hz, 1H), 4.77 (s, 2H), 4.73 (s, 2H), 3.00 (s, 3H), 1.62 (s, 6H). MS: 638.2 (M+1)⁺.

### Additional Examples

The following compounds can be prepared in the same manner by using the procedures as described above:

Structure

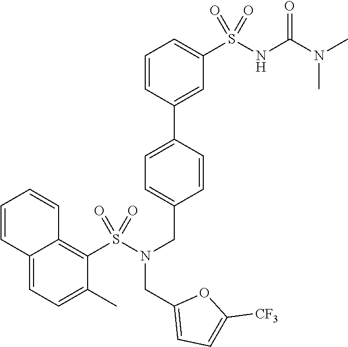 [SMILES: Cc1ccc2ccccc2c1S(=O)(=O)N(Cc1ccc(-c2cccc(S(=O)(=O)NC(=O)N(C)C)c2)cc1)Cc1ccc(C(F)(F)F)o1]

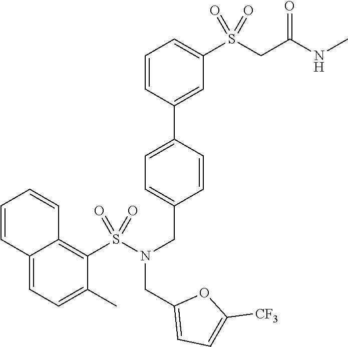 [SMILES: CNC(=O)CS(=O)(=O)c1cccc(-c2ccc(CN(Cc3ccc(C(F)(F)F)o3)S(=O)(=O)c3c(C)ccc4ccccc34)cc2)c1]

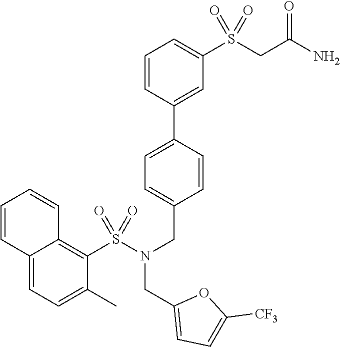 [SMILES: Cc1ccc2ccccc2c1S(=O)(=O)N(Cc1ccc(-c2cccc(S(=O)(=O)CC(N)=O)c2)cc1)Cc1ccc(C(F)(F)F)o1]

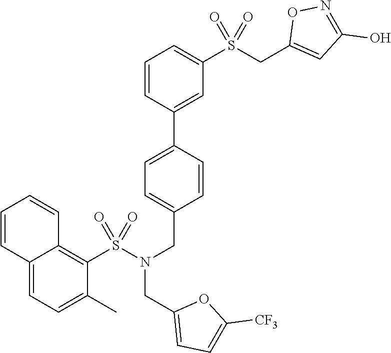 [SMILES: Cc1ccc2ccccc2c1S(=O)(=O)N(Cc1ccc(-c2cccc(S(=O)(=O)Cc3cc(O)no3)c2)cc1)Cc1ccc(C(F)(F)F)o1]

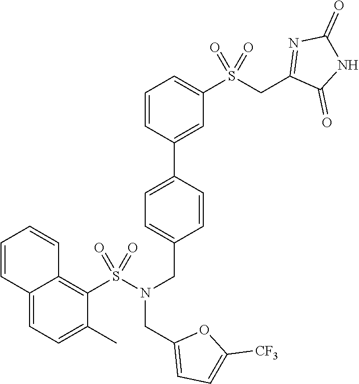 [SMILES: Cc1ccc2ccccc2c1S(=O)(=O)N(Cc1ccc(-c2cccc(S(=O)(=O)CC3=NC(=O)NC3=O)c2)cc1)Cc1ccc(C(F)(F)F)o1]

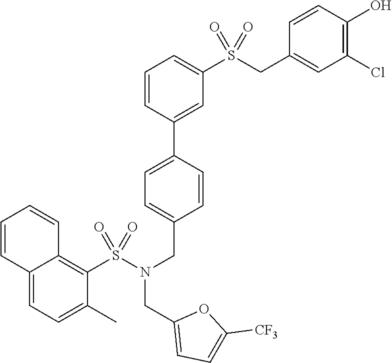 [SMILES: Cc1ccc2ccccc2c1S(=O)(=O)N(Cc1ccc(-c2cccc(S(=O)(=O)Cc3ccc(O)c(Cl)c3)c2)cc1)Cc1ccc(C(F)(F)F)o1]

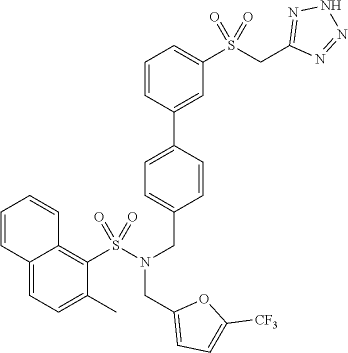 [SMILES: Cc1ccc2ccccc2c1S(=O)(=O)N(Cc1ccc(-c2cccc(S(=O)(=O)Cc3nn[nH]n3)c2)cc1)Cc1ccc(C(F)(F)F)o1]

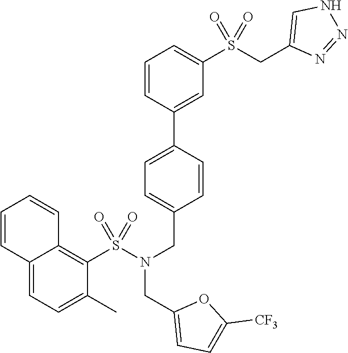 [SMILES: Cc1ccc2ccccc2c1S(=O)(=O)N(Cc1ccc(-c2cccc(S(=O)(=O)Cc3c[nH]nn3)c2)cc1)Cc1ccc(C(F)(F)F)o1]

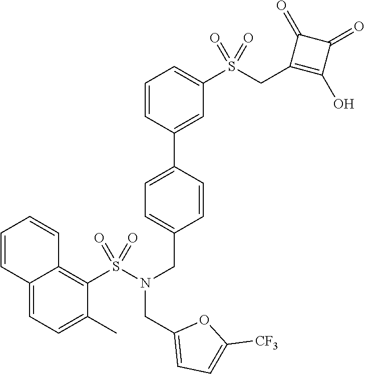 [SMILES: Cc1ccc2ccccc2c1S(=O)(=O)N(Cc1ccc(-c2cccc(S(=O)(=O)Cc3c(O)c(=O)c3=O)c2)cc1)Cc1ccc(C(F)(F)F)o1]

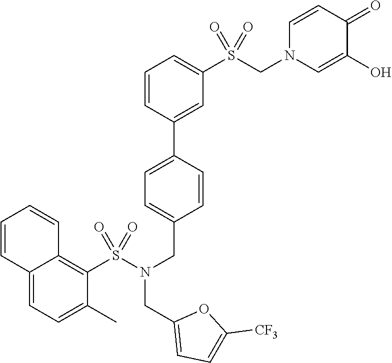 [SMILES: Cc1ccc2ccccc2c1S(=O)(=O)N(Cc1ccc(-c2cccc(S(=O)(=O)Cn3ccc(=O)c(O)c3)c2)cc1)Cc1ccc(C(F)(F)F)o1]

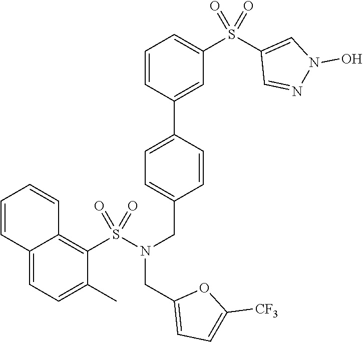 [SMILES: Cc1ccc2ccccc2c1S(=O)(=O)N(Cc1ccc(-c2cccc(S(=O)(=O)c3cnn(O)c3)c2)cc1)Cc1ccc(C(F)(F)F)o1]

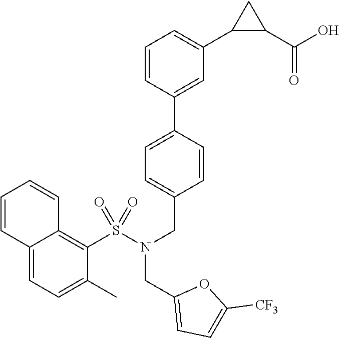 [SMILES: Cc1ccc2ccccc2c1S(=O)(=O)N(Cc1ccc(-c2cccc(C3CC3C(=O)O)c2)cc1)Cc1ccc(C(F)(F)F)o1]

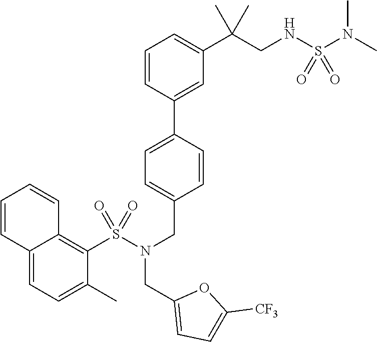 [SMILES: Cc1ccc2ccccc2c1S(=O)(=O)N(Cc1ccc(-c2cccc(C(C)(C)CNS(=O)(=O)N(C)C)c2)cc1)Cc1ccc(C(F)(F)F)o1]

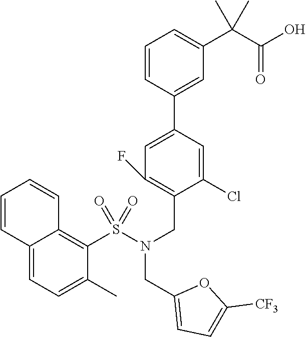 [SMILES: Cc1ccc2ccccc2c1S(=O)(=O)N(Cc1ccc(C(F)(F)F)o1)Cc1c(F)cc(-c2cccc(C(C)(C)C(=O)O)c2)cc1Cl]

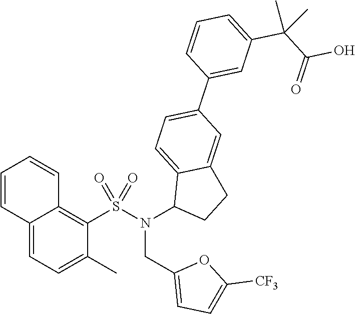 [SMILES: Cc1ccc2ccccc2c1S(=O)(=O)N(Cc1ccc(C(F)(F)F)o1)C1CCc2cc(-c3cccc(C(C)(C)C(=O)O)c3)ccc21]

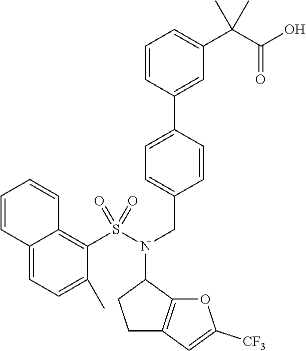 [SMILES: Cc1ccc2ccccc2c1S(=O)(=O)N(Cc1ccc(-c2cccc(C(C)(C)C(=O)O)c2)cc1)C1CCc2cc(C(F)(F)F)oc21]

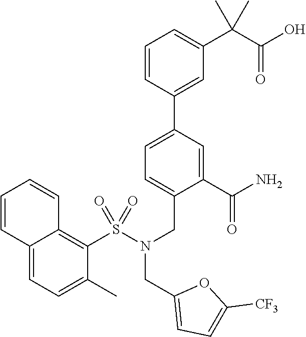 [SMILES: Cc1ccc2ccccc2c1S(=O)(=O)N(Cc1ccc(C(F)(F)F)o1)Cc1ccc(-c2cccc(C(C)(C)C(=O)O)c2)cc1C(N)=O]

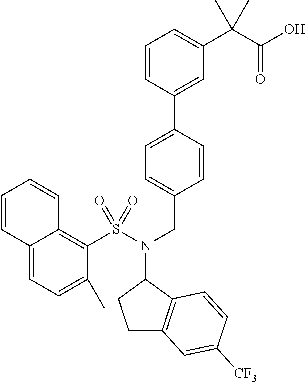 [SMILES: Cc1ccc2ccccc2c1S(=O)(=O)N(Cc1ccc(-c2cccc(C(C)(C)C(=O)O)c2)cc1)C1CCc2cc(C(F)(F)F)ccc21]

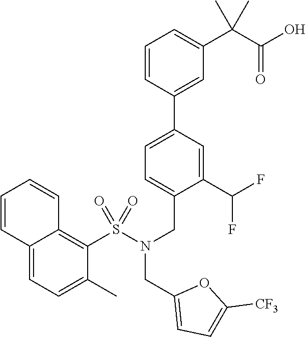 [SMILES: Cc1ccc2ccccc2c1S(=O)(=O)N(Cc1ccc(C(F)(F)F)o1)Cc1ccc(-c2cccc(C(C)(C)C(=O)O)c2)cc1C(F)F]

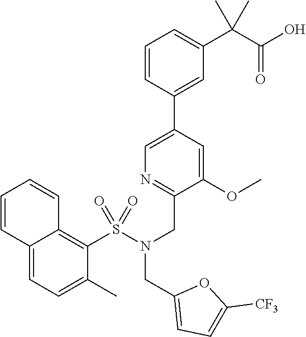 [SMILES: COc1cc(-c2cccc(C(C)(C)C(=O)O)c2)cnc1CN(Cc1ccc(C(F)(F)F)o1)S(=O)(=O)c1c(C)ccc2ccccc12]

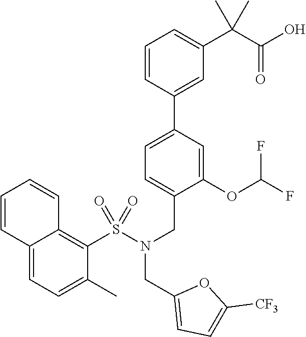 [SMILES: Cc1ccc2ccccc2c1S(=O)(=O)N(Cc1ccc(C(F)(F)F)o1)Cc1ccc(-c2cccc(C(C)(C)C(=O)O)c2)cc1OC(F)F]

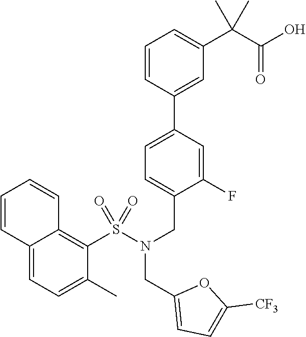 [SMILES: Cc1ccc2ccccc2c1S(=O)(=O)N(Cc1ccc(C(F)(F)F)o1)Cc1ccc(-c2cccc(C(C)(C)C(=O)O)c2)cc1F]

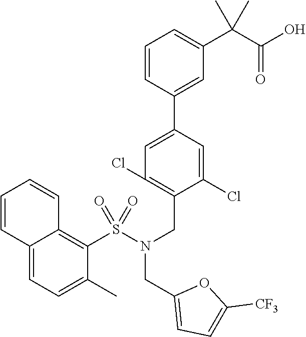 [SMILES: Cc1ccc2ccccc2c1S(=O)(=O)N(Cc1ccc(C(F)(F)F)o1)Cc1c(Cl)cc(-c2cccc(C(C)(C)C(=O)O)c2)cc1Cl]

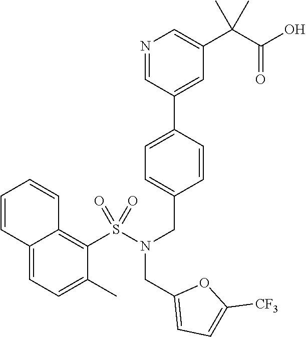 [SMILES: Cc1ccc2ccccc2c1S(=O)(=O)N(Cc1ccc(-c2cncc(C(C)(C)C(=O)O)c2)cc1)Cc1ccc(C(F)(F)F)o1]

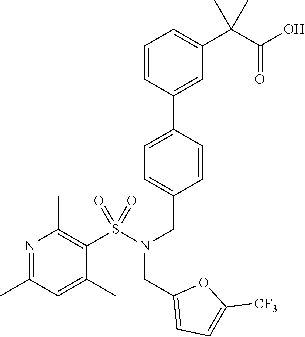 [SMILES: Cc1cc(C)c(S(=O)(=O)N(Cc2ccc(-c3cccc(C(C)(C)C(=O)O)c3)cc2)Cc2ccc(C(F)(F)F)o2)c(C)n1]

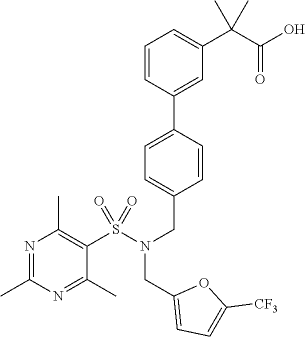 [SMILES: Cc1nc(C)c(S(=O)(=O)N(Cc2ccc(-c3cccc(C(C)(C)C(=O)O)c3)cc2)Cc2ccc(C(F)(F)F)o2)c(C)n1]

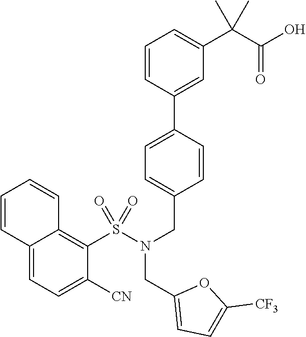 [SMILES: CC(C)(C(=O)O)c1cccc(-c2ccc(CN(Cc3ccc(C(F)(F)F)o3)S(=O)(=O)c3c(C#N)ccc4ccccc34)cc2)c1]

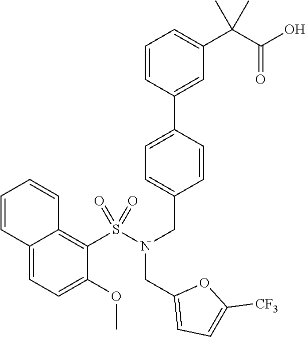 [SMILES: COc1ccc2ccccc2c1S(=O)(=O)N(Cc1ccc(-c2cccc(C(C)(C)C(=O)O)c2)cc1)Cc1ccc(C(F)(F)F)o1]

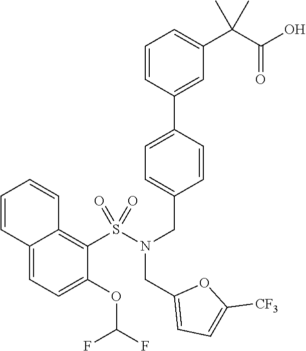 [SMILES: CC(C)(C(=O)O)c1cccc(-c2ccc(CN(Cc3ccc(C(F)(F)F)o3)S(=O)(=O)c3c(OC(F)F)ccc4ccccc34)cc2)c1]

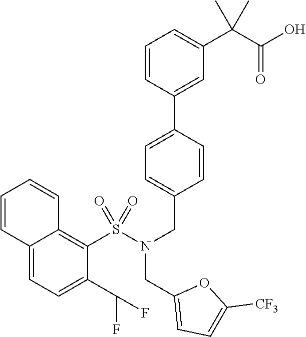 [SMILES: CC(C)(C(=O)O)c1cccc(-c2ccc(CN(Cc3ccc(C(F)(F)F)o3)S(=O)(=O)c3c(C(F)F)ccc4ccccc34)cc2)c1]

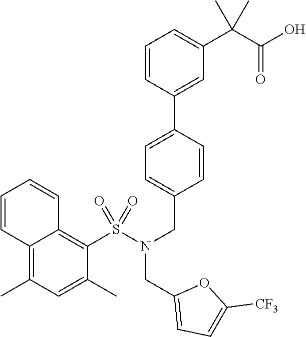 [SMILES: Cc1cc(C)c2ccccc2c1S(=O)(=O)N(Cc1ccc(-c2cccc(C(C)(C)C(=O)O)c2)cc1)Cc1ccc(C(F)(F)F)o1]

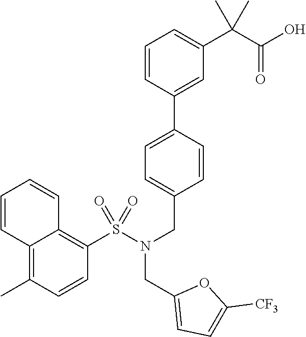 [SMILES: Cc1ccc(S(=O)(=O)N(Cc2ccc(-c3cccc(C(C)(C)C(=O)O)c3)cc2)Cc2ccc(C(F)(F)F)o2)c2ccccc12]

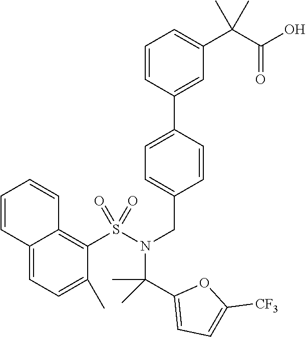 [SMILES: Cc1ccc2ccccc2c1S(=O)(=O)N(Cc1ccc(-c2cccc(C(C)(C)C(=O)O)c2)cc1)C(C)(C)c1ccc(C(F)(F)F)o1]

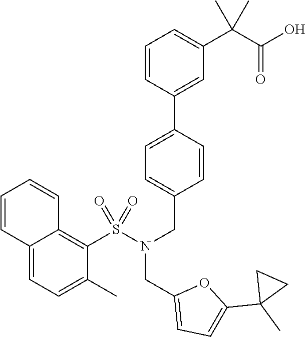 [SMILES: Cc1ccc2ccccc2c1S(=O)(=O)N(Cc1ccc(-c2cccc(C(C)(C)C(=O)O)c2)cc1)Cc1ccc(C2(C)CC2)o1]

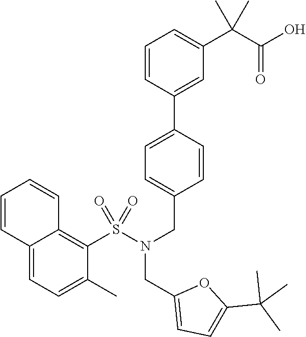 [SMILES: Cc1ccc2ccccc2c1S(=O)(=O)N(Cc1ccc(-c2cccc(C(C)(C)C(=O)O)c2)cc1)Cc1ccc(C(C)(C)C)o1]

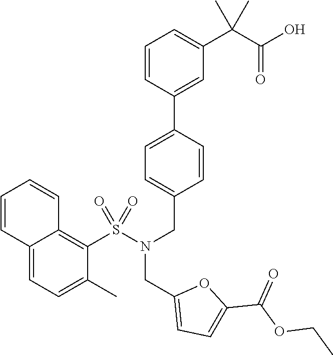 [SMILES: CCOC(=O)c1ccc(CN(Cc2ccc(-c3cccc(C(C)(C)C(=O)O)c3)cc2)S(=O)(=O)c2c(C)ccc3ccccc23)o1]

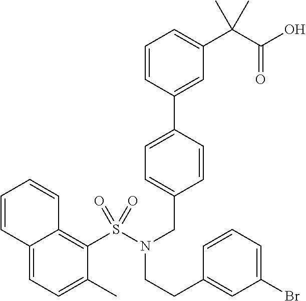 [SMILES: Cc1ccc2ccccc2c1S(=O)(=O)N(CCc1cccc(Br)c1)Cc1ccc(-c2cccc(C(C)(C)C(=O)O)c2)cc1]

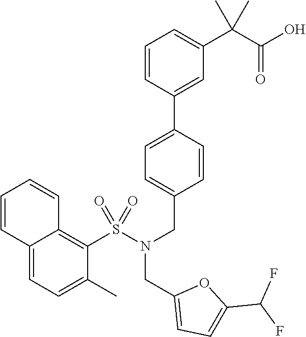 [SMILES: Cc1ccc2ccccc2c1S(=O)(=O)N(Cc1ccc(-c2cccc(C(C)(C)C(=O)O)c2)cc1)Cc1ccc(C(F)F)o1]

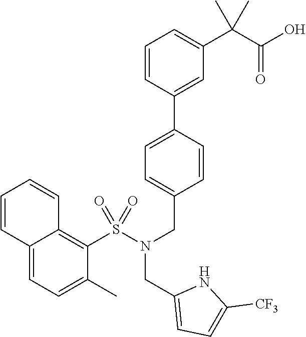 [SMILES: Cc1ccc2ccccc2c1S(=O)(=O)N(Cc1ccc(-c2cccc(C(C)(C)C(=O)O)c2)cc1)Cc1ccc(C(F)(F)F)[nH]1]

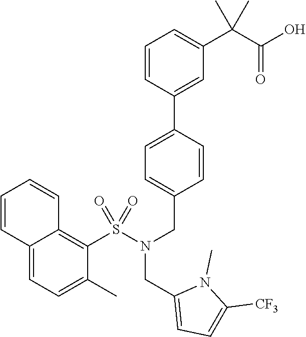 [SMILES: Cc1ccc2ccccc2c1S(=O)(=O)N(Cc1ccc(-c2cccc(C(C)(C)C(=O)O)c2)cc1)Cc1ccc(C(F)(F)F)n1C]

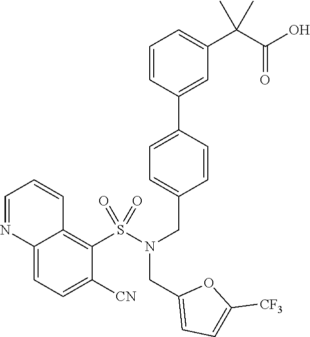 [SMILES: CC(C)(C(=O)O)c1cccc(-c2ccc(CN(Cc3ccc(C(F)(F)F)o3)S(=O)(=O)c3c(C#N)ccc4ncccc34)cc2)c1]

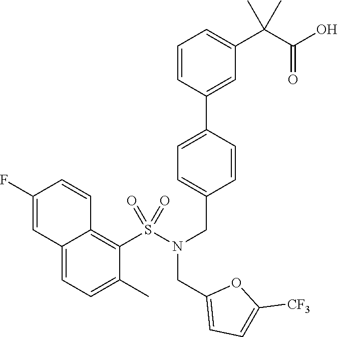 [SMILES: Cc1ccc2cc(F)ccc2c1S(=O)(=O)N(Cc1ccc(-c2cccc(C(C)(C)C(=O)O)c2)cc1)Cc1ccc(C(F)(F)F)o1]

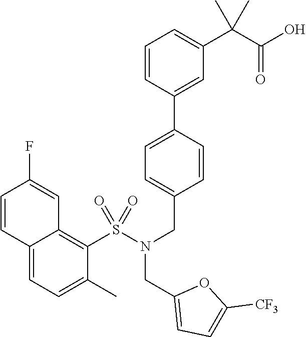 [SMILES: Cc1ccc2ccc(F)cc2c1S(=O)(=O)N(Cc1ccc(-c2cccc(C(C)(C)C(=O)O)c2)cc1)Cc1ccc(C(F)(F)F)o1]

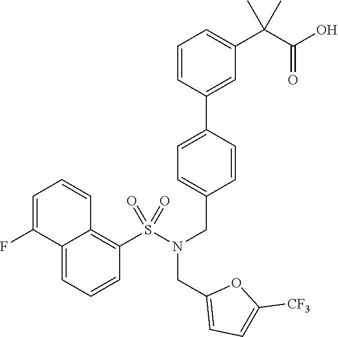 [SMILES: CC(C)(C(=O)O)c1cccc(-c2ccc(CN(Cc3ccc(C(F)(F)F)o3)S(=O)(=O)c3cccc4c(F)cccc34)cc2)c1]

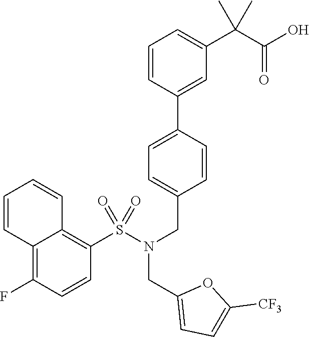 [SMILES: CC(C)(C(=O)O)c1cccc(-c2ccc(CN(Cc3ccc(C(F)(F)F)o3)S(=O)(=O)c3ccc(F)c4ccccc34)cc2)c1]

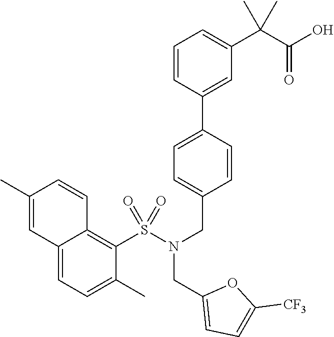 [SMILES: Cc1ccc2c(S(=O)(=O)N(Cc3ccc(-c4cccc(C(C)(C)C(=O)O)c4)cc3)Cc3ccc(C(F)(F)F)o3)c(C)ccc2c1]

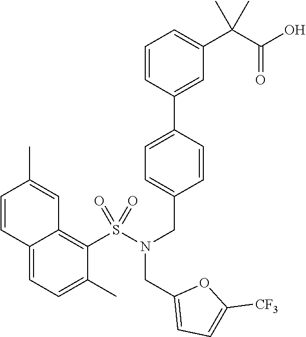 [SMILES: Cc1ccc2ccc(C)c(S(=O)(=O)N(Cc3ccc(-c4cccc(C(C)(C)C(=O)O)c4)cc3)Cc3ccc(C(F)(F)F)o3)c2c1]

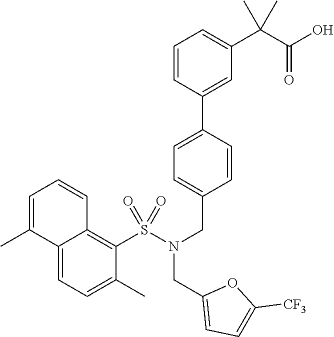 [SMILES: Cc1ccc2c(C)cccc2c1S(=O)(=O)N(Cc1ccc(-c2cccc(C(C)(C)C(=O)O)c2)cc1)Cc1ccc(C(F)(F)F)o1]

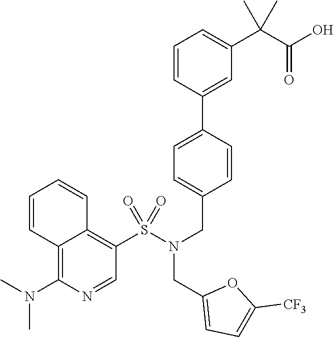 [SMILES: CN(C)c1ncc(S(=O)(=O)N(Cc2ccc(-c3cccc(C(C)(C)C(=O)O)c3)cc2)Cc2ccc(C(F)(F)F)o2)c2ccccc12]

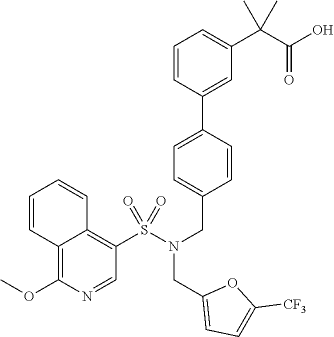 [SMILES: COc1ncc(S(=O)(=O)N(Cc2ccc(-c3cccc(C(C)(C)C(=O)O)c3)cc2)Cc2ccc(C(F)(F)F)o2)c2ccccc12]

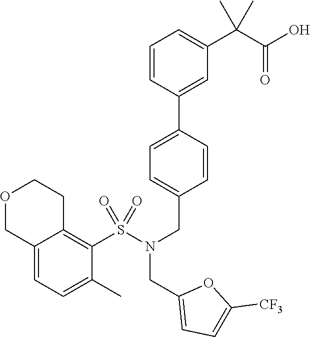 [SMILES: Cc1ccc2c(c1S(=O)(=O)N(Cc1ccc(-c3cccc(C(C)(C)C(=O)O)c3)cc1)Cc1ccc(C(F)(F)F)o1)CCOC2]

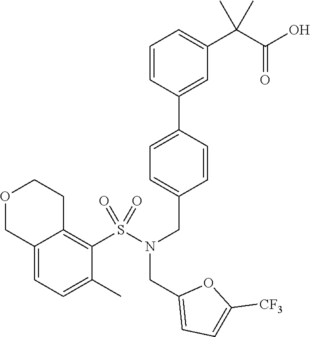 [SMILES: Cc1ccc2c(c1S(=O)(=O)N(Cc1ccc(-c3cccc(C(C)(C)C(=O)O)c3)cc1)Cc1ccc(C(F)(F)F)o1)CCOC2]

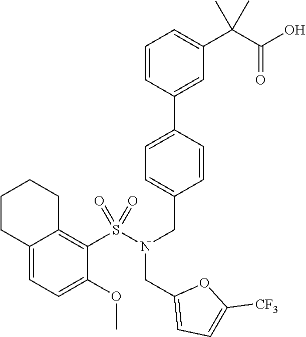 [SMILES: COc1ccc2c(c1S(=O)(=O)N(Cc1ccc(-c3cccc(C(C)(C)C(=O)O)c3)cc1)Cc1ccc(C(F)(F)F)o1)CCCC2]

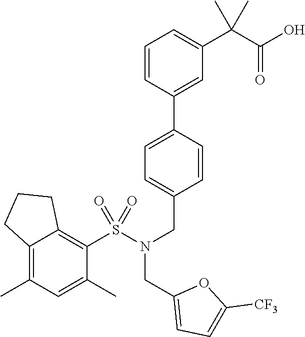 [SMILES: Cc1cc(C)c(S(=O)(=O)N(Cc2ccc(-c3cccc(C(C)(C)C(=O)O)c3)cc2)Cc2ccc(C(F)(F)F)o2)c2c1CCC2]

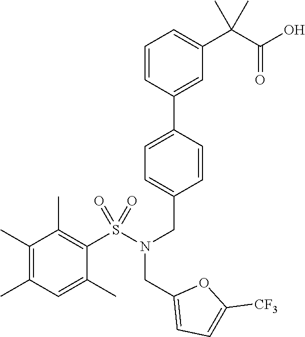 [SMILES: Cc1cc(C)c(S(=O)(=O)N(Cc2ccc(-c3cccc(C(C)(C)C(=O)O)c3)cc2)Cc2ccc(C(F)(F)F)o2)c(C)c1C]

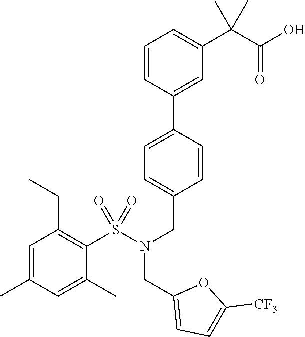 [SMILES: CCc1cc(C)cc(C)c1S(=O)(=O)N(Cc1ccc(-c2cccc(C(C)(C)C(=O)O)c2)cc1)Cc1ccc(C(F)(F)F)o1]

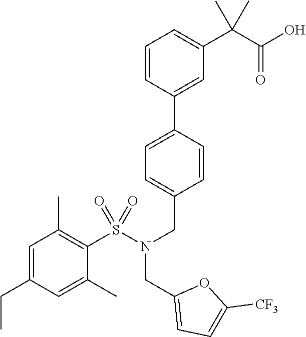 [SMILES: CCc1cc(C)c(S(=O)(=O)N(Cc2ccc(-c3cccc(C(C)(C)C(=O)O)c3)cc2)Cc2ccc(C(F)(F)F)o2)c(C)c1]

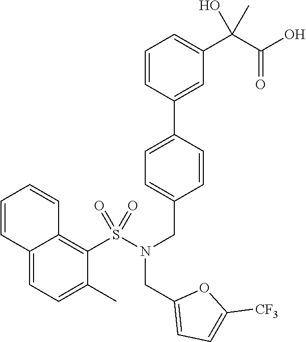 [SMILES: Cc1ccc2ccccc2c1S(=O)(=O)N(Cc1ccc(-c2cccc(C(C)(O)C(=O)O)c2)cc1)Cc1ccc(C(F)(F)F)o1]

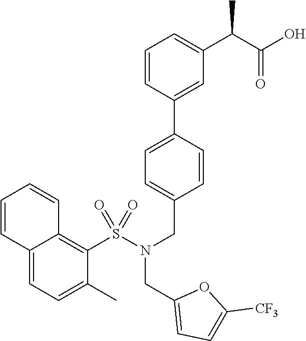 [SMILES: Cc1ccc2ccccc2c1S(=O)(=O)N(Cc1ccc(-c2cccc([C@@H](C)C(=O)O)c2)cc1)Cc1ccc(C(F)(F)F)o1]

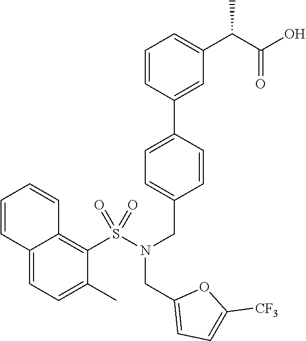 [SMILES: Cc1ccc2ccccc2c1S(=O)(=O)N(Cc1ccc(-c2cccc([C@H](C)C(=O)O)c2)cc1)Cc1ccc(C(F)(F)F)o1]

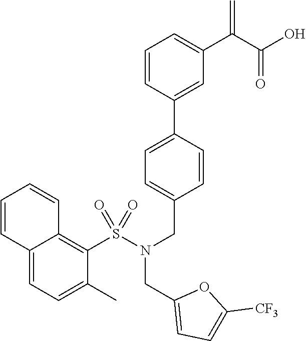 [SMILES: C=C(C(=O)O)c1cccc(-c2ccc(CN(Cc3ccc(C(F)(F)F)o3)S(=O)(=O)c3c(C)ccc4ccccc34)cc2)c1]

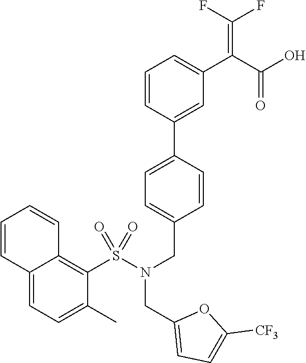 [SMILES: Cc1ccc2ccccc2c1S(=O)(=O)N(Cc1ccc(-c2cccc(C(C(=O)O)=C(F)F)c2)cc1)Cc1ccc(C(F)(F)F)o1]

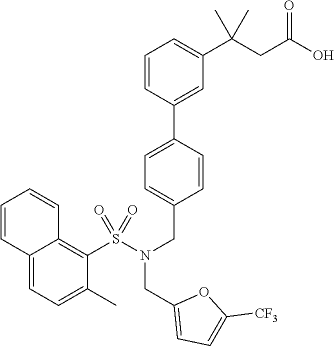 [SMILES: Cc1ccc2ccccc2c1S(=O)(=O)N(Cc1ccc(-c2cccc(C(C)(C)CC(=O)O)c2)cc1)Cc1ccc(C(F)(F)F)o1]

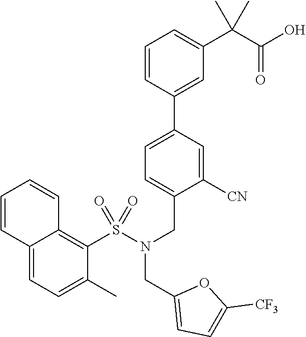 [SMILES: Cc1ccc2ccccc2c1S(=O)(=O)N(Cc1ccc(C(F)(F)F)o1)Cc1ccc(-c2cccc(C(C)(C)C(=O)O)c2)cc1C#N]

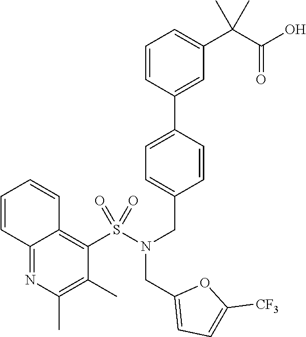 [SMILES: Cc1nc2ccccc2c(S(=O)(=O)N(Cc2ccc(-c3cccc(C(C)(C)C(=O)O)c3)cc2)Cc2ccc(C(F)(F)F)o2)c1C]

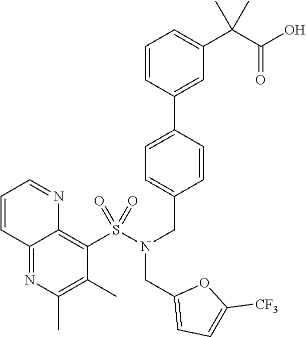 [SMILES: Cc1nc2cccnc2c(S(=O)(=O)N(Cc2ccc(-c3cccc(C(C)(C)C(=O)O)c3)cc2)Cc2ccc(C(F)(F)F)o2)c1C]

Compound Stock Solutions

The tested compounds were usually dissolved, tested and stored as 20 mM stock solutions in DMSO. Since sulfonyl acetic acid derivatives tend to decarboxylate under these conditions, these stock solutions were prepared, tested and stored as 20 mM DMSO stock solutions containing 100 mM trifluoroacetic acid (5 equivalents). Sulfonyl acetic acid derivatives are shelf stable as solid at rt for long time as reported by Griesbrecht et al. (Synlett 2010:374) or Faucher et al. (J. Med. Chem. 2004; 47:18).

TR-FRETβ Activity Assay

Recombinant GST-LXRβ ligand-binding domain (LBD; amino acids 156-461; NP009052; SEQ ID NO:2) was expressed in E. coli and purified via gluthatione-sepharose affinity chromatography. N-terminally biotinylated NCoA3 coactivator peptide (SEQ ID NO:1) was chemically synthesized (Eurogentec). Assays were done in 384 well format (final assay volume of 25 μL/well) in a Tris/HCl buffer (pH 6.8) containing KCl, bovine serum albumin, Triton-X-100 and 1 μM 24(S)-25-epoxycholesterol as LXR-prestimulating agonist. Assay buffer was provided and test articles (potential LXR inverse agonists) were titrated to yield final assay concentrations of 50 μM, 16.7 μM, 5.6 μM, 1.9 μM, 0.6 μM, 0.2 μM, 0.07 μM, 0.02 μM, 0.007 μM, 0.002 μM with one vehicle control. Finally, a detection mix was added containing anti GST-Tb cryptate (CisBio; 610SAXLB) and Streptavidin-XL665 (CisBio; 610SAXLB) as fluorescent donor and acceptor, respectively, as well as the coactivator peptide and LXRβ-LBD protein (SEQ ID NO:2). The reaction was mixed thoroughly, equilibrated for 1 h at 4° C. and vicinity of LXRβ and coactivator peptide was detected by measurement of fluorescence in a VictorX4 multiplate reader (PerkinElmer Life Science) using 340 nm as excitation and 615 and 665 nm as emission wavelengths. Assays were performed in triplicates.

### Final Assay Concentrations of Components:

240 mM KCl, 1 μg/μL BSA, 0.002% Triton-X-100, 125 μg/μL anti GST-Tb cryptate, 2.5 ng/μL Streptavidin-XL665, coactivator peptide (400 nM), LXRβ protein (530 μg/mL, i.e. 76 nM)

### LXR Gal4 Reporter Transient Transfection Assays

LXRα and LXRβ activity status was determined via detection of interaction with coactivator and corepressor proteins in mammalian two-hybrid experiments (M2H). For this, via transient transfection the full length (FL) proteins of LXRα (amino acids 1-447; NP005684; SEQ ID NO:7) or LXRβ-(amino acids 1-461; NP009052; SEQ ID NO:8) or the ligand-binding domains (LBD) of LXRα (amino acids 155-447 SEQ ID NO:3) or LXRβ (amino acids 156-461; SEQ ID NO:4) were expressed from pCMV-AD (Stratagene) as fusions to the transcriptional activation domain of NFkB. As cofactors, domains of either the steroid receptor coactivator 1 (SRC1; amino acids 552-887; SEQ ID NO:5) or of the corepressor NCoR (amino acids 1903-2312 SEQ ID NO:6) were expressed as fusions to the DNA binding domain of the yeast transcription factor GAL4 (from pCMV-BD; Stratagene). Interaction was monitored via activation of a coexpressed Firefly Luciferase Reporter gene under control of a promoter containing repetitive GAL4 response elements (vector pFRLuc; Stratagene). Transfection efficiency was controlled via cotransfection of constitutively active pRL-CMV Renilla reniformis luciferase reporter (Promega). HEK293 cells were grown in minimum essential medium (MEM) with 2 mM L-glutamine and Earle's balanced salt solution supplemented with 8.3% fetal bovine serum, 0.1 mM non-essential amino acids, 1 mM sodium pyruvate, at 37° C. in 5% CO₂. 3.5×10⁴ cells/well were plated in 96-well cell culture plates in growth medium supplemented with 8.3% fetal bovine serum for 16-20 h to ˜90% confluency. For transfection, medium was taken off and LXR and cofactor expressing plasmids as well as the reporter plasmids are added in 30 μL OPTIMEM/well including polyethylene-imine (PEI) as vehicle. Typical amounts of plasmids transfected/well: pCMV-AD-LXR (5 ng), pCMV-BD-cofactor (5 ng), pFR-Luc (100 ng), pRL-CMV (0.5 ng). Compound stocks were prepared in DMSO, prediluted in MEM to a total volume of 120 μL, and added 4 h after addition of the transfection mixture (final vehicle concentration not exceeding 0.2%). Cells were incubated for additional 16 h, lysed for 10 min in 1× Passive Lysis Buffer (Promega) and Firefly and Renilla luciferase activities were measured sequentially in the same cell extract using buffers containing D-luciferine and coelenterazine, respectively. Measurements of luminescence were done in a BMG-Iuminometer.

TABLE 1

| Materials | Company | Cat.No. |
|---|---|---|
| HEK293 cells | DSMZ | ACC305 |
| MEM | Sigma-Aldrich | M2279 |
| OPTIMEM | LifeTechnologies | 11058-021 |
| FCS | Sigma-Aldrich | F7542 |
| Glutamax | lnvitrogen | 35050038 |
| Pen/Strep | Sigma Aldrich | P4333 |
| Sodium Pyruvate | Sigma Aldrich | S8636 |
| Non Essential Amino Acids | Sigma Aldrich | M7145 |
| Trypsin | Sigma-Aldrich | T3924 |
| PBS | Sigma Aldrich | D8537 |
| PEI | Sigma Aldrich | 40.872-7 |
| Passive Lysis Buffer (5x) | Promega | E1941 |
| D-Luciferine | PJK | 260150 |
| Coelentrazine | PJK | 260350 |

Activity ranges (EC₅₀): A: >10 μM, B: 1 μM to <10 μM, C: 100 nM to <1 μM, D: <100 nM; behavior in FRET assay: ag=agonist, ia=inverse agonist; italic bold capital letters in the M2H assay indicate that efficacy (compared to GW2033) is below 40%.

| | | | M2H | M2H | M2H | M2H |
|---|---|---|---|---|---|---|
| Ex. | | be- | Gal4α | Gal4β | Gal4α | Gal4β |
| # | FRETβ | havior | LBD | LBD | FL | FL |
| 1 | A | ia | C | D | | |
| 2 | C | ia | C | D | D | D |
| 2/1 | B | ia | inactive | inactive | | |
| 2/2 | C | ia | D | D | D | D |
| 2/3 | B | ia | C | C | | |
| 2/4 | C | ia | C | D | | |
| 3 | B | ia | inactive | inactive | | |
| 3/1 | A | ia | C | D | | |
| C3/2 | D | ia | D | D | D | D |
| 4 | D | ia | B | C | | |
| 5 | B | ia | | | B | C |
| 5/1 | B | ia | | | C | C |
| 5/2 | B | ia | C | C | C | C |
| 5/3 | B | ia | C | C | | |
| 5/4 | C | ia | C | C | | |
| 5/5 | A | ia | | | B | C |
| 5/7 | B | ia | | | C | C |
| C6 | A | ia | B | C | | |
| C7 | B | ia | C | C | | |
| 7/1 | B | ia | C | D | C | C |
| 7/2 | B | ia | C | C | | |
| 7/4 | C | ia | D | D | | |
| 7/5 | C | ia | D | D | D | D |
| 7/6 | C | ia | C | D | | |
| 7/7 | C | ia | C | D | | |
| 7/8 | A | ia | C | C | | |
| 7/9 | B | ia | C | D | | |
| 7/10 | B | ia | B | C | | |
| C7/11 | B | ia | C | C | | |
| 9 | B | ia | | | C | C |
| 10 | C | ia | | | C | C |
| 10/1 | B | ag | | | inactive | C |
| 10/2 | B | ia | | | B | C |
| 10/3 | B | ag | | | B | C |
| 10/4 | D | ia | D | D | D | D |
| 10/5 | D | ia | D | D | D | D |
| 10/6 | B | ag | | | C | D |
| 10/7 | C | ag | | | C | D |
| 10/8 | B | ag | | | B | B |
| 10/9 | B | ia | | | B | C |
| 10/10 | B | ia | | | C | C |
| 10/11 | B | ag | | | B | C |
| 10/12 | B | ia | | | B | C |
| 10/13 | B | ia | | | B | C |
| 10/14 | C | ia | | | D | D |
| 10/15 | D | ia | | | D | D |
| 10/16 | A | ia | | | B | C |
| 10/17 | B | ia | | | C | D |
| 10/18 | D | ia | | | D | D |
| 10/19 | C | ag | | | D | D |
| 10/20 | C | ag | | | C | D |
| 11 | B | ia | | | B | B |
| 11/3 | A | ia | | | B | B |
| 11/5 | A | ia | | | B | C |
| 11/6 | C | ag | | | C | D |
| 11/9 | B | ia | | | B | B |
| 11/10 | B | ia | | | B | B |
| 11/11 | C | ag | | | B | C |
| 11/12 | C | ag | | | C | D |
| 11/13 | B | ia | | | B | C |
| 11/15 | B | ia | | | B | C |
| 11/16 | inactive | | | | B | C |
| 11/17 | B | ia | B | B | | |
| 11/18 | C | ia | C | D | | |
| 11/19 | B | ia | | | C | C |
| 14/1 | B | ag | | | inactive | B |
| 14/2 | B | ia | | | B | C |
| 16 | A | ia | inactive | B | | |
| 16/1 | A | ia | B | C | | |
| 16/2 | A | ia | C | C | | |
| 17 | B | ia | | | B | B |
| 17/1 | B | ia | | | B | B |
| 17/2 | C | ia | | | D | D |
| 17/3 | D | ia | | | D | D |
| 19 | C | ia | C | D | | |
| 19/1 | B | ia | inactive | C | | |
| 19/2 | B | ia | C | C | | |
| 22 | B | ia | B | D | | |
| 23 | C | ia | D | D | | |
| 24 | D | ia | D | D | D | D |
| 25 | C | ia | C | D | | |
| 25/1 | B | ia | B | B | | |
| 25/3 | B | ia | C | C | | |
| 26 | C | ia | | | D | D |
| 27 | C | ia | | | D | D |
| 29 | inactive | | | | C | C |
| 30 | C | ag | | | D | D |

Pharmacokinetics

The pharmacokinetics of different sulfonamides was assessed in mice after single dosing and oral and intraperitoneal administrations. Blood and liver exposure was measured via LC-MS.

The study design was as follows:

Animals: C57BL/6J (Janvier) males

Diet: standard rodent chow

Vehicle for i.p. injection: 0.5% HPMC (w:v) in water, injection volume: <5 mL/kg

Animal handling: animals were withdrawn from food at least 12 h before administration

Design: single dose oral and bid ip administration, n=3 animals per group

Sacrifice: at t=4 h after administration

Bioanalytics: LC-MS of liver and blood samples

Study Results

| | Dose | blood exposure, | liver exposure, | liver/blood ratio, |
|---|---|---|---|---|
| Example # | (mg) | 4 h | 4 h | 4 h |
| GSK2033 (neutral | 20 | po: below LLOQ | po: below LLOQ | — |
| comparative example) | | (14.4 ng/mL) | (9.6 ng/mL) | |
| SR9238 (comparative | 20 | po: below LLOQ | po: below LLOQ | — |
| example with ester moiety) | | | | |
| C3/2 (neutral | 20 | po: 115 ng/mL | po: 64 ng/mL | po: 0.56 |
| comparative example) | | | | |
| 5 | 20 | po: 0.15 μM | po: 4.6 μM | po: 31 |
| | | ip: 0.34 μM | ip: 9.3 μM | ip: 27 |
| 7/5 | 20 | po: 300 ng/mL | po: 5398 ng/mL | po: 18 |
| 10/4 | 20 | po: 189 ng/mL | po: 2136 ng/mL | po: 11 |
| 10/5 | 20 | po: 242 ng/mL | po: 5120 ng/mL | po: 21 |
| 11/19 | 20 | po: 0.01 μM | po: 1.07 μM | po: 125 |
| 24 | 20 | po: 231 ng/mL | po: 5882 ng/mL | po: 25 |

We confirmed that neutral sulfonamide GSK2033 and SR9238 are not orally bioavailable. Surprisingly we found, that when an acid moiety or acidic bioisostere is installed at another area of the molecule, i.e. instead or near the methylsulfone moiety of GSK2033/SR9238, these acidic compounds maintained to be potent on LXR and in addition are now orally bioavailable. The target tissue liver was effectively reached by compounds of the present invention (5, 7/5, 10/4, 10/5, 11/19 and 24) and a systemic exposure, which is not desired, could be minimized.

In addition, the compounds of the present invention are more hepatotropic due to the acid moiety or acidic bioisosteric moiety (liver/blood ratios of 11 to 125). For comparison, neutral example C/2 showed a liver/blood ratios of 0.56.

Short Term HFD Mouse Model:

The in vivo transcriptional regulation of several LXR target genes by LXR modulators was assessed in mice.

For this, C57BL/6J were purchased from Elevage Janvier (Rennes, France) at the age of 8 weeks. After an acclimation period of two weeks, animals were preferred on a high fat diet (HFD) (Ssniff Spezialdiaten GmbH, Germany, Surwit EF D12330 mod, Cat. No. E15771-34), with 60 kcal % from fat plus 1% (w/w) extra cholesterol (Sigma-Aldrich, St. Louis, Mo.) for 5 days. Animals were maintained on this diet during treatment with LXR modulators. The test compounds were formulated in 0.5% hydroxypropylmethylcellulose (HPMC) and administered in three doses (20 mg/kg each) by oral gavage according to the following schedule: on day one, animals received treatment in the morning and the evening (ca. 17:00), on day two animals received the final treatment in the morning after a 4 h fast and were sacrificed 4 h thereafter. Animal work was conducted according to the national guidelines for animal care in Germany.

Upon termination, liver was collected, dipped in ice cold PBS for 30 seconds and cut into appropriate pieces. Pieces were snap frozen in liquid nitrogen and stored at −80° C. For the clinical chemistry analysis from plasma, alanine aminotransferase (ALT, IU/mL), cholesterol (CHOL, mg/dL) and triglycerides (TG, mg/dL) were determined using a fully-automated bench top analyzer (Respons®910, DiaSys Greiner GmbH, Flacht, Germany) with system kits provided by the manufacturer.

Analysis of Gene Expression in Liver Tissue.

To obtain total RNA from frozen liver tissue, samples (25 mg liver tissue) were first homogenized with RLA buffer (4M guanidin thiocyanate, 10 mM Tris, 0.97% w:v β-mercapto-ethanol). RNA was prepared using a SV 96 total RNA Isolation system (Promega, Madison, Wis., USA) following the manufacturer's instructions. cDNAs were synthesized from 0.8-1 μg of total RNA using All-in-One cDNA Supermix reverse transcriptase (Absource Diagnostics, Munich, Germany). Quantitative PCR was performed and analyzed using Prime time Gene expression master mix (Integrated DNA Technologies, Coralville, Iowa, USA) and a 384-format ABI 7900HT Sequence Detection System (Applied Biosystems, Foster City, USA). The expression of the following genes was analysed: Stearoyl-CoA desaturase1 (Scd1), fatty acid synthase (Fas) and sterol regulatory element-binding protein1 (Srebp1). Specific primer and probe sequences (commercially available) are listed in Table 2. qPCR was conducted at 95° C. for 3 min, followed by 40 cycles of 95° C. for 15 s and 60° C. for 30 s. All samples were run in duplicates from the same RT-reaction. Gene expression was expressed in arbitrary units and normalized relative to the mRNA of the housekeeping gene TATA box binding protein (Tbp) using the comparative Ct method.

TABLE 2
Primers used for quantitative PCR.

| | Forward | Reverse | Sequence |
|---|---|---|---|
| Gene | Primer | Primer | Probe |
| Fasn | CCCCTCTGTTA | TTGTGGAAGTGC | CAGGCTCAGGGTG |
| | ATTGGCTCC | AGGTTAGG | TCCCATGTT |
| | (SEQ ID | (SEQ ID | (SEQ ID |
| | NO: 9) | NO: 10) | NO: 11) |
| Scd1 | CTGACCTGAAA | AGAAGGTGCTAA | TGTTTACAAAAGT |
| | GCCGAGAAG | CGAACAGG | CTCGCCCCAGCA |
| | (SEQ ID | (SEQ ID | (SEQ ID |
| | NO: 12) | NO: 13) | NO: 14) |
| Srebp1c | CCATCGACTAC | GCCCTCCATAGA | TCTCCTGCTTGAG |
| | ATCCGCTTC | CACATCTG | CTTCTGGTTGC |
| | (SEQ ID | (SEQ ID | (SEQ ID |
| | NO: 15) | NO: 16) | NO: 17) |
| Tbp | CACCAATGACT | CAAGTTTACAGC | ACTCCTGCCACAC |
| | CCTATGACCC | CAAGATTCACG | CAGCCTC |
| | (SEQ ID | (SEQ ID | (SEQ ID |
| | NO: 18) | NO: 19) | NO: 20) |

Study Results

| Example | plasma exposure, | liver exposure, | liver/plasma ratio, |
|---|---|---|---|
| # | 4 h | 4 h | 4 h |
| 10/5 | 131 nM | 4372 nM | 33.3 |
| 24 | 102 nM | 5359 nM | 52.4 |

| Example | Fasn suppression | Scd1 suppression | Srebp1c suppression |
|---|---|---|---|
| # | compared to vehicle | compared to vehicle | compared to vehicle |
| 10/5 | 0.41 | 0.38 | 0.33 |
| 24 | 0.23 | 0.25 | 0.25 |

Multiple oral dosing of compounds 10/5 and 24 in mice lead to a high liver exposure with a favourable liver to plasma ratio. Hepatic LXR target genes were effectively suppressed. These genes are related to hepatic de-novo lipogenesis. A suppression of these genes will reduce liver fat (liver triglycerides).

1. A compound represented by Formula (I) 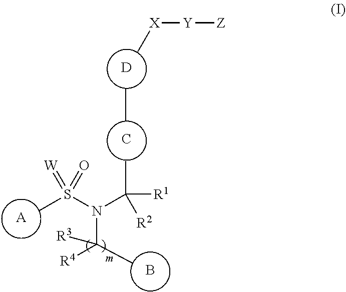
an enantiomer, diastereomer, tautomer, N-oxide, solvate, prodrug and pharmaceutically acceptable salt thereof, wherein
R¹, R² are independently selected from H and C₁₋₄-alkyl,
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R¹ and R² together are oxo, a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
or R¹ and an adjacent residue from ring C form a saturated or partially saturated 5- to 8-membered cycloalkyl or a 5- to 8-membered heterocycloalkyl containing 1 to 4 heteroatoms independently selected from N, O and S,
wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
R³, R⁴ are independently selected from H, C₁₋₄-alkyl and halo-C₁₋₄-alkyl;
wherein alkyl is unsubstituted or substituted with 1 to 3 substituents independently selected from halogen, CN, OH, oxo, C₁₋₄-alkyl, halo-C₁₄-alkyl, O—C₁₋₄-alkyl, O-halo-C₁₋₄-alkyl;
or R³ and R⁴ together are oxo, a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
or R³ and an adjacent residue from ring B form a partially saturated 5- to 8-membered cycloalkyl or a 5- to 8-membered heterocycloalkyl containing 1 to 4 heteroatoms independently selected from N, O and S,
wherein the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
\textcircled{A} is selected from the group consisting of 3- to 10-membered cycloalkyl, 3- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
\textcircled{B} is selected from the group consisting of 6- or 10-membered aryl and 5- to 10-membered heteroaryl containing 1 to 4 heteroatoms independently selected from N, O and S, wherein aryl and heteroaryl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkylene-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
\textcircled{C} is selected from the group consisting of 3- to 10-membered cycloalkyl, 3- to 10-membered heterocycloalkyl containing 1 to 4 heteratoms independently selected from N, O and S, 6- or 10-membered aryl and 5- to 10-membered heteroaryl containing 1 to 4 heteratoms independently selected from N, O and S,
wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
\textcircled{D} is selected from the group consisting of 3- to 10-membered cycloalkyl, 3- to 10-membered heterocycloalkyl containing 1 to 4 heteratoms independently selected from N, O and S, 6- or 10-membered aryl and 5- to 10-membered heteroaryl containing 1 to 4 heteratoms independently selected from N, O and S,
wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-(3- to 6-membered heterocycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹ and C₀₋₆-alkylene-NR⁸¹R⁸²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially saturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
W is selected from O, NR¹¹ or absent;
the residue X—Y—Z on ring D is linked in 1,3-orientation regarding the connection towards ring C;
X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—;
Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S,
wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═O)H(OH), —B(OH)₂; 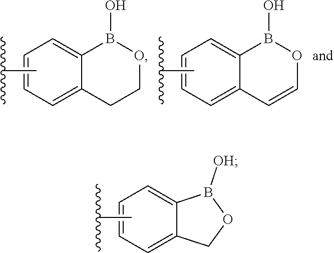
or X—Y—Z is selected from —SO₃H and —SO₂NHCOR⁹⁰;
or when X is not a bond then Z in addition can be selected from —CONR⁹¹R⁹², —S(═O)₂NR⁹¹R⁹², 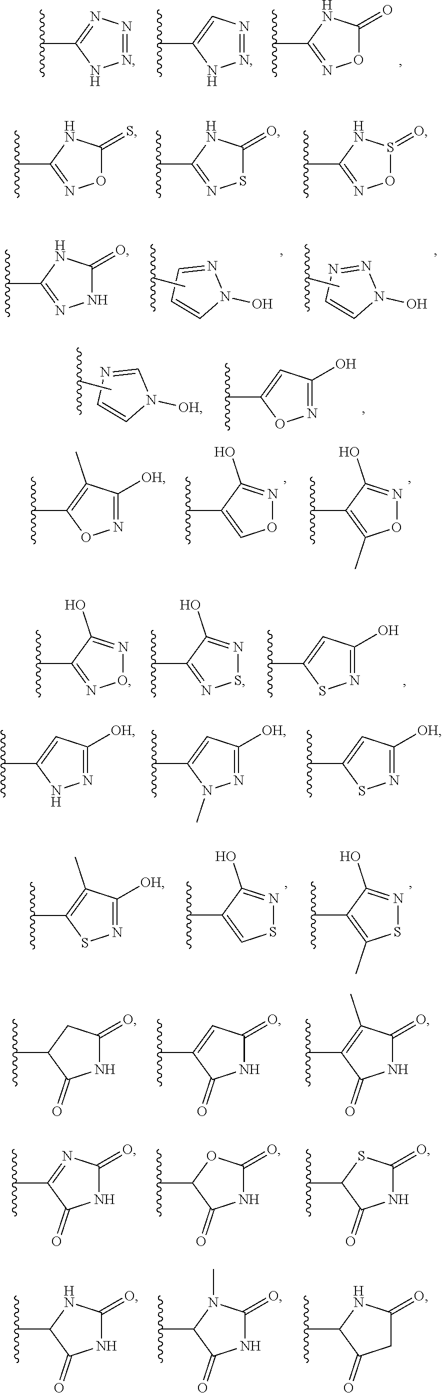 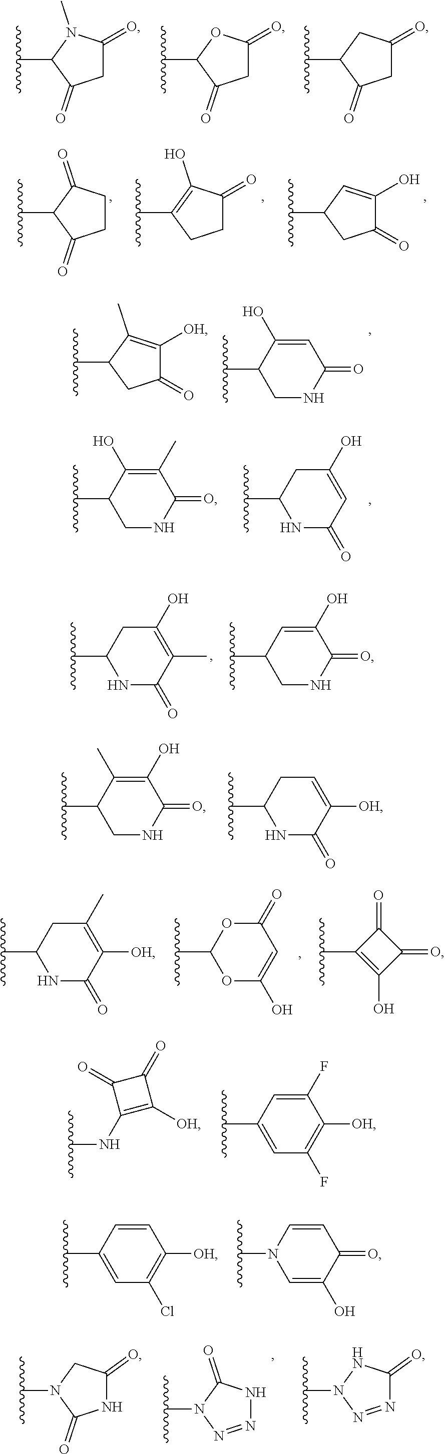 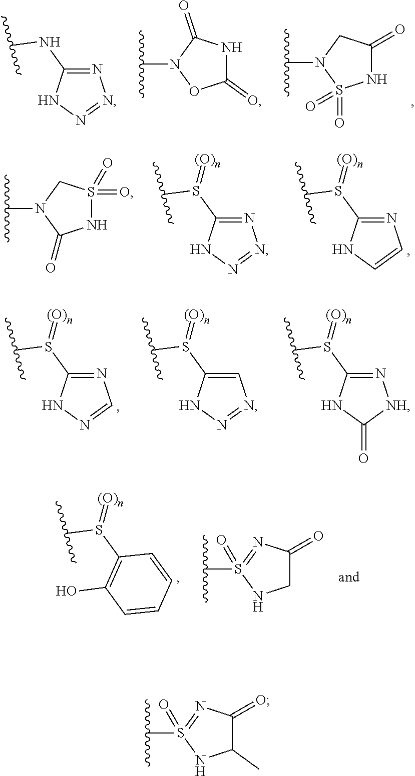
R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl and C(═O)—O-halo-C₁₋₄-alkyl;
R⁵¹, R⁵², R⁶¹, R⁶², R⁷¹, R⁷², R⁸¹, R⁸² are independently selected from H and C₁₋₄-alkyl, wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
or R⁵¹ and R⁵², R⁶¹ and R⁶², R⁷¹ and R⁷², R⁸¹ and R⁸², respectively, when taken together with the nitrogen to which they are attached complete a 3- to 6-membered ring containing carbon atoms and optionally containing 1 or 2 heteroatoms independently selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
R⁹⁰ is independently selected from C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
R⁹¹, R⁹² are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
or R⁹¹ and R⁹² when taken together with the nitrogen to which they are attached complete a 3- to 6-membered ring containing carbon atoms and optionally containing 1 or 2 heteroatoms selected from O, S or N; and wherein the new formed cycle is unsubstituted or substituted with to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
n and m are independently selected from 0 to 2.

2. The compound according to claim 1 wherein
R¹, R², R³ and R⁴ are independently selected from H or Me;
W is O;
m is 1.

3. The compound according to claim 1 wherein
\textcircled{A} is selected from the group consisting of 6- or 10-membered aryl and 5- to 10-membered heteroaryl optionally containing 1 to 4 heteroatoms independently selected from N, O and S,
wherein the 6-membered aryl and the 5- to 6-membered heteroaryl are substituted with 2 to 4 substituents independently selected from the group consisting of F, Cl, CN, C₁₋₄-alkyl, —OC₁₋₄-alkyl, fluoro-C₁₋₄-alkyl and —O-fluoro-C₁₋₄-alkyl;
and wherein optionally two adjacent substituents in the aryl or heteroaryl moiety form a 5- to 6-membered partially saturated cycle optionally containing 1 to 3 heteroatoms independently selected from O, S or N, wherein this additional cycle is unsubstituted or substituted with 1 to 4 substituents independently selected from fluoro, CN, oxo, OH, Me, CF₃, CHF₂, OMe, OCF₃ and OCHF₂;
or wherein the 10-membered aryl and the 8- to 10-membered heteroaryl are unsubstituted or substituted with 1 to 4 substituents independently selected from the group consisting of F, Cl, CN, C₁₋₄-alkyl, —O—C₁₋₄-alkyl, fluoro-C₁₋₄-alkyl and —O-fluoro-C₁₋₄-alkyl.

4. A compound according to claim 1 wherein
\textcircled{B} is selected from the group consisting of phenyl, pyridinyl, pyrrolyl, thiazolyl, thiofuranyl and furanyl,
wherein phenyl, pyridinyl, pyrrolyl, thiazolyl, thiofuranyl or furanyl are substituted with 1 to 2 substituents independently selected from the group consisting of fluoro, chloro, bromo, CN, C₁₋₄-alkyl, —O—C₁₋₄-alkyl, fluoro-C₁₋₄-alkyl, —O-fluoro-C₁₋₄-alkyl, CONH₂, CONH(C₁₋₄-alkyl), CONH(fluoro-C₁₋₄-alkyl) and CON(C₁₋₄-alkyl)₂.

5. The compound according to claim 1 wherein
\textcircled{C} is selected from the group consisting of phenyl, thiophenyl, thiazolyl and pyridinyl, wherein phenyl, thiophenyl, thiazolyl and pyridinyl are unsubstituted or substituted with 1 to 2 substituents independently selected from the group consisting of fluoro, chloro, CN, C₁₋₄-alkyl, —O—C₁₋₄-alkyl, fluoro-C₁₋₄-alkyl and —O-fluoro-C₁₋₄-alkyl.

6. The compound according to claim 1 wherein
\textcircled{D} is selected from the group consisting of phenyl, pyridinyl, thiophenyl or thiazolyl
wherein phenyl, pyridinyl, thiophenyl or thiazolyl are unsubstituted or substituted with 1 to 2 substituents independently selected from the group consisting of fluoro, chloro, CN, OH, C₁₋₄-alkyl, —O—C₁₋₄-alkyl, fluoro-C₁₋₄-alkyl, —O-fluoro-C₁₋₄-alkyl and C₁₋₃-alkylene-OH.

7. The compound according to claim 1 wherein
X is selected from a bond, O, S(═O) and S(═O)₂;
Y is selected from C₁₋₃-alkylene, 3- to 6-membered cycloalkylene and 3- to 6-membered heterocycloalkylene containing 1 to 4 heteroatoms independently selected from N, O and S,
wherein alkylene, cycloalkylene or heterocycloalkylene is unsubstituted or substituted with 1 to 2 substituents independently selected from fluoro, CN, C₁₋₄-alkyl, halo-C₁₋₄-alkyl, OH, oxo, O—C₁₋₄-alkyl and O-halo-C₁₋₄-alkyl; and
Z is selected from —CO₂H and —CONHOH.

8. The compound according to claim 1 wherein
X is selected from O, S(═O) and S(═O)₂;
Y is selected from C₁₋₃-alkylene, 3- to 6-membered cycloalkylene and 3- to 6-membered heterocycloalkylene containing 1 to 4 heteroatoms independently selected from N, O and S,
wherein alkylene, cycloalkylene or heterocycloalkylene is unsubstituted or substituted with 1 to 2 substituents independently selected from fluoro, CN, C₁₋₄-alkyl, halo-C₁₋₄-alkyl, OH, oxo, O—C₁₋₄-alkyl and O-halo-C₁₋₄-alkyl; and
Z is selected from —CO₂H, —CONHOH, —CONR⁹¹R⁹², —S(═O)₂NR⁹¹R⁹², 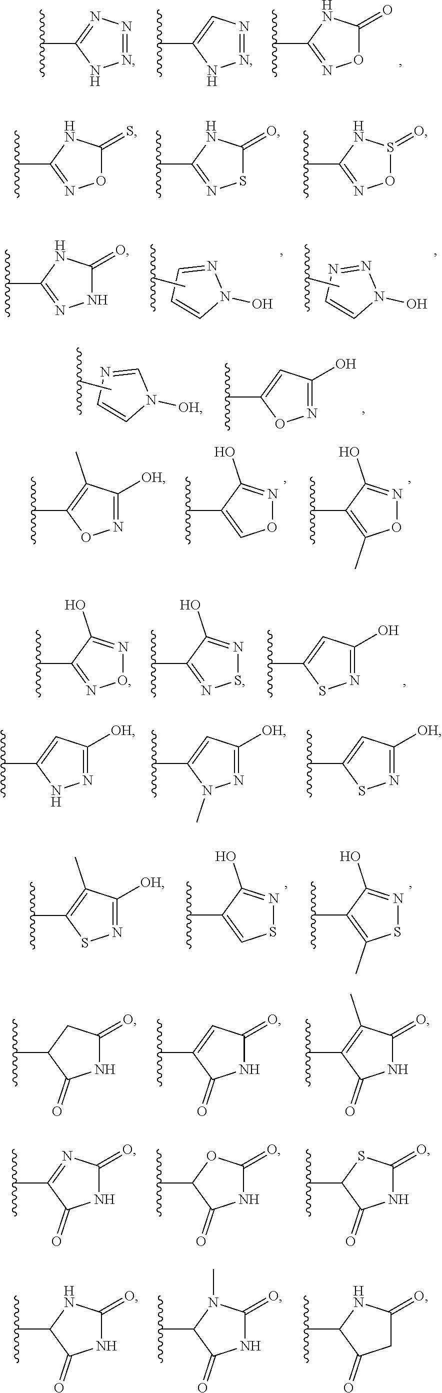 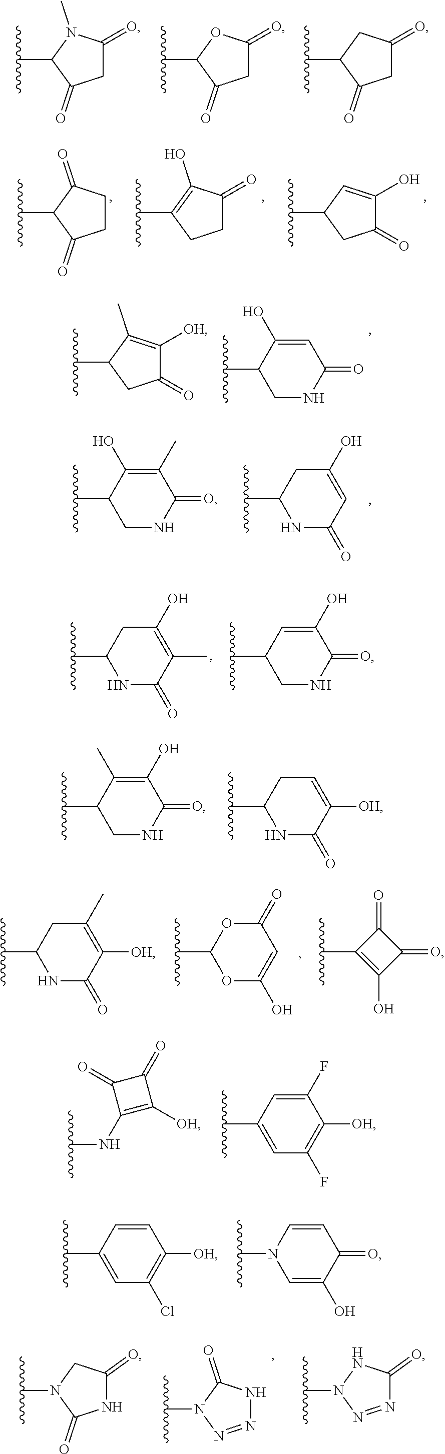 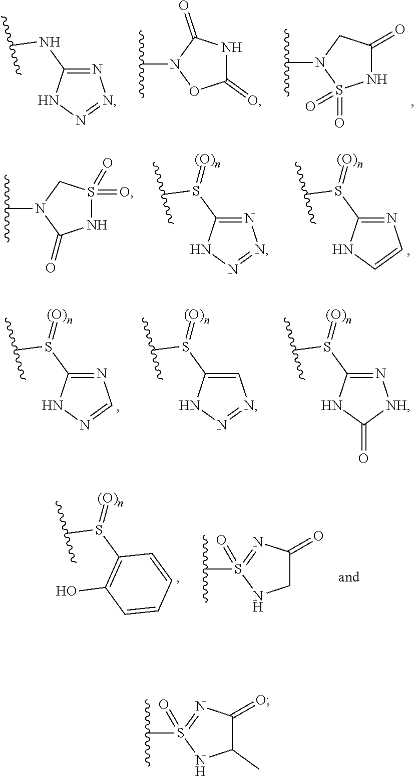
R⁹¹, R⁹² are independently selected from H and C₁₋₄-alkyl,
wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl.

9. The compound according to claim 1 wherein
\textcircled{A} is selected from 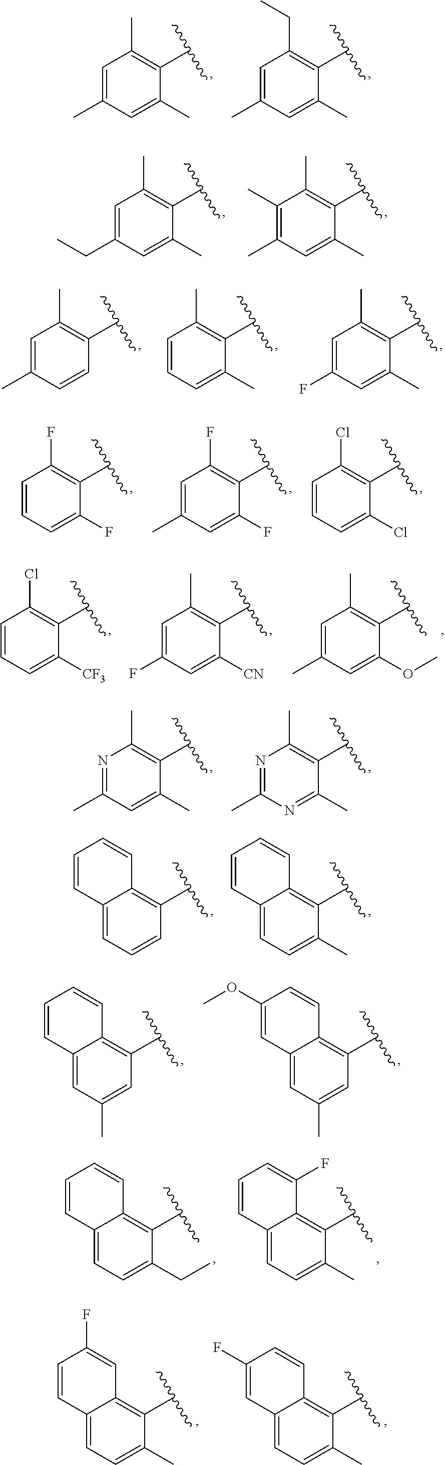 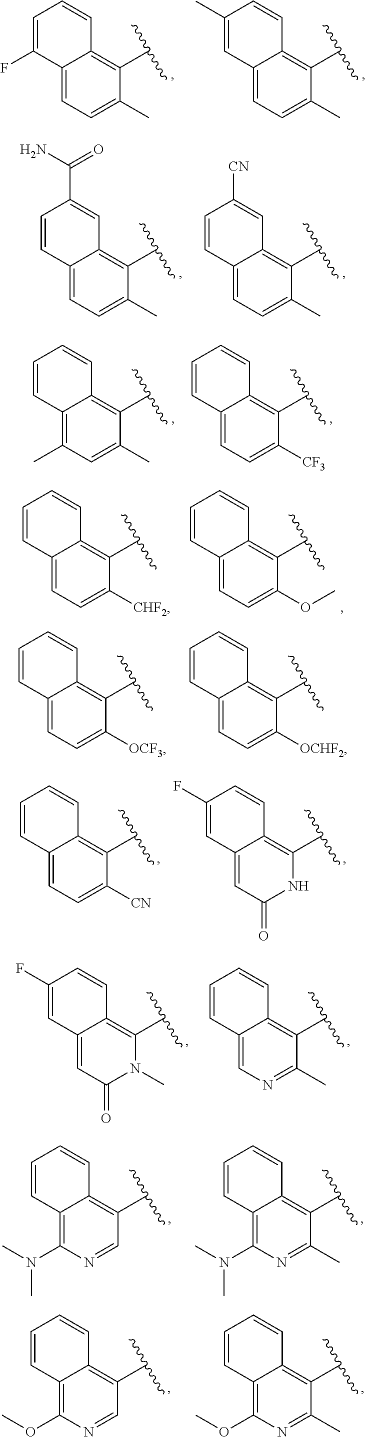 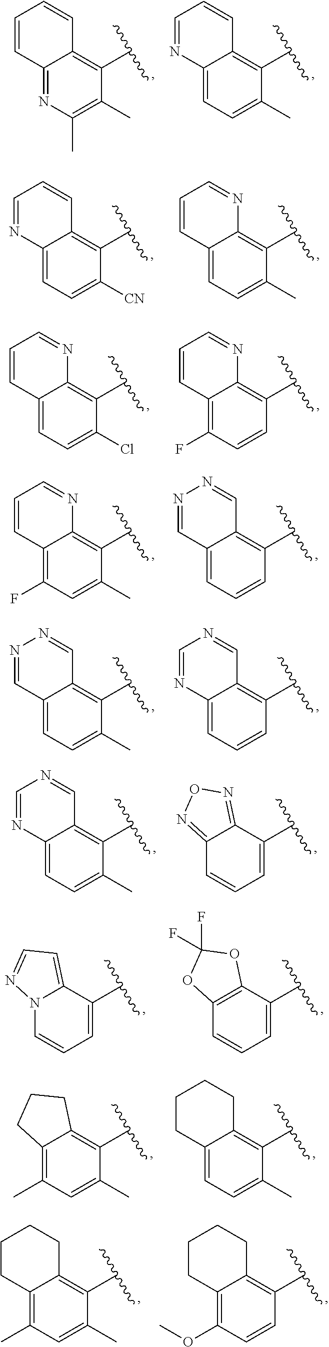 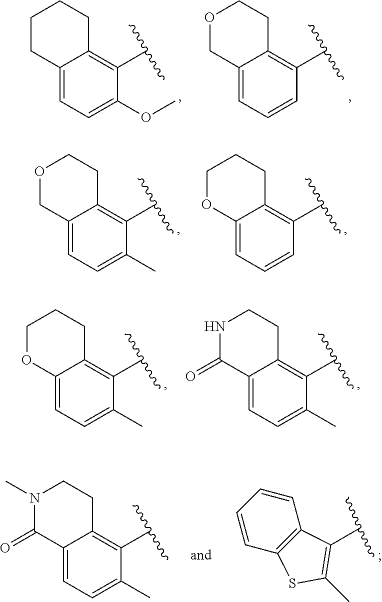
\textcircled{B} is selected from 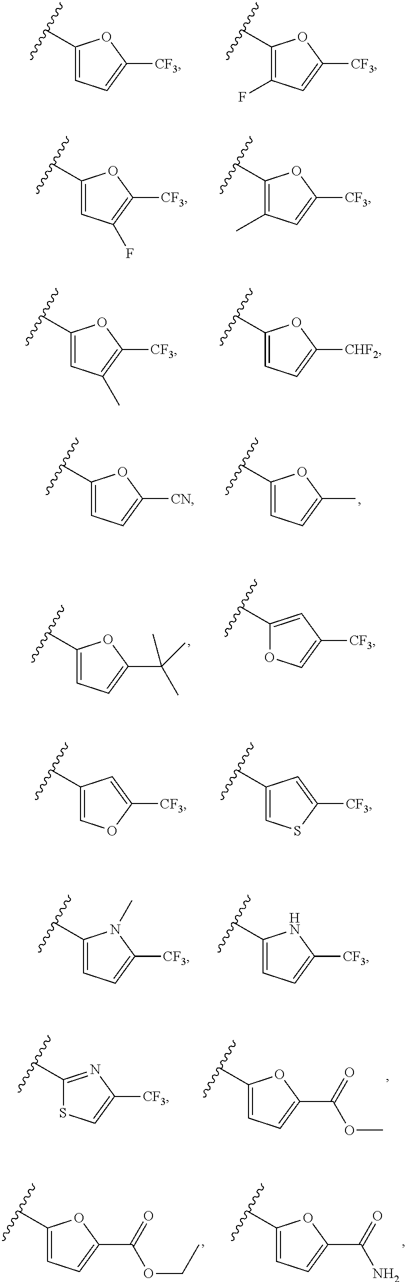 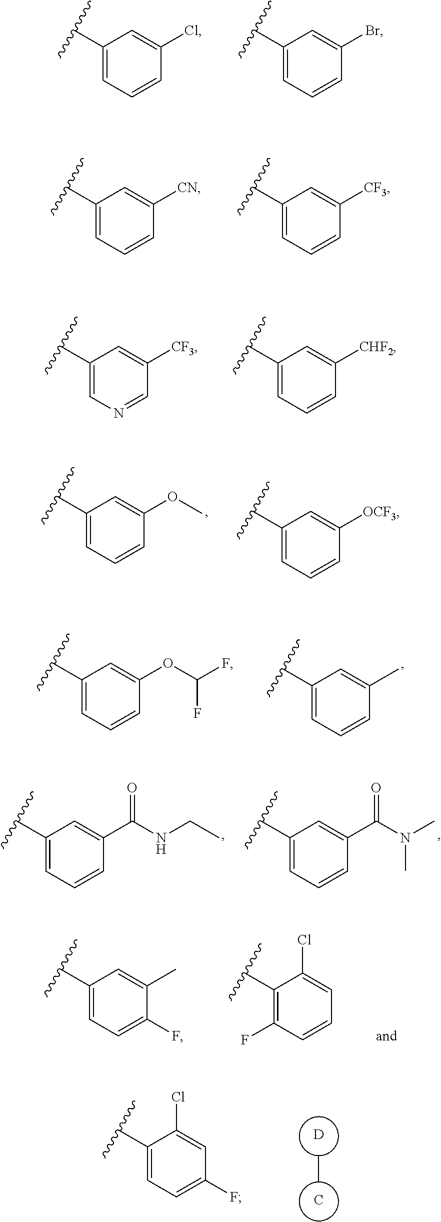
is selected from 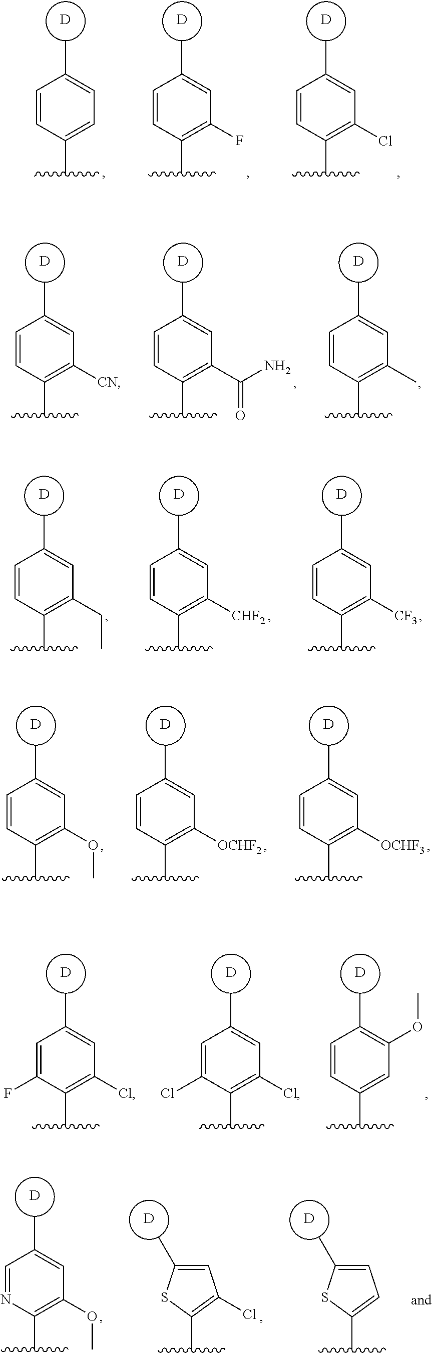 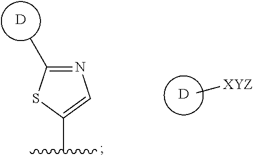
is selected from 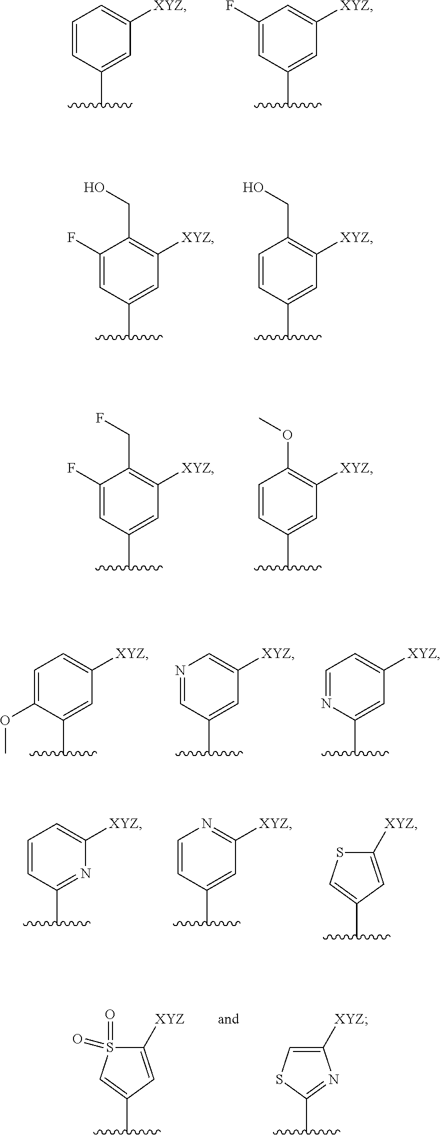
XYZ is selected from 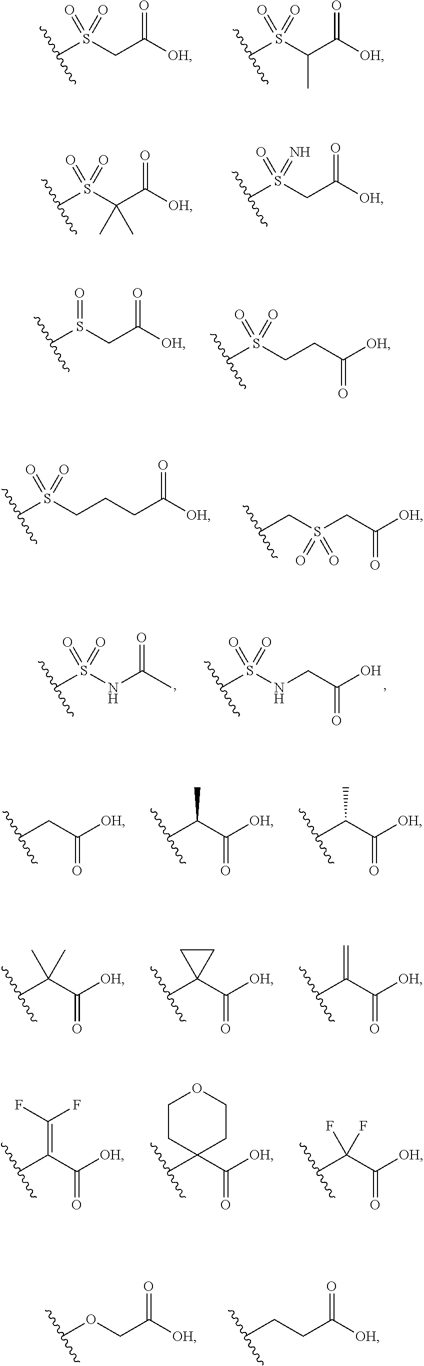 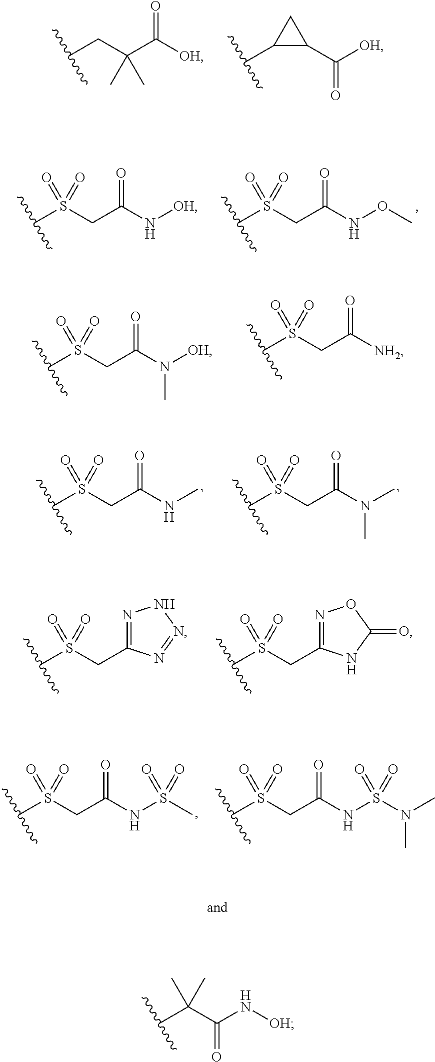
R¹, R², R³ and R⁴ are independently selected from H and Me;
W is O; and
m is selected from 1 and 2.

10. The compound according to claim 1 wherein
\textcircled{A} is selected from 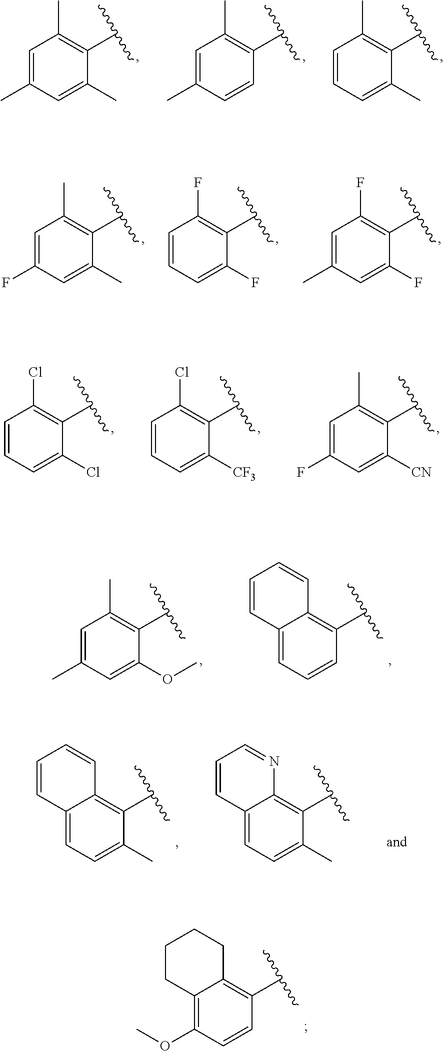
\textcircled{B} is selected from 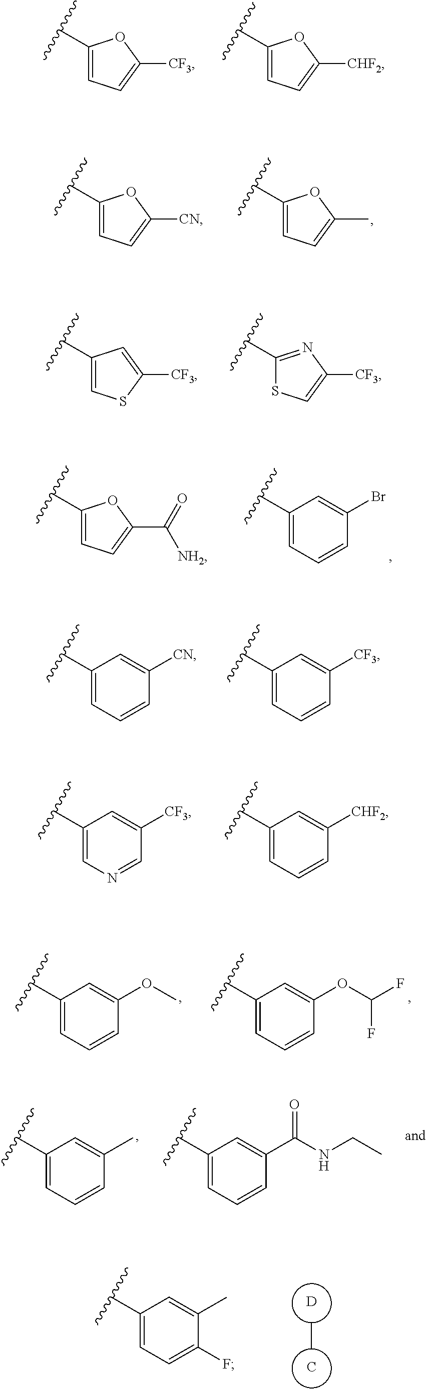
is selected from 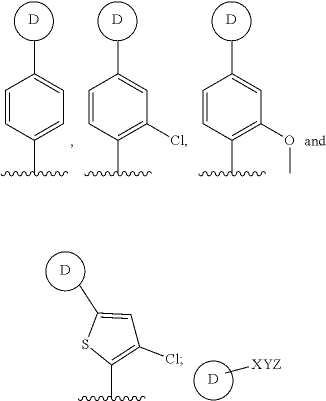
is selected from 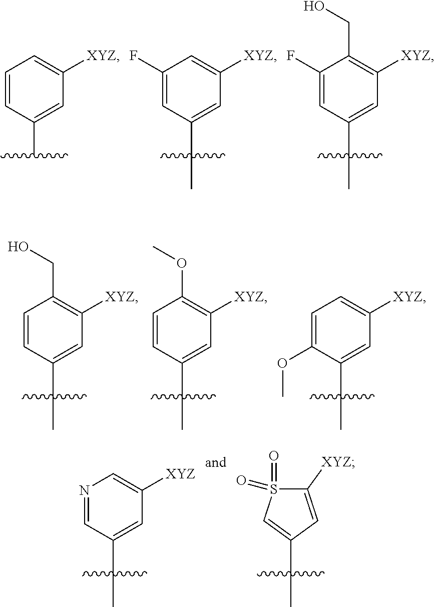
XYZ is selected from 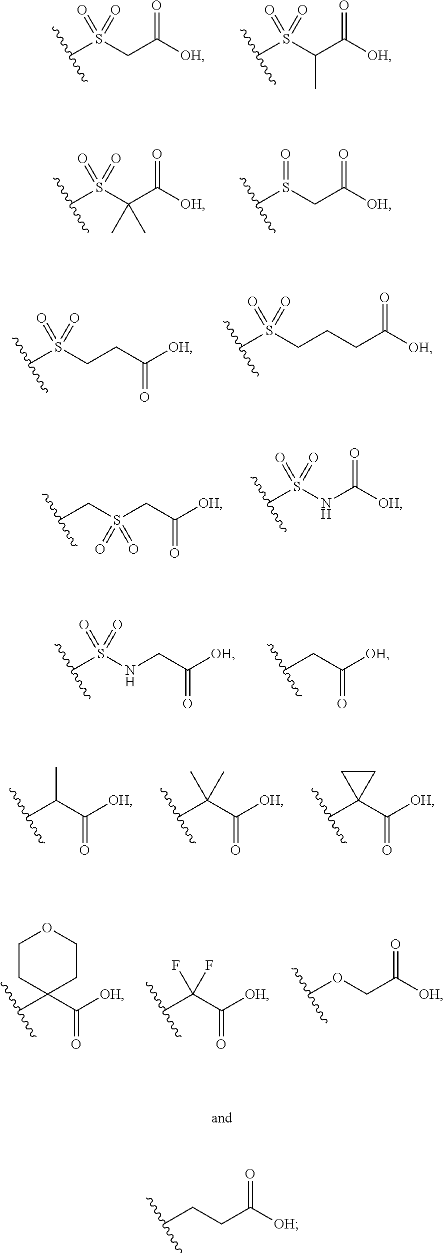
R¹, R², R³ and R⁴ are independently selected from H and Me;
W is O; and
m is selected from 1 and 2.

11. The compound according to claim 1 wherein
\textcircled{A} is selected from 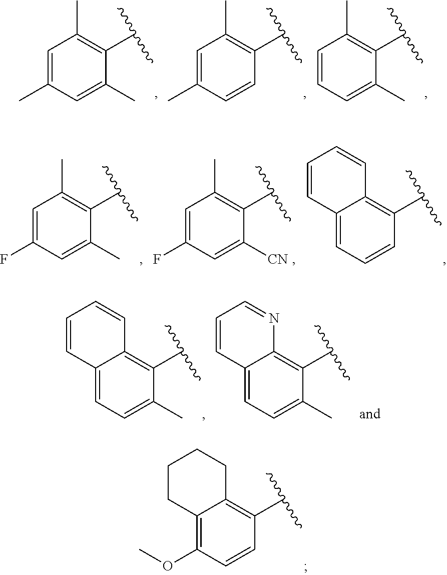
\textcircled{B} is selected from 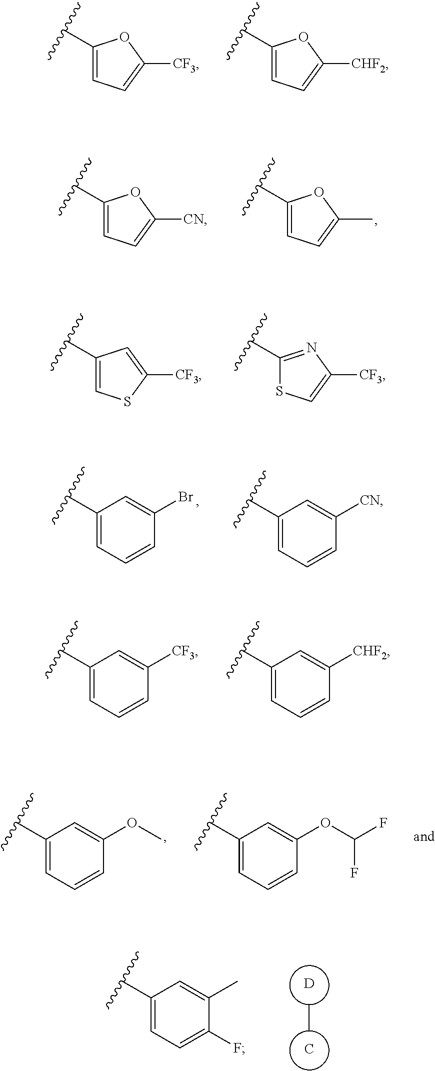
is selected from 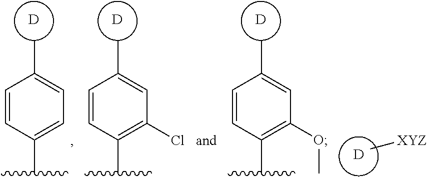
is selected from 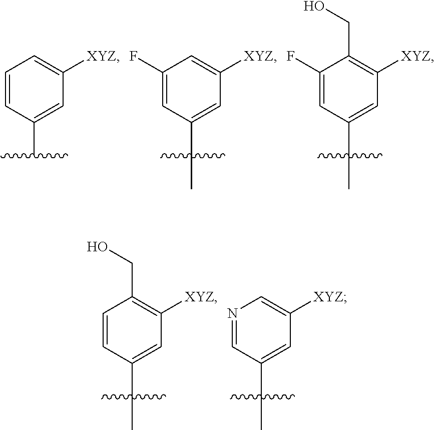
XYZ is selected from 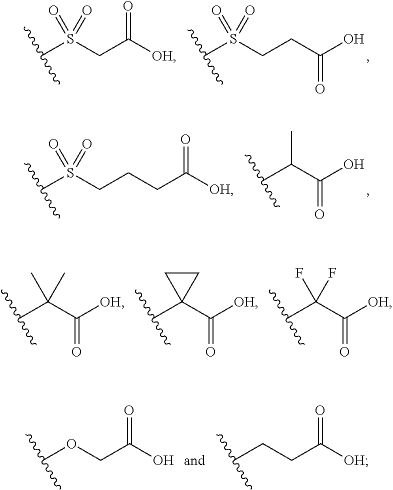
R¹, R², R³ and R⁴ are independently selected from H and Me;
W is O; and
m is 1.

12. The compound according to claim 1 selected from

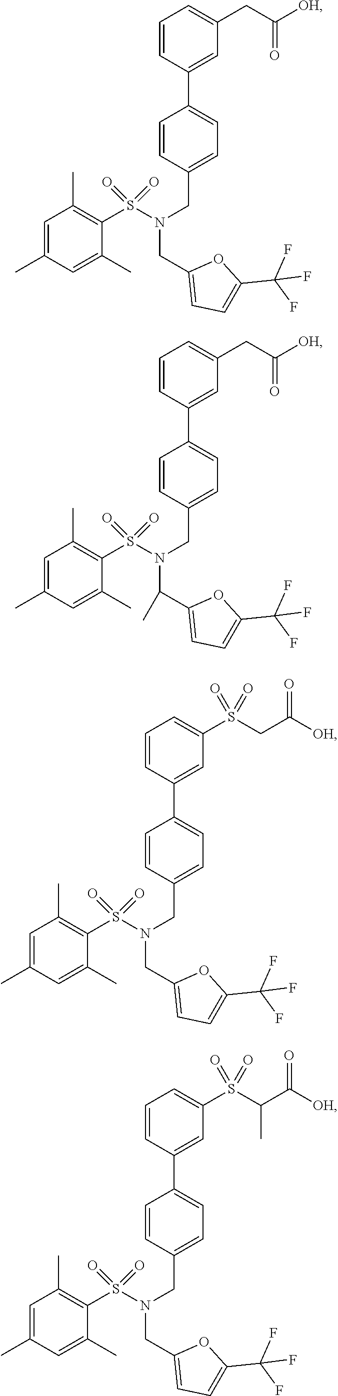

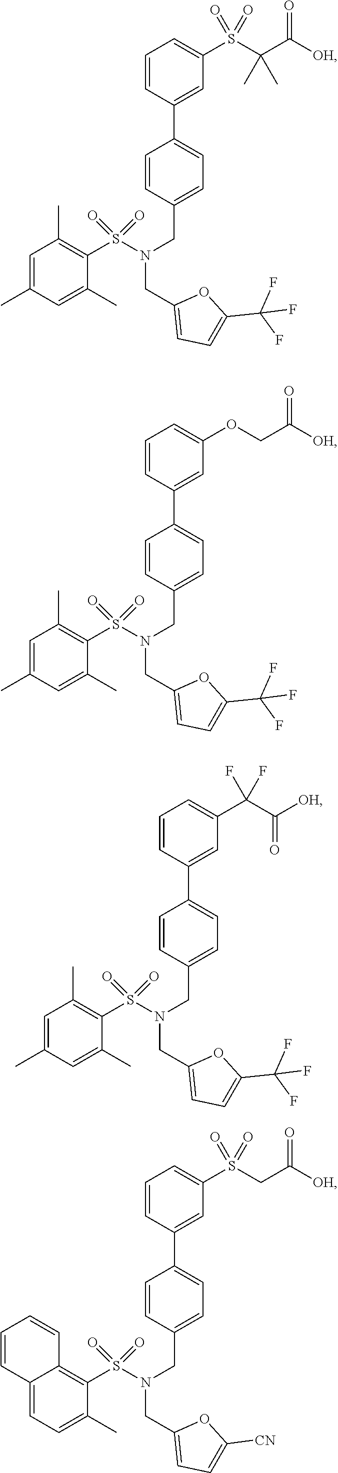

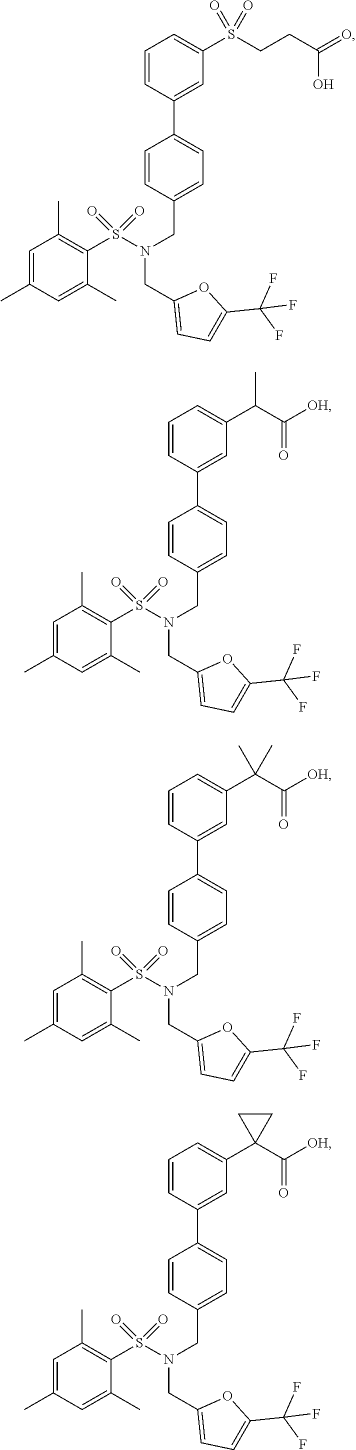

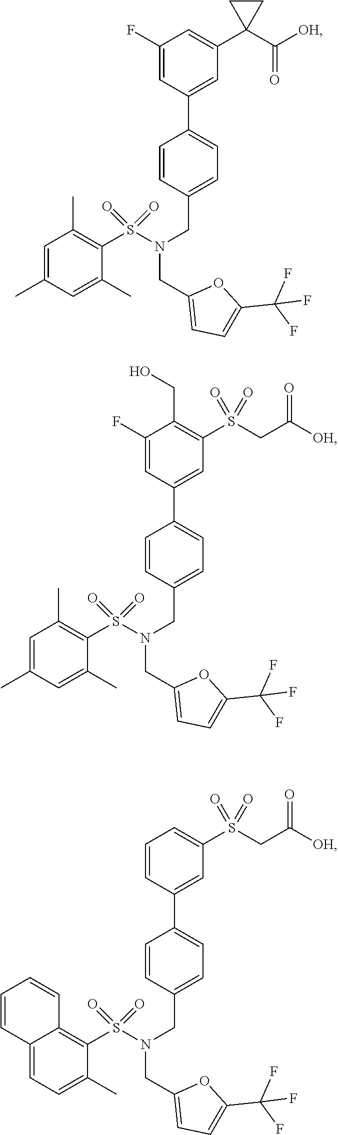 [SMILES: Cc1cc(C)c(S(=O)(=O)N(Cc2ccc(-c3cc(F)cc(C4(C(=O)O)CC4)c3)cc2)Cc2ccc(C(F)(F)F)o2)c(C)c1 ; Cc1cc(C)c(S(=O)(=O)N(Cc2ccc(-c3cc(F)c(CO)c(S(=O)(=O)CC(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1ccc(C(F)(F)F)o1]

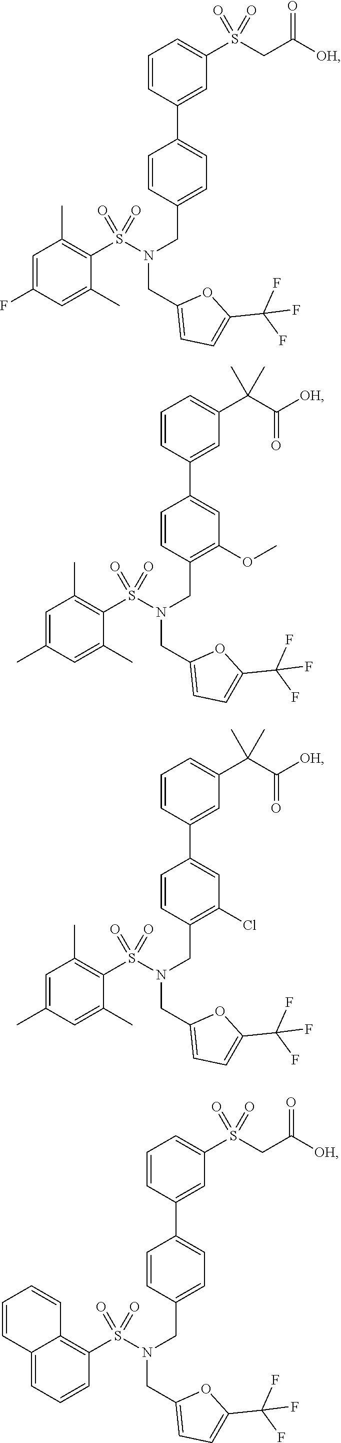

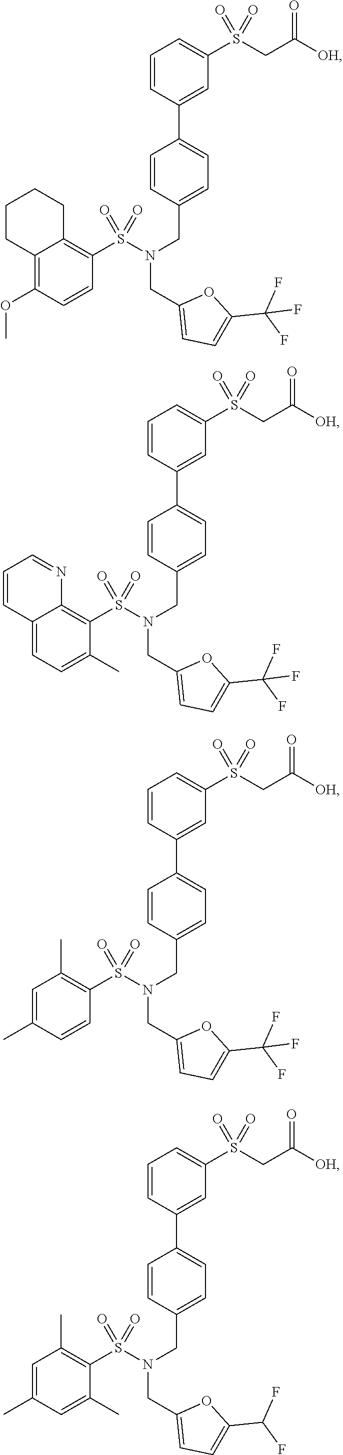

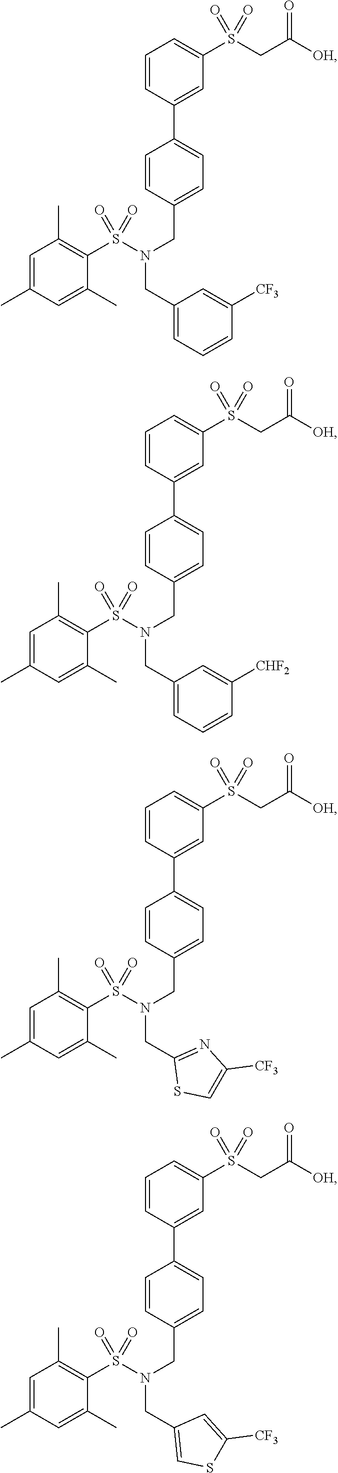

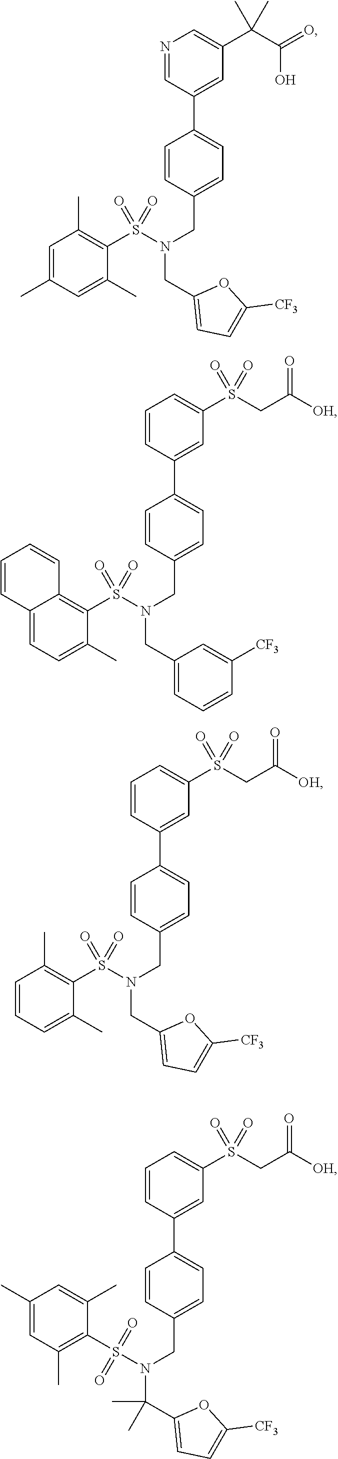

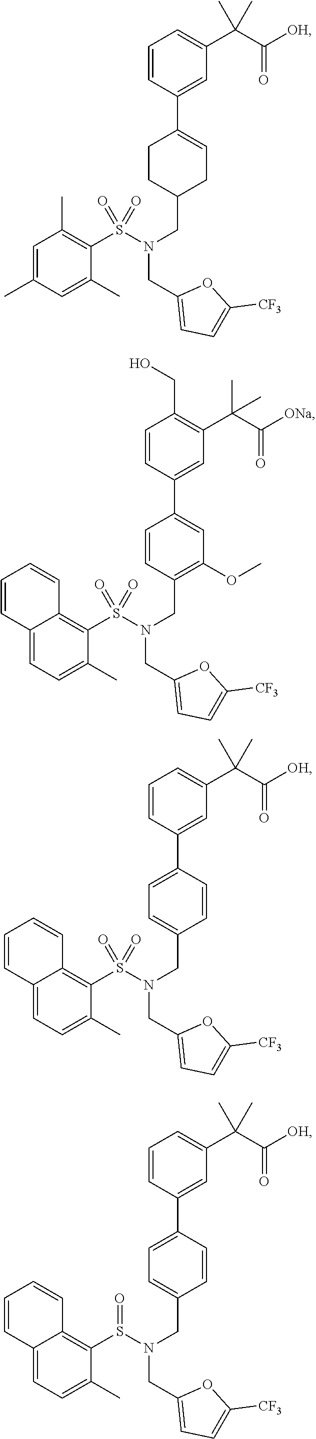

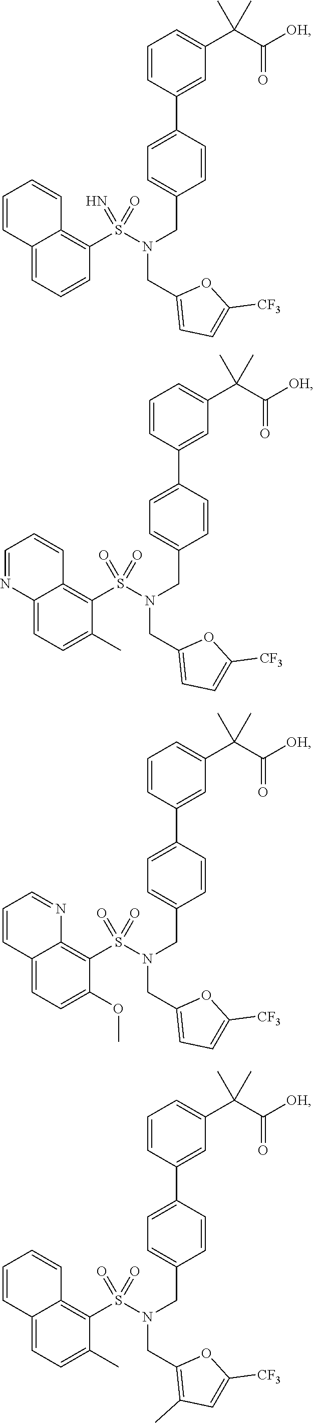

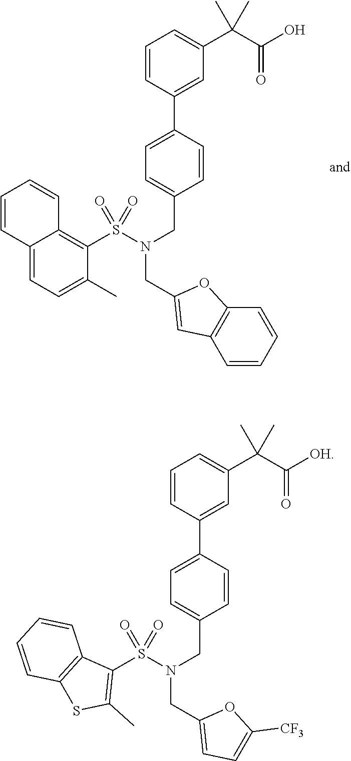 [SMILES: Cc1ccc2ccccc2c1S(=O)(=O)N(Cc1ccc(-c2cccc(C(C)(C)C(=O)O)c2)cc1)Cc1cc2ccccc2o1 ; "and" ; Cc1sc2ccccc2c1S(=O)(=O)N(Cc1ccc(-c2cccc(C(C)(C)C(=O)O)c2)cc1)Cc1ccc(C(F)(F)F)o1]

13. (canceled)

14. A method for the prophylaxis and/or treatment of diseases mediated by LXRs, comprising administering a therapeutically effective amount of a compound of claim 1 to a subject in need thereof.

15. The method according to claim 14 wherein the disease is selected from non-alcoholic fatty liver disease, non-alcoholic steatohepatitis, liver inflammation, liver fibrosis, obesity, insulin resistance, type II diabetes, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

16. A pharmaceutical composition comprising a compound according to claim 1 and a pharmaceutically acceptable carrier or excipient.
